SEQUENCE LISTING (35 sequences)

SEQ ID NO: 1 - protein, 305 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 3 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 4 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 5 - protein, 12 aa, synthetic construct
ESKYGPPCPPCP

SEQ ID NO: 6 - protein, 25 aa, synthetic construct
TTTPAPRPPTPAPTIASQPLSLRPE

SEQ ID NO: 7 - protein, 39 aa, synthetic construct
IEVMYPPPYLDNEKSNGTIIHVKGKHLCPSPLFPGPSKP

SEQ ID NO: 8 - protein, 44 aa, synthetic construct
ACRPAAGGAVHTRGLDFACDIYIWAPLAGTCGVLLLSLVITLYC

SEQ ID NO: 9 - protein, 27 aa, synthetic construct
FWVLVVVGGVLACYSLLVTVAFIIFWV

SEQ ID NO: 10 - protein, 27 aa, synthetic construct
FWVLVVVGGVLACYSLLVTVAFIIFWV

SEQ ID NO: 11 - protein, 41 aa, synthetic construct
RSKRSRLLHSDYMNMTPRRPGPTRKHYQPYAPPRDFAAYRS

SEQ ID NO: 12 - protein, 42 aa, synthetic construct
KRGRKKLLYIFKQPFMRPVQTTQEEDGCSCRFPEEEEGGCEL

SEQ ID NO: 13 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 18 aa, synthetic construct
EGRGSLLTCGDVEENPGP

SEQ ID NO: 16 - protein, 24 aa, synthetic construct
LEGGGEGRGSLLTCGDVEENPGPR

SEQ ID NO: 17 - protein, 22 aa, synthetic construct
GSGATNFSLLKQAGDVEENPGP

SEQ ID NO: 18 - protein, 19 aa, synthetic construct
ATNFSLLKQAGDVEENPGP

SEQ ID NO: 19 - protein, 20 aa, synthetic construct
QCTNYALLKLAGDVESNPGP

SEQ ID NO: 20 - protein, 22 aa, synthetic construct
VKQTLNFDLLKLAGDVESNPGP

SEQ ID NO: 21 - protein, 21 aa, synthetic construct
GSGEGRGSLLTCGDVEENPGP

SEQ ID NO: 22 - protein, 23 aa, synthetic construct
AAGSGEGRGSLLTCGDVEENPGP

SEQ ID NO: 23 - RNA, 12 nt, synthetic construct
  source 1..12: mol_type = other RNA
gttttagagcta

SEQ ID NO: 24 - RNA, 63 nt, synthetic construct
  source 1..63: mol_type = other RNA
tagcaagttaaaataaggctagtccgttatcaacttgaaaaagtggcaccgagtcggtgcttt

SEQ ID NO: 25 - skipped

SEQ ID NO: 26 - RNA, 20 nt, synthetic construct
  source 1..20: mol_type = other RNA
accccacagtggggccacta

SEQ ID NO: 27 - RNA, 20 nt, synthetic construct
  source 1..20: mol_type = other RNA
tgttggaaggatgaggaaat

SEQ ID NO: 28 - RNA, 20 nt, synthetic construct
  source 1..20: mol_type = other RNA
tcactatgctgccgcccagt

SEQ ID NO: 29 - RNA, 20 nt, synthetic construct
  source 1..20: mol_type = other RNA
tctctccatgtgcagtagga

SEQ ID NO: 30 - RNA, 20 nt, synthetic construct
  source 1..20: mol_type = other RNA
ctggatgtcggaggagtacg

SEQ ID NO: 31 - RNA, 20 nt, synthetic construct
  source 1..20: mol_type = other RNA
gtctccggaaactcgaggtg

SEQ ID NO: 32 - RNA, 20 nt, synthetic construct
  source 1..20: mol_type = other RNA
acagtgtagacttgattgac

SEQ ID NO: 33 - RNA, 20 nt, synthetic construct
  source 1..20: mol_type = other RNA
cgtgagtaaacctgaatctt

SEQ ID NO: 34 - RNA, 20 nt, synthetic construct
  source 1..20: mol_type = other RNA
gatattggcataagcctccc

SEQ ID NO: 35 - RNA, 20 nt, synthetic construct
  source 1..20: mol_type = other RNA
agagtctctcagctggtaca

(19) United States
(12) Patent Application Publication
(10) Pub. No.: US 2024/0010988 A1
(43) Pub. Date: Jan. 11, 2024

(54) GENETICALLY MODIFIED PRIMARY CELLS FOR ALLOGENEIC CELL THERAPY

(71) Applicant: Sana Biotechnology, Inc., Seattle, WA (US)

(72) Inventors: Sonja SCHREPFER, San Mateo, CA (US); Xiaomeng HU, San Mateo, CA (US)

(73) Assignee: Sana Biotechnology, Inc., Seattle, WA (US)

(21) Appl. No.: 18/449,625

(22) Filed: Aug. 14, 2023

Related U.S. Application Data
(63) Continuation of application No. PCT/US22/74878, filed on Aug. 11, 2022.
(60) Provisional application No. 63/232,161, filed on Aug. 11, 2021.
(60) Provisional application No. 63/297,694, filed on Jan. 7, 2022.
(60) Provisional application No. 63/344,502, filed on May 20, 2022.
(60) Provisional application No. 63/348,990, filed on Jun. 3, 2022.
(60) Provisional application No. 63/353,531, filed on Jun. 17, 2022.

(51) Int. Cl. C12N 5/071; C12N 9/22; C12N 15/11; C12N 15/90; A61K 35/39; A61P 3/10

(52) U.S. Cl. CPC C12N 5/0676; C12N 9/22; C12N 15/111; C12N 15/907; A61K 35/39; A61P 3/10; C12N 2510/00; C12N 2527/00; C12N 2310/20

(57) ABSTRACT

Provided are engineered cells, such as engineered primary cells, containing one or more modifications, such as genetic modifications, for use in allogeneic cell therapy. In some embodiments, the engineered primary cells are hypoimmunogenic cells.

### CROSS-REFERENCE TO RELATED APPLICATIONS

This application is a continuation of International Application No. PCT/US2022/074878 filed Aug. 11, 2022, which claims priority to U.S. Provisional Patent Application No. 63/232,161 filed Aug. 11, 2021, U.S. Provisional Patent Application No. 63/297,694 filed Jan. 7, 2022, U.S. Provisional Patent Application No. 63/344,502 filed May 20, 2022, U.S. Provisional Patent Application No. 63/348,990 filed Jun. 3, 2022, and U.S. Provisional Patent Application No. 63/353,531 filed Jun. 17, 2022, the contents of each of which are herein incorporated by reference in their entireties for all purposes.

### REFERENCE TO AN ELECTRONIC SEQUENCE LISTING

The contents of the electronic sequence listing (186152005401SEQLIST.xml; Size: 32,413 bytes; and Date of Creation: Aug. 11, 2023) are herein incorporated by reference in their entirety.

### FIELD

In certain aspects, the present disclosure is directed to engineered cells, such as engineered primary cells, containing one or more modifications, such as genetic modifications, for use in allogeneic cell therapy. In some embodiments, the engineered primary cells are hypoimmunogenic cells.

### SUMMARY

In some aspects, provided herein is an engineered cell, such as an engineered primary cell, comprising modifications that (i) increase expression of one or more tolerogenic factor, and (ii) reduce expression of one or more major histocompatibility complex (MHC) class I molecules and/or one or more MHC class II molecules, wherein the increased expression of (i) and the reduced expression of (ii) is relative to a cell of the same cell type that does not comprise the modifications.

In some embodiments, the modification in (ii) reduces expression of one or more MHC class I molecules. In some embodiments, the modifications in (ii) reduces expression of one or more MHC class I molecules and one or more MHC class II molecules. In some of any of the provided embodiments, the one or more tolerogenic factors is A20/TNFAIP3, C1-Inhibitor, CCL21, CCL22, CD16, CD16 Fc receptor, CD24, CD27, CD35, CD39, CD46, CD52, CD55, CD59, CD200, CR1, CTLA4-Ig, DUX4, FasL, H2-M3, HLA-C, HLA-E, HLA-E heavy chain, HLA-G, IDO1, IL-10, IL15-RF, IL-35, MANF, Mfge8, PD-1, PD-L1 and/or Serpinb9. In some embodiments, the one or more tolerogenic factor is selected from the group consisting of CD47, CD27, CD200, HLA-C, HLA-E, HLA-E heavy chain, HLA-G, PD-L1, IDO1, CTLA4-Ig, C1-Inhibitor, IL-10, IL-35, FASL, CCL21, MFGE8, and SERPINB9, and any combination thereof. In some embodiments, the one or more tolerogenic factor is selected from the group consisting of CD47, PD-L1, HLA-E, HLA-G, CCL21, FASL, SERPINB9, CD200, MFGE8, and any combination thereof. In some embodiments, at least one of the one or more tolerogenic factor is CD47.

In some of any embodiments, the one or more tolerogenic factors is selected from the group consisting of CD47; HLA-E; CD24; PD-L1; CD46; CD55; CD59; CR1; MANF; A20/TNFAIP3; HLA-E and CD47; CD24, CD47, PD-L1, and any combination thereof; HLA-E, CD24, CD47, and PD-L1, and any combination thereof; CD46, CD55, CD59, and CR1, and any combination thereof; HLA-E, CD46, CD55, CD59, and CR1, and any combination thereof; HLA-E, CD24, CD47, PDL1, CD46, CD55, CD59, and CR1, and any combination thereof; HLA-E and PDL1; HLA-E, PDL1, and A20/TNFAIP, and any combination thereof; HLA-E, PDL1, and MANF, and any combination thereof; HLA-E, PDL1, A20/TNFAIP, and MANF, and any combination thereof; and CD47, PD-L1, HLA-E, HLA-G, CCL21, FASL, SERPINB9, CD200, MFGE8, and any combination thereof.

In some of any embodiments, the modifications are selected from modifications that reduce expression of MHC I and/or MHC II; reduce expression of MIC-A and/or MIC-B; increase expression of CD47, and optionally CD24 and PD-L1; and increase expression of CD46, CD55, CD59 and CR1.

In some of any embodiments, the modifications are selected from modifications that reduce expression of MHC class I molecule; reduce expression of MIC-A and/or MIC-B; reduce expression of TXNIP; increase expression of PD-L1 and HLA-E; and optionally A20/TNFAIP3 and MANF.

In some of any embodiments, the modifications are selected from modifications that increase the expression of CCL21, PD-L1, FASL, SERPINB9, HLA-G, CD47, CD200, and MFGE8; and reduce the expression of a MICA and/or MICB.

In some embodiments, the modifications are selected from modifications that reduce expression of MHC I and/or MHC II; and increase expression of CD47.

In some embodiments, any of the above modifications are present in a provided engineered cells along with one or more additional edits that increase or decrease expression of a gene in the cell. In some embodiments, any one or more of the further modifications can be a modification that that reduces expression, such as disrupts, inactivates or knockout expression, of B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B, NFY-C, CTLA-4, PD-1, IRF1, MIC-A, MIC-B. In some embodiments, any one or more of the further modifications can be a modification that reduces expression of a protein that is involved in oxidative or ER stress, TRAC, TRB, CD142, ABO, CD38, PCDH11Y, NLGN4Y and/or RHD. In some embodiments, proteins that are is involved in oxidative or ER stress include thioredoxin-interacting protein (TXNIP), PKR-like ER kinase (PERK), inositol-requiring enzyme 1α (IRE1α), and DJ-1 (PARK7).

In some embodiments, for any of the provided embodiments in which there is reduced expression of any of the above described target genes (e.g. B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B, NFY-C, CTLA-4, PD-1, IRF1, MIC-A, MIC-B) the target gene is not expressed by the engineered cell. In some embodiments, the protein encoded by the target gene is not expressed on the surface of the cell. In some embodiments, the MHC class I complex and/or MHC class II complex is not expressed on the surface of the cell.

In some aspects, provided herein is an engineered primary cell comprising modifications that (i) increase expression of CD47, and (ii) reduce expression of one or more major histocompatibility complex (MHC) class I molecules and/or one or more MHC class II molecules, wherein the increased expression of (i) and the reduced expression of (ii) is relative to a cell of the same cell type that does not comprise the modifications.

In some embodiments, the modification(s) that increase expression comprise increased surface expression, and/or the modifications that reduce expression comprise reduced surface expression. In some embodiments, the modification that increases expression of CD47 comprises an exogenous polynucleotide encoding the CD47 protein. In some embodiments, the exogenous polynucleotide encoding CD47 encodes a sequence of amino acids having at least 85% identity to the amino acid sequence of SEQ ID NO: 2, and reduces innate immune killing of the engineered primary cell. In some embodiments, the exogenous polynucleotide encoding CD47 encodes a sequence set forth in SEQ ID NO: 2. In some embodiments, the exogenous polynucleotide encoding CD47 is operably linked to a promoter. In some embodiments, the promoter is a constitutive promoter. In some embodiments, the promoter is selected from the group consisting of the CAG promoter, the cytomegalovirus (CMV) promoter, the EF1a promoter, the PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, and the Rous sarcoma virus (RSV) promoter. In some embodiments, the exogenous polynucleotide encoding CD47 is integrated into the genome of the engineered primary cell. In some embodiments, the exogenous polynucleotide is a multicistronic vector encoding CD47 and an additional transgene encoding a second transgene. In some embodiments, the integration is by non-targeted insertion into the genome of the engineered primary cell, optionally by introduction of the exogenous polynucleotide into the cell using a lentiviral vector. In some embodiments, the integration is by targeted insertion into a target genomic locus of the cell. In some embodiments, the target genomic locus is a safe harbor locus, a B2M gene locus, a CIITA gene locus, a TRAC gene locus, or a TRBC gene locus. In some embodiments, the target genomic locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

In some embodiments, the modification that reduces expression of one or more MHC class I molecules reduces one or more MHC class I molecules protein expression. In some embodiments, the modification that reduces expression of one or more MHC class I molecules is a modification that reduces expression of B-2 microglobulin (B2M). In some embodiments, the modification that reduces expression of one or more MHC class I molecules comprises reduced mRNA expression of B2M. In some embodiments, the modification that reduces expression of one or more MHC class I molecules comprises reduced protein expression of B2M. In some embodiments, the modification eliminates B2M gene activity. In some embodiments, the modification comprises inactivation or disruption of both alleles of the B2M gene. In some embodiments, the modification comprises inactivation or disruption of all B2M coding sequences in the cell. In some embodiments, the inactivation or disruption comprises an indel in the B2M gene. In some embodiments, the modification is a frameshift mutation or a deletion of a contiguous stretch of genomic DNA of the B2M gene. In some embodiments, the B2M gene is knocked out. In some embodiments, the modification is by nuclease-mediated gene editing. In some embodiments, the nuclease-mediated gene editing is by a zinc finger nuclease (ZFN), a TAL-effector nuclease (TALEN), or a CRISPR-Cas combination that targets the B2M gene, optionally wherein the Cas is Cas9. In some embodiments, the nuclease-mediated gene editing is by a CRISPR-Cas combination and the CRISPR-Cas combination comprises a guide RNA (gRNA) having a targeting domain that is complementary to at least one target site within the B2M gene. In some embodiments, the CRISPR-Cas combination is a ribonucleoprotein (RNP) complex comprising the gRNA and a Cas protein. In some embodiments, the modification that reduces expression of one or more MHC class I molecules is a modification that reduces expression of an HLA-A protein, an HLA-B protein, or HLA-C protein, optionally wherein a gene encoding said HLA-A protein, an HLA-B protein, or HLA-C protein is knocked out.

In some embodiments, the modification that reduces expression of one or more MHC class II molecules reduces one or more MHC class II molecules protein expression. In some embodiments, the modification that reduces expression of one or more MHC class II molecules is a modification that reduces expression of CIITA. In some embodiments, the modification that reduces expression of one or more MHC class II molecules comprises reduced mRNA expression of CIITA. In some embodiments, the modification that reduces expression of one or more MHC class II molecules comprises reduced protein expression of CIITA. In some embodiments, the modification eliminates CIITA gene activity. In some embodiments, the modification comprises inactivation or disruption of both alleles of the CIITA gene. In some embodiments, the modification comprises inactivation or disruption of all CIITA coding sequences in the cell. In some embodiments, the inactivation or disruption comprises an indel in the CIITA gene. In some embodiments, the indel is a frameshift mutation or a deletion of a contiguous stretch of genomic DNA of the CIITA gene. In some embodiments, the CIITA gene is knocked out. In some embodiments, the modification is by nuclease-mediated gene editing. In some embodiments, the nuclease-mediated gene editing is by a zinc finger nuclease (ZFN), a TAL-effector nuclease (TALEN), or a CRISPR-Cas combination that targets the CIITA gene, optionally wherein the Cas is Cas9. In some embodiments, the nuclease-mediated gene editing is by a CRISPR-Cas combination and the CRISPR-Cas combination comprises a guide RNA (gRNA) having a targeting domain that is complementary to at least one target site within the CIITA gene. In some embodiments, the CRISPR-Cas combination is a ribonucleoprotein (RNP) complex comprising the gRNA and a Cas protein. In some embodiments, the modification that reduces expression of one or more MHC class II molecules is a modification that reduces expression of an HLA-DP protein, an HLA-DR protein, or HLA-DQ protein, optionally wherein a gene encoding said HLA-DP protein, an HLA-DR protein, or HLA-DQ protein is knocked out.

In some embodiments, the engineered primary cell is a human cell or an animal cell. In some embodiments, the animal cell is a pig (porcine), cow (bovine) or sheep (ovine) cell. In some embodiments, the engineered primary cell is a human cell. In some embodiments, the primary cell is a cell type that is exposed to the blood. In some embodiments, the engineered primary cell is a primary cell isolated from a donor subject. In some embodiments, the donor subject is healthy or is not suspected of having a disease or condition at the time the donor sample is obtained from the donor subject. In some embodiments, the engineered primary cell is selected from an islet cell, a beta islet cell, B cell, T cell, NK cell, retinal pigmented epithelium cell, glial progenitor cell, endothelial cell, hepatocyte, thyroid cell, skin cell, and blood cell. In some embodiments, the engineered primary cell is an endothelial cell. In some embodiments, the engineered primary cell is an epithelial cell. In some embodiments, the engineered primary cell is a T cell. In some embodiments, the engineered primary cell is an NK cell. In some embodiments, the engineered primary cell comprises a chimeric antigen receptor (CAR). In some embodiments, the engineered primary cell is an islet cell. In some embodiments, the islet cell is a beta islet cell. In some embodiments, the engineered primary cell is a hepatocyte. In some embodiments, the engineered primary cell is ABO blood group type O. In some embodiments, the engineered primary cell is Rhesus factor negative (Rh−).

In some aspects, provided herein is a method of generating an engineered primary cell, the method comprising: a) reducing or eliminating the expression of one or more MHC class I molecules and/or one or more MHC class II molecules in a primary cell; and b) increasing the expression of one or more tolerogenic factors in the primary cell. In some embodiments, the one or more tolerogenic factor is selected from the group consisting of CD47, CD27, CD200, HLA-C, HLA-E, HLA-E heavy chain, HLA-G, PD-L1, IDO1, CTLA4-Ig, C1-Inhibitor, IL-10, IL-35, FASL, CCL21, MFGE8, and SERPINB9, and any combination thereof. In some embodiments, the one or more tolerogenic factor is selected from the group consisting of CD47, PD-L1, HLA-E, HLA-G, CCL21, FASL, SERPINB9, CD200, MFGE8, and any combination thereof. In some embodiments, at least one of the one or more tolerogenic factor is CD47. In some embodiments, the method comprises reducing or eliminating the expression of one or more MHC class I molecules. In some embodiments, the method comprises reducing or eliminating the expression of one or more MHC class I molecules and one or more MHC class II molecules.

In some aspects, provided herein is a method of generating an engineered primary cell, the method comprising: a. reducing or eliminating the expression of one or more MHC class I and/or one or more MHC class II molecules in the cell; and, b. increasing the expression of CD47 in the cell. In some embodiments, the method comprises reducing or eliminating the expression of one or more MHC class I molecules. In some embodiments, the method comprises reducing or eliminating the expression of one or more MHC class I molecules and one or more MHC class II molecules.

In some embodiments, the modification(s) that increase expression comprise increased surface expression, and/or the modifications that reduce expression comprise reduced surface expression. In some embodiments, the modification that increases expression of CD47 comprises an exogenous polynucleotide encoding the CD47 protein. In some embodiments, the exogenous polynucleotide encoding CD47 encodes a sequence of amino acids having at least 85% identity to the amino acid sequence of SEQ ID NO: 2, and reduces innate immune killing of the engineered primary cell. In some embodiments, the exogenous polynucleotide encoding CD47 encodes a sequence set forth in SEQ ID NO: 2. In some embodiments, the exogenous polynucleotide encoding CD47 is operably linked to a promoter. In some embodiments, the exogenous polynucleotide encoding CD47 is integrated into the genome of the engineered primary cell. In some embodiments, the integration is by non-targeted insertion into the genome of the engineered primary cell, optionally by introduction of the exogenous polynucleotide into the engineered primary cell using a lentiviral vector. In some embodiments, the integration is by targeted insertion into a target genomic locus of the cell, optionally wherein the targeted insertion is by nuclease-mediated gene editing with homology-directed repair. In some embodiments, the target genomic locus is a B2M gene locus, a CIITA gene locus, a CD142 gene locus, a TRAC gene locus, or a TRBC gene locus. In some embodiments, the target genomic locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus. In some embodiments, the nuclease-mediated gene editing is by a zinc finger nuclease (ZFN), a TAL-effector nuclease (TALEN), or a CRISPR-Cas combination that targets the target genomic locus, optionally wherein the Cas is Cas9. In some embodiments, the nuclease-mediated gene editing is by a CRISPR-Cas combination and the CRISPR-Cas combination comprises a guide RNA (gRNA) having a targeting domain that is complementary to a target sequence of the target genomic locus and a homology-directed repair template comprising the exogenous polynucleotide encoding CD47. In some embodiments, the CRISPR-Cas combination is a ribonucleoprotein (RNP) complex comprising the gRNA and a Cas protein. In some embodiments, the engineered primary cell is a hypo-immunogenic primary cell.

In some embodiments, reducing or eliminating expression of one or more MHC class I molecules comprises introducing a modification that reduces one or more MHC class I molecules protein expression. In some embodiments, the modification that reduces one or more MHC class I molecules protein expression comprises reduced expression of B2M. In some embodiments, the modification that reduces one or more MHC class I molecules protein expression comprises reduced protein expression of B2M. In some embodiments, the modification that reduces one or more MHC class I molecules protein expression eliminates B2M gene activity. In some embodiments, the modification that reduces one or more MHC class I molecules expression comprises inactivation or disruption of both alleles of the B2M gene. In some embodiments, the modification that reduces one or more MHC class I molecules protein expression comprises inactivation or disruption of all B2M coding sequences in the cell. In some embodiments, the inactivation or disruption comprises an indel in the endogenous B2M gene or a deletion of a contiguous stretch of genomic DNA of the endogenous B2M gene. In some embodiments, the indel is a frameshift mutation or a deletion of a contiguous stretch of genomic DNA of the B2M gene. In some embodiments, the endogenous B2M gene is knocked out. In some embodiments, the modification reduces one or more MHC class I molecules protein expression is by nuclease-mediated gene editing. In some embodiments, the nuclease-mediated gene editing is by a zinc finger nuclease (ZFN), a TAL-effector nuclease (TALEN), or a CRISPR-Cas combination that targets the B2M gene, optionally wherein the Cas is Cas9. In some embodiments, the nuclease-mediated gene editing is by a CRISPR-Cas combination and the CRISPR-Cas combination comprises a guide RNA (gRNA) having a targeting domain that is complementary to at least one target site within the B2M gene. In some embodiments, the CRISPR-Cas combination is a ribonucleoprotein (RNP) complex comprising the gRNA and a Cas protein. In some embodiments, the modification that reduces expression of one or more MHC class I molecules reduces HLA-A protein expression, HLA-B protein expression, or HLA-C protein expression, optionally wherein the protein expression is reduced by knocking out a gene encoding said HLA-A protein, HLA-B protein, or HLA-C protein.

In some embodiments, reducing or eliminating expression of one or more MHC class II molecules comprises introducing a modification that reduces one or more MHC class II molecules protein expression. In some embodiments, the genetic modification that reduces one or more MHC class II molecules protein expression comprises reduced expression of CIITA. In some embodiments, the genetic modification that reduces one or more MHC class II molecules protein expression comprises reduced protein expression of CIITA. In some embodiments, the modification that reduces one or more MHC class II molecules protein expression eliminates CIITA. In some embodiments, the modification that reduces one or more MHC class II molecules protein expression comprises inactivation or disruption of both alleles of the CIITA gene. In some embodiments, the modification comprises inactivation or disruption of all CIITA coding sequences in the cell. In some embodiments, the inactivation or disruption comprises an indel in the CIITA gene or a deletion of a contiguous stretch of genomic DNA of the CIITA gene. In some embodiments, the indel is a frameshift mutation or a deletion of a contiguous stretch of genomic DNA of the CIITA gene. In some embodiments, the CIITA gene is knocked out. In some embodiments, the genetic modification that reduces expression of one or more MHC class II reduces the expression of a HLA-DP protein, a HLA-DR protein, or a HLA-DQ protein, optionally wherein said HLA-DP protein expression, said HLA-DR protein expression, or said HLA-DQ protein expression is reduced by knocking out a gene encoding said HLA-DP protein, said HLA-DR protein, or said HLA-DQ protein.

In some embodiments, the engineered primary cell is a human cell or an animal cell. In some embodiments, the animal cell is a pig (porcine) cell, cow (bovine) cell, or sheep (ovine) cell. In some embodiments, the engineered primary cell is a human cell. In some embodiments, the engineered primary cell is a cell type that is exposed to the blood. In some embodiments, the engineered primary cell is isolated from a donor subject. In some embodiments, the engineered primary cell is selected from an islet cell, a beta islet cell, B cell, T cell, NK cell, retinal pigmented epithelium cell, glial progenitor cell, endothelial cell, hepatocyte, thyroid cell, skin cell, and blood cell. In some embodiments, the engineered primary cell is an islet cell.

In some embodiments, the primary islet cell has been dissociated from a primary islet cluster. In some embodiments, the primary islet cluster is a human primary cadaveric islet cluster. In some embodiments, after step a) and/or after step b) the primary islet cell is incubated under conditions for re-clustering into a modified primary islet cluster, wherein at least a portion of the incubating is carried out with motion. In some embodiments, the incubating further comprises a least a portion of incubating under static conditions. In some embodiments, the incubating comprises a first incubation under static conditions followed by the incubating with motion. In some embodiments, the incubating comprises the incubating with motion followed by a second incubation under static conditions. In some embodiments, prior to the incubating under conditions for reclustering, the method comprises selecting for islet cells that have been modified. In some embodiments, the selecting is by fluorescence-activated cell sorting (FACS).

In some embodiments, the method comprises: i) dissociating a primary islet cluster into a suspension of primary beta islet cells; ii) modifying primary beta islet cells of the suspension to reduce or eliminate the expression of one or more MHC class I and/or one or more MHC class II HLA in primary beta islet cells; iii) incubating the modified primary beta islet cells under conditions for re-clustering into a first modified primary islet cluster, wherein at least a portion of the incubating is carried out with motion; iv) dissociating the modified] primary islet cluster into a suspension of modified primary beta islet cells; v) further modifying the modified primary islet cells of the suspension to increase the expression of one or more tolerogenic factors in the primary cell; and vi) incubating the further modified primary beta islet cells under conditions for re-clustering into a second modified primary islet cluster, wherein at least a portion of the incubating is carried out with motion.

In some embodiments, the one or more MHC class I HLA is an HLA-A protein, an HLA-B protein, or HLA-C protein. In some embodiments, the one or more MHC class II HLA is an HLA-DP protein, an HLA-DR protein, or an HLA-DQ protein. In some embodiments, the modifying is by genetic engineering. In some embodiments, the motion is shaking. In some embodiments, the shaking comprises orbital motion. In some embodiments, the shaking comprises bidirectional linear movement. In some embodiments, the shaking is with an orbital shaker. In some embodiments, the incubating in (iii) and/or the incubating in vi) further comprises a least a portion of incubating under static conditions. In some embodiments, the incubating in iii) and/or the incubating in vi) comprises a first incubation under static conditions followed by the incubating with motion. In some embodiments, the incubating comprises the incubating with motion followed by a second incubation under static conditions.

In some embodiments, prior to v), the method comprises selecting, from the dissociated islet cells in iv), beta islet cells that have been modified, and optionally repeating steps iii) and iv) on the selected islet cells. In some embodiments, after the incubating in vi), the method comprises dissociating the second modified primary islet cluster into a suspension of modified primary beta islet cells and selecting for islet cells that have been modified. In some embodiments, incubating the selected modified primary beta islet cells under conditions for re-clustering into a modified primary islet cluster, wherein at least a portion of the incubating is carried out with motion.

In some embodiments, provided herein is use of motion to promote modification of a population of cells, wherein the population of cells has been contacted with one or more reagents to modify gene expression in cells of the population before subjecting to the motion.

In some embodiments, provided herein is a method of enhancing modification of a population of cells, in which the method includes: i) contacting a population of cells with one or more reagents to modify gene expression in cells of the population; and ii) subjecting the population of cells to motion after contact with the one or more reagents.

In some embodiments, provided herein is a method of enhancing viability of a population of cells, in which the method includes: i) contacting a population of cells with one or more reagents to modify gene expression in cells of the population; and ii) subjecting the population of cells to motion after contact with the one or more reagents.

In some embodiments, provided herein is a method of modification of a population of cells, in which the method includes i) contacting a population of cells with one or more reagents to modify gene expression in cells of the population; and ii) subjecting the population of cells to motion after contact with the one or more reagents.

In some of any embodiments of a provided use or method, the population of cells are primary cells. In some f any embodiments of a provided use or methods, the population of cells are cells derived from stem cells. In some embodiments, the stem cells are selected from the group consisting of a pluripotent stem cell (PSC), an induced pluripotent stem cell, an embryonic stem cell, a hematopoietic stem cell, a mesenchymal stem cell, an endothelial stem cell, an epithelial stem cell, an adipose stem cell, a germline stem cell, a lung stem cell, a cord blood stem cell, and a multipotent stem cell. In some embodiments, the stem cells are pluripotent stem cell. In some embodiments, the stem cells are induced pluripotent stem cells, mesenchymal stem cells (MSCs), hematopoietic stem cells (HSCs), or embryonic stem cells (ESCs). In some embodiments, the population of cells are selected from the group consisting of islet cells, immune cells, B cells, T cells, natural killer (NK) cells, natural killer T (NKT) cells, macrophages, endothelial cells, muscle cells, cardiac muscle cells, smooth muscle cells, skeletal muscle cells, hepatocytes, a glial progenitor cells, dopaminergic neurons, retinal pigment epithelial cells, thyroid cells, skin cells, glial progenitor cells, neural cells, cardiac cells, and blood cells.

In some of any embodiments of a provided use or method, the population of cells are naturally present in a 3D network.

In some of any embodiments of a provided use or method, the population of cells are in suspension. In some embodiments, the population of cells are in a vessel that has a low-attachment surface. In some embodiments, the population of cells are maintained in a vessel in a minimum volume of media sufficient to cover the cells. In some embodiments, the population of cells in suspension are produced by dissociating cells from an adherent culture or a cell cluster prior to the contacting.

In some of any embodiments of a provided use or method, the population of cells are islet cells. In some embodiments, the population of cells comprise beta islet cells. In some embodiments, the population of cells comprising beta islet cells are produced by dissociating a primary islet cluster into a suspension of cells comprising primary beta islet cells.

In some of any embodiments of a provided use or method, the contacting is carried out for less than two days prior to subjecting the cells to motion. In some embodiments, the contacting is carried out for 30 seconds to 24 hours prior to subjecting the cells to motion. In some embodiments, the contacting is carried out for 1 minute to 60 minutes, 2 minutes to 30 minutes, 5 minutes to 15 minutes prior to subjecting the cells to motion.

In some of any embodiments of a provided use or method, subjecting the population of cells to motion promotes the formation of cell aggregates. In some embodiments, subjecting the population of cells to motion forms cell clusters.

In some of any embodiments of a provided use or method, the method or use further includes incubating the cells under static conditions after subjecting the cells to motion. In some of any embodiments of a provided use or method, the method or use further includes incubating the cells under static conditions after the contacting and before subjecting the cells to motion.

In some of any embodiments of a provided use or method, the one or more reagents comprise at least two different reagents. In some embodiments, each of the at least two different reagents is for modulating expression of a different gene.

In some of any embodiments of a provided use or method, the steps of the method are repeated. In some embodiments, the one or more reagents in the first iteration of the method are different from the the one or more reagents in the repeated iteration of the method. In some embodiments, the or more reagents in the first iteration of the method are different from the one or more reagents in the second iteration of the method.

In some of any embodiments of a provided use or method, such may further comprise before subjecting the cells to motion after the contacting with the one or more reagents, selecting for cells that have modified gene expression. In some of any embodiments, selecting cells that have modified gene expression may be relative to the cells before the contacting. In some embodiments, the modified gene expression is increased, such as relative to the expression of the gene in the cell before the contacting. In some embodiments, the modified gene expression is decreased, such as relative to the expression of the gene in the cell before the contacting.

Provided herein is A method for modifying primary islet cells, the method comprising: i) dissociating a primary islet cluster into a suspension of primary islet cells; ii) contacting the suspension of primary islet cells with one or more reagents to modify gene expression; and iii) after the contacting incubating the modified islet cells under conditions for re-clustering the cells into an islet, wherein at least a portion of the incubating is carried out with motion.

In some aspects, provided herein is a method for gene editing primary islet cells, the method comprising: i) dissociating a primary islet cluster into a suspension of primary beta islet cells; ii) modifying primary beta islet cells of the suspension; and iii) incubating the modified primary beta islet cells under conditions for re-clustering the modified primary beta islet cells into an islet, wherein at least a portion of the incubating is carried out with shaking. In some embodiments, the primary islet cluster is a human primary cadaveric islet cluster. In some embodiments, the modifying comprises introducing one more modifications into the cell to reduce expression of one or more genes encoding an endogenous protein in the cell or to increase expression of one or more heterologous proteins in the cell.

In some embodiments, the incubating in (iii) and/or the incubating in vi) further comprises a least a portion of incubating under static conditions. In some embodiments, the incubating comprises a first incubation under static conditions followed by the incubating with motion. In some embodiments, the incubating comprises the incubating with motion followed by a second incubation under static conditions. In some embodiments, steps i)-iii) are repeated. In some embodiments, the modifying in the first iteration of the method is different from the modifying in the repeated iteration of the method. In some embodiments the one or more reagents in the first iteration of the method are different from the one or more reagents in the repeated iteration of the method.

In some embodiments, the re-clustered islet cells are a first modified primary islet cluster and wherein the method further comprises: iv) dissociating the first modified primary islet cluster into a suspension of modified primary beta islet cells; v) further modifying the modified primary islet cells of the suspension; and vi) incubating the further modified primary beta islet cells under conditions for re-clustering into a second modified primary islet cluster, wherein at least a portion of the incubating is carried out with motion.

In some embodiments, the one or more reagents are one or more first reagents and the re-clustered islet cells are a first modified primary islet cluster and wherein the method further comprises: iv) dissociating the first modified primary islet cluster into a suspension of modified primary islet cells; v) further contacting the modified primary islet cells of the suspension with one or more further reagents to modify gene expression; and vi) incubating the further modified primary islet cells after the further contacting under conditions for re-clustering into a second modified primary islet cluster, wherein at least a portion of the incubating is carried out with motion.

In some embodiments, prior to the incubating in iii), the method comprises selecting for islet cells that have been modified. In some embodiments, prior to v), the method comprises selecting, from the dissociated islet cells in iv), beta islet cells that have been modified, and optionally repeating steps iii) and iv) on the selected islet cells. In some embodiments, after the incubating in vi), the method comprises dissociating the second modified primary islet cluster into a suspension of modified primary islet cells and selecting for islet cells that have been modified. In some embodiments, cells that have been modified have modified gene expression, such as modified relative to the primary islet cells before the contacting.

In some embodiments, incubating the selected modified primary beta islet cells under conditions for re-clustering into a modified primary islet cluster, wherein at least a portion of the incubating is carried out with motion. In some embodiments, the selecting comprises fluorescence-activated cell sorting (FACS).

In some embodiments, the suspension is a single cell suspension.

In some embodiments, the suspension of primary islet cells is present in a vessel that has a low-attachment surface. In some embodiments, the vessel has a minimum volume of media sufficient to cover the cells.

In some of any embodiments, the motion is at a speed of between about 20 rpm and about 180 rpm, between about 40 rpm and about 125 rpm or between about 60 rpm and about 100 rpm, each inclusive. In some embodiments, the motion is at a speed of between about 85 rpm and about 95 rpm, inclusive.

In some embodiments, the motion is shaking. In some embodiments, the shaking comprises orbital motion. In some embodiments, the motion is an undulating motion. In some embodiments, the undulating motion is with a shaker device that combines orbital movement and rocking movement. In some embodiments, the shaking comprises bidirectional linear movement. In some embodiments, the shaking is with an orbital shaker. In some embodiments, the motion is with a tilt angle. In some embodiments, the tilt angle is between 1° and 8°.

In some embodiments, one of the first modifying or further modifying comprises reducing expression of one or more genes encoding an endogenous protein in the cell and the other of the first modifying or the further modifying comprises increasing expression of one or more exogenous proteins in the cell. In some embodiments, the first modifying comprises reducing expression of one or more genes encoding an endogenous protein in the cell and the further modifying comprises increasing expression of one or more exogenous proteins in the cell.

In some embodiments, the one or more reagents reduce expression of one or more genes encoding an endogenous protein in the cell or increase expression of one or more heterologous proteins in the cell. In some embodiments, at least one of the first one or more reagents is for reducing expression of one or more genes encoding an endogenous protein in the cell and at least one of the further one or more reagents is for increasing expression of one or more exogenous proteins in the cell. In some embodiments, at least one of the first one or more reagents is for increasing expression of one or more exogenous proteins in the cell and at least one of the further one or more reagents is for reducing expression of one or more genes encoding an endogenous protein in the cell. In some embodiments, the first one or more reagents are for reducing expression of one or more genes encoding an endogenous protein in the cell and the further one or more reagents are for increasing expression of one or more exogenous proteins in the cell. In some embodiments, the one or more reagents comprise a genome-modifying protein for gene editing a target gene encoding an endogenous protein and/or an agent comprising an exogenous polynucleotide encoding an exogenous protein.

In some embodiments, reducing expression of one or more genes encoding an endogenous protein in the cell is by introducing a gene-editing system into the cell. In some embodiments, the gene-editing system comprises a sequence specific nuclease. In some of any embodiments, the one or more reagents for reducing expression of one or more genes encoding an endogenous protein in the cell comprise a genome-modifying protein. the genome-modifying protein is associated with gene editing by a sequence-specific nuclease, a CRISPR-associated transposase (CAST), prime editing, or Programmable Addition via Site-specific Targeting Elements (PASTE). In some embodiments, the genome-modifying protein is a sequence specific nuclease. In some embodiments, the sequence specific nuclease is an RNA-guided nuclease.

In some embodiments, the sequence specific nuclease is selected from the group consisting of a RNA-guided DNA endonuclease, a meganuclease, a transcription activator-like effector nuclease (TALEN), and a zinc-finger nuclease (ZFN). In some embodiments, the gene-editing system comprises an RNA-guided nuclease. In some embodiments, the RNA-guided nuclease comprises a Cas nuclease and a guide RNA. In some embodiments, the RNA-guided-nuclease is a Type II or Type V Cas protein. In some embodiments, the RNA-guided-nuclease is a Cas9 homologue or a Cpf1 homologue. In some embodiments, the genome-modifying protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. In some embodiments, the Cas is a Cas9. In some embodiments, the Cas is a Cas12.

In some embodiments, the one or more reagents are for reducing expression of one or more major histocompatibility complex (MHC) class I molecules and/or for reducing expression of one or more MHC class II molecules. In some embodiments, the first modifying comprises reducing expression of one or more major histocompatibility complex (MHC) class I molecules and/or one or more MHC class II molecules. In some embodiments, the modifying is genetic engineering. In some embodiments, the one or more MHC class I HLA is an HLA-A protein, an HLA-B protein, or HLA-C protein. In some embodiments, the one or more MHC class II HLA is an HLA-DP protein, an HLA-DR protein, or an HLA-DQ protein.

In some embodiments, the one or more reagents for reducing expression of one or more MHC class I molecule or MHC class II molecule reduces expression of one or more of B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B and/or NFY-C. In some embodiments, reducing expression of one or more MHC class I is by reducing expression of B-2 microglobulin (B2M). In some embodiments, reducing expression of one or more MHC class II is by reducing expression of CIITA.

In some embodiments, the further modifying comprises increasing expression of one or more tolerogenic factor in the cell. In some embodiments, the one or more exogenous proteins is one or more tolerogenic factors. In some embodiments, the one or more reagents comprise an agent for increasing expression of one or more tolerogenic factors. In some embodiments, the agent is a lipid particle or a a viral vector. In some embodiments, the viral vector is a lentiviral vector.

In some embodiments, the one or more tolerogenic factors is CD47, A20/TNFAIP3, C1-Inhibitor, CCL21, CCL22, CD16, CD16 Fc receptor, CD24, CD27, CD35, CD39, CD46, CD52, CD55, CD59, CD200, CR1, CTLA4-Ig, DUX4, FasL, H2-M3, HLA-C, HLA-E, HLA-E heavy chain, HLA-G, PD-L1, IDO1, IL-10, IL15-RF, IL-35, MANF, Mfge8, PD-1, PD-L1, or Serpinb9. In some embodiments, the one or more tolerogenic factor is selected from the group consisting of CD47, CD27, CD200, HLA-C, HLA-E, HLA-E heavy chain, HLA-G, PD-L1, IDO1, CTLA4-Ig, C1-Inhibitor, IL-10, IL-35, FASL, CCL21, MFGE8, and SERPINB9, and any combination thereof. In some embodiments, the one or more tolerogenic factor is selected from the group consisting of CD47, PD-L1, HLA-E, HLA-G, CCL21, FASL, SERPINB9, CD200, MFGE8, and any combination thereof. In some embodiments, at least one of the one or more tolerogenic factor is CD47.

In some embodiments, increasing expression of one or more exogenous proteins in the cell is by introducing an exogenous polynucleotide. In some embodiments, the exogenous polynucleotide is operably linked to a promoter. In some embodiments, the promoter is a constitutive promoter. In some embodiments, the promoter is selected from the group consisting of the CAG promoter, the cytomegalovirus (CMV) promoter, the EF1a promoter, the PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, the Rous sarcoma virus (RSV) promoter and the UBC promoter. In some embodiments, the exogenous polynucleotide is integrated into the genome of the cell. In some embodiments, the exogenous polynucleotide is a multicistronic vector. In some embodiments, the integration is by non-targeted insertion into the genome of the cell, optionally by introduction of the exogenous polynucleotide into the cell using a lentiviral vector. In some embodiments, the integration is by targeted insertion into a target genomic locus of the cell. In some embodiments, the islet cell is a beta islet cell.

In some embodiments, the engineered primary cell is a hepatocyte. In some embodiments, the engineered primary cell is a T cell. In some embodiments, the engineered primary cell is an endothelial cell. In some embodiments, the engineered primary cell is a thyroid cell. In some embodiments, the engineered primary cell is a skin cell. In some embodiments, the engineered primary cell is a retinal pigmented epithelium cell.

In some aspects, provided herein is an engineered cell produced according to the methods described herein. In some embodiments, the engineered cell is any cell as described herein. In some embodiments, the engineered cell is a primary cell. In some of any embodiments, the engineered primary cell is selected from an islet cell, a beta islet cell, B cell, T cell, NK cell, retinal pigmented epithelium cell, glial progenitor cell, endothelial cell, hepatocyte, thyroid cell, skin cell, and blood cell. In some embodiments, the primary cell is an islet cell. In some embodiments, the islet cell is a beta islet cell.

In some embodiments, the viability of the cells produced by the method is greater than about 30%, 40%, 50%, 55%, 60%, 65%, 70%, 75%, 80%, 85% or more. In some embodiments, the percentage of cells of the population modified by the method is greater than about 30%, 40%, 50%, 55%, 60%, 65%, 70%, 75%, 80%, 85% or more.

In some of any embodiments, the modifications as described in any of the provided engineered cells reduces innate immune killing of the engineered cell.

In some embodiments, the engineered primary cell is capable of evading NK cell mediated cytotoxicity upon administration to a recipient patient. In some embodiments, the engineered primary cell is protected from cell lysis by mature NK cells upon administration to a recipient patient. In some embodiments, the engineered primary cell does not induce an immune response to the cell upon administration to a recipient patient. In some embodiments, the engineered primary cell does not induce a systemic inflammatory response to the cell upon administration to a recipient patient. In some embodiments, the engineered primary cell does not induce a local inflammatory response to the cell upon administration to a recipient patient.

In some aspects, provided herein is a population of engineered primary cells comprising a plurality of any of the engineered primary cells described herein. In some embodiments, the plurality of the engineered primary cells are derived from cells pooled from more than one donor subject. In some embodiments, each of the more than one donor subjects are healthy subjects or are not suspected of having a disease or condition at the time the donor sample is obtained from the donor subject. In some embodiments, at least 50%, 60%, 70%, 80%, 90%, 95%, 96%, 97%, 98%, 99%, 99.9%, or 99.99% of cells in the population comprise the modifications. In some embodiments, at least 50%, 60%, 70%, 80%, 90%, 95%, 96%, 97%, 98%, 99%, 99.9%, or 99.99% of cells in the population comprise an exogenous polynucleotide encoding CD47. In some embodiments, at least 50%, 60%, 70%, 80%, 90%, 95%, 96%, 97%, 98%, 99%, 99.9%, or 99.99% of cells in the population comprise reduced expression of one or more MHC class I molecules and/or one or more MHC class II molecules relative to a cell of the same cell type that does not comprises the modification(s). In some embodiments, at least 50%, 60%, 70%, 80%, 90%, 95%, 96%, 97%, 98%, 99%, 99.9%, or 99.99% of cells in the population comprise reduced expression of B2M and/or CIITA relative to a cell of the same cell type that does not comprises the modification(s). In some embodiments, at least 50%, 60%, 70%, 80%, 90%, 95%, 96%, 97%, 98%, 99%, 99.9%, or 99.99% of cells in the population comprise reduced expression of B2M relative to a cell of the same cell type that does not comprises the modification(s). In some embodiments, at least 50%, 60%, 70%, 80%, 90%, 95%, 96%, 97%, 98%, 99%, 99.9%, or 99.99% of cells in the population comprise reduced expression of B2M and CIITA relative to a cell of the same cell type that does not comprises the modification(s). In some embodiments, at least 50%, 60%, 70%, 80%, 90%, 95%, 96%, 97%, 98%, 99%, 99.9%, or 99.99% of cells in the population comprise one or more alterations that inactivate both alleles of an endogenous B2M gene. In some embodiments, at least 50%, 60%, 70%, 80%, 90%, 95%, 96%, 97%, 98%, 99%, 99.9%, or 99.99% of cells in the population comprise one or more alterations that inactivate both alleles of an endogenous CIITA gene.

In some aspects, provided herein is a composition comprising any one of the populations described herein.

In some aspects, provided herein is a composition comprising an engineered primary islet cluster produced by any one of the methods described herein.

In some aspects, provided herein is a composition comprising a population of engineered primary islet cells, wherein the engineered primary islet cells comprise: (i) a transgene comprising an exogenous polynucleotide encoding CD47 and (ii) inactivation or disruption of both alleles of a B2M gene. In some embodiments, the population of engineered primary islet cells is a cluster of primary islet cells. In some embodiments, the population of engineered primary islet cells is a population of engineered primary beta islet cells.

In some aspects, provided herein is a composition comprising a population of engineered primary T cells, wherein the engineered primary T cells comprise: (i) a transgene comprising an exogenous polynucleotide encoding CD47 and (ii) inactivation or disruption of both alleles of a B2M gene.

In some aspects, provided herein is a composition comprising a population of engineered primary thyroid cells, wherein the engineered primary thyroid cells comprise: (i) a transgene comprising an exogenous polynucleotide encoding CD47 and (ii) inactivation or disruption of both alleles of a B2M gene.

In some aspects, provided herein is a composition comprising a population of engineered primary skin cells, wherein the engineered primary skin cells comprise: (i) a transgene comprising an exogenous polynucleotide encoding CD47 and (ii) inactivation or disruption of both alleles of a B2M gene.

In some aspects, provided herein is a composition comprising a population of engineered primary endothelial cells, wherein the engineered primary endothelial cells comprise: (i) a transgene comprising an exogenous polynucleotide encoding CD47 and (ii) inactivation or disruption of both alleles of a B2M gene.

In some aspects, provided herein is a composition comprising a population of engineered primary retinal pigmented epithelium cells, wherein the engineered primary retinal pigmented epithelium cells comprise: (i) a transgene comprising an exogenous polynucleotide encoding CD47 and (ii) inactivation or disruption of both alleles of a B2M gene.

In some embodiments, engineered primary cells of the population of engineered primary cells comprise an indel in both alleles of the B2M gene. In some embodiments, the engineered primary cells of the population of engineered primary cells further comprise inactivation or disruption of both alleles of a CIITA gene. In some embodiments, engineered primary cells of the population of engineered primary cells comprise an indel in both alleles of the CIITA gene. In some embodiments, the engineered primary cells of the population of engineered primary cells have the phenotype B2M^{indel/indel}; CIITA^{indel/indel}; CD47tg.

In some embodiments, the composition is a pharmaceutical composition. In some embodiments, the composition comprises a pharmaceutically acceptable excipient. In some embodiments, the pharmaceutically acceptable excipient is a buffered solution, such as saline. In some embodiments, the composition is formulated in a serum-free cryopreservation medium comprising a cryoprotectant. In some embodiments, the cryoprotectant is DMSO and the cryopreservation medium is 5% to 10% DMSO (v/v). In some embodiments, the cryoprotectant is or is about 10% DMSO (v/v). In some embodiments, the composition is sterile. In some embodiments, provided herein is a container, comprising any of the compositions described herein. In some embodiments, the container is a sterile bag. In some embodiments, the bag is a cryopreservation-compatible bag.

In some aspects, provided herein is a method of treating a disease, condition, or cellular deficiency in a patient in need thereof comprising administering to the patient an effective amount of any of the populations, compositions, or the pharmaceutical compositions described herein. In some embodiments, the population is formulated as a pharmaceutical composition comprising a pharmaceutically acceptable excipient. In some embodiments, the population of cells comprises islet cells, including beta islet cells. In some embodiments, the population of islet cells is administered as a cluster of islet cells. In some embodiments, the population of islet cells is administered as a cluster of beta islet cells. In some embodiments, the population of cells are hepatocytes. In some embodiments, the population of cells comprises T cells. In some embodiments, the population of cells comprises thyroid cells. In some embodiments, the population of cells comprises skin cells. In some embodiments, the population of cells comprises endothelial cells. In some embodiments, the population of cells comprises retinal pigmented epithelium cells.

In some embodiments, the condition or disease is selected from the group consisting of diabetes, cancer, vascularization disorders, ocular disease, thyroid disease, skin diseases, and liver diseases. In some embodiments, the cellular deficiency is associated with diabetes or the cellular therapy is for the treatment of diabetes, optionally wherein the diabetes is Type I diabetes.

In some embodiments, the population of cells is a population of islet cells, including beta islet cells. In some embodiments, the population of cells is administered as a cluster of islet cells.

In some aspects provided herein is a method of treating diabetes in a patient in need thereof, the method comprising administering to the patient an effective amount of any of the populations of islet cells, compositions, or the pharmaceutical compositions described herein. In some embodiments, the cluster of islet cells is a cluster of beta islet cells. In some embodiments, the cellular deficiency is associated with a vascular condition or disease or the cellular therapy is for the treatment of a vascular condition or disease. In some embodiments, the population of cells is a population of endothelial cells. In some embodiments, the cellular deficiency is associated with autoimmune thyroiditis or the cellular therapy is for the treatment of autoimmune thyroiditis. In some embodiments, the cellular deficiency is associated with a liver disease or the cellular therapy is for the treatment of liver disease. In some embodiments, the liver disease comprises cirrhosis of the liver. In some embodiments, the population of cells is a population of hepatocytes. In some embodiments, the cellular deficiency is associated with a corneal disease or the cellular therapy is for the treatment of corneal disease. In some embodiments, the corneal disease is Fuchs dystrophy or congenital hereditary endothelial dystrophy. In some embodiments, the population of cells is a population of corneal endothelial cells. In some embodiments, the cellular deficiency is associated with a kidney disease or the cellular therapy is for the treatment of a kidney disease. In some embodiments, the population of cells is a population of renal cells. In some embodiments, the cellular therapy is for the treatment of a cancer. In some embodiments, the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer. In some embodiments, the population of cells is a population of T cells or NK cells. In some embodiments, the cells are expanded and cryopreserved prior to administration.

In some embodiments, administering the population comprises intravenous injection, intramuscular injection, intravascular injection, or transplantation of the population. In some embodiments, the population is transplanted via kidney capsule transplant or intramuscular injection. In some embodiments, the population is derived from a donor subject, wherein the HLA type of the donor does not match the HLA type of the patient. In some embodiments, the population is a human cell population and the patient is a human patient. In some embodiments, the beta islet cells improve glucose tolerance in the subject. In some embodiments, the subject is a diabetic patient. In some embodiments, the diabetic patient has type I diabetes or type II diabetes. In some embodiments, glucose tolerance is improved relative to the subject's glucose tolerance prior to administration of the islet cells. In some embodiments, the beta islet cells reduce exogenous insulin usage in the subject. In some embodiments, glucose tolerance is improved as measured by HbA1c levels. In some embodiments, the subject is fasting. In some embodiments, the islet cells improve insulin secretion in the subject. In some embodiments, insulin secretion is improved relative to the subject's insulin secretion prior to administration of the islet cells.

In some embodiments of the method of treating a disease, the method further comprises administering one or more immunosuppressive agents to the patient. In some embodiments, the patient has been administered one or more immunosuppressive agents. In some embodiments, the one or more immunosuppressive agents are a small molecule or an antibody. In some embodiments, the one or more immunosuppressive agents are selected from the group consisting of cyclosporine, azathioprine, mycophenolic acid, mycophenolate mofetil, a corticosteroids, prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin (thymosin-α), and an immunosuppressive antibody. In some embodiments, the one or more immunosuppressive agents comprise cyclosporine. In some embodiments, the one or more immunosuppressive agents comprise mycophenolate mofetil. In some embodiments, the one or more immunosuppressive agents comprise a corticosteroid. In some embodiments, the one or more immunosuppressive agents comprise cyclophosphamide. In some embodiments, the one or more immunosuppressive agents comprise rapamycin. In some embodiments, the one or more immunosuppressive agents comprise tacrolimus (FK-506). In some embodiments, the one or more immunosuppressive agents comprise anti-thymocyte globulin. In some embodiments, the one or more immunosuppressive agents are one or more immunomodulatory agents.

In some embodiments of the method of treating a disease, the one or more immunomodulatory agents are a small molecule or an antibody. In some embodiments, the antibody binds to one or more of receptors or ligands selected from the group consisting of p75 of the IL-2 receptor, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, CD58, and antibodies binding to any of their ligands.

In some embodiments of the method of treating a disease, the one or more immunosuppressive agents are or have been administered to the patient prior to administration of the engineered cells. In some embodiments, the one or more immunosuppressive agents are or have been administered to the patient at least 1, 2, 3, 4, 5, 6, 7, 8, 9, 10, 11, 12, 13, or 14 days prior to administration of the engineered cells. In some embodiments, the one or more immunosuppressive agents are or have been administered to the patient at least 1 week, 2 weeks, 3 weeks, 4 weeks, 5 weeks, 6 weeks, 7 weeks, 8 weeks, 9 weeks, 10 weeks or more prior to administration of the engineered cells. In some embodiments, the one or more immunosuppressive agents are or have been administered to the patient at least 1, 2, 3, 4, 5, 6, 7, 8, 9, 10, 11, 12, 13, or 14 days after administration of the engineered cells. In some embodiments, the one or more immunosuppressive agents are or have been administered to the patient at least 1 week, 2 weeks, 3 weeks, 4 weeks, 5 weeks, 6 weeks, 7 weeks, 8 weeks, 9 weeks, 10 weeks, or more, after administration of the engineered cells. In some embodiments, the one or more immunosuppressive agents are or have been administered to the patient on the same day as the first administration of the engineered cells. In some embodiments, the one or more immunosuppressive agents are or have been administered to the patient after administration of the engineered cells. In some embodiments, the one or more immunosuppressive agents are or have been administered to the patient after administration of a first and/or second administration of the engineered cells. In some embodiments, the one or more immunosuppressive agents are or have been administered to the patient prior to administration of a first and/or second administration of the engineered cells. In some embodiments, the one or more immunosuppressive agents are or have been administered to the patient at least 1, 2, 3, 4, 5, 6, 7, 8, 9, 10, 11, 12, 13, or 14 days prior to administration of a first and/or second administration of the engineered cells. In some embodiments, the one or more immunosuppressive agents are or have been administered to the patient at least 1 week, 2 weeks, 3 weeks, 4 weeks, 5 weeks, 6 weeks, 7 weeks, 8 weeks, 9 weeks, 10 weeks or more prior to administration of a first and/or second administration of the engineered cells. In some embodiments, the one or more immunosuppressive agents are or have been administered to the patient at least 1, 2, 3, 4, 5, 6, 7, 8, 9, 10, 11, 12, 13, or 14 days after administration of a first and/or second administration of the engineered cells. In some embodiments, the one or more immunosuppressive agents are or have been administered to the patient at least 1 week, 2 weeks, 3 weeks, 4 weeks, 5 weeks, 6 weeks, 7 weeks, 8 weeks, 9 weeks, 10 weeks, or more, after administration of a first and/or second administration of the engineered cells. In some embodiments, the one or more immunosuppressive agents are administered at a lower dosage compared to the dosage of one or more immunosuppressive agents administered to reduce immune rejection of immunogenic cells that do not comprise the modifications of the engineered cells.

In some embodiments of the method of treating a disease, the engineered cell is capable of controlled killing of the engineered cell. In some embodiments, the engineered cell comprises a suicide gene or a suicide switch. In some embodiments, the suicide gene or the suicide switch induces controlled cell death in the presence of a drug or prodrug, or upon activation by a selective exogenous compound. In some embodiments, the suicide gene or the suicide switch is an inducible protein capable of inducing apoptosis of the engineered cell. In some embodiments, the inducible protein capable of inducing apoptosis of the engineered cell is a caspase protein. In some embodiments, the caspase protein is caspase 9. In some embodiments, the suicide gene or suicide switch is selected from the group consisting of cytosine deaminase (CyD), herpesvirus thymidine kinase (HSV-Tk), an inducible caspase 9 (iCaspase9), and rapamycin-activated caspase 9 (rapaCasp9). In some embodiments, the suicide gene or the suicide switch is activated to induce controlled cell death after the administration of the one or more immunosuppressive agents to the patient. In some embodiments, the suicide gene or the suicide switch is activated to induce controlled cell death prior to the administration of the one or more immunosuppressive agents to the patient. In some embodiments, the suicide gene or the suicide switch is activated to induce controlled cell death after the administration of the engineered cell to the patient. In some embodiments, the suicide gene or the suicide switch is activated to induce controlled cell death in the event of cytotoxicity or other negative consequences to the patient.

In some embodiments of the method of treating a disease, the method comprises administering an agent that allows for depletion of an engineered cell of the population of engineered cells. In some embodiments, the agent that allows for depletion of the engineered cell is an antibody that recognizes a protein expressed on the surface of the engineered cell. In some embodiments, the antibody is selected from the group consisting of an antibody that recognizes CCR4, CD16, CD19, CD20, CD30, EGFR, GD2, HER1, HER2, MUC1, PSMA, and RQR8. In some embodiments, the antibody is selected from the group consisting of mogamulizumab, AFM13, MOR208, obinutuzumab, ublituximab, ocaratuzumab, rituximab, rituximab-Rllb, tomuzotuximab, RO5083945 (GA201), cetuximab, Hu14.18K322A, Hu14.18-IL2, Hu3F8, dinituximab, c.60C3-Rllc, and biosimilars thereof.

In some embodiments of the method of treating a disease, the method comprises administering an agent that recognizes the one or more tolerogenic factors on the surface of the engineered cell. In some embodiments, the engineered cell is engineered to express the one or more tolerogenic factors. In some embodiments, the one or more tolerogenic factors is CD47.

In some embodiments of the method of treating a disease, the method further comprises administering one or more additional therapeutic agents to the patient. In some embodiments, the patient has been administered one or more additional therapeutic agents.

In some embodiments of the method of treating a disease, the method further comprises monitoring the therapeutic efficacy of the method. In some embodiments, the method further comprises monitoring the prophylactic efficacy of the method. In some embodiments, the method is repeated until a desired suppression of one or more disease symptoms occurs.

In some embodiments of the engineered cell, the engineered cell comprises an exogenous polynucleotide encoding a suicide gene or a suicide switch. In some embodiments, the suicide gene or suicide switch is selected from the group consisting of cytosine deaminase (CyD), herpesvirus thymidine kinase (HSV-Tk), an inducible caspase 9 (iCaspase9), and rapamycin-activated caspase 9 (rapaCasp9). In some embodiments, the suicide gene or suicide switch and genes associated with the suicide gene or the safety switch are expressed from a bicistronic cassette integrated into the genome of the engineered cell. In some embodiments, the suicide gene or suicide switch and the one or more tolerogenic factors are expressed from a bicistronic cassette integrated into the genome of the engineered cell. In some embodiments, the bicistronic cassette is integrated by non-targeted insertion into the genome of the engineered cell, optionally by introduction of the exogenous polynucleotide into the cell using a lentiviral vector. In some embodiments, the bicistronic cassette is integrated by targeted insertion into a target genomic locus of the engineered cell, optionally wherein the targeted insertion is by nuclease-mediated gene editing with homology-directed repair. In some embodiments, the one or more tolerogenic factors is CD47.

In some embodiments of the method of generating an engineered cell, the engineered cell comprises an exogenous polynucleotide encoding a suicide gene or suicide switch. In some embodiments, the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9). In some embodiments, the suicide gene or suicide switch and genes associated with the suicide gene or the safety switch are expressed from a bicistronic cassette integrated into the genome of the engineered cell. In some embodiments, the suicide gene or suicide switch and the one or more tolerogenic factors are expressed from a bicistronic cassette integrated into the genome of the engineered cell. In some embodiments, the bicistronic cassette is integrated by non-targeted insertion into the genome of the engineered cell. In some embodiments, the bicistronic cassette is integrated by targeted insertion into a target genomic locus of the engineered cell. In some embodiments, the one or more tolerogenic factors is CD47.

In some embodiments of the composition of engineered cells, engineered cells of the population of engineered cells comprise an exogenous polynucleotide encoding a suicide gene or a suicide switch. In some embodiments, the suicide gene or suicide switch is selected from the group consisting of cytosine deaminase (CyD), herpesvirus thymidine kinase (HSV-Tk), an inducible caspase 9 (iCaspase9), and rapamycin-activated caspase 9 (rapaCasp9). In some embodiments, the suicide gene and genes associated with the suicide gene or the safety switch are expressed from a bicistronic cassette integrated into the genome of engineered cells of the population of engineered cells. In some embodiments, the suicide gene or suicide switch and the exogenous CD47 are expressed from a bicistronic cassette integrated into the genome of the engineered cell. In some embodiments, the bicistronic cassette is integrated by non-targeted insertion into the genome, optionally by introduction of the exogenous polynucleotide into engineered cells of the population of engineered cells using a lentiviral vector. In some embodiments, the bicistronic cassette is integrated by targeted insertion into a target genomic locus of engineered cells of the population of engineered cells, optionally wherein the targeted insertion is by nuclease-mediated gene editing with homology-directed repair.

In some of any of the provided methods or cells, the cell is an autologous cell.

In some of any of the provided methods or cells, the cell is an allogeneic cell.

### BRIEF DESCRIPTION OF THE DRAWINGS

FIG. 1 shows that B2M^{-/-} primary beta islet cells isolated from C57BL/6 (B6) mice do not express major histocompatibility complex class I (MHC-I) or class II (MHC-II) molecules. Expression was analyzed before transplant and in cells isolated after transplant.

FIGS. 2A-2B show CD47 expression in mouse B2M^{-/-} primary beta islet cells (FIG. 2A) or B2M^{-/-} primary beta islet cells transduced with a lentiviral vector for overexpression of CD47 (FIG. 2B).

FIGS. 3A-3F provide results of intramuscular (i.m.) injection transplant studies of engineered and wild type (WT) primary beta islet cells. Quantification of bioluminescent imaging (BLI) of luciferase expression is provided for transplanted mouse WT primary beta islet cells (FIG. 3A quantification; FIG. 3B corresponding BLI image) and for transplanted mouse B2M^{-/-}; CD47^{tg} primary beta islet cells (FIG. 3C quantification; FIG. 3D corresponding BLI image). Blood glucose measurements are provided for diabetic mice transplanted with mouse WT primary beta islet cells (FIG. 3E) and diabetic mice transplanted with mouse B2M^{-/-}; CD47^{tg} primary beta islet cells (FIG. 3F).

FIGS. 4A-4F provide results of studies of engineered and mouse WT primary beta islet cells injected into a kidney capsule. Quantification of BLI of luciferase expression is provided for transplanted mouse WT primary beta islet cells (FIG. 4A quantification; FIG. 4B corresponding BLI image) and for transplanted mouse B2M^{-/-}; CD47^{tg} primary beta islet cells (FIG. 4C quantification; FIG. 4D corresponding BLI image). Blood glucose measurements are provided for diabetic mice transplanted with mouse WT primary beta islet cells (FIG. 4E) and diabetic mice transplanted with mouse B2M^{-/-}; CD47^{tg} primary beta islet cells (FIG. 4F).

FIGS. 5A-5C provide results of i.m. injection allogeneic transplant studies evaluating immune response. Blood glucose measurements are provided for diabetic mice transplanted with allogeneic mouse WT primary beta islet cells and diabetic mice transplanted with allogeneic mouse B2M^{-/-}; CD47tg primary beta islet cells (FIG. 5A). Interferon gamma (IFNg) levels are provided for diabetic mice transplanted with allogeneic mouse WT primary beta islet cells and diabetic mice transplanted with allogeneic mouse B2M^{-/-}; CD47tg primary beta islet cells (FIG. 5B). Donor-specific antibodies (DSA) IgG levels are provided for diabetic mice transplanted with allogeneic mouse WT primary beta islet cells and diabetic mice transplanted with allogeneic mouse B2M^{-/-}; CD47tg primary beta islet cells (FIG. 5C).

FIGS. 6A-6F provide results of Natural Killer (NK) cell mediated cell killing in vitro of engineered and mouse WT primary beta islet cells. NK cell mediated cell killing is provided for mouse WT primary beta islet cells (FIG. 6A), mouse B2M^{-/-} primary beta islet cells (FIG. 6B), and mouse B2M^{-/-}; CD47tg primary beta islet cells (FIG. 6C). NK cell mediated cell killing in the presence of an anti-CD47 antibody is also provided for mouse WT primary beta islet cells (FIG. 6D), mouse B2M^{-/-} primary beta islet cells (FIG. 6E), and mouse B2M^{-/-}; CD47tg primary beta islet cells (FIG. 6F).

FIGS. 7A-7F provide results of macrophage cell mediated cell killing in vitro of engineered mouse and mouse WT primary beta islet cells. Macrophage cell mediated cell killing is provided for mouse WT primary beta islet cells (FIG. 7A), mouse B2M^{-/-} primary beta islet cells (FIG. 7B), and mouse B2M^{-/-}; CD47tg primary beta islet cells (FIG. 7C). Macrophage cell mediated cell killing in the presence of an anti-CD47 antibody is also provided for mouse WT primary beta islet cells (FIG. 7D), mouse B2M^{-/-} primary beta islet cells (FIG. 7E), and mouse B2M^{-/-}; CD47tg primary beta islet cells (FIG. 7F).

FIGS. 8A-8N show CD47 expression in mouse B2M^{-/-} (B2M^{-/-}; CD47tg) primary beta islet cells transduced with a lentiviral vector for overexpression of CD47 (FIGS. 8A, 8C, 8E, 8G, 8I, 8K, 8M), and provide corresponding results of NK cell mediated cell killing (FIGS. 8B, 8D, 8F, 8H, 8J, 8L, 8N) in vitro of engineered mouse primary beta islet cells for various multiplicities of infection (MOI).

FIG. 9A depicts the cellular composition of a representative mouse transplanted with WT primary human beta islet cells and B2M^{-/-}; CD47tg primary human beta islet cells.

FIGS. 9B-9G depict flow cytometry staining of WT primary human beta islet cells and B2M^{-/-}; CD47tg engineered human beta islet cells generated from the WT primary human beta islet cells from a representative donor for surface expression of human leukocyte antigen (HLA) class-I (FIGS. 9B-9C), HLA class-II (FIGS. 9D-9E), and CD47 expression (FIGS. 9F-9G).

FIG. 9H depicts insulin secretion from the WT primary human beta islet cells and the B2M^{-/-}; CD47tg engineered human beta islet cells.

FIG. 9I-9K depict NK cell mediated cell killing in vitro for the WT primary human beta islet cells (FIGS. 9I), B2M^{-/-} primary human beta islet cells (FIG. 9J), and the B2M^{-/-}; CD47tg primary human beta islet cells (FIG. 9K), from a representative donor.

FIG. 9L-9N depict macrophage mediated cell killing in vitro for WT primary human beta islet cells (FIGS. 9L), the B2M^{-/-} primary human beta islet cells (FIG. 9M), and the B2M^{-/-}; CD47tg primary human beta islet cells (FIG. 9N), from a representative donor.

FIGS. 10A-10D provide results of studies of allogeneic transplant studies evaluating the recipient's immune response to the allogeneic human primary islet cells. Quantification of BLI of luciferase expression is provided for transplanted B2M^{-/-}; CD47⁹ human primary islet cells (FIG. 10A quantification; FIG. 10B corresponding BLI image) and for transplanted WT human primary islet cells (FIG. 10C quantification; FIG. 10D corresponding BLI image) from a representative donor.

FIGS. 10E and 10F depict blood glucose measurements for diabetic mice transplanted with allogeneic B2M^{-/-}; CD47tg human primary islet cells (FIG. 10E) and diabetic mice transplanted with allogeneic WT human primary islet cells (FIG. 10F) harvested from a representative donor.

FIG. 10G provides BLI of luciferase expression for diabetic mice transplanted with allogeneic B2M^{-/-}; human primary islet cells from a representative donor.

FIG. 10H depicts blood glucose measurements for diabetic mice transplanted with allogeneic B2M^{-/-} human primary islet cells harvested from a representative donor.

FIGS. 10I-10J provide results of i.m. injection allogeneic transplant studies evaluating immune response. Interferon gamma (IFNg) levels are provided for diabetic mice transplanted with WT human primary islet cells, B2M^{-/-} human primary islet cells, and B2M^{-/-}; CD47tg human primary islet cells (FIG. 10I). Donor-specific antibodies (DSA) IgG levels are provided for diabetic mice transplanted with WT human primary islet cells, B2M^{-/-} human primary islet cells, and B2M^{-/-}; CD47tg human primary islet cells (FIG. 10J).

FIGS. 11A-11C depict c-protein measurements for diabetic mice transplanted with allogeneic B2M^{-/-}; CD47tg human primary islet cells (FIG. 11A), WT human primary islet cells (FIG. 11B), and B2M^{-/-} human primary islet cells (FIG. 11C), harvested from a representative donor.

FIG. 12A provides results of in vitro splenocyte-mediated cell killing of transplanted WT primary human islet cells, transplanted B2M^{-/-} human primary islet cells, and planted B2M^{-/-}; CD47tg primary human islet cells.

FIG. 12B provides results of an in vitro complement dependent cytotoxicity (CDC) assay of transplanted WT primary human islet cells, transplanted B2M^{-/-} human primary islet cells, and transplanted B2M^{-/-}; CD47tg primary human islet cells.

FIGS. 13A-13D provide results of PBMC killing assays using diabetic patient peripheral blood mononuclear cells (PBMCs). Killing assay results with diabetic PBMC are shown for WT human primary islet cells (FIGS. 13A) and B2M^{-/-}; CD47tg human primary islet cells (FIG. 13C). As a control, killing also is shown for WT human primary islet cells (FIG. 13B) and B2M^{-/-}; CD47tg human primary islet cells (FIG. 13D) target cells only in the absence of PBMCs.

FIGS. 13E-13H provide results of PBMC killing assays using healthy donor PBMC. Killing assay results with healthy PBMC are shown for WT human primary islet cells from a representative (FIGS. 13E) and B2M^{-/-}; CD47tg human primary islet cells (FIG. 13G). As a control, killing also is shown for WT human primary islet cells (FIG. 13F) and the B2M^{-/-}; CD47tg human primary islet cells (FIG. 13H) target cells only in the absence of PBMCs.

FIGS. 13I-13J depict assessment of cell killing by flow cytometry analysis of dead cells. The percentage of dead cells following in vitro incubation of WT human primary islet cells from a representative donor with diabetic patient PBMCs or healthy donor PBMCs is shown in FIG. 13I. The percentage of dead cells following in vitro incubation of B2M^{-/-}; CD47tg human primary islet cells from a representative donor with diabetic patient PBMCs or healthy donor PBMCs is shown in FIG. 13J.

FIGS. 14A-14F provide results of Natural Killer (NK) cell and macrophage cell mediated cell killing in vitro of engineered primary human beta islet cells and engineered primary human beta islet cells. NK cell mediated cell killing is provided for B2M^{-/-}; CD47tg human primary islet cells (FIG. 14A), B2M^{-/-}; CD47tg human primary islet cells in the presence of anti-CD47 IgG1Fc (FIG. 14B), and B2M^{-/-}; CD47tg human primary islet cells in the presence of anti-CD47 IgG4Fc (FIG. 14C). Macrophage cell mediated cell killing is also provided B2M^{-/-}; CD47tg human primary islet cells (FIG. 14D), B2M^{-/-}; CD47tg human primary islet cells in the presence of anti-CD47 IgG1Fc (FIG. 14E), and B2M^{-/-}; CD47tg human primary islet cells in the presence of anti-CD47 IgG4Fc (FIG. 14F).

FIGS. 15A-15C depict granzyme B (FIG. 15A), perforin (FIG. 15B), and reactive oxygen species (ROS) (FIG. 15C) measurements in vitro for B2M^{-/-}; CD47tg human primary islet cells, B2M^{-/-}; CD47tg human primary islet cells expressing anti-CD47 IgG1Fc, and B2M^{-/-}; CD47tg human primary islet cells in the presence of anti-CD47 IgG4Fc.

FIG. 16 shows CD47 expression in WT human primary islet cells, B2M^{-/-} human primary islet cells, and B2M^{-/-}; CD47tg human primary islet cells.

FIG. 17 provides results of phagocytosis of macrophages in vitro for WT human primary islet cells (whole, apoptotic, and necrotic), B2M^{-/-} human primary islet cells (whole, apoptotic, and necrotic), B2M^{-/-}; CD47tg human primary islet cells (whole, apoptotic, and necrotic), and anti-CD47 B2M^{-/-}; CD47tg human primary islet cells (whole, apoptotic, and necrotic).

FIG. 18 provides results of phagocytosis of macrophages in vitro for B2M^{-/-}; CD47tg human primary islet cells, B2M^{-/-}; CD47tg primary human beta islet cells in the presence of anti-CD47 IgG1Fc, and B2M^{-/-}; CD47tg primary human beta islet cells in the presence of anti-CD47 IgG4Fc.

FIGS. 19A-19F provide results of allogeneic transplant studies evaluating the diabetic NSG mice immune response to allogeneic human primary islet cells. BLI of luciferase expression images are provided for transplanted B2M^{-/-}; CD47^{tg} human primary islet cells (FIG. 19A) and WT primary human islet cells (FIG. 19D) from a representative donor. Blood glucose measurements are depicted for diabetic NSG mice transplanted with allogeneic B2M^{-/-}; CD47tg human primary islet cells (FIG. 19B) and diabetic mice transplanted with allogeneic WT human primary islet cells (FIG. 19E) harvested from a representative donor. C-protein measurements are also provided for diabetic NSG mice transplanted with allogeneic B2M^{-/-}; CD47tg human primary islet cells (FIG. 19C) and diabetic mice transplanted with allogeneic WT human primary islet cells (FIG. 19F) harvested from a representative donor.

FIGS. 20A-20D provide results of allogeneic transplant studies evaluating the diabetic humanized mice immune response, or lack thereof, to allogeneic human primary islet cells in the presence of locally administered anti-CD47 and isotype control. BLI of luciferase expression images are provided for diabetic humanized mice transplanted with B2M^{-/-}; CIITA^{-/-}; CD47^{tg} human primary islet cells from a representative donor, which were further administered isotype control antibodies locally (FIG. 20A) or anti-CD47 locally (FIG. 20C) on day 8 following transplantation. Blood glucose measurements are depicted for diabetic humanized mice transplanted with B2M^{-/-}; CIITA^{-/-}; CD47^{tg} human primary islet cells from a representative donor, which were further administered isotype control antibodies locally (FIG. 20B) or anti-CD47 locally (FIG. 20D) on day 8 following transplantation.

FIGS. 21A-21D provide results of allogeneic transplant studies evaluating the diabetic humanized mice immune response, or lack thereof, to allogeneic human primary islet cells in the presence of systemically administered anti-CD47 or isotype control. BLI of luciferase expression images are provided for diabetic humanized mice transplanted with B2M^{-/-}; CIITA^{-/-}; CD47^{tg} human primary islet cells from a representative donor, which were further administered isotype control antibodies systemically (FIG. 21A) or anti-CD47 systemically (FIG. 21C) on day 8 following transplantation. Blood glucose measurements are depicted for diabetic humanized mice transplanted with B2M^{-/-}; CIITA^{-/-}; CD47^{tg} human primary islet cells from a representative donor, which were further administered isotype control antibodies systemically (FIG. 21B) or anti-CD47 systemically (FIG. 21D) on day 8 following transplantation.

FIGS. 22A-22B provide results of studies of allogeneic transplant studies evaluating non-human primate (NHP) recipient's immune response to the allogeneic NHP primary islet cells. Quantification of BLI of luciferase expression is provided for transplanted B2M^{-/-}; CIITA^{-/-}; CD47^{tg} NHP primary islet cells (FIG. 22A quantification; FIG. 22B corresponding BLI image).

FIGS. 23A-23D provide results of i.m. injection allogeneic transplant studies in NHPs evaluating immune response. Interferon gamma (IFNg) levels are provided for NHPs transplanted with B2M^{-/-}; CIITA^{-/-}; CD47^{tg} NHP primary islet cells (FIG. 23A). Donor-specific antibodies (DSA) IgM levels (FIG. 23B) and IgG levels (FIG. 23C) are provided NHPs transplanted with B2M^{-/-}; CIITA^{-/-}; CD47tg NHP primary islet cells. DSA IgG levels are also provided for a sensitized NHP transplanted with B2M^{-/-}; CIITA^{-/-}; CD47tg NHP primary islet cells with elevated IgG levels prior to transplantation (FIG. 23D).

FIG. 24 provides results of Natural Killer (NK) cell mediated cell killing in vitro of B2M^{-/-}; CIITA^{-/-}; CD47tg NHP primary islet cells.

FIG. 25 shows expression of MHC-I or MHC-II molecules and CD47 on WT human primary RPE cells (top panel), double knockout (B2M^{-/-} CIITA^{-/-}) primary RPE cells (middle panel) and B2M^{-/-}; CIITA^{-/-}; CD47tg primary RPE cells (bottom panel).

FIGS. 26A-26I provide results of Natural Killer (NK) cell mediated cell killing (FIGS. 26A-26C) and macrophage cell mediated cell killing (FIGS. 26D-26F) in vitro of WT human primary RPE cells (top panel), human double knockout (B2M^{-/-} CIITA^{-/-}) primary RPE cells (middle panel) and human B2M^{-/-}; CIITA^{-/-}; CD47tg primary RPE cells (bottom panel). Target cell only mediated cell killing is provided for each cell line as a control (FIGS. 26G-26I).

### DETAILED DESCRIPTION

Provided herein, in some aspects, are methods and compositions for alleviating and/or evading the effects of immune system reactions in response to allogeneic transplants, such as allogeneic cell therapies. To overcome the problem of immune rejection of cell therapies, disclosed herein is an engineered cell that has the ability to evade the immune system (also referred to here as an engineered immune-evasive cell or an engineered hypoimmunogenic cell), or population thereof, or pharmaceutical composition thereof, that represents a viable source for any transplantable cell type. In aspects of the engineered cells provided herein, rejection of the cells by the recipient subject's immune system is diminished and the engineered cells are able to engraft and function in the host after their administration, regardless of the subject's genetic make-up, or any existing response within the subject to one or more previous allogeneic transplants, previous autologous chimeric antigen receptor (CAR) T rejection, and/or other autologous or allogeneic therapies wherein a transgene is expressed. Thus, in some aspects, engineered cells refer to engineered immune-evasive cells. The engineered cells described herein may be derived from any cells, including, but are not limited to, islet cells, beta islet cells, B cells, T cells, NK cells, retinal pigmented epithelium cells, glial progenitor cells, endothelial cells, hepatocytes, thyroid cells, skin cells, and blood cells (e.g., plasma cells or platelets). In some embodiments, the provided engineered primary cells are engineered cells (e.g., cells taken directly from living tissue, such as a biopsy).

In some embodiments, the cells, such as primary cells, are engineered to have reduced or increased expression of one or more targets relative to an unaltered or unmodified wild-type cell. In some embodiments, the cells are engineered to have constitutive reduced or increased expression of one or more targets relative to an unaltered or unmodified wild-type cell. In some embodiments, the cells are engineered to have regulatable reduced or increased expression of one or more targets relative to an unaltered or unmodified wild-type cell. In some embodiments, the cells comprise increased expression of CD47 relative to a wild-type cell or a control cell of the same cell type. By “wild-type” or “wt” or “control” in the context of a cell means any cell found in nature. Examples of wild type or control cells include primary cells, such as primary pancreatic islet cells found in nature. However, by way of example, in the context of an engineered cell, as used herein, “wild-type” or “control” can also mean an engineered cell that may contain nucleic acid changes resulting in reduced expression of one or more MHC class I molecules and/or one or more MHC class II molecules, but did not undergo the gene editing procedures to result in overexpression of CD47 proteins. In some embodiments, the wild-type cell or the control cell is a starting material. In some embodiments, a primary cell line starting material is a starting material that is considered a wild-type or control cell as contemplated herein. In some embodiments, the starting material is otherwise modified or engineered to have altered expression of one or more genes to generate the engineered cell. In some embodiments, the wild-type cell or the control cell is a starting material in the form of cells from a donor. In certain embodiments, the wild-type cell or the control cell is a starting material can be cells obtained by organ or cell donation from a live subject or from a deceased subject, e.g. cadaveric pancreatic cells or kidney cells.

In some embodiments, the technology described herein applies to islet cells.

In some embodiments, the technology described herein applies to primary pancreatic beta islet cells whether isolated from a primary pancreatic islet, derived from primary pancreatic beta islet cells within a primary pancreatic islet, or as a component of a primary pancreatic islet. For example, primary pancreatic beta islet cells can be edited as a single beta islet cell, a population of beta islet cells, or as a component of a primary pancreatic islet (e.g., primary pancreatic beta islet cells present within the primary pancreatic islet along with other cell types). As another example, primary pancreatic beta islet cells can be administered to a patient as single beta islet cells, a population of beta islet cells, or as a component of a primary pancreatic islet (e.g., primary pancreatic beta islet cells present within the primary pancreatic islet along with other cell types). In embodiments where the pancreatic beta islet cells are present within the pancreatic islet along with other cell types, the other cell types may also be edited by the methods described herein.

In some embodiments, the technology described herein also applies to primary pancreatic islet cells dissociated from a primary islet prior to or after engineering, such as genetic engineering. Such dissociated islet cells can be clustered prior to administration to a patient and clusters can include islet cells, including beta islet cells, as well as other cell types including but not limited to those from the primary islet. Numbers of islet cells in the cluster can vary, such as about 50, about 100, about 250, about 500, about 750, about 1000, about 1250, about 1500, about 1750, about 2000, about 2250, about 2500, about 2750, about 3000, about 3500, about 4000, about 4500, or about 5000 cells. Patients can be administered about 10, about 20, about 30, about 40, about 50, about 75, about 100, about 125, about 150, about 200, about 250, about 300, about 325, about 350, about 375, about 400, about 425, about 450, about 475, about 500, about 600, about 700, about 800, about 900, or about 1000 clusters.

The engineered primary cells provided herein contain modifications (e.g., gene modifications) that result in altered expression (e.g., overexpression or increased expression) of one or more tolerogenic factors (e.g., CD47), and altered expression (e.g., reduced or eliminated expression) of one or more MHC class I molecules and/or one or more MHC class II molecules. In some embodiments, the modifications present in the engineered cell provide for altered (e.g. increased or overexpressed) cell surface expression of the one of more tolerogenic factors, and altered (e.g. reduced or eliminated) cell surface expression of one or more MHC class I molecules and/or one or more MHC class II molecules, such as an increase or overexpression of the one or more tolerogenic factors on the cell surface and reduced, or in some cases eliminated, expression of one or more MHC class I molecules and/or one or more MHC class II molecules on the cell surface. In provided aspects, the altered expression is relative to a similar cell that does not contain the modifications, such as a wild-type or unmodified cell of the same cell type or a cell that otherwise is the same but that lacks the modifications herein to alter expression of the one or more tolerogenic factors and one or more MHC class I molecules and/or one or more MHC class II molecules. Exemplary methods to introduce modifications to a cell to alter expression are described herein. For instance, any of a variety of methods for overexpressing or increasing expression of a gene or protein may be used, such as by introduction or delivery of an exogenous polynucleotide encoding a protein (i.e. a transgene) or introduction of delivery of a fusion protein of a DNA-targeting domain and a transcriptional activator targeting a gene. Also any of a variety of methods for reducing or eliminating expression of a gene or protein may be used, including non-gene editing methods such as by introduction or delivery of a inhibitory nucleic acids (e.g. RNAi) or gene editing methods involving introduction or delivery of a targeted nuclease system (e.g. CRISPR/Cas). In some embodiments, the method for reducing or eliminating expression is via a nuclease-based gene editing technique.

In some embodiments, genome editing technologies utilizing rare-cutting endonucleases (e.g., the CRISPR/Cas, TALEN, zinc finger nuclease, meganuclease, and homing endonuclease systems) are used to reduce or eliminate expression of immune genes (e.g., by deleting genomic DNA of critical immune genes) in human cells. In some embodiments, the genome editing technology comprises use of nickases, base editing, prime editing, and gene writing. In certain embodiments, genome editing technologies or other gene modulation technologies are used to insert tolerance-inducing (tolerogenic) factors in human cells, (e.g., CD47), thus producing engineered cells that can evade immune recognition upon engrafting into a recipient subject. Therefore, the engineered cells, such as engineered primary cells, provided herein exhibit modulated expression (e.g., reduced or eliminated expression) of one or more genes and factors that affect one or more MHC class I molecules and/or one or more MHC class II molecules, and modulated expression (e.g., increased expression or overexpression) of tolerogenic factors, such as CD47. In some embodiments, the engineered cells, such as engineered primary cells, evade the recipient subject's immune system.

In some aspects, engineered cells provided herein are not subject to an innate immune cell rejection or an adaptive immune cell rejection (e.g., hypoimmunogenic cells). For example, in some embodiments, the engineered cells are not susceptible to NK cell-mediated lysis and macrophage engulfment. In some embodiments, the engineered cells are useful as a source of universally compatible cells or tissues (e.g., universal donor cells or tissues) that are transplanted into a recipient subject with little to no immunosuppressant agent needed. Such hypoimmunogenic cells retain cell-specific characteristics and features upon transplantation.

The present disclosure is based, at least in part, on the inventors' findings and unique perspectives regarding engineering of cells useful for administration to individuals having preexisting antibodies (and/or antibodies that develop during the circulating life of an engineered cell in an individual having the been administered the engineered cell) against one or more cell surface antigens on the engineered cell. Such engineering helps to avoid triggering an immune response in the individual against the engineered cell. Furthermore, these findings support additional disclosure provided herein, such as patient and/or treatment selection.

The engineered cells provided herein may further utilize overexpression of tolerogenic factors and modulate (e.g., reduce or eliminate) expression of reduce expression of a one or more major histocompatibility complex (MHC) class I molecules (MHC class I molecules), or a component thereof, and/or one or more MHC class II molecules (MHC class II molecules) (e.g., surface expression). In some embodiments, genome editing technologies utilizing rare-cutting endonucleases (e.g., the CRISPR/Cas, TALEN, zinc finger nuclease, meganuclease, and homing endonuclease systems) are used to reduce or eliminate expression of immune genes (e.g., by deleting genomic DNA of critical immune genes) in human cells. In certain embodiments, genome editing technologies or other gene modulation technologies are used to insert tolerance-inducing (tolerogenic) factors in human cells, (e.g., CD47), thus producing engineered cells that can evade immune recognition upon engrafting into a recipient subject. Therefore, the engineered cells provided herein exhibit modulated expression of one or more genes and factors that affect one or more MHC class I molecules, one or more MHC class II molecules, and evade the recipient subject's immune system. In some cases, the cells are T cells and the cells also are engineered to modulate (e.g. reduce or eliminate) endogenous TCR expression.

In some embodiments, the engineered cells exhibit features that allow them to evade immune recognition. In some embodiments, the provided engineered cells are hypoimmunogenic. In some aspects, engineered cells provided herein are not subject to an innate immune cell rejection. For example, in some embodiments, the engineered cells are not susceptible to NK cell-mediated lysis and macrophage engulfment. In some embodiments, the engineered cells, such as engineered primary cells, are useful as a source of universally compatible cells or tissues (e.g., universal donor cells or tissues) that are transplanted into a recipient subject with little to no immunosuppressant agent needed. Such hypoimmunogenic cells retain cell-specific characteristics and features upon transplantation.

In some aspects provided herein is an engineered primary cell comprising modifications that (i) increase expression of one or more tolerogenic factor, and (ii) reduce expression of one or more major histocompatibility complex (MHC) class I molecules and/or one or more MHC class II molecules, wherein the increased expression of (i) and the reduced expression of (ii) is relative a cell of the same cell type that does not comprise the modifications. In some embodiments, the at least one of the one or more tolerogenic factor is CD47. In some embodiments, the modification that reduces expression of one or more MHC class I molecules is a modification that reduces expression of b-2 microglobulin (B2M). In some embodiments, the modification that reduces expression of one or more MHC class II is a modification that reduces expression of CIITA. In some embodiments, the modification(s) that increase expression comprise increased surface expression, and/or the modifications that reduce expression comprise reduced surface expression. In some embodiments, the engineered primary cell is selected from an islet cell, a beta islet cell, B cell, T cell, NK cell, retinal pigmented epithelium cell, glial progenitor cell, endothelial cell, hepatocyte, thyroid cell, skin cell, and blood cell (e.g., plasma cell or platelet).

In some aspects, provided herein is a method of generating an engineered cells, such as an engineered primary cell, the method comprising: a. reducing or eliminating the expression of one or more MHC class I molecules and/or one or more MHC class II molecules in the cell; and, b. increasing the expression of one or more tolerogenic factors in the cell. In some embodiments, the at least one of the one or more tolerogenic factor is CD47. In some embodiments, the modification that reduces expression of one or more MHC class I molecules is a modification that reduces expression of b-2 microglobulin (B2M). In some embodiments, the modification that reduces expression of one or more MHC class II molecules is a modification that reduces expression of CIITA. In some embodiments, the modification(s) that increase expression comprise increased surface expression, and/or the modifications that reduce expression comprise reduced surface expression. In some embodiments, the engineered primary cell is selected from an islet cell, a beta islet cell, B cell, T cell, NK cell, retinal pigmented epithelium cell, glial progenitor cell, endothelial cell, hepatocyte, thyroid cell, skin cell, and blood cell (e.g., plasma cell or platelet).

In some aspects, provided herein is a population of engineered cells, such as engineered primary cells, comprising a plurality of any of the engineered cells, such as engineered primary cells, described herein. In some embodiments, at least 50%, 60%, 70%, 80%, 90%, 95%, 96%, 97%, 98%, 99%, 99.9%, or 99.99% of cells in the population comprise an exogenous polynucleotide encoding CD47. In some embodiments, at least 50%, 60%, 70%, 80%, 90%, 95%, 96%, 97%, 98%, 99%, 99.9%, or 99.99% of cells in the population comprise reduced expression of B2M and/or CIITA relative to unaltered or unmodified wild type cells. In some embodiments, provided herein is a composition (e.g., a pharmaceutical composition any of the populations of engineered cells described herein. In some embodiments, provided herein is a composition (e.g., a pharmaceutical composition any of the populations of engineered primary cells described herein. In some embodiments, the population of engineered primary cells comprises a population of engineered primary cells selected from the group consisting of engineered primary islet cells, engineered primary beta islet cells, engineered primary T cells, engineered primary thyroid cells, engineered primary skin cells, engineered primary endothelial cells, and engineered primary retinal pigmented epithelium cells.

Also provided herein are methods for treating a disorder comprising administering the engineered cells (e.g., engineered primary cells) that evade immune rejection in an MHC-mismatched allogeneic recipient. In some embodiments, the engineered cells produced from any one of the methods described herein evade immune rejection when repeatedly administered (e.g., transplanted or grafted) to MHC-mismatched allogeneic recipient.

In some aspects, provided herein is a method of treating a disease, condition, or cellular deficiency in a patient in need thereof comprising administering to the patient an effective amount of the population of any of the populations of engineered cells, such as engineered primary cells, described herein. In some aspects, provided herein is a method of treating a disease, condition, or cellular deficiency in a patient in need thereof comprising administering to the patient an effective amount of any of the compositions of engineered cells, such as engineered primary cells described herein. In some aspects, provided herein is a method of treating a disease, condition, or cellular deficiency in a patient in need thereof comprising administering to the patient an effective amount of any of the pharmaceutical compositions of engineered cells, such as engineered primary cells, described herein.

The practice of the particular embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); and, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described. Those skilled in the art will recognize that several embodiments are possible within the scope and spirit of the present disclosure. The following description illustrates the disclosure and, of course, should not be construed in any way as limiting the scope of the inventions described herein.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “about” as used herein when referring to a measurable value, such as an amount or concentration and the like, is meant to encompass variations of 20%, 10%, 5%, 1%, 0.5%, or even 0.1% of the specified amount.

As used herein, including in the appended claims, the singular forms “a,” “or,” and “the” include plural referents unless the context clearly dictates otherwise. For example, “a” or “an” means “at least one” or “one or more.” It is understood that aspects and variations described herein include embodiments “consisting” and/or “consisting essentially of” such aspects and variations.

As used herein, the term “and/or” includes any and all combinations of one or more of the associated listed items.

As used herein, the term “exogenous” with reference to a polypeptide or a polynucleotide is intended to mean that the referenced molecule is introduced into the cell of interest. The exogenous molecule, such as exogenous polynucleotide, can be introduced, for example, by introduction of an exogenous encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell. In some cases, an “exogenous” molecule is a molecule, construct, factor and the like that is not normally present in a cell, but can be introduced into a cell by one or more genetic, biochemical or other methods.

The term “endogenous” refers to a referenced molecule, such as a polynucleotide (e.g. gene), or polypeptide, that is present in a native or unmodified cell. For instance, the term when used in reference to expression of an endogenous gene refers to expression of a gene encoded by an endogenous nucleic acid contained within the cell and not exogenously introduced.

A “gene,” includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. The sequence of a gene is typically present at a fixed chromosomal position or locus on a chromosome in the cell.

The term “locus” refers to a fixed position on a chromosome where a particular gene or genetic marker is located. Reference to a “target locus” refers to a particular locus of a desired gene in which it is desired to target a genetic modification, such as a gene edit or integration of an exogenous polynucleotide.

The term “expression” with reference to a gene or “gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or can be a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation. Hence, reference to expression or gene expression includes protein (or polypeptide) expression or expression of a transcribable product of a gene such as mRNA. The protein expression may include intracellular expression or surface expression of a protein. Typically, expression of a gene product, such as mRNA or protein, is at a level that is detectable in the cell.

As used herein, a “detectable” expression level, means a level that is detectable by standard techniques known to a skilled artisan, and include for example, differential display, RT (reverse transcriptase)-coupled polymerase chain reaction (PCR), Northern Blot, and/or RNase protection analyses as well as immunoaffinity-based methods for protein detection, such as flow cytometry, ELISA, or western blot. The degree of expression levels need only be large enough to be visualized or measured via standard characterization techniques.

As used herein, the term “increased expression”, “enhanced expression” or “overexpression” means any form of expression that is additional to the expression in an original or source cell that does not contain the modification for modulating a particular gene expression, for instance a wild-type expression level (which can be absence of expression or immeasurable expression as well). Reference herein to “increased expression,” “enhanced expression” or “overexpression” is taken to mean an increase in gene expression and/or, as far as referring to polypeptides, increased polypeptide levels and/or increased polypeptide activity, relative to the level in a cell that does not contain the modification, such as the original source cell prior to the engineering to introduce the modification, such as an unmodified cell or a wild-type cell. The increase in expression, polypeptide levels or polypeptide activity can be at least 5%, 10%, 20%, 30%, 40% or 50%, 60%, 70%, 80%, 85%, 90%, or 100% or even more. In some cases, the increase in expression, polypeptide levels or polypeptide activity can be at least 2-fold, 5-fold, 10-fold, 20-fold, 30-fold, 40-fold, 50-fold, 60-fold, 70-fold, 80-fold, 90-fold, 100-fold, 200-fold or more.

The term “hypoimmunogenic” refers to a cell that is less prone to immune rejection by a subject to which such cells are transplanted. For example, relative to a similar cell of the same cell type but that does not contain modifications, such as an unaltered or unmodified wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cells are transplanted. Typically, the hypoimmunogenic cells are allogeneic to the subject and a hypoimmunogenic cell evades immune rejection in an MHC-mismatched allogeneic recipient. In some embodiments, a hypoimmunogenic cell is protected from T cell-mediated adaptive immune rejection and/or innate immune cell rejection.

Hypoimmunogenicity of a cell can be determined by evaluating the immunogenicity of the cell such as the cell's ability to elicit adaptive and innate immune responses. Such immune response can be measured using assays recognized by those skilled in the art.

The term “tolerogenic factor” as used herein include immunosuppressive factors or immune-regulatory factors that modulate or affect the ability of a cell to be recognized by the immune system of a host or recipient subject upon administration, transplantation, or engraftment. Typically a tolerogenic factor is a factor that induces immunological tolerance to an engineered cell, such as engineered primary cell, so that the engineered cell, such as engineered primary cell is not targeted, such as rejected, by the host immune system of a recipient. Hence, a tolerogenic factor may be a hypoimmunity factor. Examples of tolerogenic factors include immune cell inhibitory receptors (e.g. CD47), proteins that engage immune cell inhibitory receptors, checkpoint inhibitors and other molecules that reduce innate or adaptive immune recognition

The terms “decrease,” “reduced,” “reduction,” and “decrease” are all used herein generally to mean a decrease by a statistically significant amount. However, for avoidance of doubt, “decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased,” “increase” or “enhance” or “activate” are all used herein to generally mean an increase by a statically significant amount; for the avoidance of any doubt, the terms “increased,” “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modification” refers to any change or alteration in a cell that impacts gene expression in the cell. In some embodiments, the modification is a genetic modification that directly changes the gene or regulatory elements thereof encoding a protein product in a cell, such as by gene editing, mutagenesis or by genetic engineering of an exogenous polynucleotide or transgene.

As used herein, “indel” refers to a mutation resulting from an insertion, deletion, or a combination thereof, of nucleotide bases in the genome. Thus, an indel typically inserts or deletes nucleotides from a sequence. As will be appreciated by those skilled in the art, an indel in a coding region of a genomic sequence will result in a frameshift mutation, unless the length of the indel is a multiple of three. A CRISPR/Cas system of the present disclosure can be used to induce an indel of any length in a target polynucleotide sequence.

In some embodiments, the alteration is a point mutation. As used herein, “point mutation” refers to a substitution that replaces one of the nucleotides. A CRISPR/Cas system of the present disclosure can be used to induce an indel of any length or a point mutation in a target polynucleotide sequence.

As used herein, “knock out” includes deleting all or a portion of the target polynucleotide sequence in a way that interferes with the function of the target polynucleotide sequence. For example, a knock out can be achieved by altering a target polynucleotide sequence by inducing an indel in the target polynucleotide sequence in a functional domain of the target polynucleotide sequence (e.g., a DNA binding domain). Those skilled in the art will readily appreciate how to use the CRISPR/Cas systems of the present disclosure to knock out a target polynucleotide sequence or a portion thereof based upon the details described herein.

In some embodiments, the alteration results in a knock out of the target polynucleotide sequence or a portion thereof. Knocking out a target polynucleotide sequence or a portion thereof using a CRISPR/Cas system of the present disclosure can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject).

By “knock in” herein is meant a process that adds a genetic function to a host cell. This causes increased levels of the knocked in gene product, e.g., an RNA or encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

In some embodiments, an alteration or modification described herein results in reduced expression of a target or selected polynucleotide sequence. In some embodiments, an alteration or modification described herein results in reduced expression of a target or selected polypeptide sequence.

In some embodiments, an alteration or modification described herein results in increased expression of a target or selected polynucleotide sequence. In some embodiments, an alteration or modification described herein results in increased expression of a target or selected polypeptide sequence.

“Modulation” of gene expression refers to a change in the expression level of a gene. Modulation of expression can include, but is not limited to, gene activation and gene repression. Modulation may also be complete, i.e. wherein gene expression is totally inactivated or is activated to wildtype levels or beyond; or it may be partial, wherein gene expression is partially reduced, or partially activated to some fraction of wildtype levels.

The term “operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

The terms “polypeptide” and “protein,” as used herein, may be used interchangeably to refer to a series of amino acid residues joined by peptide bonds (i.e. a polymer of amino acid residues), and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Thus, a protein or polypeptide includes include those with modified amino acids (e.g., phosphorylated, glycated, glycosylated, etc.) and amino acid analogs. Full-length polypeptides or proteins, and fragments thereof, are encompassed by this definition. The terms also include modified species thereof, e.g., post-translational modifications of one or more residues, for example, methylation, phosphorylation glycosylation, sialylation, or acetylation.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For instance, where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure. In some embodiments, two opposing and open ended ranges are provided for a feature, and in such description it is envisioned that combinations of those two ranges are provided herein. For example, in some embodiments, it is described that a feature is greater than about 10 units, and it is described (such as in another sentence) that the feature is less than about 20 units, and thus, the range of about 10 units to about 20 units is described herein.

As used herein, a “subject” or an “individual,” which are terms that are used interchangeably, is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject or individual is human. In some embodiments, the subject is a patient that is known or suspected of having a disease, disorder or condition.

As used herein, the term “treating” and “treatment” includes administering to a subject an effective amount of cells described herein so that the subject has a reduction in at least one symptom of the disease or an improvement in the disease, for example, beneficial or desired clinical results. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a disease or disorder are alleviated by at least 5%, at least 10%, at least 20%, at least 30%, at least 40%, or at least 50% upon treatment of the disease.

For purposes of this technology, beneficial or desired clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

A “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

### II. Engineered Cells and Methods of Engineering Cells

Provided herein are engineered cells, such as engineered primary cells, that comprise a modification that regulates the expression of one or more target polynucleotide sequences, such as regulates the expression of one or more MHC class I molecules, one or more MHC class II molecules, or one or more MHC class I molecules and one or more MHC class II molecules.

In some embodiments, the provided engineered cells, such as engineered primary cells, also include a modification to modulate (e.g., increase) expression of one or more tolerogenic factor. In some embodiments, the modulation of expression of the tolerogenic factor (e.g., increased expression), and the modulation of expression of the one or more MHC class I molecules and/or one or more MHC class II molecules (e.g., reduced or eliminated expression) is relative to the amount of expression of said molecule(s) in a cell that does not comprise the modification(s). In some embodiments, the modulation of expression is relative to the amount of expression of said molecule(s) in a wildtype cell. In some embodiments, the unmodified or wildtype cell is a cell of the same cell type as the provided engineered primary cells. In some embodiments, the unmodified cell or wildtype cell expresses the tolerogenic factor, the one or more MHC class I molecules, and/or the one or more MHC class II molecules. In some embodiments, the unmodified cell or wildtype cell does not express the one or more tolerogenic factor, the one or more MHC class I molecules, and/or the one or more MHC class II molecules. In some embodiments wherein the unmodified cell or wildtype cell does not express the tolerogenic factor is used to generate the engineered primary cell, the provided engineered primary cells include a modification to overexpress the one or more tolerogenic factor or increase the expression of the one or more tolerogenic factor from 0%. It is understood that if the cell prior to the engineering does not express a detectable amount of the tolerogenic factor, then a modification that results in any detectable amount of an expression of the tolerogenic factor is an increase in the expression compared to the similar cell that does not contain the modifications.

In some embodiments, modulation of expression of the tolerogenic factor (e.g., increased expression), and the modulation of expression of the one or more MHC class I molecules and/or one or more MHC class II molecules (e.g., reduced or eliminated expression) is relative to the amount of expression of said molecule(s) in a cell of the same cell type that does comprise not the modification(s). In some embodiments wherein a cell of the same cell type that does not express the one or more tolerogenic factor is used to generate the engineered primary cell, the provided engineered primary cells include a modification to overexpress the one or more tolergenic factor or increase the expression of the one or more tolgenic factor from 0%/It is understood that if the cell prior to the engineering does not express a detectable amount of the tolerogenic factor, then a modification that results in any detectable amount of an expression of the tolerogenic factor is an increase in the expression compared to the similar cell that does not contain the modifications.

In some embodiments, the provided engineered cells, such as engineered primary cells, include a modification to increase expression of one or more tolerogenic factors. In some embodiments, the tolerogenic factor is one or more of DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, C1-Inhibitor, IL-10, IL-35, FASL, CCL21, MFGE8, SERPINB9, CD35, IL-39, CD16 Fc Receptor, IL15-RF, and H2-M3 (including any combination thereof). In some embodiments, the tolerogenic factor is one or more of CD47, PD-L1, HLA-E or HLA-G, CCL21, FasL, Serpinb9, CD200, and Mfge8 (including any combination thereof). In some embodiments, the modification to increase expression of one or more tolerogenic factors is or includes increased expression of CD47. In some embodiments, the modification to increase expression of one or more tolerogenic factors is or includes increased expression of PD-L. In some embodiments, the modification to increase expression of one or more tolerogenic factors is or includes increased expression of HLA-E. In some embodiments, the modification to increase expression of one or more tolerogenic factors is or includes increased expression of HLA-G. In some embodiments, the modification to increase expression of one or more tolerogenic factors is or includes increased expression of CCL21, PD-L1, FasL, Serpinb9, H2-M3 (HLA-G), CD47, CD200, and Mfge8.

In some embodiments, the cells include one or more modifications, such as genomic modifications, that reduce expression of one or more MHC class I molecules and a modification that increases expression of CD47. In other words, the engineered cells, such as engineered primary cells, comprise exogenous CD47 proteins and exhibit reduced or silenced surface expression of one or more one or more MHC class I molecules. In some embodiments, the cells include one or more genomic modifications that reduce expression of one or more MHC class II molecules and a modification that increases expression of CD47. In some instances, the engineered cells, such as engineered primary cells, comprise exogenous CD47 nucleic acids and proteins and exhibit reduced or silenced surface expression of one or more MHC class I molecules. In some embodiments, the cells include one or more genomic modifications that reduce or eliminate expression of one or more MHC class II molecules, one or more genomic modifications that reduce or eliminate expression of one or more MHC class II molecules, and a modification that increases expression of CD47. In some embodiments, the engineered cells, such as engineered primary cells, comprise exogenous CD47 proteins, exhibit reduced or silenced surface expression of one or more MHC class I molecules and exhibit reduced or lack surface expression of one or more MHC class II molecules. In many embodiments, the cells are B2M^{indel/indel}, CIITA^{indel/indel}, CD47tg cells.

In some embodiments, the population of engineered cells, such as engineered primary cells, described elicits a reduced level of immune activation or no immune activation upon administration to a recipient subject. In some embodiments, the cells elicit a reduced level of systemic TH1 activation or no systemic TH1 activation in a recipient subject. In some embodiments, the cells elicit a reduced level of immune activation of peripheral blood mononuclear cells (PBMCs) or no immune activation of PBMCs in a recipient subject. In some embodiments, the cells elicit a reduced level of donor-specific IgG antibodies or no donor specific IgG antibodies against the cells upon administration to a recipient subject. In some embodiments, the cells elicit a reduced level of IgM and IgG antibody production or no IgM and IgG antibody production against the cells in a recipient subject. In some embodiments, the cells elicit a reduced level of cytotoxic T cell killing of the cells upon administration to a recipient subject.

In some embodiments, the engineered cells, such as engineered primary cells, provided herein comprise a “suicide gene” or “suicide switch”. A suicide gene or suicide switch can be incorporated to function as a “safety switch” that can cause the death of the engineered cell (e.g. primary engineered cell), such as after the engineered cell, such as engineered primary cell, is administered to a subject and if they cells should grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al, Mol. Therap. 20(10): 1932-1943 (2012), Xu et al, Cell Res. 8:73-8 (1998), both incorporated herein by reference in their entirety).

In other embodiments, the suicide gene is an inducible Caspase protein. An inducible Caspase protein comprises at least a portion of a Caspase protein capable of inducing apoptosis. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, API 903. Thus, the suicide function of iCasp9 in the instant invention is triggered by the administration of a chemical inducer of dimerization (CID). In some embodiments, the CID is the small molecule drug API 903. Dimerization causes the rapid induction of apoptosis. (See WO2011146862; Stasi et al, N. Engl. J. Med 365; 18 (2011); Tey et al, Biol. Blood Marrow Transplant. 13:913-924 (2007), each of which are incorporated by reference herein in their entirety.)

Inclusion of a safety switch or suicide gene allows for controlled killing of the cells in the event of cytotoxicity or other negative consequences to the recipient, thus increasing the safety of cell-based therapies, including those using tolerogenic factors.

In some embodiments, a safety switch can be incorporated into, such as introduced, into the engineered cells, such as engineered primary cells, provided herein to provide the ability to induce death or apoptosis of engineered cells, such as engineered primary cells, containing the safety switch, for example if the cells grow and divide in an undesired manner or cause excessive toxicity to the host. Thus, the use of safety switches enables one to conditionally eliminate aberrant cells in vivo and can be a critical step for the application of cell therapies in the clinic. Safety switches and their uses thereof are described in, for example, Duzgune§, Origins of Suicide Gene Therapy (2019); Duzgune§ (eds), Suicide Gene Therapy. Methods in Molecular Biology, vol. 1895 (Humana Press, New York, NY) (for HSV-tk, cytosine deaminase, nitroreductase, purine nucleoside phosphorylase, and horseradish peroxidase); Zhou and Brenner, Exp Hematol 44(11):1013-1019 (2016) (for iCaspase9); Wang et al., Blood 18(5):1255-1263 (2001) (for huEGFR); U.S. Patent Application Publication No. 20180002397 (for HER1); and Philip et al., Blood 124(8):1277-1287 (2014) (for RQR8).

In some embodiments, the safety switch can cause cell death in a controlled manner, for example, in the presence of a drug or prodrug or upon activation by a selective exogenous compound. In some embodiments, the safety switch is selected from the group consisting of herpes simplex virus thymidine kinase (HSV-tk), cytosine deaminase (CyD), nitroreductase (NTR), purine nucleoside phosphorylase (PNP), horseradish peroxidase, inducible caspase 9 (iCasp9), rapamycin-activated caspase 9 (rapaCasp9), CCR4, CD16, CD19, CD20, CD30, EGFR, GD2, HER1, HER2, MUC1, PSMA, and RQR8.

In some embodiments, the safety switch may be a transgene encoding a product with cell killing capabilities when activated by a drug or prodrug, for example, by turning a non-toxic prodrug to a toxic metabolite inside the cell. In these embodiments, cell killing is activated by contacting an engineered cell, such as an engineered primary cell, with the drug or prodrug. In some cases, the safety switch is HSV-tk, which converts ganciclovir (GCV) to GCV-triphosphate, thereby interfering with DNA synthesis and killing dividing cells. In some cases, the safety switch is CyD or a variant thereof, which converts the antifungal drug 5-fluorocytosine (5-FC) to cytotoxic 5-fluorouracil (5-FU) by catalyzing the hydrolytic deamination of cytosine into uracil. 5-FU is further converted to potent anti-metabolites (5-FdUMP, 5-FdUTP, 5-FUTP) by cellular enzymes. These compounds inhibit thymidylate synthase and the production of RNA and DNA, resulting in cell death. In some cases, the safety switch is NTR or a variant thereof, which can act on the prodrug CB 1954 via reduction of the nitro groups to reactive N-hydroxylam ine intermediates that are toxic in proliferating and nonproliferating cells. In some cases, the safety switch is PNP or a variant thereof, which can turn prodrug 6-methylpurine deoxyriboside or fludarabine into toxic metabolites to both proliferating and nonproliferating cells. In some cases, the safety switch is horseradish peroxidase or a variant thereof, which can catalyze indole-3-acetic acid (IAA) to a potent cytotoxin and thus achieve cell killing.

In some embodiments, the safety switch may be an iCasp9. Caspase 9 is a component of the intrinsic mitochondrial apoptotic pathway which, under physiological conditions, is activated by the release of cytochrome C from damaged mitochondria. Activated caspase 9 then activates caspase 3, which triggers terminal effector molecules leading to apoptosis. The iCasp9 may be generated by fusing a truncated caspase 9 (without its physiological dimerization domain or caspase activation domain) to a FK506 binding protein (FKBP), FKBP12-F36V, via a peptide linker. The iCasp9 has low dimer-independent basal activity and can be stably expressed in host cells (e.g., human T cells) without impairing their phenotype, function, or antigen specificity. However, in the presence of chemical inducer of dimerization (CID), such as rimiducid (AP1903), AP20187, and rapamycin, iCasp9 can undergo inducible dimerization and activate the downstream caspase molecules, resulting in apoptosis of cells expressing the iCasp9. See, e.g., PCT Application Publication No. WO2011/146862; Stasi et al., N. Engl. J. Med. 365; 18 (2011); Tey et al., Biol. Blood Marrow Transplant 13:913-924 (2007). In particular, the rapamycininducible caspase 9 variant is called rapaCasp9. See Stavrou et al., Mal. Ther. 26(5):1266-1276 (2018). Thus, iCasp9 can be used as a safety switch to achieve controlled killing of the host cells.

In some embodiments, the safety switch may be a membrane-expressed protein which allows for cell depletion after administration of a specific antibody to that protein. Safety switches of this category may include, for example, one or more transgene encoding CCR4, CD16, CD19, CD20, CD30, EGFR, GD2, HER1, HER2, MUC1, PSMA, or RQR8 for surface expression thereof. These proteins may have surface epitopes that can be targeted by specific antibodies. In some embodiments, the safety switch comprises CCR4, which can be recognized by an anti-CCR4 antibody. Non-limiting examples of suitable anti-CCR4 antibodies include mogamulizumab and biosimilars thereof. In some embodiments, the safety switch comprises CD16 or CD30, which can be recognized by an anti-CD16 or anti-CD30 antibody. Non-limiting examples of such antiCD16 or anti-CD30 antibody include AFM13 and biosimilars thereof. In some embodiments, the safety switch comprises CD19, which can be recognized by an anti-CD19 antibody. Non-limiting examples of such anti-CD19 antibody include MOR208 and biosimilars thereof. In some embodiments, the safety switch comprises CD20, which can be recognized by an anti-CD20 antibody. Non-limiting examples of such anti-CD20 antibody include obinutuzumab, ublituximab, ocaratuzumab, rituximab, rituximab-Rllb, and biosimilars thereof. Cells that express the safety switch are thus CD20-positive and can be targeted for killing through administration of an anti-CD20 antibody as described. In some embodiments, the safety switch comprises EGFR, which can be recognized by an anti-EGFR antibody. Non-limiting examples of such anti-EGFR antibody include tomuzotuximab, RO5083945 (GA201), cetuximab, and biosimilars thereof. In some embodiments, the safety switch comprises GD2, which can be recognized by an anti-GD2 antibody. Non-limiting examples of such anti-GD2 antibody include Hu14.18K322A, Hu14.18-IL2, Hu3F8, dinituximab, c.60C3-Rllc, and biosimilars thereof.

In some embodiments, the safety switch may be an exogenously administered agent that recognizes one or more tolerogenic factor on the surface of the engineered cell, such as engineered primary cell. In some embodiments, the exogenously administered agent is an antibody directed against or specific to a tolerogenic agent, e.g. an anti-CD47 antibody. By recognizing and blocking a tolerogenic factor on the engineered cell, such as engineered primary cell, an exogenously administered antibody may block the immune inhibitory functions of the tolerogenic factor thereby re-sensitizing the immune system to the engineered cells, such as engineered primary cells. For instance, for an engineered cell, such as an engineered primary cell, that overexpresses CD47 an exogenously administered anti-CD47 antibody may be administered to the subject, resulting in masking of CD47 on the engineered cell, such as engineered primary cell, and triggering of an immune response to the engineered primary cell.

In some embodiments, the method further comprises introducing an expression vector comprising an inducible suicide switch into the cell.

In some embodiments, the tolerogenic factor is CD47 and the cell includes an exogenous polynucleotide encoding a CD47 protein. In some embodiments, the cell expresses an exogenous CD47 polypeptide.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent comprises a CD47-binding domain. In some embodiments, the CD47-binding domain comprises signal regulatory protein alpha (SIRPα) or a fragment thereof. In some embodiments, the CD47-SIRPα blockade agent comprises an immunoglobulin G (IgG) Fc domain. In some embodiments, the IgG Fc domain comprises an IgG1 Fc domain. In some embodiments, the IgG1 Fc domain comprises a fragment of a human antibody. In some embodiments, the CD47-SIRPα blockade agent is selected from the group consisting of TTI-621, TTI-622, and ALX148. In some embodiments, the CD47-SIRPα blockade agent is TTI-621, TTI-622, and ALX148. In some embodiments, the CD47-SIRPα blockade agent is TTI-622. In some embodiments, the CD47-SIRPα blockade agent is ALX148. In some embodiments, the IgG Fc domain comprises an IgG4 Fc domain. In some embodiments, the CD47-SIRPα blockade agent is an antibody. In some embodiments, the antibody is selected from the group consisting of MIAP410, B6H12, and Magrolimab. In some embodiments, the antibody is MIAP410. In some embodiments, the antibody is B6H12. In some embodiments, the antibody is Magrolimab. In some embodiments, the antibody is selected from the group consisting of AO-176, IBI188 (letaplimab), STI-6643, and ZL-1201. In some embodiments, the antibody is AO-176 (Arch). In some embodiments, the antibody is IBI188 (letaplimab) (Innovent). In some embodiments, the antibody is STI-6643 (Sorrento). In some embodiments, the antibody is ZL-1201 (Zai).

In some embodiments, useful antibodies or fragments thereof that bind CD47 can be selected from a group that includes magrolimab ((Hu5F9-G4)) (Forty Seven, Inc.; Gilead Sciences, Inc.), urabrelimab, CC-90002 (Celgene; Bristol-Myers Squibb), IBI-188 (Innovent Biologics), IBI-322 (Innovent Biologics), TG-1801 (TG Therapeutics; also known as NI-1701, Novimmune SA), ALX148 (ALX Oncology), TJ011133 (also known as TJC4, I-Mab Biopharma), FA3M3, ZL-1201 (Zai Lab Co., Ltd), AKi 17 (Akesbio Australia Pty, Ltd.), AO-176 (Arch Oncology), SRF231 (Surface Oncology), GenSci-059 (GeneScience), C47B157 (Janssen Research and Development), C47B161 (Janssen Research and Development), C47B167 (Janssen Research and Development), C47B222 (Janssen Research and Development), C47B227 (Janssen Research and Development), Vx-1004 (Corvus Pharmaceuticals), HMBD004 (Hummingbird Bioscience Pte Ltd), SHR-1603 (Hengrui), AMMS4-G4 (Beijing Institute of Biotechnology), RTX-CD47 (University of Groningen), and IMC-002. (Samsung Biologics; ImmuneOncia Therapeutics). In some embodiments, the antibody or fragment thereof does not compete for CD47 binding with an antibody selected from a group that includes magrolimab, urabrelimab, CC-90002, IBI-188, IBI-322, TG-1801 (NI-1701), ALX148, TJ011133, FA3M3, ZL1201, AKl17, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof competes for CD47 binding with an antibody selected from magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof that binds CD47 is selected from a group that includes a single-chain Fv fragment (scFv) against CD47, a Fab against CD47, a VHH nanobody against CD47, a DARPin against CD47, and variants thereof. In some embodiments, the scFv against CD47, a Fab against CD47, and variants thereof are based on the antigen binding domains of any of the antibodies selected from a group that includes magrolimab, urabrelimab, CC-90002, IBI-188, IBI-322, TG-1801 (NI-1701), ALX148, TJ011133, FA3M3, ZL1201, AKl17, AO-176, SRF231, GenSci-059, C47B157, C47B161, C47B167, C47B222, C47B227, Vx-1004, HMBD004, SHR-1603, AMMS4-G4, RTX-CD47, and IMC-002.

In some embodiments, the CD47 antagonist provides CD47 blockade. Methods and agents for CD47 blockade are described in PCT/US2021/054326, which is incorporated by reference in its entirety.

In some embodiments, the engineered cell, such as engineered primary cell, is derived from a source cell already comprising one or more of the desired modifications. In some embodiments, in view of the teachings provided herein one of ordinary skill in the art will readily appreciate how to assess what modifications are required to arrive at the desired final form of an engineered cell, such as engineered primary cell, and that not all reduced or increased levels of target components are achieved via active engineering. In some embodiments, the modifications of the engineered primary cell may be in any order, and not necessarily the order listed in the descriptive language provided herein.

Once altered, the presence of expression of any of the molecule described herein can be assayed using known techniques, such as Western blots, ELISA assays, FACS assays, flow cytometry, and the like.

### A. Targets Having Reduced Expression Genes

In some embodiments, the engineered cells, such as engineered primary cells, comprise a modification (e.g. genetic modifications) of one or more target polynucleotide or protein sequences (also interchangeably referred to as a target gene) that regulate (e.g. reduce or eliminate) the expression of one or more of: one or more MHC class I molecules, one or more MHC class II molecules, MIC-A, MIC-B, TXIP, CTLA-4 and/or PD-1. In some embodiments, the engineered cells, such as engineered primary cells, comprise a modification of one or more gene that regulates (e.g. reduce or eliminate) one or more MHC class I molecules and/or one or more MHC class II molecules. In some embodiments, the one or more MHC class I molecules and/or one or more MHC class II molecules is any one or more of HLA-A, HLA-B, HLA-C, HLA-DP, HLA-DM, HLA-DOA, HLA-DOB, HLA-DQ and/or HLA-DR. In some embodiments, the modification to the target gene is a modification that reduces or eliminates any one or more of B2M, TAP I, NLRC5, CIITA, RFX5, RFXANK, RFXAP, NFY-A, NFY-B or NFY-C. In some embodiments, the engineered cell, such as a engineered primary cell, has a modification that reduces or eliminates expression of one or more of B2M, TAP I, NLRC5, CIITA, RFX5, RFXANK, RFXAP, NFY-A, NFY-B, NFY-C, MIC-A, MIC-B, TXIP, CTLA-4 and/or PD-1. Any of a variety of methods known to a skilled artisan can be used to reduce or eliminate expression of any such target genes, including any of variety of known gene editing technologies.

In some embodiments, the provided engineered cells, such as engineered primary cells, comprises a modification (e.g. genetic modifications) of one or more target polynucleotide or protein sequences (also interchangeably referred to as a target gene) that regulate (e.g. reduce or eliminate) the expression of either one or more MHC class I molecules, one or more MHC class II molecules, or one or more MHC class I molecules and one or more MHC class II molecules. In some embodiments, the primary cell to be modified or engineered is an unmodified cell or non-engineered cell, such as non-engineered primary cell, that has not previously been introduced with the one or more modifications. In some embodiments, a genetic editing system is used to modify one or more target polynucleotide sequences that regulate (e.g. reduce or eliminate) the expression of either one or more MHC class I molecules, one or more MHC class II molecules, or one or more MHC class I molecules and one or more MHC class II molecules. In certain embodiments, the genome of the cell has been altered to reduce or delete components required or involved in facilitating HLA expression, such as expression of one or more MHC class I molecules and/or one or more MHC class II molecules on the surface of the cell. For instance, in some embodiments, expression of a beta-2-microgloublin (B2M), a component of one or more MHC class I molecules, is reduced or eliminated in the cell, thereby reducing or elimination the protein expression (e.g. cell surface expression) of one or more MHC class I molecules by the engineered cell. Thus, in some embodiments, expression can be reduced via a gene, and/or function thereof, RNA expression and function, protein expression and function, localization (such as cell surface expression), and longevity.

In some embodiments, an MHC in humans is also called a human leukocyte antigen (HLA). For instance, a human MHC class I is also known as an HLA class I and a human MHC class II is also known as an HLA class II. Thus, reference to MHC is intended to include the corresponding human HLA molecules, unless stated otherwise.

In some embodiments, reduced expression of a target is such that expression in an engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a corresponding level of expression (e.g., protein expression compared with protein expression) of the target in a source cell prior to being engineered to reduce expression of the target. In some embodiments, reduced expression of a target is such that expression in an engineered cell is reduced to a level that is about 60% or less (such as any of about 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a corresponding level of expression (e.g., protein expression compared with protein expression) of the target in a reference cell or a reference cell population (such as a cell or population of the same cell type or a cell having reduced or eliminated immunogenic response). In some embodiments, reduced expression of a target is such that expression in an engineered cell is reduced to a level that is at or less than a measured level of expression (such as a level known to exhibit reduced or eliminated immunogenic response due to the presence of the target). In some embodiments, the level of a target is assessed in an engineered cell, a reference cell, or reference cell population in a stimulated or non-stimulated state. In some embodiments, the level of a target is assessed in an engineered cell, a reference cell, or reference cell population in a stimulated state such that the target is expressed (or will be if it is a capability of the cell in response to the stimulus). In some embodiments, the stimulus represents an in vivo stimulus.

In some embodiments, the provided engineered cells comprises a modification, such as a genetic modification, of one or more target polynucleotide sequences (also interchangeably referred to as a target gene) that regulate (e.g., reduce or eliminate) the expression of either one or more MHC class I molecules, one or more MHC class II molecules, or one or more MHC class I molecules and one or more MHC class II molecules. In some embodiments, an MHC in humans is also called a human leukocyte antigen. For instance, a human MHC class I molecule is also known as an HLA class I molecule and a human MHC class II molecules is also known as an HLA class II molecule. In some embodiments, the cell to be modified or engineered is an unmodified cell or non-engineered cell that has not previously been introduced with the one or more modifications. In some embodiments, a genetic editing system is used to modify one or more target polynucleotide sequences that regulate the expression of either one or more MHC class I molecules, one or more MHC class II molecules, or one or more MHC class I molecules and one or more MHC class II molecules. In certain embodiments, the genome of the cell has been altered to reduce or delete components require or involved in facilitating HLA expression, such as expression of one or more MHC class I molecules and/or one or more MHC class II molecules on the surface of the cell. For instance, in some embodiments, expression of a beta-2-microglobulin (B2M), a component of one or more MHC class I molecules, is reduced or eliminated in the cell, thereby reducing or elimination the protein expression (e.g. cell surface expression) of one or more MHC class I molecules by the engineered cell.

In some embodiments, any of the described modifications in the engineered cell that regulate (e.g. reduce or eliminate) expression of one or more target polynucleotide or protein in the engineered cell may be combined together with one or more modifications to overexpress a polynucleotide (e.g. tolerogenic factor, such as CD47) described in Section I.B.

In some embodiments, reduction of one or more MHC class I molecules and/or one or more MHC class II molecules expression can be accomplished, for example, by one or more of the following: (1) targeting the polymorphic HLA alleles (HLA-A, HLA-B, HLA-C) and one or more MHC class II molecules genes directly; (2) removal of B2M, which will reduce surface trafficking of all MHC class I molecules; and/or (3) deletion of one or more components of the MHC enhanceosomes, such as LRC5, RFX-5, RFXANK, RFXAP, IRF1, NF-Y (including NFY-A, NFY-B, NFY-C), and CIITA that are critical for HLA expression.

In certain embodiments, HLA expression is interfered with. In some embodiments, HLA expression is interfered with by targeting individual HLAs (e.g., knocking out expression of HLA-A, HLA-B and/or HLA-C), targeting transcriptional regulators of HLA expression (e.g., knocking out expression of NLRC5, CIITA, RFX5, RFXAP, RFXANK, NFY-A, NFY-B, NFY-C and/or IRF-1), blocking surface trafficking of one or more MHC class I molecules (e.g., knocking out expression of B2M and/or TAP1), and/or targeting with HLA-Razor (see, e.g., WO2016183041).

The human leukocytes antigen (HLA) complex is synonymous with human MHC. In some embodiments, the engineered cells disclosed herein are human cells. In certain aspects, the engineered cells disclosed herein do not express one or more human leukocyte antigens (e.g., HLA-A, HLA-B and/or HLA-C) corresponding to one or more MHC class I molecules and/or one or more MHC class II molecules and are thus characterized as being hypoimmunogenic. For example, in certain aspects, the engineered cells disclosed herein have been modified such that the cells do not express or exhibit reduced expression of one or more of the following MHC class I molecules: HLA-A, HLA-B and HLA-C. In some embodiments, one or more of HLA-A, HLA-B and HLA-C may be “knocked-out” of a cell. A cell that has a knocked-out HLA-A gene, HLA-B gene, and/or HLA-C gene may exhibit reduced or eliminated expression of each knocked-out gene.

In certain embodiments, the expression of one or more MHC class I molecules and/or one or more MHC class II molecules is modulated by targeting and deleting a contiguous stretch of genomic DNA, thereby reducing or eliminating expression of a target gene selected from the group consisting of B2M, CIITA, and NLRC5.

In some embodiments, the provided engineered cells comprise a modification, such as a genetic modification, of one or more target polynucleotide sequence that regulate one or more MHC class I. Exemplary methods for reducing expression of one or more MHC class I molecules are described in sections below. In some embodiments, the targeted polynucleotide sequence is one or both of B2M and NLRC5. In some embodiments, the cell comprises a genetic editing modification to the B2M gene. In some embodiments, the cell comprises a genetic editing modification to the NLRC5 gene. In some embodiments, the cell comprises genetic editing modifications to the B2M and CIITA genes.

In some embodiments, the provided engineered cells comprise a modification, such as a genetic modification, of one or more target polynucleotide sequence that regulate one or more MHC class II molecules. Exemplary methods for reducing expression of one or more MHC class II molecules are described in sections below. In some embodiments, the cell comprises a genetic editing modification to the CIITA gene.

In some embodiments, the provided engineered cells comprise a modification, such as a genetic modification, of one or more target polynucleotide sequence that regulate one or more MHC class I molecules and one or more MHC class II molecules. Exemplary methods for reducing expression of one or more MHC class I molecules and one or more MHC class II molecules are described in sections below. In some embodiments, the cell comprises genetic editing modifications to the B2M and NLRC5 genes. In some embodiments, the cell comprises genetic editing modifications to the CIITA and NLRC5 genes. In particular embodiments, the cell comprises genetic editing modifications to the B2M, CIITA and NLRC5 genes.

In some embodiments, the modification that reduces B2M, CIITA and/or NLRC5 expression reduces B2M, CIITA and/or NLRC5 mRNA expression. In some embodiments, the reduced mRNA expression of B2M, CIITA and/or NLRC5 is relative to an unmodified or wild-type cell of the same cell type that does not comprise the modification. In some embodiments, the mRNA expression of B2M is reduced by more than about 5%, such as reduced by more than about any of 10%, 20%, 30%, 40%, 50%, 60%, 70%, 80%, 90%, or more. In some embodiments, the mRNA expression of B2M, CIITA and/or NLRC5 is reduced by up to about 100%, such as reduced by up to about any of 90%, 80%, 70%, 60%, 50%, 40%, 30%, 20%, 10%, 5%, or less. In some embodiments, the mRNA expression of B2M, CIITA and/or NLRC5 is reduced by any of about 5%, 10%, 20%, 30%, 40%, 50%, 60%, 70%, 80%, 90%, or 100%. In some embodiments, the mRNA expression of B2M, CIITA and/or NLRC5 is eliminated (e.g., 0% expression of B2M, CIITA and/or NLRC5 mRNA). In some embodiments, the modification that reduces B2M, CIITA and/or NLRC5 mRNA expression eliminates B2M, CIITA and/or NLRC5 gene activity.

In some embodiments, the modification that reduces B2M, CIITA and/or NLRC5 expression reduces B2M, CIITA and/or NLRC5 protein expression. In some embodiments, the reduced protein expression of B2M, CIITA and/or NLRC5 is relative to an unmodified or wild-type cell of the same cell type that does not comprise the modification. In some embodiments, the protein expression of B2M, CIITA and/or NLRC5 is reduced by more than about 5%, such as reduced by more than about any of 10%, 20%, 30%, 40%, 50%, 60%, 70%, 80%, 90%, or more. In some embodiments, the protein expression of B2M, CIITA and/or NLRC5 is reduced by up to about 100%, such as reduced by up to about any of 90%, 80%, 70%, 60%, 50%, 40%, 30%, 20%, 10%, 5%, or less. In some embodiments, the protein expression of B2M, CIITA and/or NLRC5 is reduced by any of about 5%, 10%, 20%, 30%, 40%, 50%, 60%, 70%, 80%, 90%, or 100%. In some embodiments, the protein expression of B2M, CIITA and/or NLRC5 is eliminated (e.g., 0% expression of B2M, CIITA and/or NLRC5 protein). In some embodiments, the modification that reduces B2M, CIITA and/or NLRC5 protein expression eliminates B2M, CIITA and/or NLRC5 gene activity.

In some embodiments, the modification that reduces B2M, CIITA and/or NLRC5 expression comprises inactivation or disruption of the B2M, CIITA and/or NLRC5 gene. In some embodiments, the modification that reduces B2M, CIITA and/or NLRC5 expression comprises inactivation or disruption of one allele of the B2M, CIITA and/or NLRC5 gene. In some embodiments, the modification that reduces B2M, CIITA and/or NLRC5 expression comprises inactivation or disruption comprises inactivation or disruption of both alleles of the B2M, CIITA and/or NLRC5 gene.

In some embodiments, the modification comprises inactivation or disruption of one or more B2M, CIITA and/or NLRC5 coding sequences in the cell. In some embodiments, the modification comprises inactivation or disruption of all B2M, CIITA and/or NLRC5 coding sequences in the cell. In some embodiments, the modification comprises inactivation or disruption comprises an indel in the B2M, CIITA and/or NLRC5 gene. In some embodiments, the modification is a frameshift mutation of genomic DNA of the B2M, CIITA and/or NLRC5 gene. In some embodiments, the modification is a deletion of genomic DNA of the B2M, CIITA and/or NLRC5 gene. In some embodiments, the modification is a deletion of a contiguous stretch of genomic DNA of the B2M, CIITA and/or NLRC5 gene. In some embodiments, the B2M, CIITA and/or NLRC5 gene is knocked out.

In some embodiments, the engineered cell comprises reduced expression of one or more MHC class I, or a component thereof, wherein reduced is as described herein, such as relative to prior to engineering to reduce expression of one or more MHC class I molecules or a component thereof, a reference cell or a reference cell population (such as a cell having a desired lack of an immunogenic response), or a measured value. In some embodiments, the engineered cell is engineered to reduce cell surface expression of the one or more MHC class I polypeptides, or a component thereof (such as B2M). In some embodiments, cell surface expression of the one or more MHC class I polypeptides, or a component thereof (such as B2M), on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class I polypeptides, or a component thereof (such as B2M), cell surface expression prior to being engineered to reduce cell surface presentation of the one or more MHC class I polypeptides, or a component thereof (such as B2M). In some embodiments, cell surface expression of the one or more MHC class I polypeptides, or a component thereof (such as B2M), on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class I polypeptides, or a component thereof (such as B2M), cell surface expression on a reference cell or a reference cell population (such as an average amount of one or more MHC class I polypeptides, or a component thereof (such as B2M), cell surface expression). In some embodiments, there is no cell surface presentation of the one or more MHC class I polypeptides, or a component thereof (such as B2M), on the engineered cell (including no detectable cell surface expression, including as measured using known techniques, e.g., flow cytometry). In some embodiments, the engineered cell exhibits reduced protein expression of the one or more MHC class I polypeptides, or a component thereof (such as B2M). In some embodiments, protein expression of the one or more MHC class I polypeptides, or a component thereof (such as B2M), of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class I polypeptides, or a component thereof (such as B2M), protein expression prior to being engineered to reduce protein expression of the one or more MHC class I polypeptides, or a component thereof (such as B2M). In some embodiments, protein expression of the one or more MHC class I polypeptides, or a component thereof (such as B2M), of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class I polypeptides, or a component thereof (such as B2M), prior to being engineered to reduce protein expression of the one or more MHC class I polypeptides, or a component thereof (such as B2M). In some embodiments, the engineered cell exhibits no protein expression of the one or more MHC class I polypeptides, or a component thereof (such as B2M), (including no detectable protein expression, including as measured using known techniques, e.g., western blot or mass spectrometry). In some embodiments, the engineered cell does not comprise the one or more MHC class I polypeptides, or a component thereof (such as B2M) (including no detectable protein, including as measured using known techniques, e.g., western blot or mass spectrometry). In some embodiments, the engineered cell exhibits reduced mRNA expression encoding the one or more MHC class I polypeptides, or a component thereof (such as B2M). In some embodiments, mRNA expression encoding the one or more MHC class I polypeptides, or a component thereof (such as B2M), of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression encoding the one or more MHC class I polypeptides, or a component thereof (such as B2M), prior to being engineered to reduce mRNA expression of the one or more MHC polypeptides, or a component thereof (such as B2M). In some embodiments, mRNA expression encoding the one or more MHC class I polypeptides, or a component thereof (such as B2M), of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression of a reference cell or a reference cell population. In some embodiments, the engineered cell does not express mRNA encoding one or more MHC class I polypeptides, or a component thereof (including no detectable mRNA expression, including as measured using known techniques, e.g., sequencing techniques or PCR). In some embodiments, the engineered cell does not comprise mRNA encoding one or more MHC class I polypeptides, or a component thereof (including no detectable mRNA, including as measured using known techniques, e.g., sequencing techniques or PCR). In some embodiments, the engineered cell comprises a gene inactivation or disruption of the one or more MHC class I molecules gene. In some embodiments, the engineered cell comprises a gene inactivation or disruption of the one or more MHC class I molecules gene in both alleles. In some embodiments, the engineered cell comprises a gene inactivation or disruption of the one or more MHC class I molecules gene in all alleles. In some embodiments, the engineered cell is a one or more MHC class I molecules knockout or a one or more MHC class I molecules component (such as B2M) knockout.

In some embodiments, the engineered cell comprises reduced expression of one or more MHC class II molecules, wherein reduced is as described herein, such as relative to prior to engineering to reduce one or more MHC class II molecules expression, a reference cell or a reference cell population (such as a cell having a desired lack of an immunogenic response), or a measured value. In some embodiments, the engineered cell is engineered to reduced cell surface expression of the one or more MHC class II polypeptides. In some embodiments, cell surface expression of the one or more MHC class II polypeptides on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class II polypeptides cell surface expression prior to being engineered to reduce cell surface presentation of the one or more MHC class II polypeptides. In some embodiments, cell surface expression of the one or more MHC class II polypeptides on the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class II polypeptides cell surface expression on a reference cell or a reference cell population (such as an average amount of one or more MHC class II polypeptides cell surface expression). In some embodiments, there is no cell surface presentation of the one or more MHC class II polypeptides on the engineered cell (including no detectable cell surface expression, including as measured using known techniques, e.g., flow cytometry). In some embodiments, the engineered cell exhibits reduced protein expression of the one or more MHC class II polypeptides. In some embodiments, protein expression of the one or more MHC class II polypeptides of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class II polypeptides protein expression prior to being engineered to reduce protein expression of the one or more MHC class II polypeptides. In some embodiments, protein expression of the MHC class II polypeptides of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of the one or more MHC class II polypeptides prior to being engineered to reduce protein expression of the one or more MHC class II polypeptides. In some embodiments, the engineered cell exhibits no protein expression of the one or more MHC class II polypeptides (including no detectable protein expression, including as measured using known techniques, e.g., western blot or mass spectrometry). In some embodiments, the engineered cell does not comprise the one or more MHC class II polypeptides (including no detectable protein, including as measured using known techniques, e.g., western blot or mass spectrometry). In some embodiments, the engineered cell exhibits reduced mRNA expression encoding the one or more MHC class II polypeptides. In some embodiments, mRNA expression encoding the one or more MHC class II polypeptides of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression encoding the one or more MHC class II polypeptides prior to being engineered to reduce mRNA expression of the one or more MHC class II polypeptides. In some embodiments, mRNA expression encoding the one or more MHC class II polypeptides of the engineered cell is reduced to a level that is about 60% or less (such as about any of 55% or less, 50% or less, 45% or less, 40% or less, 35% or less, 30% or less, 25% or less, 20% or less, 15% or less, 10% or less, 5% or less, 4% or less, 3% or less, 2% or less, or 1% or less) than a level of mRNA expression of a reference cell or a reference cell population. In some embodiments, the engineered cell does not express mRNA encoding one or more MHC class II polypeptides (including no detectable mRNA expression, including as measured using known techniques, e.g., sequencing techniques or PCR). In some embodiments, the engineered cell does not comprise mRNA encoding one or more MHC class II polypeptides (including no detectable mRNA, including as measured using known techniques, e.g., sequencing techniques or PCR). In some embodiments, the engineered cell comprises a gene inactivation or disruption of the one or more MHC class 1 molecules gene. In some embodiments, the engineered cell comprises a gene inactivation or disruption of the one or more MHC class 11 molecules gene in both alleles. In some embodiments, the engineered cell comprises a gene inactivation or disruption of the one or more MHC class 1 molecules in all alleles. In some embodiments, the engineered cell is a one or more MHC class II molecules knockout.

1. Methods of Reducing Expression

In some embodiments, the cells provided herein are modified, such as genetically modified, to reduce expression of the one or more target polynucleotides as described. In some embodiments, the cell that is engineered with the one or more modifications to reduce (e.g. eliminate) expression of a polynucleotide or protein is any source cell as described herein. In some embodiments, the source cell is any cell described herein. In certain embodiments, the cells (e.g., primary cells) disclosed herein comprise one or more modifications, such as genetic modifications, to reduce expression of one or more target polynucleotides. Non-limiting examples of the one or more target polynucleotides include any as described above, such as one or more of MHC class I molecules, or a component thereof, one or more MHC class II molecules, CIITA, B2M, NLRC5, HLA-A, HLA-B, HLA-C, LRC5, RFX-ANK, RFX5, RFX-AP, NFY-A, NFY-B, NFY-C, IRF1, and TAP1. In some embodiments, the one or more modifications, such as genetic modifications, to reduce expression of the one or more target polynucleotides is combined with one or more modifications to increase expression of a desired transgene, such as any described herein. In some embodiments, the one or more modifications, such as genetic modifications, create engineered cells that are immune-privileged or hypoimmunogenic cells. By modulating (e.g., reducing or deleting) expression of one or a plurality of the target polynucleotides, such cells exhibit decreased immune activation when engrafted into a recipient subject. In some embodiments, the cell is considered hypoimmunogenic, e.g., in a recipient subject or patient upon administration.

Any method for reducing expression of a target polynucleotide may be used. In some embodiments, the modifications (e.g., genetic modifications) result in permanent elimination or reduction in expression of the target polynucleotide. For instance, in some embodiments, the target polynucleotide or gene is disrupted by introducing a DNA break in the target polynucleotide, such as by using a targeting endonuclease. In other embodiments, the modifications (e.g., genetic modifications) result in transient reduction in expression of the target polynucleotide. For instance, in some embodiments gene repression is achieved using an inhibitory nucleic acid that is complementary to the target polynucleotide to selectively suppress or repress expression of the gene, for instance using antisense techniques, such as by RNA interference (RNAi), short interfering RNA (siRNA), short hairpin (shRNA), and/or ribozymes.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, any of gene editing technologies can be used to reduce expression of the one or more target polynucleotides or target proteins as described. In some embodiments, the gene editing technology can include systems involving nucleases, integrases, transposases, recombinases. In some embodiments, the gene editing technologies can be used for knock-out or knock-down of genes. In some embodiments, the gene-editing technologies can be used for knock-in or integration of DNA into a region of the genome. In some embodiments, the gene editing technology mediates single-strand breaks (SSB). In some embodiments, the gene editing technology mediates double-strand breaks (DSB), including in connection with non-homologous end-joining (NHEJ) or homology-directed repair (HDR). In some embodiments, the gene editing technology can include DNA-based editing or prime-editing. In some embodiments, the gene editing technology can include Programmable Addition via Site-specific Targeting Elements (PASTE).

In some embodiments, gene disruption is carried out by induction of one or more double-stranded breaks and/or one or more single-stranded breaks in the gene, typically in a targeted manner. In some embodiments, the double-stranded or single-stranded breaks are made by a nuclease, e.g., an endonuclease, such as a gene-targeted nuclease. In some embodiments, the targeted nuclease is selected from zinc finger nucleases (ZFN), transcription activator-like effector nucleases (TALENs), and RNA-guided nucleases such as a CRISPR-associated nuclease (Cas), specifically designed to be targeted to the sequence of a gene or a portion thereof. In some embodiments, the targeted nuclease generates double-stranded or single-stranded breaks that then undergo repair through error prone non-homologous end joining (NHEJ) or, in some cases, precise homology directed repair (HDR) in which a template is used. In some embodiments, the targeted nuclease generates DNA double strand breaks (DSBs). In some embodiments, the process of producing and repairing the breaks is typically error prone and results in insertions and deletions (indels) of DNA bases from NHEJ repair. In some embodiments, the genetic modification may induce a deletion, insertion or mutation of the nucleotide sequence of the target gene. In some cases, the genetic modification may result in a frameshift mutation, which can result in a premature stop codon. In examples of nuclease-mediated gene editing the targeted edits occur on both alleles of the gene resulting in a biallelic disruption or edit of the gene. In some embodiments, all alleles of the gene are targeted by the gene editing. In some embodiments, genetic modification with a targeted nuclease, such as using a CRISPR/Cas system, leads to complete knockout of the gene.

In some embodiments, the nuclease, such as a rare-cutting endonuclease, is introduced into a cell containing the target polynucleotide sequence. The nuclease may be introduced into the cell in the form of a nucleic acid encoding the nuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid that is introduced into the cell is DNA. In some embodiments, the nuclease is introduced into the cell in the form of a protein. For instance, in the case of a CRISPR/Cas system a ribonucleoprotein (RNP) may be introduced into the cell.

In some embodiments, the modification (e.g., genetic modification) occurs using a CRISPR/Cas system. Any CRISPR/Cas system that is capable of altering a target polynucleotide sequence in a cell can be used. Such CRISPR-Cas systems can employ a variety of Cas proteins (Haft et al. PLoS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The CRISPR/Cas systems includes targeted systems that can be used to alter any target polynucleotide sequence in a cell. In some embodiments, a CRISPR/Cas system provided herein includes a Cas protein and one or more, such as at least one to two, ribonucleic acids (e.g., guide RNA (gRNA)) that are capable of directing the Cas protein to and hybridizing to a target motif of a target polynucleotide sequence.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein. Exemplary Cas core proteins include, but are not limited to, Cas1, Cas2, Cas3, Cas4, Cas5, Cas6, Cas7, Cas8, Cas9, Cas12a, and Cas13. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019).

In some embodiments, the methods for genetically modifying cells to knock out, knock down, or otherwise modify one or more genes comprise using a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Nal. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39:e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29(18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29(18):3757-3774. Members of the NHN family are defined by motifs containing two pairs of conserved histidines surrounded by asparagine residues. See Chevalier et al., Nucleic Acids Res. (2001) 29(18):3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363(2):283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

Transposases are enzymes that bind to the end of a transposon and catalyze its movement to another part of the genome by a cut and paste mechanism or a replicative transposition mechanism. By linking transposases to other systems such as the CRISPER/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. The most widely used Cas9 is a type II Cas protein and is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA:tracrRNA complex. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 1a below.

TABLE 1a
Exemplary Cas nuclease variants and their PAM
sequences

| CRISPR | | PAM Sequence |
|---|---|---|
| Nuclease | Source Organism | (5′→3′) |
| SpCas9 | Streptococcus pyogenes | NGG or NAG |
| SaCas9 | Staphylococcus aureus | NGRRT or |
| | | NGRRN |
| NmeCas9 | Neisseria meningitidis | NNNNGATT |
| CjCas9 | Campylobacter jejuni | NNNNRYAC |
| StCas9 | Streptococcus thermophilus | NNAGAAW |
| TdCas9 | Treponema denticola | NAAAAC |
| LbCas12a | Lachnospiraceae bacterium | TTTV |
| (Cpf1) | | |
| AsCas12a | Acidaminococcus sp. | TTTV |
| (Cpf1) | | |
| AacCas | Alicyclobacillus | TTN |
| 12b | acidiphilus | |
| BhCas 12b | Bacillus hisashii | ATTN, TTTN, |
| v4 | | or GTTN |

R = A or G; Y = C or T; W = A or T; V = A or C or G; N = any base

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example the Cas nuclease may have one or more mutations that alter its PAM specificity.

In some embodiments, a Cas protein comprises any one of the Cas proteins described herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas12a protein comprises a functional portion of a RuvC-like domain.

In some embodiments, suitable Cas proteins include, but are not limited to, Cas0, Cas12a (i.e. Cpf1), Cas12b, Cas12i, CasX, and Mad7.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5(8):747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetratin domain. In some embodiments, the Cas9 protein comprises a Cas9 polypeptide fused to a superpositively charged GFP. In some embodiments, the Cas12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas12a polypeptide fused to a tat domain. In some embodiments, the Cas12a protein comprises a Cas12a polypeptide fused to an oligoarginine domain. In some embodiments, the Cas12a protein comprises a Cas12a polypeptide fused to a penetratin domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In some embodiments, the Cas protein is complexed with one to two ribonucleic acids (e.g., guide RNA (gRNA)). In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

In provided embodiments, a CRISPR/Cas system generally includes two components: one or more guide RNA (gRNA) and a Cas protein. In some embodiments, the Cas protein is complexed with the one or more, such as one to two, ribonucleic acids (e.g., guide RNA (gRNA)). In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

In some embodiments, gRNAs are short synthetic RNAs composed of a scaffold sequence for Cas binding and a user-designed spacer or complementary portion designated crRNA. The cRNA is composed of a crRNA targeting sequence (herein after also called a gRNA targeting sequence; usually about 20 nucleotides in length) that defines the genomic target to be modified and a region of crRNA repeat (e.g. GUUUUAGAGCUA; SEQ ID NO: 23). One can change the genomic target of the Cas protein by simply changing the complementary portion sequence (e.g. gRNA targeting sequence) present in the gRNA. In some embodiments the scaffold sequence for Cas binding is made up of a tracrRNA sequence (e.g. UAGCAAGUUAAAAUAAGGCUAGUCCGUUAUCAACUUGAAAAAGUGGCACCGAGUCG GUGCUUU; SEQ ID NO: 24) that hybridizes to the crRNA through its anti-repeat sequence. The complex between crRNA:tracrRNA recruits the Cas nuclease (e.g. Cas9) and cleaves upstream of a protospacer-adjacent motif (PAM). In order for the Cas protein to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease, derived from S. pyogenes, recognizes a PAM sequence of NGG. Other Cas9 variants and other nucleases with alternative PAMs have also been characterized and successfully used for genome editing. Thus, the CRISPR/Cas system can be used to create targeted DSBs at specified genomic loci that are complementary to the gRNA designed for the target loci. The crRNA and tracrRNA can be linked together with a loop sequence (e.g. a tetraloop; GAAA, SEQ ID NO: 25) for generation of a gRNA that is a chimeric single guide RNA (sgRNA; Hsu et al. 2013). sgRNA can be generated for DNA-based expression or by chemical synthesis.

In some embodiments, the complementary portion sequences (e.g. gRNA targeting sequence) of the gRNA will vary depending on the target site of interest. In some embodiments, the gRNAs comprise complementary portions specific to a sequence of a gene set forth in Table 1b. In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

The methods disclosed herein contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids provided herein can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs. In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

Exemplary gRNA targeting sequences useful for CRISPR/Cas-based targeting of genes described herein are provided in Table 1b. The sequences can be found in WO2016183041 filed May 9, 2016, the disclosure including the Tables, Appendices, and Sequence Listing is incorporated herein by reference in its entirety.

TABLE 1b
Exemplary gRNA targeting sequences useful for targeting genes

| Gene Name | SEQ ID NO: | WO2016183041 |
|---|---|---|
| HLA-A | SEQ ID NOs: 2-1418 | Table 8, Appendix 1 |
| HLA-B | SEQ ID NOs: 1419-3277 | Table 9, Appendix 2 |
| HLA-C | SEQ ID NOS: 3278-5183 | Table 10, Appendix 3 |
| RFX-ANK | SEQ ID NOs: 95636-102318 | Table 11, Appendix 4 |
| NFY-A | SEQ ID NOs: 102319-121796 | Table 13, Appendix 6 |
| RFX5 | SEQ ID NOs: 85645-90115 | Table 16, Appendix 9 |
| RFX-AP | SEQ ID NOs: 90116-95635 | Table 17, Appendix 10 |
| NFY-B | SEQ ID NOs: 121797-135112 | Table 20, Appendix 13 |
| NFY-C | SEQ ID NOs: 135113-176601 | Table 22, Appendix 15 |
| IRF1 | SEQ ID NOs: 176602-182813 | Table 23, Appendix 16 |
| TAP1 | SEQ ID NOs: 182814-188371 | Table 24, Appendix 17 |
| CIITA | SEQ ID NOS: 5184-36352 | Table 12, Appendix 5 |
| B2M | SEQ ID NOS: 81240-85644 | Table 15, Appendix 8 |
| NLRC5 | SEQ ID NOS: 36353-81239 | Table 14, Appendix 7 |
| CD47 | SEQ ID NOS: 200784-231885 | Table 29, Appendix 22 |
| HLA-E | SEQ ID NOS: 189859-193183 | Table 19, Appendix 12 |
| HLA-F | SEQ ID NOS: 688808-699754 | Table 45, Appendix 38 |
| HLA-G | SEQ ID NOS: 188372-189858 | Table 18, Appendix 11 |
| PD-L1 | SEQ ID NOS: 193184-200783 | Table 21, Appendix 14 |

In some embodiments, it is within the level of a skilled artisan to identify new loci and/or gRNA targeting sequences for use in methods of genetic disruption to reduce or eliminate expression of a gene as described. For example, for CRISPR/Cas systems, when an existing gRNA targeting sequence for a particular locus (e.g., within a target gene, e.g. set forth in Table 1b) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in genetic disruption methods. Although the CRISPR/Cas system is described as illustrative, any gene-editing approaches as described can be used in this method of identifying new loci, including those using ZFNs, TALENS, meganucleases and transposases.

Additional exemplary Cas9 guide RNA sequences useful for CRISPR/Cas-based targeting of genes described herein are provided in Table 2.

TABLE 2
Additional exemplary Cas9 guide RNA sequences
useful for targeting genes

| | | | Target | gRNA cut | SEQ |
|---|---|---|---|---|---|
| Gene | guide sequence | PAM | site | location | ID NO |
| ABO | UCUCUCCAUGUGCAGUAGGA | AGG | Exon 7 | chr9:133,257,541 | 29 |
| FUT1 | CUGGAUGUCGGAGGAGUACG | CGG | Exon 4 | chr19:48,750,822 | 30 |
| RHD | GUCUCCGGAAACUCGAGGUG | AGG | Exon 2 | chr1:25,284,622 | 31 |
| F3 | ACAGUGUAGACUUGAUUGAC | GGG | Exon 2 | chr1:94,540,281 | 32 |
| (CD142) | | | | | |
| B2M | CGUGAGUAAACCUGAAUCUU | TGG | Exon 2 | chr15:44,715,434 | 33 |
| CIITA | GAUAUUGGCAUAAGCCUCCC | TGG | Exon 3 | chr16:10,895,747 | 34 |
| TRAC | AGAGUCUCUCAGCUGGUACA | CGG | Exon 1 | chr14:22,5547,533 | 35 |

In some embodiments, the cells described herein are made using Transcription Activator-Like Effector Nucleases (TALEN) methodologies. By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In a particular embodiment, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues coordinated with zinc along with the two cysteine residues of a single beta turn (see, e.g., Berg & Shi, Science 271:1081-1085 (1996)).

In some embodiments, the cells described herein are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease may for example correspond to a LAGLIDADG endonuclease, to an HNH endonuclease, or to a GIY-YIG endonuclease. In some embodiments, the homing endonuclease can be an I-CreI variant.

In some embodiments, the cells described herein are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

In some embodiments, the gene editing technology is associated with base editing. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some cases, base editors may also include proteins or domains that alter cellular DNA repair processes to increase the efficiency and/or stability of the resulting single-nucleotide change.

In some aspects, currently available base editors include cytidine base editors (e.g., BE4) that convert target C-G to T-A and adenine base editors (e.g., ABE7.10) that convert target A-T to G-C. In some aspects, Cas9-targeted deamination was first demonstrated in connection with a Base Editor (BE) system designed to induce base changes without introducing double-strand DNA breaks. Further Rat deaminase APOBEC1 (rAPOBEC1) fused to deactivated Cas9 (dCas9) was used to successfully convert cytidines to thymidines upstream of the PAM of the sgRNA. In some aspects, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T:A during DNA replication.

In some embodiments, the base editor is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, the base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, the base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, the base editor is a adenine-to-thymine or “ATBE” (or thymine-to-adenine or “TABE”) transversion base editors. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, the gene editing technology is target-primed reverse transcription (TPRT) or “prime editing”. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, the homologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, the prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, the gene editing technology is associated with a prime editor that is a reverse transcriptase, or any DNA polymerase known in the art. Thus, in one aspect, the prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety.

In some embodiments, the gene editing technology is Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allows for integration of sequences as large as ˜36 kb. In some embodiments, the serine integrase can be any known in the art. In some embodiments, the serine integrase has sufficient orthogonality such that PASTE can be used for multiplexed gene integration, simultaneously integrating at least two different genes at at least two genomic loci. In some embodiments, PASTE has editing efficiencies comparable to or better than those of homology directed repair or non-homologous end joining based integration, with activity in nondividing cells and fewer detectable off-target events.

In some embodiments, the cells provided herein are made using RNA silencing or RNA interference (RNAi) to knockdown (e.g., decrease, eliminate, or inhibit) the expression of a polypeptide. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available. For instance, a target polynucleotide, such as any described above, e.g. CIITA, B2M, or NLRC5, can be knocked down in a cell by RNA interference by introducing an inhibitory nucleic acid complementary to a target motif of the target polynucleotide, such as an siRNA, into the cells. In some embodiments, a target polynucleotide, such as any described above, e.g. CITA, B2M, or NLRC5, can be knocked down in a cell by transducing a shRNA-expressing virus into the cell. In some embodiments, RNA interference is employed to reduce or inhibit the expression of at least one selected from the group consisting of CIITA, B2M, and NLRC5.

In some embodiments, the modification, such as the genetic modification, reduces or eliminates, such as knocks out, the expression of one or more MHC class I molecules genes and/or one or more MHC class II molecule genes by targeting the accessory chain B2M. B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B and/or NFY-C and any combination thereof. In some embodiments, decreased or eliminated expression of one or more MHC class I molecules and/or one or more MHC class II molecules is a modification that reduces expression of, such as knockout, one or more of the following B2M. B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B and/or NFY-C.

2. Exemplary Target Polynucleotides and Methods for Reducing Expression

a. MHC Class I Molecules

In certain embodiments, the modification, such as the genetic modification, reduces or eliminates, such as knocks out, the expression of one or more MHC class I molecules genes by targeting the accessory chain B2M. In some embodiments, the genetic modification occurs using a CRISPR/Cas system. By reducing or eliminating, such as knocking out, expression of B2M, surface trafficking of one or more MHC class I molecules is blocked and such cells exhibit immune tolerance when engrafted into a recipient subject. In some embodiments, the cell is considered hypoimmunogenic, e.g., in a recipient subject or patient upon administration.

In some embodiments, the target polynucleotide sequence provided herein is a variant of B2M. In some embodiments, the target polynucleotide sequence is a homolog of B2M. In some embodiments, the target polynucleotide sequence is an ortholog of B2M.

In some embodiments, decreased or eliminated expression of one or more MHC class I molecules is a modification that reduces expression of one or more of the following MHC class I molecules—HLA-A, HLA-B, and HLA-C. In some embodiments, decreased or eliminated expression of B2M reduces or eliminates expression of one or more of the following MHC class I molecules—HLA-A, HLA-B, and HLA-C. In some embodiments, decreased or eliminated expression of B2M reduces or eliminates expression of an HLA-A protein. In some embodiments, decreased or eliminated expression of B2M reduces or eliminates expression of an HLA-B protein. In some embodiments, decreased or eliminated expression of B2M reduces or eliminates expression of an HLA-C protein. In some embodiments, decreased or eliminated expression of B2M reduces or eliminates expression of one or more of the following MHC class I molecules—HLA-A, HLA-B, and HLA-C, by knocking out a gene encoding said molecule. In some embodiments, the gene encoding an HLA-A protein is knocked out to reduce or eliminate expression of said HLA-A protein. In some embodiments, the gene encoding an HLA-B protein is knocked out to reduce or eliminate expression of said HLA-B protein. In some embodiments, the gene encoding an HLA-C protein is knocked out to reduce or eliminate expression of said HLA-C protein.

In some embodiments, the engineered cell, such as engineered primary cell, comprises a modification (e.g., genetic modification) targeting the B2M gene. In some embodiments, the modification (e.g., genetic modification) targeting the B2M gene is by using a targeted nuclease system that comprises a Cas protein or a polynucleotide encoding a Cas protein, and at least one guide ribonucleic acid sequence for specifically targeting the B2M gene. In some embodiments, the at least one guide ribonucleic acid sequence (e.g. gRNA targeting sequence) for specifically targeting the B2M gene is selected from the group consisting of SEQ ID NOS:81240-85644 of Appendix 2 or Table 15 of WO2016/183041, the disclosure is incorporated by reference in its entirety. In some embodiments, the gRNA targeting sequence for specifically targeting the B2M gene is CGUGAGUAAACCUGAAUCUU (SEQ ID NO: 33).

In some embodiments, an exogenous nucleic acid or transgene encoding a polypeptide as disclosed herein (e.g., a chimeric antigen receptor, CD47, or another tolerogenic factor disclosed herein) is inserted at the B2M gene. Exemplary transgenes for targeted insertion at the B2M locus include any as described herein.

Assays to test whether the B2M gene has been inactivated are known and described herein. In one embodiment, the resulting genetic modification of the B2M gene is assessed by PCR. In some embodiments, the reduction of one or more MHC class I, such as HLA-I, expression can be assays by flow cytometry, such as by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating modification, such as genetic modification. In some embodiments, the reduction in one or more MHC class I molecules expression is assessed using an immunoaffinity technique, such as immunohistochemistry or immunocytochemistry.

In some embodiments, the reduction of one or more MHC class I molecules expression or function (HLA I when the cells are derived from human cells) in the engineered cells can be measured using techniques known in the art; for example, FACS techniques using labeled antibodies that bind the HLA complex; for example, using commercially available HLA-A, B, C antibodies that bind to the alpha chain of the human major histocompatibility HLA Class I antigens. In addition, the cells can be tested to confirm that the HLA I complex is not expressed on the cell surface. This may be assayed by FACS analysis using antibodies to one or more HLA cell surface components as discussed above. In addition to the reduction of HLA I (or MHC class I), the engineered cells provided herein have a reduced susceptibility to macrophage phagocytosis and NK cell killing. Methods to assay for hypoimmunogenic phenotypes of the engineered cells are described further below.

In some embodiments, the modification (e.g., genetic modification) that reduces B2M expression reduces B2M mRNA expression. In some embodiments, the reduced mRNA expression of B2M is relative to an unmodified or wild-type cell of the same cell type that does not comprise the modification. In some embodiments, the mRNA expression of B2M is reduced by more than about 5%, such as reduced by more than about any of 10%, 20%, 30%, 40%, 50%, 60%, 70%, 80%, 90%, or more. In some embodiments, the mRNA expression of B2M is reduced by up to about 100%, such as reduced by up to about any of 90%, 80%, 70%, 60%, 50%, 40%, 30%, 20%, 10%, 5%, or less. In some embodiments, the mRNA expression of B2M is reduced by any of about 5%, 10%, 20%, 30%, 40%, 50%, 60%, 70%, 80%, 90%, or 100%. In some embodiments, the mRNA expression of B2M is eliminated (e.g., 0% expression of B2M mRNA). In some embodiments, the modification that reduces B2M mRNA expression eliminates B2M gene activity.

In some embodiments, the modification (e.g., genetic modification) that reduces B2M expression reduces B2M protein expression. In some embodiments, the reduced protein expression of B2M is relative to an unmodified or wild-type cell of the same cell type that does not comprise the modification. In some embodiments, the protein expression of B2M is reduced by more than about 5%, such as reduced by more than about any of 10%, 20%, 30%, 40%, 50%, 60%, 70%, 80%, 90%, or more. In some embodiments, the protein expression of B2M is reduced by up to about 100%, such as reduced by up to about any of 90%, 80%, 70%, 60%, 50%, 40%, 30%, 20%, 10%, 5%, or less. In some embodiments, the protein expression of B2M is reduced by any of about 5%, 10%, 20%, 30%, 40%, 50%, 60%, 70%, 80%, 90%, or 100%. In some embodiments, the protein expression of B2M is eliminated (e.g., 0% expression of B2M protein). In some embodiments, the modification that reduces B2M protein expression eliminates B2M gene activity.

In some embodiments, the modification (e.g., genetic modification) that reduces B2M expression comprises inactivation or disruption of the B2M gene. In some embodiments, the modification that reduces B2M expression comprises inactivation or disruption of one allele of the B2M gene. In some embodiments, the modification that reduces B2M expression comprises inactivation or disruption comprises inactivation or disruption of both alleles of the B2M gene.

In some embodiments, the modification (e.g., genetic modification) comprises inactivation or disruption of one or more B2M coding sequences in the cell. In some embodiments, the modification comprises inactivation or disruption of all B2M coding sequences in the cell. In some embodiments, the modification comprises inactivation or disruption comprises an indel in the B2M gene. In some embodiments, the modification is a frameshift mutation of genomic DNA of the B2M gene. In some embodiments, the modification is a deletion of genomic DNA of the B2M gene. In some embodiments, the modification is a deletion of a contiguous stretch of genomic DNA of the B2M gene. In some embodiments, the B2M gene is knocked out.

b. MHC Class H Molecules

In certain aspects, the modification, such as genetic modification, reduces or eliminates, such as knocks out, the expression of one or more MHC class II molecules genes by targeting Class II molecules transactivator (CIITA) expression. In some embodiments, the genetic modification occurs using a CRISPR/Cas system. CIITA is a member of the LR or nucleotide binding domain (NBD) leucine-rich repeat (LRR) family of proteins and regulates the transcription of one or more MHC class II molecules by associating with the MHC enhanceosome. By reducing or eliminating, such as knocking out, expression of CIITA, expression of one or more MHC class II molecules is reduced thereby also reducing surface expression. In some cases, such cells exhibit immune tolerance when engrafted into a recipient subject. In some embodiments, the cell is considered hypoimmunogenic, e.g., in a recipient subject or patient upon administration.

In some embodiments, the target polynucleotide sequence is a variant of CIITA. In some embodiments, the target polynucleotide sequence is a homolog of CITA. In some embodiments, the target polynucleotide sequence is an ortholog of CIITA.

In some embodiments, decreased or eliminated expression of one or more MHC class II molecules is a modification that reduces expression of one or more of the following MHC class II molecules—HLA-DP, HLA-DM, HLA-DOA, HLA-DOB, HLA-DQ, and HLA-DR. In some embodiments, reduced or eliminated expression of CIITA reduces or eliminates expression of one or more of the following MHC class II molecules—HLA-DP, HLA-DM, HLA-DOA, HLA-DOB, HLA-DQ, and HLA-DR. In some embodiments, decreased or eliminated expression of CIITA reduces or eliminates expression of an HLA-DP protein. In some embodiments, decreased or eliminated expression of CITA reduces or eliminates expression of an HLA-DM protein. In some embodiments, decreased or eliminated expression of CITA reduces or eliminates expression of an HLA-DOA protein. In some embodiments, decreased or eliminated expression of CIITA reduces or eliminates expression of an HLA-DOB protein. In some embodiments, decreased or eliminated expression of CIITA reduces or eliminates expression of an HLA-DQ protein. In some embodiments, decreased or eliminated expression of CITA reduces or eliminates expression of an HLA-DR protein. In some embodiments, decreased or eliminated expression of CITA reduces or eliminates expression of one or more of the following MHC class II molecules—HLA-DP, HLA-DM, HLA-DOA, HLA-DOB, HLA-DQ, and HLA-DR, by knocking out a gene encoding said molecule. In some embodiments, the gene encoding an HLA-DP protein is knocked out to reduce or eliminate expression of said HLA-DP protein. In some embodiments, the gene encoding an HLA-DM protein is knocked out to reduce or eliminate expression of said HLA-DM protein. In some embodiments, the gene encoding an HLA-DOA protein is knocked out to reduce or eliminate expression of said HLA-DOA protein. In some embodiments, the gene encoding an HLA-DOB protein is knocked out to reduce or eliminate expression of said HLA-DOB protein. In some embodiments, the gene encoding an HLA-DQ protein is knocked out to reduce or eliminate expression of said HLA-DQ protein. In some embodiments, the gene encoding an HLA-DR protein is knocked out to reduce or eliminate expression of said HLA-DR protein.

In some embodiments, the engineered cell, such as engineered primary cell, comprises a modification (e.g., genetic modification) targeting the CIITA gene. In some embodiments, the modification targeting the CIITA gene is by a targeted nuclease system that comprises a Cas protein or a polynucleotide encoding a Cas protein, and at least one guide ribonucleic acid sequence for specifically targeting the CIITA gene. In some embodiments, the at least one guide ribonucleic acid sequence (e.g. gRNA targeting sequence) for specifically targeting the CIITA gene is selected from the group consisting of SEQ ID NOS:5184-36352 of Appendix 1 or Table 12 of WO2016183041, the disclosure is incorporated by reference in its entirety. In some embodiments, the gRNA targeting sequence for specifically targeting the CIITA gene is GAUAUUGGCAUAAGCCUCCC (SEQ ID NO: 34).

In some embodiments, an exogenous nucleic acid or transgene encoding a polypeptide as disclosed herein (e.g., a chimeric antigen receptor, CD47, or another tolerogenic factor disclosed herein) is inserted at the CIITA gene. Exemplary transgenes for targeted insertion at the B2M locus include any as described in herein.

Assays to test whether the CIITA gene has been inactivated are known and described herein. In one embodiment, the resulting genetic modification of the CIITA gene is assessed by PCR. In some embodiments, the reduction of one or more MHC class II molecules, such as HLA-II, expression can be assays by flow cytometry, such as by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating modification, such as genetic modification. In some embodiments, the reduction in one or more MHC class II molecules expression is assessed using an immunoaffinity technique, such as immunohistochemistry or immunocytochemistry.

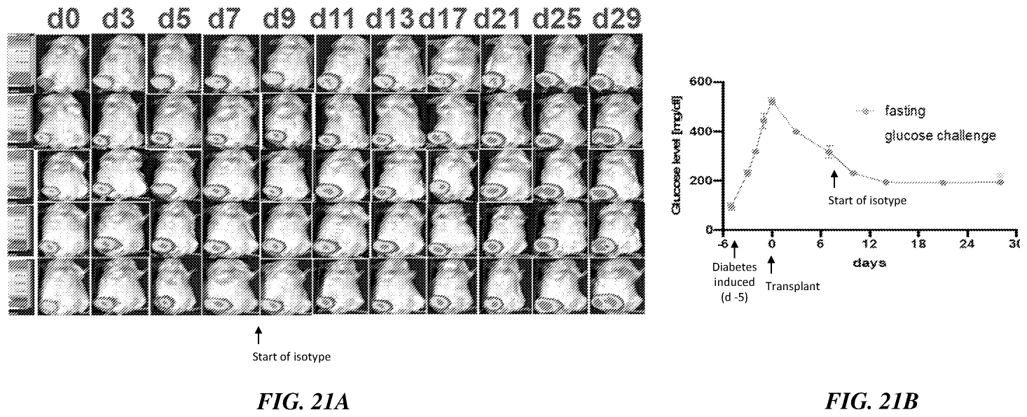

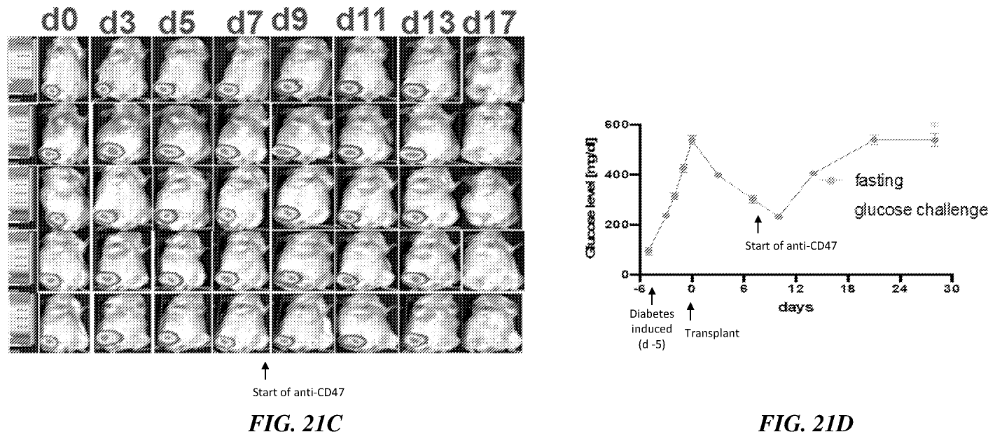

In some embodiments, the reduction of the one or more MHC class II molecules expression or function (HLA II when the cells are derived from human cells) in the engineered cells can be measured using techniques known in the art, such as Western blotting using antibodies to the protein, FACS techniques, RT-PCR techniques, etc. In some embodiments, the engineered cells can be tested to confirm that the HLA II complex is not expressed on the cell surface. Methods to assess surface expression include methods known in the art (See FIG. 21 of WO2018132783, for example) and generally is done using either Western Blots or FACS analysis based on commercial antibodies that bind to human HLA Class II HLA-DR, DP and most DQ antigens. In addition to the reduction of one or more HLA class II molecules (or one or more MHC class II molecules), the engineered cells provided herein have a reduced susceptibility to macrophage phagocytosis and NK cell killing. Methods to assay for hypoimmunogenic phenotypes of the engineered cells are described further below.

In some embodiments, the modification (e.g., genetic modification) that reduces CIITA expression reduces CIITA mRNA expression. In some embodiments, the reduced mRNA expression of CIITA is relative to an unmodified or wild-type cell of the same cell type that does not comprise the modification. In some embodiments, the mRNA expression of CIITA is reduced by more than about 5%, such as reduced by more than about any of 10%, 20%, 30%, 40%, 50%, 60%, 70%, 80%, 90%, or more. In some embodiments, the mRNA expression of CIITA is reduced by up to about 100%, such as reduced by up to about any of 90%, 80%, 70%, 60%, 50%, 40%, 30%, 20%, 10%, 5%, or less. In some embodiments, the mRNA expression of CIITA is reduced by any of about 5%, 10%, 20%, 30%, 40%, 50%, 60%, 70%, 80%, 90%, or 100%. In some embodiments, the mRNA expression of CIITA is eliminated (e.g., 0% expression of CIITA mRNA). In some embodiments, the modification that reduces CIITA mRNA expression eliminates CIITA gene activity.

In some embodiments, the modification (e.g., genetic modification) that reduces CIITA expression reduces CIITA protein expression. In some embodiments, the reduced protein expression of CIITA is relative to an unmodified or wild-type cell of the same cell type that does not comprise the modification. In some embodiments, the protein expression of CIITA is reduced by more than about 5%, such as reduced by more than about any of 10%, 20%, 30%, 40%, 50%, 60%, 70%, 80%, 90%, or more. In some embodiments, the protein expression of CIITA is reduced by up to about 100%, such as reduced by up to about any of 90%, 80%, 70%, 60%, 50%, 40%, 30%, 20%, 10%, 5%, or less. In some embodiments, the protein expression of CIITA is reduced by any of about 5%, 10%, 20%, 30%, 40%, 50%, 60%, 70%, 80%, 90%, or 100%. In some embodiments, the protein expression of CIITA is eliminated (e.g., 0% expression of CIITA protein). In some embodiments, the modification that reduces CIITA protein expression eliminates CIITA gene activity.

In some embodiments, the modification (e.g., genetic modification) that reduces CIITA expression comprises inactivation or disruption of the CIITA gene. In some embodiments, the modification that reduces CIITA expression comprises inactivation or disruption of one allele of the CIITA gene. In some embodiments, the modification that reduces CIITA expression comprises inactivation or disruption comprises inactivation or disruption of both alleles of the CIITA gene.

In some embodiments, the modification (e.g., genetic modification) comprises inactivation or disruption of one or more CIITA coding sequences in the cell. In some embodiments, the modification comprises inactivation or disruption of all CIITA coding sequences in the cell. In some embodiments, the modification comprises inactivation or disruption comprises an indel in the CIITA gene. In some embodiments, the modification is a frameshift mutation of genomic DNA of the CIITA gene. In some embodiments, the modification is a deletion of genomic DNA of the CIITA gene. In some embodiments, the modification is a deletion of a contiguous stretch of genomic DNA of the CIITA gene. In some embodiments, the CIITA gene is knocked out.

In some embodiments, the engineered cell, such as engineered primary cell, comprises a modification (e.g., genetic modification) targeting the T cell receptor alpha constant (TRAC) gene. In some embodiments, the modification targeting the TRAC gene is by a targeted nuclease system that comprises a Cas protein or a polynucleotide encoding a Cas protein, and at least one guide ribonucleic acid sequence for specifically targeting the TRAC gene. In some embodiments, the at least one guide ribonucleic acid sequence for specifically targeting the TRAC gene is selected from the group consisting of SEQ ID NOS:532-609 and 9102-9797 of US20160348073, the disclosure is incorporated by reference in its entirety. In some embodiments, the gRNA targeting sequence for specifically targeting the TRAC gene is AGAGUCUCUCAGCUGGUACA (SEQ ID NO: 35).

In some embodiments, an exogenous nucleic acid or transgene encoding a polypeptide as disclosed herein (e.g., a chimeric antigen receptor, CD47, or another tolerogenic factor disclosed herein) is inserted at the TRAC gene. Exemplary transgenes for targeted insertion at the TRAC locus include any as described in Section II.B.

Assays to test whether the TRAC gene has been inactivated are known and described herein. In one embodiment, the resulting modification of the TRAC gene by PCR and the reduction of HLA-II expression can be assays by flow cytometry, such as by FACS analysis. In another embodiment, TRAC protein expression is detected using a Western blot of cells lysates probed with antibodies to the TRAC protein. In another embodiment, reverse transcriptase polymerase chain reactions (RT-PCR) are used to confirm the presence of the inactivating modification.

In some embodiments, the modification (e.g., genetic modification) that reduces TRAC expression reduces TRAC mRNA expression. In some embodiments, the reduced mRNA expression of TRAC is relative to an unmodified or wild-type cell of the same cell type that does not comprise the modification. In some embodiments, the mRNA expression of TRAC is reduced by more than about 5%, such as reduced by more than about any of 10%, 20%, 30%, 40%, 50%, 60%, 70%, 80%, 90%, or more. In some embodiments, the mRNA expression of TRAC is reduced by up to about 100%, such as reduced by up to about any of 90%, 80%, 70%, 60%, 50%, 40%, 30%, 20%, 10%, 5%, or less. In some embodiments, the mRNA expression of TRAC is reduced by any of about 5%, 10%, 20%, 30%, 40%, 50%, 60%, 70%, 80%, 90%, or 100%. In some embodiments, the mRNA expression of TRAC is eliminated (e.g., 0% expression of TRAC mRNA). In some embodiments, the modification that reduces TRAC mRNA expression eliminates TRAC gene activity.

In some embodiments, the modification (e.g., genetic modification) that reduces TRAC expression reduces TRAC protein expression. In some embodiments, the reduced protein expression of TRAC is relative to an unmodified or wild-type cell of the same cell type that does not comprise the modification. In some embodiments, the protein expression of TRAC is reduced by more than about 5%, such as reduced by more than about any of 10%, 20%, 30%, 40%, 50%, 60%, 70%, 80%, 90%, or more. In some embodiments, the protein expression of TRAC is reduced by up to about 100%, such as reduced by up to about any of 90%, 80%, 70%, 60%, 50%, 40%, 30%, 20%, 10%, 5%, or less. In some embodiments, the protein expression of TRAC is reduced by any of about 5%, 10%, 20%, 30%, 40%, 50%, 60%, 70%, 80%, 90%, or 100%. In some embodiments, the protein expression of TRAC is eliminated (e.g., 0% expression of TRAC protein). In some embodiments, the modification that reduces TRAC protein expression eliminates TRAC gene activity.

In some embodiments, the modification (e.g., genetic modification) that reduces TRAC expression comprises inactivation or disruption of the TRAC gene. In some embodiments, the modification that reduces TRAC expression comprises inactivation or disruption of one allele of the TRAC gene. In some embodiments, the modification that reduces TRAC expression comprises inactivation or disruption comprises inactivation or disruption of both alleles of the TRAC gene.

In some embodiments, the modification (e.g., genetic modification) comprises inactivation or disruption of one or more TRAC coding sequences in the cell. In some embodiments, the modification comprises inactivation or disruption of all TRAC coding sequences in the cell. In some embodiments, the modification comprises inactivation or disruption comprises an indel in the TRAC gene. In some embodiments, the modification is a frameshift mutation of genomic DNA of the TRAC gene. In some embodiments, the modification is a deletion of genomic DNA of the TRAC gene. In some embodiments, the modification is a deletion of a contiguous stretch of genomic DNA of the TRAC gene. In some embodiments, the TRAC gene is knocked out.

In some embodiments, the engineered cell, such as engineered primary cell, comprises a modification (e.g., genetic modification) targeting the T cell receptor beta constant (TRBC) gene. In some embodiments, the modification targeting the TRBC gene is by a targeted nuclease system that comprises a Cas protein or a polynucleotide encoding a Cas protein, and at least one guide ribonucleic acid sequence for specifically targeting the TRBC gene.

In some embodiments, an exogenous nucleic acid or transgene encoding a polypeptide as disclosed herein (e.g., a chimeric antigen receptor, CD47, or another tolerogenic factor disclosed herein) is inserted at the TRBC gene. Exemplary transgenes for targeted insertion at the TRBC locus include any as described in Section II.B. In some embodiments, the at least one guide ribonucleic acid sequence for specifically targeting the TRBC gene is selected from the group consisting of SEQ ID NOS:610-765 and 9798-10532 of US20160348073, the disclosure is incorporated by reference in its entirety.

Assays to test whether the TRBC gene has been inactivated are known and described herein. In one embodiment, the resulting modification of the TRBC gene by PCR and the reduction of HLA-II expression can be assays by flow cytometry, such as by FACS analysis. In another embodiment, TRBC protein expression is detected using a Western blot of cells lysates probed with antibodies to the TRBC protein. In another embodiment, reverse transcriptase polymerase chain reactions (RT-PCR) are used to confirm the presence of the inactivating modification.

In some embodiments, the modification (e.g., genetic modification) that reduces TRBC expression reduces TRBC mRNA expression. In some embodiments, the reduced mRNA expression of TRAC is relative to an unmodified or wild-type cell of the same cell type that does not comprise the modification. In some embodiments, the mRNA expression of TRBC is reduced by more than about 5%, such as reduced by more than about any of 10%, 20%, 30%, 40%, 50%, 60%, 70%, 80%, 90%, or more. In some embodiments, the mRNA expression of TRBC is reduced by up to about 100%, such as reduced by up to about any of 90%, 80%, 70%, 60%, 50%, 40%, 30%, 20%, 10%, 5%, or less. In some embodiments, the mRNA expression of TRBC is reduced by any of about 5%, 10%, 20%, 30%, 40%, 50%, 60%, 70%, 80%, 90%, or 100%. In some embodiments, the mRNA expression of TRBC is eliminated (e.g., 0% expression of TRBC mRNA). In some embodiments, the modification that reduces TRBC mRNA expression eliminates TRBC gene activity.

In some embodiments, the modification (e.g., genetic modification) that reduces TRBC expression reduces TRBC protein expression. In some embodiments, the reduced protein expression of TRAC is relative to an unmodified or wild-type cell of the same cell type that does not comprise the modification. In some embodiments, the protein expression of TRBC is reduced by more than about 5%, such as reduced by more than about any of 10%, 20%, 30%, 40%, 50%, 60%, 70%, 80%, 90%, or more. In some embodiments, the protein expression of TRBC is reduced by up to about 100%, such as reduced by up to about any of 90%, 80%, 70%, 60%, 50%, 40%, 30%, 20%, 10%, 5%, or less. In some embodiments, the protein expression of TRBC is reduced by any of about 5%, 10%, 20%, 30%, 40%, 50%, 60%, 70%, 80%, 90%, or 100%. In some embodiments, the protein expression of TRBC is eliminated (e.g., 0% expression of TRBC protein). In some embodiments, the modification that reduces TRBC protein expression eliminates TRBC gene activity.

In some embodiments, the modification (e.g., genetic modification) that reduces TRBC expression comprises inactivation or disruption of the TRBC gene. In some embodiments, the modification that reduces TRBC expression comprises inactivation or disruption of one allele of the TRBC gene. In some embodiments, the modification that reduces TRBC expression comprises inactivation or disruption comprises inactivation or disruption of both alleles of the TRBC gene.

In some embodiments, the modification (e.g., genetic modification) comprises inactivation or disruption of one or more TRBC coding sequences in the cell. In some embodiments, the modification comprises inactivation or disruption of all TRBC coding sequences in the cell. In some embodiments, the modification comprises inactivation or disruption comprises an indel in the TRBC gene. In some embodiments, the modification is a frameshift mutation of genomic DNA of the TRBC gene. In some embodiments, the modification is a deletion of genomic DNA of the TRBC gene. In some embodiments, the modification is a deletion of a contiguous stretch of genomic DNA of the TRBC gene. In some embodiments, the TRBC gene is knocked out.

### B. Overexpression of Polynucleotides

In some embodiments, the engineered cells, such as engineered primary cells, provided herein are genetically modified or engineered, such as by introduction of one or more modifications into a cell, such as a primary cell, to overexpress a desired polynucleotide in the cell. In some embodiments, the cell, such as a primary cell, to be modified or engineered is an unmodified cell or non-engineered cell (e.g. unmodified primary cell or non-engineered primary cell) that has not previously been introduced with the one or more modifications. In some embodiments, the engineered cells, such as engineered primary cells, provided herein are genetically modified to include one or more exogenous polynucleotides encoding an exogenous protein (also interchangeably used with the term “transgene”). As described, in some embodiments, the cells, such as primary cells, are modified to increase expression of certain genes that are tolerogenic (e.g., immune) factors that affect immune recognition and tolerance in a recipient. In some embodiments, the provided engineered primary cells, such as T cells or NK cells, also express a chimeric antigen receptor (CAR). The one or more polynucleotides, e.g. exogenous polynucleotides, may be expressed (e.g. overexpressed) in the engineered cell together with one or more genetic modifications to reduce expression of a target polynucleotide described in Section I.A above, such as an one or more MHC class I molecules and/or one or more MHC class II molecules. In some embodiments, the provided engineered cell, such as engineered primary cells, do not trigger or activate an immune response upon administration to a recipient subject.

In some embodiments, the engineered cell, such as engineered primary cell, includes 1, 2, 3, 4, 5, 6, 7, 8, 9, 10 or more different overexpressed polynucleotides. In some embodiments, the engineered cell, such as engineered primary cell, includes 1, 2, 3, 4, 5, 6, 7, 8, 9, 10 or more different overexpressed polynucleotides. In some embodiments, the overexpressed polynucleotide is an exogenous polynucleotide. In some embodiments, the engineered cell, such as engineered primary cell, includes 1, 2, 3, 4, 5, 6, 7, 8, 9, 10 or more different exogenous polynucleotides. In some embodiments, the engineered primary cell includes 1, 2, 3, 4, 5, 6, 7, 8, 9, 10 or more different exogenous polynucleotides. In some embodiments, the overexpreesed polynucleotide is an exogenous polynucleotide that is expressed episomally in the primary cells. In some embodiments, the overexpressed polynucleotide is an exogenous polynucleotide that is inserted or integrated into one or more genomic loci of the engineered primary cell.

In some embodiments, expression of a polynucleotide is increased, i.e. the polynucleotide is overexpressed, using a fusion protein containing a DNA-targeting domain and a transcriptional activator. Targeted methods of increasing expression using transactivator domains are known to a skilled artisan.

In some embodiments, engineered cell, such as engineered primary cell, contains one or more exogenous polynucleotides in which the one or more exogenous polynucleotides are inserted or integrated into a genomic locus of the cell by non-targeted insertion methods, such as by transduction with a lentiviral vector. In some embodiments, the lentiviral vector comprises a piggyBac transposon. During transposition, the piggyback transposon recognizes transposon-specific inverted terminal repeats (ITRs) in a lentiviral vector, to allow for the efficient movement and integration of the vector contents into TTAA chromosomal sites. In some embodiments, the one or more exogenous polynucleotides are inserted or integrated into the genome of the cell, such as primary cell, by targeted insertion methods, such as by using homology directed repair (HDR). Any suitable method can be used to insert the exogenous polynucleotide into the genomic locus of the engineered cell, such as primary cell, by HDR including the gene editing methods described herein (e.g., a CRISPR/Cas system). In some embodiments, the one or more exogenous polynucleotides are inserted into one or more genomic locus, such as any genomic locus described herein (e.g. Table 4). In some embodiments, the exogenous polynucleotides are inserted into the same genomic loci. In some embodiments, the exogenous polynucleotides are inserted into different genomic loci. In some embodiments, the two or more of the exogenous polynucleotides are inserted into the same genomic loci, such as any genomic locus described herein (e.g. Table 4). In some embodiments, two or more exogenous polynucleotides are inserted into a different genomic loci, such as two or more genomic loci as described herein (e.g., Table 4).

Exemplary polynucleotides or overexpression, and methods for overexpressing the same, are described in the following subsections.

1. Tolerogenic Factor

In some embodiments, expression of a tolerogenic factor is overexpressed or increased in the cell, e.g. primary cell. In some embodiments, the engineered cell includes increased expression, i.e. overexpression, of at least one tolerogenic factor. In some embodiments, the tolerogenic factor is any factor that promotes or contributes to promoting or inducing tolerance to the engineered cell by the immune system (e.g. innate or adaptive immune system).

In some embodiments, the one or more tolerogenic factors is t selected from the group consisting of CD47, A20/TNFAIP3, C1-Inhibitor, CCL21, CCL22, CD16, CD16 Fc receptor, CD24, CD27, CD35, CD39, CD46, CD52, CD55, CD59, CD200, CR1, CTLA4-Ig, DUX4, FasL, H2-M3, HLA-C, HLA-E, HLA-E heavy chain, HLA-G, PD-L1, IDO1, IL-10, IL15-RF, IL-35, MANF, Mfge8, PD-1, PD-L1, or Serpinb9. In some embodiments, the tolerogenic factor is DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, C1-Inhibitor, IL-10, IL-35, FASL, CCL21, MFGE8, SERPINB9, CD35, IL-39, CD16 Fc Receptor, IL15-RF, and H2-M3. In some embodiments, the tolerogenic factor is CD47, PD-L1, HLA-E or HLA-G, CCL21, FasL, Serpinb9, CD200 or Mfge8, or any combination thereof. In some embodiments, the cell, such as primary cell, includes at least one exogenous polynucleotide that includes a polynucleotide that encodes for a tolerogenic factor. For instance, in some embodiments, at least one of the exogenous polynucleotides is a polynucleotide that encodes CD47. Provided herein are cells that do not trigger or activate an immune response upon administration to a recipient subject. As described above, in some embodiments, the cells, such as primary cells, are modified to increase expression of genes and tolerogenic (e.g., immune) factors that affect immune recognition and tolerance in a recipient.

In some embodiments, the expression (e.g., surface expression) of a tolerogenic factor is increased by about 10% or higher compared to a cell of the same cell type that does not comprise the modification, such as increased by any of about 15%, 20%, 25%, 30%, 35%, 40%, 45%, 50%, 55%, 60%, 65%, 70%, 75%, 80%, 85%, 90%, 95%, 99%, or higher, compared to a cell of the same cell type that does not comprise the modification. In some embodiments, the expression of a tolerogenic factor is increased by about 99% or lower compared to a cell of the same cell type that does not comprise the modification, such as increased by any of about 95%, 90%, 85%, 80%, 75%, 70%, 65%, 60%, 55%, 50%, 45%, 40%, 35%, 30%, 25%, 20%, 15%, 10%, or lower, compared to a cell of the same cell type that does not comprise the modification. In some embodiments, the expression of a tolerogenic factor is increased by between about 10% and about 100% compared to a cell of the same cell type that does not comprise the modification, such as between any of about 10% and about 40%, about 20% and about 60%, about 50% and about 80%, and about 70% and about 100%, compared to a cell of the same cell type that does not comprise the modification.

In some embodiments, the expression (e.g., surface expression) of a tolerogenic factor is increased by about 2-fold or higher compared to a cell of the same cell type that does not comprise the modification, such as any of about 4-fold or higher, 6-fold or higher, 8-fold or higher, 10-fold or higher, 15-fold or higher, 20-fold or higher, 30-fold or higher, 40-fold or higher, 50-fold or higher, 60-fold or higher, 70-fold or higher, 80-fold or higher, 90-fold or higher, 100-fold or higher, 150-fold or higher, and 200-fold or higher compared to a cell of the same cell type that does not comprise the modification. In some embodiments, the expression of a tolerogenic factor is increased by about 200-fold or lower compared to a cell of the same cell type that does not comprise the modification, such as any of about 150-fold or lower, 100-fold or lower, 90-fold or lower, 80-fold or lower, 70-fold or lower, 60-fold or lower, 50-fold or lower, 40-fold or lower, 30-fold or lower, 15-fold or lower, 10-fold or lower, 8-fold or lower, 6-fold or lower, 4-fold or lower, and 2-fold or lower compared to a cell of the same cell type that does not comprise the modification. In some embodiments, the expression of a tolerogenic factor is increased by between about 2-fold and about 200-fold compared to a cell of the same cell type that does not comprise the modification, such as between any of about 2-fold and about 20-fold, about 10-fold and about 50-fold, about 30-fold and about 70-fold, about 50-fold and about 100-fold, about 80-fold and about 150-fold, and about 120-fold and about 200-fold, compared to a cell of the same cell type that does not comprise the modification.

In some embodiments, the present disclosure provides a cell, such as a primary cell, or population thereof that has been modified to express the tolerogenic factor (e.g., immunomodulatory polypeptide), such as CD47. In some embodiments, the present disclosure provides a method for altering a cell genome to express the tolerogenic factor (e.g. immunomodulatory polypeptide), such as CD47. In some embodiments, the engineered cell, such as engineered primary cell, expresses an exogenous tolerogenic factor (e.g. immunomodulatory polypeptide), such as an exogenous CD47. In some instances, overexpression or increasing expression of the exogenous polynucleotide is achieved by introducing into the primary cell (e.g. transducing the cell) with an expression vector comprising a nucleotide sequence encoding a human CD47 polypeptide. In some embodiments, the expression vector may be a viral vector, such as a lentiviral vector) or may be a non-viral vector. In some embodiments, the cell, such as a primary cell, is engineered to contain one or more exogenous polynucleotides in which at least one of the exogenous polynucleotides includes a polynucleotide that encodes for a tolerogenic factor. In some embodiments, the DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, C1-Inhibitor, IL-10, IL-35, FASL, CCL21, MFGE8, SERPINB9, CD35, IL-39, CD16 Fc Receptor, IL15-RF, and H2-M3 (including any combination thereof). In some embodiments, the tolerogenic factor is one or more of CD47, PD-L1, HLA-E or HLA-G, CCL21, FasL, Serpinb9, CD200, and Mfge8 (including any combination thereof). For instance, in some embodiments, at least one of the exogenous polynucleotides is a polynucleotide that encodes CD47.

In some embodiments, the tolerogenic factor is CD47. In some embodiments, the engineered cell, such as a primary cell, contains an exogenous polynucleotide that encodes CD47, such as human CD47. In some embodiments, CD47 is overexpressed in the cell, e.g. primary cell. In some embodiments, the expression of CD47 is overexpressed or increased in the engineered cell, such as engineered primary cell, compared to a similar cell of the same cell type that has not been engineered with the modification, such as a reference or unmodified cell, e.g. a primary cell not engineered with an exogenous polynucleotide encoding CD47. CD47 is a leukocyte surface antigen and has a role in cell adhesion and modulation of integrins. It is normally expressed on the surface of a cell and signals to circulating macrophages not to eat the cell. Useful genomic, polynucleotide and polypeptide information about human CD47 are provided in, for example, the NP_001768.1, NP_942088.1, NM_001777.3 and NM_198793.2.

In some embodiments, the expression (e.g., surface expression) of CD47 is increased by about 10% or higher compared to a cell of the same cell type that does not comprise the modification, such as increased by any of about 15%, 20%, 25%, 30%, 35%, 40%, 45%, 50%, 55%, 60%, 65%, 70%, 75%, 80%, 85%, 90%, 95%, 99%, or higher, compared to a cell of the same cell type that does not comprise the modification. In some embodiments, the expression of CD47 is increased by about 99% or lower compared to a cell of the same cell type that does not comprise the modification, such as increased by any of about 95%, 90%, 85%, 80%, 75%, 70%, 65%, 60%, 55%, 50%, 45%, 40%, 35%, 30%, 25%, 20%, 15%, 10%, or lower, compared to a cell of the same cell type that does not comprise the modification. In some embodiments, the expression of CD47 is increased by between about 10% and about 100% compared to a cell of the same cell type that does not comprise the modification, such as between any of about 10% and about 40%, about 20% and about 60%, about 50% and about 80%, and about 70% and about 100%, compared to a cell of the same cell type that does not comprise the modification.

In some embodiments, the expression (e.g., surface expression) of CD47 is increased by about 2-fold or higher compared to a cell of the same cell type that does not comprise the modification, such as any of about 4-fold or higher, 6-fold or higher, 8-fold or higher, 10-fold or higher, 15-fold or higher, 20-fold or higher, 30-fold or higher, 40-fold or higher, 50-fold or higher, 60-fold or higher, 70-fold or higher, 80-fold or higher, 90-fold or higher, 100-fold or higher, 150-fold or higher, and 200-fold or higher compared to a cell of the same cell type that does not comprise the modification. In some embodiments, the expression of CD47 is increased by about 200-fold or lower compared to a cell of the same cell type that does not comprise the modification, such as any of about 150-fold or lower, 100-fold or lower, 90-fold or lower, 80-fold or lower, 70-fold or lower, 60-fold or lower, 50-fold or lower, 40-fold or lower, 30-fold or lower, 15-fold or lower, 10-fold or lower, 8-fold or lower, 6-fold or lower, 4-fold or lower, and 2-fold or lower compared to a cell of the same cell type that does not comprise the modification. In some embodiments, the expression of CD47 is increased by between about 2-fold and about 200-fold compared to a cell of the same cell type that does not comprise the modification, such as between any of about 2-fold and about 20-fold, about 10-fold and about 50-fold, about 30-fold and about 70-fold, about 50-fold and about 100-fold, about 80-fold and about 150-fold, and about 120-fold and about 200-fold, compared to a cell of the same cell type that does not comprise the modification.

In some embodiments, the cell outlined herein comprises a nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a nucleotide sequence encoding a CD47 polypeptide having an amino acid sequence as set forth in NCBI Ref. Sequence Nos. NP_001768.1 and NP_942088.1. In some embodiments, the cell comprises a nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises a nucleotide sequence for CD47 as set forth in NCBI Ref. Sequence Nos. NM_001777.3 and NM_198793.2.

In some embodiments, the cell comprises a CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises a CD47 polypeptide having an amino acid sequence as set forth in NCBI Ref. Sequence Nos. NP_001768.1 and NP_942088.1.

In some embodiments, the cell comprises a CD47 polypeptide having at least 95% sequence identity (e.g., 95%, 96%, 97%, 98%, 99%, or more) to an amino acid sequence as set forth in SEQ ID NO: 1. In some embodiments, the cell comprises a CD47 polypeptide having the amino acid sequence as set forth in SEQ ID NO: 1. In some embodiments, the cell comprises a CD47 polypeptide having at least 95% sequence identity (e.g., 95%, 96%, 97%, 98%, 99%, or more) to an amino acid sequence as set forth in SEQ ID NO: 2. In some embodiments, the cell comprises a CD47 polypeptide having the amino acid sequence as set forth in SEQ ID NO: 2.

In certain embodiments, the polynucleotide encoding CD47 is operably linked to a promoter.

In some embodiments, an exogenous polynucleotide encoding CD47 is integrated into the genome of the cell by targeted or non-targeted methods of insertion, such as described further below. In some embodiments, targeted insertion is by homology-dependent insertion into a target loci, such as by insertion into any one of the genomic (gene) loci. In some embodiments, each of the one or more genomic loci are selected from the group consisting of a MICA gene locus, a MICB gene locus, a B2M gene locus, a CIITA gene locus, a TRAC gene locus or a TRBC gene locus, a CD142 gene locus, a CCR5 gene locus, CXCR4 gene locus, PPP1R12C (also known as AAVS1) gene locus, albumin gene locus, SHS231 locus, CLYBL gene locus, ROSA26 gene locus, LRP1 gene locus, HMGB1 gene locus, ABO gene locus, RHD gene locus, FUT1 gene locus, and KDM5D gene locus. In some embodiments, each of the one or more genomic loci are selected from the group consisting of a B2M locus, a TAP) locus, a CIITA locus, a TRAC locus, a TRBC locus, a MIC-A locus, a MIC-B locus, and a safe harbor locus. In some embodiments, the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus.

In some embodiments, targeted insertion is by homology-dependent insertion into a target loci, such as by insertion into any one of the gene loci depicted in Table 1b, 2 or 4, e.g. a B2M gene, a CIITA gene, a TRAC gene, a TRBC gene. In some embodiments, targeted insertion is by homology-independent insertion, such as by insertion into a safe harbor locus. In some cases, the polynucleotide encoding CD47 is inserted into a safe harbor locus, such as but not limited to, a gene locus selected from AAVS1, CCR5, CLYBL, ROSA26, and SHS231. In particular embodiments, the polynucleotide encoding CD47 is inserted into the CCR5 gene locus, the PPP1R12C (also known as AAVS1) gene locus or the CLYBL gene locus. In some embodiments, the polynucleotide encoding CD47 is inserted into any one of the gene loci depicted in Table 4. In some cases, the polynucleotide encoding CD47 is inserted into a safe harbor locus.

In particular embodiments, the polynucleotide encoding CD47 is inserted into the CCR5 gene locus, the PPP1R12C (also known as AAVS1) gene locus or the CLYBL gene locus. In some embodiments, the polynucleotide encoding CD47 is inserted into a B2M gene locus or a CIITA gene locus. In some embodiments, the engineered cell, such as engineered primary cell, is a T cell and the polynucleotide encoding CD47 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding CD47, into a genomic locus of the cell. In some embodiments, the engineered cell is a T cell and the polynucleotide encoding CD47 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding CD47, into a genomic locus of the cell.

In some embodiments, CD47 protein expression is detected using a Western blot of cell lysates probed with antibodies against the CD47 protein. In another embodiment, reverse transcriptase polymerase chain reactions (RT-PCR) are used to confirm the presence of the exogenous CD47 mRNA.

In some embodiments, the engineered cell, such as engineered primary cell, contains an exogenous polynucleotide that encodes CD200, such as human CD200. In some embodiments, CD200 is overexpressed in the cell. In some embodiments, the expression of CD200 is increased in the engineered cell, such as engineered primary cell, compared to a similar reference or unmodified cell (including with any other modifications, such as genetic modifications) except that the reference or unmodified cell does not include the exogenous polynucleotide encoding CD200. Useful genomic, polynucleotide and polypeptide information about human CD200 are provided in, for example, the GeneCard Identifier GC03P112332, HGNC No. 7203, NCBI Gene ID 4345, Uniprot No. P41217, and NCBI RefSeq Nos. NP_001004196.2, NM_001004196.3, NP_001305757.1, NM_001318828.1, NP_005935.4, NM_005944.6, XP_005247539.1, and XM_005247482.2. In certain embodiments, the polynucleotide encoding CD200 is operably linked to a promoter.

In some embodiments, the polynucleotide encoding CD200 is inserted into any one of the gene loci depicted in Table 1B, 2 or 4. In some cases, the polynucleotide encoding CD200 is inserted into a safe harbor locus, such as but not limited to, a gene locus selected from AAVS1, CCR5, CLYBL, ROSA26, and SHS231. In particular embodiments, the polynucleotide encoding CD200 is inserted into the CCR5 gene locus, the PPP1R12C (also known as AAVS1) gene locus or the CLYBL gene locus. In some embodiments, the polynucleotide encoding CD200 is inserted into a B2M gene locus or a CIITA gene locus. In some embodiments, the engineered primary cell is a T cell and the polynucleotide encoding CD200 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding CD200, into a genomic locus of the cell.

In some embodiments, CD200 protein expression is detected using a Western blot of cell lysates probed with antibodies against the CD200 protein. In another embodiment, reverse transcriptase polymerase chain reactions (RT-PCR) are used to confirm the presence of the exogenous CD200 mRNA.

In some embodiments, the engineered cell, such as engineered primary cell, contains an exogenous polynucleotide that encodes HLA-E, such as human HLA-E. In some embodiments, HLA-E is overexpressed in the cell. In some embodiments, the expression of HLA-E is increased in the engineered cell, such as engineered primary cell, compared to a similar reference or unmodified cell (including with any other modifications) except that the reference or unmodified cell does not include the exogenous polynucleotide encoding HLA-E. Useful genomic, polynucleotide and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM_005516.5. In certain embodiments, the polynucleotide encoding HLA-E is operably linked to a promoter.

In some embodiments, the polynucleotide encoding HLA-E is inserted into any one of the gene loci depicted in Table 1B, 2 or 4. In some cases, the polynucleotide encoding HLA-E is inserted into a safe harbor locus, such as but not limited to, a gene locus selected from AAVS1, CCR5, CLYBL, ROSA26, SHS231. In particular embodiments, the polynucleotide encoding HLA-E is inserted into the CCR5 gene locus, the PPP1R12C (also known as AAVS1) gene locus or the CLYBL gene locus. In some embodiments, the polynucleotide encoding HLA-E is inserted into a B2M gene locus, a CIITA gene locus. In some embodiments, the engineered primary cell is a T cell and the polynucleotide encoding HLA-E is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding HLA-E, into a genomic locus of the cell.

In some embodiments, HLA-E protein expression is detected using a Western blot of cell lysates probed with antibodies against the HLA-E protein. In another embodiment, reverse transcriptase polymerase chain reactions (RT-PCR) are used to confirm the presence of the exogenous HLA-E mRNA.

In some embodiments, the engineered cell, such as engineered primary cell, contains an exogenous polynucleotide that encodes HLA-G, such as human HLA-G. In some embodiments, HLA-G is overexpressed in the cell. In some embodiments, the expression of HLA-G is increased in the engineered cell, such as engineered primary cell, compared to a similar reference or unmodified cell (including with any other modifications, such as genetic modifications) except that the reference or unmodified cell does not include the exogenous polynucleotide encoding HLA-G. Useful genomic, polynucleotide and polypeptide information about human HLA-G are provided in, for example, the GeneCard Identifier GC06P047256, HGNC No. 4964, NCBI Gene ID 3135, Uniprot No. P17693, and NCBI RefSeq Nos. NP_002118.1 and NM_002127.5. In certain embodiments, the polynucleotide encoding HLA-G is operably linked to a promoter.

In some embodiments, the polynucleotide encoding HLA-G is inserted into any one of the gene loci depicted in Table 1b, 2 or 4. In some cases, the polynucleotide encoding HLA-G is inserted into a safe harbor locus, such as but not limited to, a gene locus selected from AAVS1, CCR5, CLYBL, ROSA26, and SHS231. In particular embodiments, the polynucleotide encoding HLA-G is inserted into the CCR5 gene locus, the PPP1R12C (also known as AAVS1) gene locus or the CLYBL gene locus. In some embodiments, the polynucleotide encoding HLA-G is inserted into a B2M gene locus or; a CITA gene locus. In some embodiments, the engineered primary cell is a T cell and the polynucleotide encoding HLA-G is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding HLA-G, into a genomic locus of the cell.

In some embodiments, HLA-G protein expression is detected using a Western blot of cell lysates probed with antibodies against the HLA-G protein. In another embodiment, reverse transcriptase polymerase chain reactions (RT-PCR) are used to confirm the presence of the exogenous HLA-G mRNA.

In some embodiments, the engineered cell, such as engineered primary cell, contains an exogenous polynucleotide that encodes PD-L1, such as human PD-L1. In some embodiments, PD-L1 is overexpressed in the cell. In some embodiments, the expression of PD-L1 is increased in the engineered cell, such as engineered primary cell, compared to a similar reference or unmodified cell (including with any other modifications) except that the reference or unmodified cell does not include the exogenous polynucleotide encoding PD-L1. Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1, NM_001267706.1, NP_054862.1, and NM_014143.3. In certain embodiments, the polynucleotide encoding PD-L1 is operably linked to a promoter.

In some embodiments, the polynucleotide encoding PD-L1 is inserted into any one of the gene loci depicted in Table 1B, 2 or 4. In some cases, the polynucleotide encoding PD-L1 is inserted into a safe harbor locus, such as but not limited to, a gene locus selected from AAVS1, CCR5, CLYBL, ROSA26, and SHS231. In particular embodiments, the polynucleotide encoding PD-L1 is inserted into the CCR5 gene locus, the PPP1R12C (also known as AAVS1) gene locus or the CLYBL gene locus. In some embodiments, the polynucleotide encoding PD-L1 is inserted into a B2M gene locus, a CIITA gene locus. In some embodiments, the engineered primary cell is a T cell and the polynucleotide encoding PD-L1 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding PD-L1, into a genomic locus of the cell.

In some embodiments, PD-L1 protein expression is detected using a Western blot of cell lysates probed with antibodies against the PD-L1 protein. In another embodiment, reverse transcriptase polymerase chain reactions (RT-PCR) are used to confirm the presence of the exogenous PD-L1 mRNA.

In some embodiments, the engineered cell, such as engineered primary cell, contains an exogenous polynucleotide that encodes FasL, such as human FasL. In some embodiments, FasL is overexpressed in the cell. In some embodiments, the expression of FasL is increased in the engineered primary cell compared to a similar reference or unmodified cell (including with any other modifications, such as genetic modifications) except that the reference or unmodified cell does not include the exogenous polynucleotide encoding FasL. Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No. P48023, and NCBI RefSeq Nos. NP_000630.1, NM_000639.2, NP_001289675.1, and NM_001302746.1. In certain embodiments, the polynucleotide encoding Fas-L is operably linked to a promoter.

In some embodiments, the polynucleotide encoding Fas-L is inserted into any one of the gene loci depicted in Table 1B, 2 or 4. In some cases, the polynucleotide encoding Fas-L is inserted into a safe harbor locus, such as but not limited to, a gene locus selected from AAVS1, CCR5, CLYBL, ROSA26, and SHS231. In particular embodiments, the polynucleotide encoding Fas-L is inserted into the CCR5 gene locus, the PPP1R12C (also known as AAVS1) gene locus or the CLYBL gene locus. In some embodiments, the polynucleotide encoding Fas-L is inserted into a B2M gene locus or a CIITA gene locus. In some embodiments, the engineered primary cell is a T cell and the polynucleotide encoding Fas-L is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding Fas-L, into a genomic locus of the cell.

In some embodiments, Fas-L protein expression is detected using a Western blot of cell lysates probed with antibodies against the Fas-L protein. In another embodiment, reverse transcriptase polymerase chain reactions (RT-PCR) are used to confirm the presence of the exogenous Fas-L mRNA.

In some embodiments, the engineered cell, such as engineered primary cell, contains an exogenous polynucleotide that encodes CCL21, such as human CCL21. In some embodiments, CCL21 is overexpressed in the cell. In some embodiments, the expression of CCL21 is increased in the engineered cell, such as engineered primary cell, compared to a similar reference or unmodified cell (including with any other modifications, such as genetic modifications) except that the reference or unmodified cell does not include the exogenous polynucleotide encoding CCL21. Useful genomic, polynucleotide and polypeptide information about human CCL21 are provided in, for example, the GeneCard Identifier GC09M034709, HGNC No. 10620, NCBI Gene ID 6366, Uniprot No. 000585, and NCBI RefSeq Nos. NP_002980.1 and NM_002989.3. In certain embodiments, the polynucleotide encoding CCL21 is operably linked to a promoter.

In some embodiments, the polynucleotide encoding CCL21 is inserted into any one of the gene loci depicted in Table 1B, 2 or 4. In some cases, the polynucleotide encoding CCL21 is inserted into a safe harbor locus, such as but not limited to, a gene locus selected from AAVS1, CCR5, CLYBL, ROSA26, and SHS231. In particular embodiments, the polynucleotide encoding CCL21 is inserted into the CCR5 gene locus, the PPP1R12C (also known as AAVS1) gene locus or the CLYBL gene locus. In some embodiments, the polynucleotide encoding CCL21 is inserted into a B2M gene locus, a CIITA gene locus. In some embodiments, the engineered primary cell is a T cell and the polynucleotide encoding CCL21 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding CCL21, into a genomic locus of the cell.

In some embodiments, CCL21 protein expression is detected using a Western blot of cell lysates probed with antibodies against the CCL21 protein. In another embodiment, reverse transcriptase polymerase chain reactions (RT-PCR) are used to confirm the presence of the exogenous CCL21 mRNA.

In some embodiments, the engineered cell, such as engineered primary cell, contains an exogenous polynucleotide that encodes CCL22, such as human CCL22. In some embodiments, CCL22 is overexpressed in the cell. In some embodiments, the expression of CCL22 is increased in the engineered cell, such as engineered primary cell, compared to a similar reference or unmodified cell (including with any other modifications, such as genetic modifications) except that the reference or unmodified cell does not include the exogenous polynucleotide encoding CCL22. Useful genomic, polynucleotide and polypeptide information about human CCL22 are provided in, for example, the GeneCard Identifier GC16P057359, HGNC No. 10621, NCBI Gene ID 6367, Uniprot No. 000626, and NCBI RefSeq Nos. NP_002981.2, NM_002990.4, XP_016879020.1, and XM_017023531.1. In certain embodiments, the polynucleotide encoding CCL22 is operably linked to a promoter.

In some embodiments, the polynucleotide encoding CCL22 is inserted into any one of the gene loci depicted in Table 1B, 2 or 4. In some cases, the polynucleotide encoding CCL22 is inserted into a safe harbor locus, such as but not limited to, a gene locus selected from AAVS1, CCR5, CLYBL, ROSA26, and SHS231. In particular embodiments, the polynucleotide encoding CCL22 is inserted into the CCR5 gene locus, the PPP1R12C (also known as AAVS1) gene locus or the CLYBL gene locus. In some embodiments, the polynucleotide encoding CCL22 is inserted into a B2M gene locus, a CIITA gene locus. In some embodiments, the engineered primary cell is a T cell and the polynucleotide encoding CCL22 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding CCL22, into a genomic locus of the cell.

In some embodiments, CCL22 protein expression is detected using a Western blot of cell lysates probed with antibodies against the CCL22 protein. In another embodiment, reverse transcriptase polymerase chain reactions (RT-PCR) are used to confirm the presence of the exogenous CCL22 mRNA.

In some embodiments, the engineered cell, such as engineered primary cell, contains an exogenous polynucleotide that encodes Mfge8, such as human Mfge8. In some embodiments, Mfge8 is overexpressed in the cell. In some embodiments, the expression of Mfge8 is increased in the engineered cell, such as engineered primary cell, compared to a similar reference or unmodified cell (including with any other modifications, such as genetic modifications) except that the reference or unmodified cell does not include the exogenous polynucleotide encoding Mfge8. Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP_001297249.1, NM_001310320.1, NP_001297250.1, NM_001310321.1, NP_005919.2, and NM_005928.3. In certain embodiments, the polynucleotide encoding Mfge8 is operably linked to a promoter.

In some embodiments, the polynucleotide encoding Mfge8 is inserted into any one of the gene loci depicted in Table 1B, 2 or 4. In some cases, the polynucleotide encoding Mfge8 is inserted into a safe harbor locus, such as but not limited to, a gene locus selected from AAVS1, CCR5, CLYBL, ROSA26, and SHS231. In particular embodiments, the polynucleotide encoding Mfge8 is inserted into the CCR5 gene locus, the PPP1R12C (also known as AAVS1) gene locus or the CLYBL gene locus. In some embodiments, the polynucleotide encoding Mfge8 is inserted into a B2M gene locus, a CIITA gene locus. In some embodiments, the engineered primary cell is a T cell and the polynucleotide encoding Mfge8 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding Mfge8, into a genomic locus of the cell.

In some embodiments, Mfge8 protein expression is detected using a Western blot of cell lysates probed with antibodies against the Mfge8 protein. In another embodiment, reverse transcriptase polymerase chain reactions (RT-PCR) are used to confirm the presence of the exogenous Mfge8 mRNA.

In some embodiments, the engineered cell, such as engineered primary cell, contains an exogenous polynucleotide that encodes SerpinB9, such as human SerpinB9. In some embodiments, SerpinB9 is overexpressed in the cell. In some embodiments, the expression of SerpinB9 is increased in the engineered cell, such as engineered primary cell, compared to a similar reference or unmodified cell (including with any other modifications, such as genetic modifications) except that the reference or unmodified cell does not include the exogenous polynucleotide encoding SerpinB9. Useful genomic, polynucleotide and polypeptide information about human SerpinB9 are provided in, for example, the GeneCard Identifier GC06M002887, HGNC No. 8955, NCBI Gene ID 5272, Uniprot No. P50453, and NCBI RefSeq Nos. NP_004146.1, NM_004155.5, XP_005249241.1, and XM_005249184.4. In certain embodiments, the polynucleotide encoding SerpinB9 is operably linked to a promoter.

In some embodiments, the polynucleotide encoding SerpinB9 is inserted into any one of the gene loci depicted in Table 1B, 2 or 4. In some cases, the polynucleotide encoding SerpinB9 is inserted into a safe harbor locus, such as but not limited to, a gene locus selected from AAVS1, CCR5, CLYBL, ROSA26, and SHS231. In particular embodiments, the polynucleotide encoding SerpinB9 is inserted into the CCR5 gene locus, the PPP1R12C (also known as AAVS1) gene locus or the CLYBL gene locus. In some embodiments, the polynucleotide encoding SerpinB9 is inserted into a B2M gene locus, a CIITA gene locus. In some embodiments, the engineered primary cell is a T cell and the polynucleotide encoding SerpinB9 is inserted into a TRAC gene locus, or a TRBC gene locus. In some embodiments, a suitable gene editing system (e.g., CRISPR/Cas system or any of the gene editing systems described herein) is used to facilitate the insertion of a polynucleotide encoding SerpinB9, into a genomic locus of the cell.

In some embodiments, SerpinB9 protein expression is detected using a Western blot of cell lysates probed with antibodies against the SerpinB9 protein. In another embodiment, reverse transcriptase polymerase chain reactions (RT-PCR) are used to confirm the presence of the exogenous SerpinB9 mRNA.

In some embodiments, a provided engineered cell, such as engineered primary cell, is further modified to express a chimeric antigen receptor (CAR). In some embodiments, a polynucleotide encoding a CAR is introduced into the cell. In some embodiments, the cell is a T cell, such as a primary T cell. In some embodiments, the cells is a Natural Killer (NK) cell, such as a primary NK cell.

In some embodiments, the CAR is selected from the group consisting of a first generation CAR, a second generation CAR, a third generation CAR, and a fourth generation CAR. In some embodiments, the CAR is or comprises a first generation CAR comprising an antigen binding domain, a transmembrane domain, and at least one signaling domain (e.g., one, two or three signaling domains). In some embodiments, the CAR comprises a second generation CAR comprising an antigen binding domain, a transmembrane domain, and at least two signaling domains. In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an antibody, an antibody fragment, an scFv or a Fab.

In some embodiments, any one of the cells described herein comprises a nucleic acid encoding a CAR or a first generation CAR. In some embodiments, a first generation CAR comprises an antigen binding domain, a transmembrane domain, and signaling domain. In some embodiments, a signaling domain mediates downstream signaling during T cell activation.

In some embodiments, any one of the cells described herein comprises a nucleic acid encoding a CAR or a second generation CAR. In some embodiments, a second generation CAR comprises an antigen binding domain, a transmembrane domain, and two signaling domains. In some embodiments, a signaling domain mediates downstream signaling during T cell activation. In some embodiments, a signaling domain is a costimulatory domain. In some embodiments, a costimulatory domain enhances cytokine production, CAR T cell proliferation, and/or CAR T cell persistence during T cell activation.

In some embodiments, any one of the cells described herein comprises a nucleic acid encoding a CAR or a third generation CAR. In some embodiments, a third generation CAR comprises an antigen binding domain, a transmembrane domain, and at least three signaling domains. In some embodiments, a signaling domain mediates downstream signaling during T cell activation. In some embodiments, a signaling domain is a costimulatory domain. In some embodiments, a costimulatory domain enhances cytokine production, CAR T cell proliferation, and or CAR T cell persistence during T cell activation. In some embodiments, a third generation CAR comprises at least two costimulatory domains. In some embodiments, the at least two costimulatory domains are not the same.

In some embodiments, any one of the cells described herein comprises a nucleic acid encoding a CAR or a fourth generation CAR. In some embodiments, a fourth generation CAR comprises an antigen binding domain, a transmembrane domain, and at least two, three, or four signaling domains. In some embodiments, a signaling domain mediates downstream signaling during T cell activation. In some embodiments, a signaling domain is a costimulatory domain. In some embodiments, a costimulatory domain enhances cytokine production, CAR T cell proliferation, and or CAR T cell persistence during T cell activation.

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments, a cytokine gene encodes a pro-inflammatory cytokine. In some embodiments, a cytokine gene encodes IL-1, IL-2, IL-9, IL-12, IL-18, TNF, or IFN-gamma, or functional fragment thereof. In some embodiments, a domain which upon successful signaling of the CAR induces expression of a cytokine gene is or comprises a transcription factor or functional domain or fragment thereof. In some embodiments, a domain which upon successful signaling of the CAR induces expression of a cytokine gene is or comprises a transcription factor or functional domain or fragment thereof. In some embodiments, a transcription factor or functional domain or fragment thereof is or comprises a nuclear factor of activated T cells (NFAT), an NF-kB, or functional domain or fragment thereof. See, e.g., Zhang. C. et al., Engineering CAR-T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

A skilled artisan is familiar with CARs and different components and configurations of CARs. Any known CAR can be employed in connection with the provided embodiments. In addition to the CARs described herein, various CARs and nucleotide sequences encoding the same are known in the art and would be suitable for engineering cells as described herein. See, e.g., WO2013040557; WO2012079000; WO2016030414; Smith T, et al., Nature Nanotechnology. 2017. DOI: 10.1038/NNANO.2017.57, the disclosures of which are herein incorporated by reference. Exemplary features and components of a CAR are described in the following subsections.

2. Chimeric Antigen Receptor

In some embodiments, a provided engineered cell, such as an engineered primary cell, is further modified to express a chimeric antigen receptor (CAR). In some embodiments, a provided cell, such as a primary cell, contains a genetic modification of one or more target polynucleotide sequences that regulates the expression of one or more MHC class I molecules, one or more MHC class II molecules, or one or more MHC class I and one or more MHC class II molecules, overexpresses a tolerogenic factor as described herein (e.g. CD47), and expresses a CAR. In some embodiments, the cell, such as primary cell, is one in which: B2M is reduced or eliminated (e.g. knocked out), CIITA is reduced or eliminated (e.g. knocked out), CD47 is overexpressed, and a CAR is expressed. In some embodiments, the cell is B2M−/−, CIITA−/−, CD47tg, CAR+. In some embodiments, the primary cell (e.g. T cell) may additional be one in which TRAC is reduced or eliminated (e.g. knocked out). In some embodiments, the cell is B2−/−, CIITA−/−, CD47tg, TRAC−/− CAR+.

In some embodiments, a polynucleotide encoding a CAR is introduced into the primary cell. In some embodiments, the cell is a T cell, such as a primary T cell. In some embodiments, the cells is a Natural Killer (NK) cell, such as a primary NK cell.

In some embodiments, the CAR is selected from the group consisting of a first generation CAR, a second generation CAR, a third generation CAR, and a fourth generation CAR. In some embodiments, the CAR is or comprises a first generation CAR comprising an antigen binding domain, a transmembrane domain, and at least one signaling domain (e.g., one, two or three signaling domains). In some embodiments, the CAR comprises a second generation CAR comprising an antigen binding domain, a transmembrane domain, and at least two signaling domains. In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an antibody, an antibody fragment, an scFv or a Fab.

In some embodiments, any one of the primary cells described herein comprises a nucleic acid encoding a CAR or a first generation CAR. In some embodiments, a first generation CAR comprises an antigen binding domain, a transmembrane domain, and signaling domain. In some embodiments, a signaling domain mediates downstream signaling during T cell activation.

In some embodiments, any one of the primary cells described herein comprises a nucleic acid encoding a CAR or a second generation CAR. In some embodiments, a second generation CAR comprises an antigen binding domain, a transmembrane domain, and two signaling domains. In some embodiments, a signaling domain mediates downstream signaling during T cell activation. In some embodiments, a signaling domain is a costimulatory domain. In some embodiments, a costimulatory domain enhances cytokine production, CAR T cell proliferation, and/or CAR T cell persistence during T cell activation.

In some embodiments, any one of the primary cells described herein comprises a nucleic acid encoding a CAR or a third generation CAR. In some embodiments, a third generation CAR comprises an antigen binding domain, a transmembrane domain, and at least three signaling domains. In some embodiments, a signaling domain mediates downstream signaling during T cell activation. In some embodiments, a signaling domain is a costimulatory domain. In some embodiments, a costimulatory domain enhances cytokine production, CAR T cell proliferation, and or CAR T cell persistence during T cell activation. In some embodiments, a third generation CAR comprises at least two costimulatory domains. In some embodiments, the at least two costimulatory domains are not the same.

In some embodiments, any one of the primary cells described herein comprises a nucleic acid encoding a CAR or a fourth generation CAR. In some embodiments, a fourth generation CAR comprises an antigen binding domain, a transmembrane domain, and at least two, three, or four signaling domains. In some embodiments, a signaling domain mediates downstream signaling during T cell activation. In some embodiments, a signaling domain is a costimulatory domain. In some embodiments, a costimulatory domain enhances cytokine production, CAR T cell proliferation, and or CAR T cell persistence during T cell activation.

In some embodiments, an engineered primary cell provided herein (e.g. primary T cell or primary NK cell) includes a polynucleotide encoding a CAR, wherein the polynucleotide is inserted in a genomic locus. In some embodiments, the polynucleotide is inserted into a safe harbor locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus. In some embodiments, the polynucleotide is inserted in a B2M, CIITA, TRAC, TRB, PD1 or CTLA4 gene. Any suitable method can be used to insert the CAR into the genomic locus of the hypoimmunogenic cell including the gene editing methods described herein (e.g., a CRISPR/Cas system).

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments, a cytokine gene encodes a pro-inflammatory cytokine. In some embodiments, a cytokine gene encodes IL-1, IL-2, IL-9, IL-12, IL-18, TNF, or IFN-gamma, or functional fragment thereof. In some embodiments, a domain which upon successful signaling of the CAR induces expression of a cytokine gene is or comprises a transcription factor or functional domain or fragment thereof. In some embodiments, a domain which upon successful signaling of the CAR induces expression of a cytokine gene is or comprises a transcription factor or functional domain or fragment thereof. In some embodiments, a transcription factor or functional domain or fragment thereof is or comprises a nuclear factor of activated T cells (NFAT), an NF-kB, or functional domain or fragment thereof. See, e.g., Zhang. C. et al., Engineering CAR-T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

A skilled artisan is familiar with CARs and different components and configurations of CARs. Any known CAR can be employed in connection with the provided embodiments. In addition to the CARs described herein, various CARs and nucleotide sequences encoding the same are known in the art and would be suitable for engineering cells as described herein. See, e.g., WO2013040557; WO2012079000; WO2016030414; Smith T, et al., Nature Nanotechnology. 2017. DOI: 10.1038/NNANO.2017.57, the disclosures of which are herein incorporated by reference. Exemplary features and components of a CAR are described in the following subsections.

a. Antigen Binding Domain

In some embodiments, a CAR antigen binding domain (ABD) is or comprises an antibody or antigen-binding portion thereof. In some embodiments, a CAR antigen binding domain is or comprises an scFv or Fab.

In some embodiments, an antigen binding domain binds to a cell surface antigen of a cell. In some embodiments, a cell surface antigen is characteristic of (e.g., expressed by) a particular or specific cell type. In some embodiments, a cell surface antigen is characteristic of more than one type of cell.

In some embodiments, the antigen may be an antigen that is exclusively or preferentially expressed on tumor cells, or an antigen that is characteristic of an autoimmune or inflammatory disease. In some embodiments, the antigen binding domain (ABD) targets an antigen characteristic of a neoplastic cell. For instance, the antigen binding domain targets an antigen expressed by a neoplastic or cancer cell. In some embodiments, the ABD binds a tumor associated antigen. In some embodiments, the antigen characteristic of a neoplastic cell (e.g., antigen associated with a neoplastic or cancer cell) or a tumor associated antigen is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor.

In some embodiments, the target antigen is an antigen that includes, but is not limited to, Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (S1P1R), NMDA channel, transmembrane protein, multispan transmembrane protein, T-cell receptor motifs; T-cell alpha chains; T-cell β chains; T-cell γ chains; T-cell δ chains, CCR7, CD3, CD4, CD5, CD7, CD8, CD11b, CD11c, CD16, CD19, CD20, CD21, CD22, CD25, CD28, CD34, CD35, CD40, CD45RA, CD45RO, CD52, CD56, CD62L, CD68, CD80, CD95, CD117, CD127, CD133, CD137 (4-1 BB), CD163, F4/80, IL-4Ra, Sca-1, CTLA-4, GITR, GARP, LAP, granzyme B, LFA-1, transferrin receptor, NKp46, perforin, CD4+, Th1, Th2, Th17, Th40, Th22, Th9, Tfh, Canonical Treg, FoxP3+, Tr1, Th3, Treg17, TREG, CDCP, NT5E, EpCAM, CEA, gpA33, Mucins, TAG-72, Carbonic anhydrase IX, PSMA, Folate binding protein, Gangliosides (e.g., CD2, CD3, GM2), Lewis-γ², VEGF, VEGFR 1/2/3, αVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-1β, ALK, RANKL, mTOR, CTLA-4, IL-6, IL-6R, JAK3, BRAF, PTCH, Smoothened, PIGF, ANPEP, TIMP1, PLAUR, PTPRJ, LTBR, or ANTXR1, Folate receptor alpha (FRa), ERBB2 (Her2/neu), EphA2, IL-13Ra2, epidermal growth factor receptor (EGFR), Mesothelin, TSHR, CD19, CD123, CD22, CD30, CD171, CS-1, CLL-1, CD33, EGFRvIII, GD2, GD3, BCMA, MUC16 (CA125), L1CAM, LeY, MSLN, IL13Rα1, L1-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gplOO, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLACI, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, ETV6-AML, sperm protein 17, XAGE1, Tie 2, MAD-CT-1, MAD-CT-2, Major histocompatibility complex class I-related gene protein (MR1), urokinase-type plasminogen activator receptor (uPAR), Fos-related antigen 1, p53, p53 mutant, prostein, survivin, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB I, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIRi, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A,B,C), H2-M3, CD49f, CD151 CD340, CD200, tkrA, trkB, or trkC, or an antigenic fragment or antigenic portion thereof.

In some embodiments, exemplary target antigens include, but are not limited to, CDS, CD19, CD20, CD22, CD23, CD30, CD70, Kappa, Lambda, and B cell maturation agent (BCMA) (associated with leukemias); CS1/SLAMF7, CD38, CD138, GPRC5D, TACI, and BCMA (associated with myelomas); GD2, HER2, EGFR, EGFRvlll, B7H3, PSMA, PSCA, CAIX, CD171, CEA, CSPG4, EPHA2, FAP, FRa, IL-13Ra, Mesothelin, MUC1, MUC16, and ROR1 (associated with solid tumors).

In some embodiments, the CAR is a CD19 CAR. In some embodiments, the extracellular binding domain of the CD19 CAR comprises an antibody that specifically binds to CD19, for example, human CD19. In some embodiments, the extracellular binding domain of the CD19 CAR comprises an scFv antibody fragment derived from the FMC63 monoclonal antibody (FMC63), which comprises the heavy chain variable region (VH) and the light chain variable region (VL) of FMC63 connected by a linker peptide. In some embodiments, the linker peptide is a “Whitlow” linker peptide. FMC63 and the derived scFv have been described in Nicholson et al., Mal. Immun. 34(16-17):1157-1165 (1997) and PCT Application Publication No. WO2018/213337 A1, the entire content of each of which is incorporated by reference herein.

In some embodiments, the extracellular binding domain of the CD19 CAR comprises an antibody derived from one of the CD19-specific antibodies including, for example, SJ25C1 (Bejcek et al., Cancer Res. 55:2346-2351 (1995)), HD37 (Pezutto et al., J. Immunol. 138(9):2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Gallard et al., J. Immunology, 148(10): 2983-2987 (1992)), and CLB-CD19 (De Rie Cell. Immunol. 118:368-381(1989)).

In some embodiments, the CAR is CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR comprises an extracellular binding domain that specifically binds CD22, a transmembrane domain, an intracellular signaling domain, and/or an intracellular costimulatory domain. In some embodiments, the extracellular binding domain of the CD22 CAR comprises an scFv antibody fragment derived from the m971 monoclonal antibody (m971), which comprises the heavy chain variable region (VH) and the light chain variable region (VL) of m971 connected by a linker. In some embodiments, the extracellular binding domain of the CD22 CAR comprises an scFv antibody fragment derived from m971-L7, which an affinity matured variant of m971 with significantly improved CD22 binding affinity compared to the parental antibody m971 (improved from about 2 nM to less than 50 pM). In some embodiments, the scFv antibody fragment derived from m971-L7 comprises the VH and the VL of m971-L7 connected by a 3×G4S linker. In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the CAR is BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR comprises an extracellular binding domain that specifically binds BCMA, a transmembrane domain, an intracellular signaling domain, and/or an intracellular costimulatory domain. In some embodiments, the extracellular binding domain of the BCMA CAR comprises an antibody that specifically binds to BCMA, for example, human BCMA. CARs directed to BCMA have been described in PCT Application Publication Nos. WO2016/014789, WO2016/014565, WO2013/154760, and WO 2015/128653. BCMA-binding antibodies are also disclosed in PCT Application Publication Nos. WO2015/166073 and WO2014/068079. In some embodiments, the extracellular binding domain of the BCMA CAR comprises an scFv antibody fragment derived from a murine monoclonal antibody as described in Carpenter et al., Clin. Cancer Res. 19(8):2048-2060 (2013). In some embodiments, the scFv antibody fragment is a humanized version of the murine monoclonal antibody (Sommermeyer et al., Leukemia 31:2191-2199 (2017)). In some embodiments, the extracellular binding domain of the BCMA CAR comprises single variable fragments of two heavy chains (VHH) that can bind to two epitopes of BCMA as described in Zhao et al., J. Hematol. Oneal. 11(1):141 (2018). In some embodiments, the extracellular binding domain of the BCMA CAR comprises a fully human heavy-chain variable domain (FHVH) as described in Lam et al., Nat. Commun. 11(1):283 (2020).

In some embodiments, the antigen binding domain targets an antigen characteristic of an autoimmune or inflammatory disorder. In some embodiments, the ABD binds an antigen associated with an autoimmune or inflammatory disorder. In some instances, the antigen is expressed by a cell associated with an autoimmune or inflammatory disorder. In some embodiments, the autoimmune or inflammatory disorder is selected from chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purrpura, neuromyelits optica, Evan's syndrome, IgM mediated neuropathy, cyroglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. Allosensitization, in some instances, refers to the development of an immune response (such as circulating antibodies) against MHC molecules (e.g., human leukocyte antigens) that the immune system of the recipient subject or pregnant subject considers to be non-self antigens. In some embodiments, the antigen characteristic of an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen binding domain of a CAR binds to a ligand expressed on B cells, plasma cells, or plasmablasts. In some embodiments, an antigen binding domain of a CAR binds to CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See, US 2003/0077249; WO 2017/058753; WO 2017/058850, the contents of which are herein incorporated by reference. In some embodiments, the CAR is an anti-CD19 CAR. In some embodiments, the CAR is an anti-BCMA CAR.

In some embodiments, the antigen binding domain targets an antigen characteristic of senescent cells, e.g., urokinase-type plasminogen activator receptor (uPAR). In some embodiments, the ABD binds an antigen associated with a senescent cell. In some instances, the antigen is expressed by a senescent cell. In some embodiments, the CAR may be used for treatment or prophylaxis of disorders characterized by the aberrant accumulation of senescent cells, e.g., liver and lung fibrosis, atherosclerosis, diabetes and osteoarthritis.

In some embodiments, the antigen binding domain targets an antigen characteristic of an infectious disease. In some embodiments, the ABD binds an antigen associated with an infectious disease. In some instances, the antigen is expressed by a cell affected by an infectious disease. In some embodiments, wherein the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Epstein-Barr virus, CMV, human papillomavirus. In some embodiments, the antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, HIV Env, gp120, or CD4-induced epitope on HIV-1 Env.

In any of these embodiments, the extracellular binding domain of the CAR can be codon-optimized for expression in a host cell or have variant sequences to increase functions of the extracellular binding domain.

In some embodiments, the CAR is bispecific to two target antigens. In some embodiments, the target antigens are different target antigens. In some of any such embodiments, the two different target antigens are any two different antigens described above. In some embodiments, the extracellular binding domains are different and bind two different antigens from (i) CD19 and CD20, (ii) CD20 and L1-CAM, (iii) L1-CAM and GD2, (iv) EGFR and L1-CAM, (v) CD19 and CD22, (vi) EGFR and C-MET, (vii) EGFR and HER2, (viii) C-MET and HER2, or (ix) EGFR and ROR1. In some embodiments, each of the two different antigen binding domains is an scFv. In some embodiments, the C-terminus of one variable domain (VH or VL) of a first scFv is tethered to the N-terminus of the second scFv (VL or VH, respectively) via a polypeptide linker. In some embodiments, the linker connects the N-terminus of the VH with the C-terminus of VL or the C-terminus of VH with the N-terminus of VL. These scFvs lack the constant regions (Fc) present in the heavy and light chains of the native antibody. The scFvs, specific for at least two different antigens, are arranged in tandem and linked to the co-stimulatory domain and the intracellular signaling domain via a transmembrane domain. In an embodiment, an extracelluar spacer domain may be linked between the antigen-specific binding region and the transmembrane domain.

In a further embodiment, each antigen-specific targeting region of the CAR comprises a divalent (or bivalent) single-chain variable fragment (di-scFvs, bi-scFvs). In CARs comprising di-scFVs, two scFvs specific for each antigen are linked together by producing a single peptide chain with two VH and two VL regions, yielding tandem scFvs. (Xiong, Cheng-Yi; Natarajan, A; Shi, X B; Denardo, G L; Denardo, S J (2006). “Development of tumor targeting anti-MUC-1 multimer: effects of di-scFv unpaired cysteine location on PEGylation and tumor binding”. Protein Engineering Design and Selection 19 (8): 359-367; Kufer, Peter, Lutterbiise, Ralf; Baeuerle, Patrick A. (2004). “A revival of bispecific antibodies”. Trends in Biotechnology 22 (5): 238-244). CARs comprising at least two antigen-specific targeting regions would express two scFvs specific for each of the two antigens. The resulting antigen-specific targeting region, specific for at least two different antigens, is joined to the co-stimulatory domain and the intracellular signaling domain via a transmembrane domain. In an embodiment, an extracelluar spacer domain may be linked between the antigen-specific binding domain and the transmembrane domain.

In an additional embodiment, each antigen-specific targeting region of the CAR comprises a diabody. In a diabody, the scFvs are created with linker peptides that are too short for the two variable regions to fold together, driving the scFvs to dimerize. Still shorter linkers (one or two amino acids) lead to the formation of trimers, the so-called triabodies or tribodies. Tetrabodies may also be used.

In some embodiments, the cell is engineered to express more than one CAR, such as two different CARs, in which each CAR has an antigen-binding domain directed to a different target antigen. In some of any such embodiments, the two different target antigens are any two different antigens described above. In some embodiments, the extracellular binding domains are different and bind two different antigens from (i) CD19 and CD20, (ii) CD20 and L1-CAM, (iii) L1-CAM and GD2, (iv) EGFR and L1-CAM, (v) CD19 and CD22, (vi) EGFR and C-MET, (vii) EGFR and HER2, (viii) C-MET and HER2, or (ix) EGFR and ROR1.

In some embodiments, two different engineered cells are prepared that contain the provided modifications with each engineered with a different CAR. In some embodiments, each of the two different CARs has an antigen-binding domain directed to a different target antigen. In some of any such embodiments, the two different target antigens are any two different antigens described above. In some embodiments, the extracellular binding domains are different and bind two different antigens from (i) CD19 and CD20, (ii) CD20 and L1-CAM, (iii) L1-CAM and GD2, (iv) EGFR and L1-CAM, (v) CD19 and CD22, (vi) EGFR and C-MET, (vii) EGFR and HER2, (viii) C-MET and HER2, or (ix) EGFR and ROR1. In some embodiments, a population of engineered cells (e.g. hypoimmunogenic) expressing a first CAR directed against a first target antigen and a population of engineered cells (e.g. hypoimmunogenic) expressing a second CAR directed against a second target antigen are separately administered to the subject. In some embodiments, the first and second population of cells are administered sequentially in any order. For instance, the population of cells expressing the second CAR is administered a after administration of the population of cells expressing the first CAR.

b. Spacer

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine and serine residues such as but not limited to glycine-serine doublets. In some embodiments, the CAR comprises two or more spacers, e.g., a spacer between the antigen binding domain and the transmembrane domain and a spacer between the transmembrane domain and a signaling domain.

c. Transmembrane Domain

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD28, CD33, CD37, CD64, CD80, CD86, CD134, CD137, CD154, or functional variant thereof. In some embodiments, the transmembrane domain comprises at least a transmembrane region(s) of CD8α, CD8β, 4-1BB/CD137, CD28, CD34, CD4, FceRIγ, CD16, OX40/CD134, CD3G, CD3e, CD37, CD36, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

d. Signaling Domain(s)

In some embodiments, a CAR described herein comprises one or at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the at least one signaling domain comprises a CD3 zeta domain or an immunoreceptor tyrosine-based activation motif (ITAM), or functional variant thereof.

In some embodiments, a CAR comprises a signaling domain which is a costimulatory domain. In some embodiments, a CAR comprises a second costimulatory domain. In some embodiments, a CAR comprises at least two costimulatory domains. In some embodiments, a CAR comprises at least three costimulatory domains. In some embodiments, a CAR comprises a costimulatory domain selected from one or more of CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83. In some embodiments, if a CAR comprises two or more costimulatory domains, two costimulatory domains are different. In some embodiments, if a CAR comprises two or more costimulatory domains, two costimulatory domains are the same.

In other embodiments, the at least one signaling domain comprises (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least one signaling domain comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least two signaling domains comprise a CD3 zeta domain or an immunoreceptor tyrosine-based activation motif (ITAM), or functional variant thereof. In other embodiments, the at least two signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the at least one signaling domain comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least two signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the at least three signaling domains comprise a CD3 zeta domain or an immunoreceptor tyrosine-based activation motif (ITAM), or functional variant thereof. In other embodiments, the at least three signaling domains comprise (i) a CD3 zeta domain, or an immunoreceptor tyrosine-based activation motif (ITAM), or functional variant thereof; and (ii) a CD28 domain, or a 4-1BB domain, or functional variant thereof. In yet other embodiments, the least three signaling domains comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the at least three signaling domains comprise a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof.

In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof.

In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

e. Exemplary CARs

In some embodiments, the CAR comprises an extracellular antigen binding domain (e.g., antibody or antibody fragment, such as an scFv) that binds to an antigen (e.g. tumor antigen), a spacer (e.g. containing a hinge domain, such as any as described herein), a transmembrane domain (e.g. any as described herein), and an intracellular signaling domain (e.g. any intracellular signaling domain, such as a primary signaling domain or costimulatory signaling domain as described herein). In some embodiments, the intracellular signaling domain is or includes a primary cytoplasmic signaling domain. In some embodiments, the intracellular signaling domain additionally includes an intracellular signaling domain of a costimulatory molecule (e.g., a costimulatory domain). Any of such components can be any as described above.

Examples of exemplary components of a CAR are described in Table 3. In provided aspects, the sequences of each component in a CAR can include any combination listed in Table 3.

TABLE 3
CAR components and Exemplary Sequences

| | | SEQ |
|---|---|---|
| | | ID |
| Component | Sequence | NO |
| Extracellular | | |
| binding | | |
| domain | | |
| Anti-CD19 | DIQMTQTTSSLSASLGDRVTISCRASQDISK | 3 |
| scFv | YLNWYQQKPDGTVKLLIYHTSRLHSGVPSRF | |
| (FMC63) | SGSGSGTDYSLTISNLEQEDIATYFCQQGNT | |
| | LPYTFGGGTKLEITGSTSGSGKPGSGEGSTK | |
| | GEVKLQESGPGLVAPSQSLSVTCTVSGVSLP | |
| | DYGVSWIRQPPRKGLEWLGVIWGSETTYYNS | |
| | ALKSRLTIIKDNSKSQVFLKMNSLQTDDTAI | |
| | YYCAKHYYYGGSYAMDYWGQGTSVTVSS | |
| Anti-CD19 | DIQMTQTTSSLSASLGDRVTISCRASQDISK | 4 |
| scFv | YLNWYQQKPDGTVKLLIYHTSRLHSGVPSRF | |
| (FMC63) | SGSGSGTDYSLTISNLEQEDIATYFCQQGNT | |
| | LPYTFGGGTKLEITGGGGSGGGGSGGGGSEV | |
| | KLQESGPGLVAPSQSLSVTCTVSGVSLPDYG | |
| | VSWIRQPPRKGLEWLGVIWGSETTYYNSALK | |
| | SRLTIIKDNSKSQVFLKMNSLQTDDTAIYYC | |
| | AKHYYYGGSYAMDYWGQGTSVTVSS | |
| Spacer | | |
| (e.g. hinge) | | |
| IgG4 Hinge | ESKYGPPCPPCP | 5 |
| CD8 Hinge | TTTPAPRPPTPAPTIASQPLSLRPE | 6 |
| CD28 | IEVMYPPPYLDNEKSNGTIIHVKGKHLCPSP | 7 |
| | LFPGPSKP | |
| Transmembrane | | |
| CD8 | ACRPAAGGAVHTRGLDFACDIYIWAPLAGTC | 8 |
| | GVLLLSLVITLYC | |
| CD28 | FWVLVVVGGVLACYSLLVTVAFIIFWV | 9 |
| CD28 | FWVLVVVGGVLACYSLLVTVAFIIFWV | 10 |
| Costimulatory | | |
| domain | | |
| CD28 | RSKRSRLLHSDYMNMTPRRPGPTRKHYQPYA | 11 |
| | PPRDFAAYRS | |
| 4-1BB | KRGRKKLLYIFKQPFMRPVQTTQEEDGCSCR | 12 |
| | FPEEEEGGCEL | |
| Primary | | |
| Signaling | | |
| Domain | | |
| CD3zeta | RVKFSRSADAPAYQQGQNQLYNELNLGRREE | 13 |
| | YDVLDKRRGRDPEMGGKPRRKNPQEGLYNEL | |
| | QKDKMAEAYSEIGMKGERRRGKGHDGLYQGL | |
| | STATKDTYDALHMQALPPR | |
| CD3zeta | RVKFSRSADAPAYKQGQNQLYNELNLGRREE | 14 |
| | YDVLDKRRGRDPEMGGKPRRKNPQEGLYNEL | |
| | QKDKMAEAYSEIGMKGERRRGKGHDGLYQGL | |
| | STATKDTYDALHMQALPPR | |

3. Methods of Increasing Expression (e.g., Overexpression) of a Polynucleotide

In some embodiments, increased expression of a polynucleotide may be carried out by any of a variety of techniques. For instance, methods for modulating expression of genes and factors (proteins) include genome editing technologies, and, RNA or protein expression technologies and the like. For all of these technologies, well known recombinant techniques are used, to generate recombinant nucleic acids as outlined herein. In some embodiments, the cell that is engineered with the one or more modifications for overexpression or increased expression of a polynucleotide is any source cell as described herein. In some embodiments, the source cell is any cell described in Section II.C.

In some embodiments, expression of a gene is increased by increasing endogenous gene activity (e.g., increasing transcription of the exogenous gene). In some cases, endogenous gene activity is increased by increasing activity of a promoter or enhancer operably linked to the endogenous gene. In some embodiments, increasing activity of the promoter or enhancer comprises making one or more modifications to an endogenous promoter or enhancer that increase activity of the endogenous promoter or enhancer. In some cases, increasing gene activity of an endogenous gene comprises modifying an endogenous promoter of the gene. In some embodiments increasing gene activity of an endogenous gene comprises introducing a heterologous promoter. In some embodiments, the heterologous promoter is selected from the group consisting of a CAG promoter, cytomegalovirus (CMV) promoter, EF1a promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, Rous sarcoma virus (RSV) promoter, and UBC promoter.

a. DNA-Binding Fusion Proteins

In some embodiments, expression of a target gene (e.g., CD47, or another tolerogenic factor) is increased by expression of fusion protein or a protein complex containing (1) a site-specific binding domain specific for the endogenous CD47, or other gene and (2) a transcriptional activator.

In some embodiments, the regulatory factor is comprised of a site specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP, which are also known as zinc finger nucleases (ZFNs).

In some embodiments, the regulatory factor comprises a site-specific binding domain, such as using a DNA binding protein or DNA-binding nucleic acid, which specifically binds to or hybridizes to the gene at a targeted region. In some embodiments, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease or an RNA-guided nuclease such as a clustered regularly interspersed short palindromic nucleic acid (CRISPR)-Cas system, such as CRISPR-Cas9 system. In some embodiments, the nuclease is modified to lack nuclease activity. In some embodiments, the modified nuclease is a catalytically dead dCas9.

In some embodiments, the site specific binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. See also U.S. Pat. Nos. 5,420,032; 6,833,252; Belfort et al., (1997) Nucleic Acids Res. 25:3379-3388; Dujon et al., (1989) Gene 82:115-118; Perler et al, (1994) Nucleic Acids Res. 22, 1125-1127; Jasin (1996) Trends Genet. 12:224-228; Gimble et al., (1996) J. Mol. Biol. 263:163-180; Argast et al, (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al, (2002) Molec. Cell 10:895-905; Epinat et al, (2003) Nucleic Acids Res. 31:2952-2962; Ashworth et al, (2006) Nature 441:656-659; Paques et al, (2007) Current Gene Therapy 7:49-66; U.S. Patent Publication No. 2007/0117128.

Zinc finger, TALE, and CRISPR system binding domains can be “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073, incorporated by reference in its entirety herein.

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a guide sequence includes a targeting domain (e.g. targeting sequence) comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

In some embodiments, the gRNA may be any as described herein. In particular embodiments, the gRNA has a targeting sequence that is complementary to a target site of CD47, such as set forth in any one of SEQ ID NOS:200784-231885 (Table 29, Appendix 22 of WO2016183041); HLA-E, such as set forth in any one of SEQ ID NOS:189859-193183 (Table 19, Appendix 12 of WO2016183041); HLA-F, such as set forth in any one of SEQ ID NOS: 688808-699754 (Table 45, Appendix 38 of WO2016183041); HLA-G, such as set forth in any one of SEQ ID NOS:188372-189858 (Table 18, Appendix 11 of WO2016183041); or PD-L1, such as set forth in any one of SEQ ID NOS: 193184-200783 (Table 21, Appendix 14 of WO2016183041).

In some embodiments, the target site is upstream of a transcription initiation site of the target gene. In some embodiments, the target site is adjacent to a transcription initiation site of the gene. In some embodiments, the target site is adjacent to an RNA polymerase pause site downstream of a transcription initiation site of the gene.

In some embodiments, the targeting domain is configured to target the promoter region of the target gene to promote transcription initiation, binding of one or more transcription enhancers or activators, and/or RNA polymerase. One or more gRNA can be used to target the promoter region of the gene. In some embodiments, one or more regions of the gene can be targeted. In certain aspects, the target sites are within 600 base pairs on either side of a transcription start site (TSS) of the gene.

It is within the level of a skilled artisan to design or identify a gRNA sequence (i.e. gRNA targeting sequence) that is or comprises a sequence targeting a gene, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; www.e-crisp.org/E-CRISP/; crispr.mit.edu/). In some embodiments, the gRNA sequence is or comprises a targeting sequence with minimal off-target binding to a non-target gene.

In some embodiments, the regulatory factor further comprises a functional domain, e.g., a transcriptional activator.

In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of the target gene. In some cases, the transcriptional activator, can be or contain all or a portion of a heterologous transactivation domain. For example, in some embodiments, the transcriptional activator is selected from Herpes simplex-derived transactivation domain, Dnmt3a methyltransferase domain, p65, VP16, and VP64.

In some embodiments, the regulatory factor is a zinc finger transcription factor (ZF-TF). In some embodiments, the regulatory factor is VP64-p65-Rta (VPR).

In certain embodiments, the regulatory factor further comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases) and their associated factors and modifiers. See, e.g., U.S. Publication No. 2013/0253040, incorporated by reference in its entirety herein.

Suitable domains for achieving activation include the HSV VP 16 activation domain (see, e.g., Hagmann et al, J. Virol. 71, 5952-5962 (1 97)) nuclear hormone receptors (see, e.g., Torchia et al., Curr. Opin. Cell. Biol. 10:373-383 (1998)); the p65 subunit of nuclear factor kappa B (Bitko & Bank, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Spl, AP-2, and CTF1 (Seipel et al, EMBOJ. 11, 4961-4968 (1992) as well as p300, CBP, PCAF, SRC1 PvALF, AtHD2A and ERF-2. See, for example, Robyr et al, (2000) Mol. Endocrinol. 14:329-347; Collingwood et al, (1999) J. Mol. Endocrinol 23:255-275; Leo et al, (2000) Gene 245:1-11; Manteuffel-Cymborowska (1999) Acta Biochim. Pol. 46:77-89; McKenna et al, (1999) J. Steroid Biochem. Mol. Biol. 69:3-12; Malik et al, (2000) Trends Biochem. Sci. 25:277-283; and Lemon et al, (1999) Curr. Opin. Genet. Dev. 9:499-504. Additional exemplary activation domains include, but are not limited to, OsGAI, HALF-1, C1, AP1, ARF-5, -6,-1, and -8, CPRF1, CPRF4, MYC-RP/GP, and TRAB1, See, for example, Ogawa et al, (2000) Gene 245:21-29; Okanami et al, (1996) Genes Cells 1:87-99; Goff et al, (1991) Genes Dev. 5:298-309; Cho et al, (1999) Plant Mol Biol 40:419-429; Ulmason et al, (1999) Proc. Natl. Acad. Sci. USA 96:5844-5849; Sprenger-Haussels et al, (2000) Plant J. 22:1-8; Gong et al, (1999) Plant Mol. Biol. 41:33-44; and Hobo et al., (1999) Proc. Natl. Acad. Sci. USA 96:15,348-15,353.

Exemplary repression domains that can be used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MeCP2. See, for example, Bird et al, (1999) Cell 99:451-454; Tyler et al, (1999) Cell 99:443-446; Knoepfler et al, (1999) Cell 99:447-450; and Robertson et al, (2000) Nature Genet. 25:338-342. Additional exemplary repression domains include, but are not limited to, ROM2 and AtHD2A. See, for example, Chem et al, (1996) Plant Cell 8:305-321; and Wu et al, (2000) Plant J. 22:19-27.

In some instances, the domain is involved in epigenetic regulation of a chromosome. In some embodiments, the domain is a histone acetyltransferase (HAT), e.g. type-A, nuclear localized such as MYST family members MOZ, Ybf2/Sas3, MOF, and Tip60, GNAT family members Gcn5 or pCAF, the p300 family members CBP, p300 or Rtt109 (Bemdsen and Denu (2008) Curr Opin Struct Biol 18(6):682-689). In other instances, the domain is a histone deacetylase (HD AC) such as the class I (HDAC-1, 2, 3, and 8), class II molecules (HDAC IIA (HDAC-4, 5, 7 and 9), HD AC IIB (HDAC 6 and 10)), class IV (HDAC-11), class III (also known as sirtuins (SIRTs); SIRT1-7) (see Mottamal et al., (2015) Molecules 20(3):3898-3941). Another domain that is used in some embodiments is a histone phosphorylase or kinase, where examples include MSK1, MSK2, ATR, ATM, DNA-PK, Bubl, VprBP, IKK-a, PKCpi, Dik/Zip, JAK2, PKC5, WSTF and CK2. In some embodiments, a methylation domain is used and may be chosen from groups such as Ezh2, PRMT1/6, PRMT5n, PRMT 2/6, CARM1, set7/9, MLL, ALL-1, Suv 39h, G9a, SETDB1, Ezh2, Set2, Dotl, PRMT 1/6, PRMT 5/7, PR-Set7 and Suv4-20h, Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al, (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

b. Exogenous Polypeptide

In some embodiments, increased expression (i.e. overexpression) of the polynucleotide is mediated by introducing into the primary cell an exogenous polynucleotide encoding the polynucleotide to be overexpressed. In some embodiments, the exogenous polynucleotide is a recombinant nucleic acid. Well-known recombinant techniques can be used to generate recombinant nucleic acids as outlined herein.

In certain embodiments, the recombinant nucleic acids encoding an exogenous polynucleotide, such as a tolerogenic factor or a chimeric antigen receptor, may be operably linked to one or more regulatory nucleotide sequences in an expression construct. Regulatory nucleotide sequences will generally be appropriate for the host cell and recipient subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, and/or the expression of any other protein encoded by the vector, such as antibiotic markers.

In some embodiments, the exogenous polynucleotide is operably linked to a promoter for expression of the exogenous polynucleotide in the engineered cell. Examples of suitable mammalian promoters include, for example, promoters from the following genes: elongation factor 1 alpha (EF1α) promoter, ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al, Nature 273: 113-120 (1978)). The immediate early promoter of the human cytomegalovirus is conveniently obtained as a HindIII restriction enzyme fragment (Greenaway et al, Gene 18: 355-360 (1982)). The foregoing references are incorporated by reference in their entirety.

In some embodiments, the expression vector is a bicistronic or multicistronic expression vector. Bicistronic or multicistronic expression vectors may include (1) multiple promoters fused to each of the open reading frames; (2) insertion of splicing signals between genes; (3) fusion of genes whose expressions are driven by a single promoter; and (4) insertion of proteolytic cleavage sites between genes (self-cleavage peptide) or insertion of internal ribosomal entry sites (IRESs) between genes.

In some embodiments, an expression vector or construct herein is a multicistronic construct. The terms “multicistronic construct” and “multicistronic vector” are used interchangeably herein and refer to a recombinant DNA construct that is to be transcribed into a single mRNA molecule, wherein the single mRNA molecule encodes two or more genes (e.g., two or more transgenes). The multi-cistronic construct is referred to as bicistronic construct if it encodes two genes, and tricistronic construct if it encodes three genes, and quadrocistronic construct if it encodes four genes, and so on.

In some embodiments, two or more exogenous polynucleotides comprised by a vector or construct (e.g., a transgene) are each separated by a multicistronic separation element. In some embodiments, the multicistronic separation element is an IRES or a sequence encoding a cleavable peptide or ribosomal skip element. In some embodiments, the multicistronic separation element is an IRES, such as an encephalomyocarditis (EMCV) virus IRES. In some embodiments, the multicistronic separation element is a cleavable peptide such as a 2A peptide. Exemplary 2A peptides include a P2A peptide, a T2A peptide, an E2A peptide, and an F2A peptide. In some embodiments, the cleavable peptide is a T2A. In some embodiments, the two or more exogenous polynucleotides (e.g. the first exogenous polynucleotide and second exogenous polynucleotide) are operably linked to a promoter. In some embodiments, the first exogenous polynucleotide and the second exogenous polynucleotide are each operably linked to a promoter. In some embodiments, the promoter is the same promoter. In some embodiments, the promoter is an EF1 promoter.

In some cases, an exogenous polynucleotide encoding an exogenous polypeptide (e.g., an exogenous polynucleotide encoding a tolerogenic factor or complement inhibitor described herein) encodes a cleavable peptide or ribosomal skip element, such as T2A at the N-terminus or C-terminus of an exogenous polypeptide encoded by a multicistronic vector. In some embodiments, inclusion of the cleavable peptide or ribosomal skip element allows for expression of two or more polypeptides from a single translation initiation site. In some embodiments, the cleavable peptide is a T2A. In some embodiments, the T2A is or comprises the amino acid sequence set forth by SEQ ID NO: 15. In some embodiments, the T2A is or comprises the amino acid sequence set forth by SEQ ID NO: 16. In some embodiments, the T2A is or comprises the amino acid sequence set forth by SEQ ID NO: 21. In some embodiments, the T2A is or comprises the amino acid sequence set forth by SEQ ID NO: 22.

In some embodiments, the vector or construct includes a single promoter that drives the expression of one or more transcription units of an exogenous polynucleotide. In some embodiments, such vectors or constructs can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g. one or more tolerogenic factors such as CD47) from an RNA transcribed from a single promoter. In some embodiments, the vectors or constructs provided herein are bicistronic, allowing the vector or construct to express two separate polypeptides. In some cases, the two separate polypeptides encoded by the vector or construct are tolerogenic factors (e.g., two factors selected from DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, C1-Inhibitor, IL-10, IL-35, FASL, CCL21, MFGE8, SERPINB9, CD35, IL-39, CD16 Fc Receptor, IL15-RF, and H2-M3 (including any combination thereof). In some embodiments, the tolerogenic factor is two or more of CD47, PD-L1, HLA-E or HLA-G, CCL21, FasL, Serpinb9, CD200, and Mfge8 (including any combination thereof). In some embodiments, the two separate polypeptides encoded by the vector or construct are a tolerogenic factor (e.g., CD47). In some embodiments, the vectors or constructs provided herein are tricistronic, allowing the vector or construct to express three separate polypeptides. In some cases, the three nucleic acid sequences of the tricistronic vector or construct are a tolerogenic factor such as CD47. In some cases, the three nucleic acid sequences of the tricistronic vector or construct are three tolerogenic factors selected from DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, C1-Inhibitor, IL-10, IL-35, FASL, CCL21, MFGE8, SERPINB9, CD35, IL-39, CD16 Fc Receptor, IL15-RF, and H2-M3 (including any combination thereof). In some embodiments, the three tolerogenic factor are selected from CD47, PD-L1, HLA-E or HLA-G, CCL21, FasL, Serpinb9, CD200, and Mfge8 (including any combination thereof). In some embodiments, the vectors or constructs provided herein are quadrocistronic, allowing the vector or construct to express four separate polypeptides. In some cases, the four separate polypeptides of the quadrocistronic vector or construct are four tolerogenic factors selected from DUX4, B2M-HLA-E, CD35, CD52, CD16, CD52, CD47, CD46, CD55, CD59, CD27, CD200, HLA-C, HLA-E, HLA-E heavy chain, HLA-G, PD-L1, IDO1, CTLA4-Ig, C1-Inhibitor, IL-10, IL-35, FASL, CCL21, MFGE8, SERPINB9, CD35, IL-39, CD16 Fc Receptor, IL15-RF, and H2-M3 (including any combination thereof). In some embodiments, the four tolerogenic factor are selected from CD47, PD-L1, HLA-E or HLA-G, CCL21, FasL, Serpinb9, CD200, and Mfge8 (including any combination thereof). In some embodiments, the cell comprises one or more vectors or constructs, wherein each vector or construct is a monocistronic or a multicistronic construct as described above, and the monocistronic or multicistronic constructs encode one or more tolerogenic factors, in any combination or order.

In some embodiments, the cell comprises one or more vectors or constructs, wherein each vector or construct is a monocistronic or a multicistronic construct as described above, and the monocistronic or multicistronic constructs encode one or more tolerogenic factors, in any combination or order.

In some embodiments, a single promoter directs expression of an RNA that contains, in a single open reading frame (ORF), two, three, or four genes (e.g. encoding a tolerogenic factor (e.g., CD47)) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein include, without limitation, 2A sequences from the foot-and-mouth disease virus (F2A, e.g., SEQ ID NO: 20), equine rhinitis A virus (E2A, e.g., SEQ ID NO: 19), thosea asigna virus (T2A, e.g., SEQ ID NO: 15, 16, 21, or 22), and porcine teschovirus-1 (P2A, e.g., SEQ ID NO: 17 or 18) as described in U.S. Patent Publication No. 20070116690.

In cases where the vector or construct (e.g., transgene) contains more than one nucleic acid sequence encoding a protein, e.g., a first exogenous polynucleotide encoding CD47, and second exogenous polynucleotide encoding a second transgene, the vector or construct (e.g., transgene) may further include a nucleic acid sequence encoding a peptide between the first and second exogenous polynucleotide sequences. In some cases, the nucleic acid sequence positioned between the first and second exogenous polynucleotides encodes a peptide that separates the translation products of the first and second exogenous polynucleotides during or after translation. In some embodiments, the peptide contains a self-cleaving peptide or a peptide that causes ribosome skipping (a ribosomal skip element), such as a T2A peptide. In some embodiments, inclusion of the cleavable peptide or ribosomal skip element allows for expression of two or more polypeptides from a single translation initiation site. In some embodiments, the peptide is a self-cleaving peptide that is a T2A peptide. In some embodiments, the T2A is or comprises the amino acid sequence set forth by SEQ ID NO: 15. In some embodiments, the T2A is or comprises the amino acid sequence set forth by SEQ ID NO: 16. In some embodiments, the T2A is or comprises the amino acid sequence set forth by SEQ ID NO: 21. In some embodiments, the T2A is or comprises the amino acid sequence set forth by SEQ ID NO: 22.

The process of introducing the polynucleotides described herein into primary cells can be achieved by any suitable technique. Suitable techniques include calcium phosphate or lipid-mediated transfection, electroporation, transposase-mediated delivery, and transduction or infection using a viral vector. In some embodiments, the polynucleotides are introduced into a cell via viral transduction (e.g., lentiviral transduction) or otherwise delivered on a viral vector (e.g., fusogen-mediated delivery). In some embodiments, vectors that package a polynucleotide encoding an exogenous polynucleotide may be used to deliver the packaged polynucleotides to a cell or population of cells. These vectors may be of any kind, including DNA vectors, RNA vectors, plasmids, viral vectors and particles. In some embodiments, lipid nanoparticles can be used to deliver an exogenous polynucleotide to a cell. In some embodiments, viral vectors can be used to deliver an exogenous polynucleotide to a cell. Viral vector technology is well known and described in Sambrook et al. (2001, Molecular Cloning: A Laboratory Manual, Cold Spring Harbor Laboratory, New York). Viruses, which are useful as vectors include, but are not limited to lentiviral vectors, adenoviral vectors, adeno-associated viral (AAV) vectors, herpes simplex viral vectors, retroviral vectors, oncolytic viruses, and the like. In some embodiments, the introduction of the exogenous polynucleotide into the cell can be specific (targeted) or non-specific (e.g. non-targeted). In some embodiments, the introduction of the exogenous polynucleotide into the cell can result in integration or insertion into the genome in the cell. In other embodiments, the introduced exogenous polynucleotide may be non-integrating or episomal in the cell. A skilled artisan is familiar with methods of introducing nucleic acid transgenes into a cell, including any of the exemplary methods described herein, and can choose a suitable method.
1) Non-Targeted Delivery

In some embodiments, an exogenous polynucleotide is introduced into a primary cell (e.g. source cell) by any of a variety of non-targeted methods. In some embodiments, the exogenous polynucleotide is inserted into a random genomic locus of a host cell. As known to a person skilled in the art, viral vectors, including, for example, retroviral vectors and lentiviral vectors are commonly used to deliver genetic material into host cells and randomly insert the foreign or exogenous gene into the host cell genome to facilitate stable expression and replication of the gene. In some embodiments, the non-targeted introduction of the exogenous polynucleotide into the cell is under conditions for stable expression of the exogenous polynucleotide in the cell. In some embodiments, methods for introducing a nucleic acid for stable expression in a cell involves any method that results in stable integration of the nucleic acid into the genome of the cell, such that it may be propagated if the cell it has integrated into divides.

In some embodiments, the viral vector is a lentiviral vector. Lentiviral vectors are particularly useful means for successful viral transduction as they permit stable expression of the gene contained within the delivered nucleic acid transcript. Lentiviral vectors express reverse transcriptase and integrase, two enzymes required for stable expression of the gene contained within the delivered nucleic acid transcript. Reverse transcriptase converts an RNA transcript into DNA, while integrase inserts and integrates the DNA into the genome of the target cell. Once the DNA has been integrated stably into the genome, it divides along with the host. The gene of interest contained within the integrated DNA may be expressed constitutively or it may be inducible. As part of the host cell genome, it may be subject to cellular regulation, including activation or repression, depending on a host of factors in the target cell.

Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia, virus, visna-maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope (Naldini L et al., Curr. Opin. Bioiecknol, 1998, 9: 457-463). Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells.

Lentiviral particles may be generated by co-expressing the virus packaging elements and the vector genome itself in a producer cell such as human HEK293T cells. These elements are usually provided in three (in second generation lentiviral systems) or four separate plasmids (in third generation lentiviral systems). The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neomyocin (neo), dihydrofolate reductase (DHFR), glutamine synthetase or adenosine deaminase (ADA), followed by selection in the presence of the appropriate drug and isolation of clones.

The producer cell produces recombinant viral particles that contain the foreign gene, for example, the polynucleotides encoding the exogenous polynucleotide. The recombinant viral particles are recovered from the culture media and titrated by standard methods used by those of skill in the art. The recombinant lentiviral vehicles can be used to infect target cells, such source cells including any described in Section II.C.

Cells that can be used to produce high-titer lentiviral particles may include, but are not limited to, HEK293T cells, 293G cells, STAR cells (Relander et al., Mol Ther. 2005, 11: 452-459), FreeStyle™ 293 Expression System (ThermoFisher, Waltham, MA), and other HEK293T-based producer cell lines (e.g., Stewart et al., Hum Gene Ther. 2011, 2,2.(3):357-369; Lee et al, Biotechnol Bioeng, 2012, 10996): 1551-1560; Throm et al. Blood. 2009, 113(21): 5104-5110).

Additional elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof. Other elements include central polypurine tract (cPPT) sequence to improve transduction efficiency in non-dividing cells, Woodchuck Hepatitis Virus (WHP) Posttranscriptional Regulatory Element (WPRE) which enhances the expression of the transgene, and increases titer.

Methods for generating recombinant lentiviral particles are known to a skilled artisan, for example, U.S. Pat. Nos. 8,846,385; 7,745,179; 7,629,153; 7,575,924; 7,179,903; and 6,808,905. Lentivirus vectors used may be selected from, but are not limited to pLVX, pLenti, pLenti6, pLJMI, FUGW, pWPXL, pWPI, pLenti CMV puro DEST, pLJMI-EGFP, pULTRA, pInducer2Q, pHIV-EGFP, pCW57.1, pTRPE, pELPS, pRRL, and pLionII, Any known lentiviral vehicles may also be used (See, U.S. Pat. Nos. 9,260,725: 9,068,199: 9,023,646: 8,900,858: 8,748,169; 8,709,799; 8,420,104; 8,329,462; 8,076,106; 6,013,516: and 5,994, 136; International Patent Publication NO.: WO2012079000).

In some embodiments, the exogenous polynucleotide is introduced into the cell under conditions for transient expression of the cell, such as by methods that result in episomal delivery of an exogenous polynucleotide.

In some embodiments, polynucleotides encoding the exogenous polynucleotide may be packaged into recombinant adeno-associated viral (rAAV) vectors. Such vectors or viral particles may be designed to utilize any of the known serotype capsids or combinations of serotype capsids. The serotype capsids may include capsids from any identified AAV serotypes and variants thereof, for example, AAV1, AAV2, AAV2G9, AAV3, AAV4, AAV4-4, AAV5, AAV6, AAV7, AAV8, AAV9, AAV10, AAV 11, AAV12 and AAVrh10. In some embodiments, the AAV serotype may be or have a sequence as described in United States Publication No. US20030138772; Pulicherla et al. Molecular Therapy, 2011, 19(6): 1070-1078; U.S. Pat. Nos. 6,156,303; 7,198,951; U.S. Patent Publication Nos.: US2015/0159173 and US2014/0359799: and International Patent Publication NOs.: WO1998/011244, WO2005/033321 and WO2014/14422.

AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell. The rAAV vectors may be manufactured by standard methods in the art such as by triple transfection, in sf9 insect cells or in suspension cell cultures of human cells such as HEK293 cells.

In some embodiments, non-viral based methods may be used. For instance, in some aspects, vectors comprising the polynucleotides may be transferred to cells by non-viral methods by physical methods such as needles, electroporation, sonoporation, hyrdoporation; chemical carriers such as inorganic particles (e.g. calcium phosphate, silica, gold) and/or chemical methods. In other aspects, synthetic or natural biodegradable agents may be used for delivery such as cationic lipids, lipid nano emulsions, nanoparticles, peptide-based vectors, or polymer-based vectors.

2) Targeted Delivery

The exogenous polynucleotide can be inserted into any suitable target genomic loci of the primary cell. In some embodiments, the exogenous polynucleotide is introduced into the cell by targeted integration into a target loci. In some embodiments, targeted integration can be achieved by gene editing using one or more nucleases and/or nickases and a donor template in a process involving homology-dependent or homology-independent recombination.

A number of gene editing methods can be used to insert an exogenous polynucleotide into the specific genomic locus of choice, including for example homology-directed repair (HOR), homology-mediated end-joining (HMEJ), homology-independent targeted integration (HITI), obligate ligation-gated recombination (ObliGaRe), or precise integration into target chromosome (PITCh).

In some embodiments, the nucleases create specific double-strand breaks (DSBs) at desired locations (e.g. target sites) in the genome, and harness the cell's endogenous mechanisms to repair the induced break. The nickases create specific single-strand breaks at desired locations in the genome. In one non-limiting example, two nickases can be used to create two single-strand breaks on opposite strands of a target DNA, thereby generating a blunt or a sticky end. Any suitable nuclease can be introduced into a cell to induce genome editing of a target DNA sequence including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. In some embodiments, when a nuclease or a nickase is introduced with a donor template containing an exogenous polynucleotide sequence (also called a transgene) flanked by homology sequences (e.g. homology arms) that are homologous to sequences at or near the endogenous genomic target locus, e.g. a safe harbor locus, DNA damage repair pathways can result in integration of the transgene sequence at the target site in the cell. This can occur by a homology-dependent process. In some embodiments, the donor template is a circular double-stranded plasmid DNA, single-stranded donor oligonucleotide (ssODN), linear double-stranded polymerase chain reaction (PCR) fragments, or the homologous sequences of the intact sister chromatid. Depending on the form of the donor template, the homology-mediated gene insertion and replacement can be carried out via specific DNA repair pathways such as homology-directed repair (HDR), synthesis-dependent strand annealing (SDSA), microhomology-mediated end joining (MMEJ), and homology-mediated end joining (HMEJ) pathways.

For instance, DNA repair mechanisms can be induced by a nuclease after (i) two SSBs, where there is a SSB on each strand, thereby inducing single strand overhangs; or (ii) a DSB occurring at the same cleavage site on both strands, thereby inducing a blunt end break. Upon cleavage by one of these agents, the target locus with the SSBs or the DSB undergoes one of two major pathways for DNA damage repair: (1) the error-prone non-homologous end joining (NHEJ), or (2) the high-fidelity homology-directed repair (HDR) pathway. In some embodiments, a donor template (e.g. circular plasmid DNA or a linear DNA fragment, such as a ssODN) introduced into cells in which there are SSBs or a DSB can result in HDR and integration of the donor template into the target locus. In general, in the absence of a donor template, the NHEJ process re-ligates the ends of the cleaved DNA strands, which frequently results in nucleotide deletions and insertions at the cleavage site.

In some embodiments, site-directed insertion of the exogenous polynucleotide into a cell may be achieved through HDR-based approaches. HDR is a mechanism for cells to repair double-strand breaks (DSBs) in DNA and can be utilized to modify genomes in many organisms using various gene editing systems, including clustered regularly interspaced short palindromic repeat (CRISPR)/Cas systems, zinc finger nucleases (ZFNs), transcription activator-like effector nucleases (TALENs), meganucleases, and transposases.

In some embodiments, the targeted integration is carried by introducing one or more sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas) system, specifically designed to be targeted to at least one target site(s) sequence of a target gene. Exemplary ZFNs, TALEs, and TALENs are described in, e.g., Lloyd et al., Frontiers in Immunology, 4(221): 1-7 (2013). In particular embodiments, targeted genetic disruption at or near the target site is carried out using clustered regularly interspaced short palindromic repeats (CRISPR) and CRISPR-associated (Cas) proteins. See Sander and Joung, (2014) Nature Biotechnology, 32(4): 347-355.

Any of the systems for gene disruption described in Section II. A.1 can be used and, when also introduced with an appropriate donor template having with an exogenous polynucleotide, e.g. transgene sequences, can result in targeted integration of the exogenous polynucleotide at or near the target site of the genetic disruption. In particular embodiments, the genetic disruption is mediated using a CRISPR/Cas system containing one or more guide RNAs (gRNA) and a Cas protein. Exemplary Cas proteins and gRNA are described in Section II.A above, any of which can be used in HDR mediated integration of an exogenous polynucleotide into a target locus to which the Crispr/Cas system is specific for. It is within the level of a skilled artisan to choose an appropriate Cas nuclease and gRNA, such as depending on the particular target locus and target site for cleavage and integration of the exogenous polynucleotide by HDR. Further, depending on the target locus a skilled artisan can readily prepare an appropriate donor template, such as described further below.

In some embodiments, the DNA editing system is an RNA-guided CRISPR/Cas system (such as RNA-based CRISPR/Cas system), wherein the CRISPR/Cas system is capable of creating a double-strand break in the target locus (e.g. safe harbor locus) to induce insertion of the transgene into the target locus. In some embodiments, the nuclease system is a CRISPR/Cas9 system. In some embodiments, the CRISPR/Cas9 system comprises a plasmid-based Cas9. In some embodiments, the CRISPR/Cas9 system comprises a RNA-based Cas9. In some embodiments, the CRISPR/Cas9 system comprises a Cas9 mRNA and gRNA. In some embodiments, the CRISPR/Cas9 system comprises a protein/RNA complex, or a plasmid/RNA complex, or a protein/plasmid complex. In some embodiments, there are provided methods for generating engineered cells, which comprises introducing into a source cell (e.g. a primary cell) a donor template containing a transgene or exogenous polynucleotide sequence and a DNA nuclease system including a DNA nuclease system (e.g. Cas9) and a locus-specific gRNA. In some embodiments, the Cas9 is introduced as an mRNA. In some embodiments, the Cas9 is introduced as a ribonucleoprotein complex with the gRNA.

Generally, the donor template to be inserted would comprise at least the transgene cassette containing the exogenous polynucleotide of interest (e.g., the tolerogenic factor or CAR) and would optionally also include the promoter. In certain of these embodiments, the transgene cassette containing the exogenous polynucleotide and/or promoter to be inserted would be flanked in the donor template by homology arms with sequences homologous to sequences immediately upstream and downstream of the target cleavage site, i.e., left homology arm (LHA) and right homology arm (RHA). Typically, the homology arms of the donor template are specifically designed for the target genomic locus to serve as template for HDR. The length of each homology arm is generally dependent on the size of the insert being introduced, with larger insertions requiring longer homology arms.

In some embodiments, a donor template (e.g., a recombinant donor repair template) comprises: (i) a transgene cassette comprising an exogenous polynucleotide sequence (for example, a transgene operably linked to a promoter, for example, a heterologous promoter); and (ii) two homology arms that flank the transgene cassette and are homologous to portions of a target locus (e.g. safe harbor locus) at either side of a DNA nuclease (e.g., Cas nuclease, such as Cas9 or Cas12) cleavage site. The donor template can further comprise a selectable marker, a detectable marker, and/or a purification marker.

In some embodiments, the homology arms are the same length. In other embodiments, the homology arms are different lengths. The homology arms can be at least about 10 base pairs (bp), e.g., at least about 10 bp, 15 bp, 20 bp, 25 bp, 30 bp, 35 bp, 45 bp, 55 bp, 65 bp, 75 bp, 85 bp, 95 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1.1 kilobases (kb), 1.2 kb, 1.3 kb, 1.4 kb, 1.5 kb, 1.6 kb, 1.7 kb, 1.8 kb, 1.9 kb, 2.0 kb, 2, 1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, or longer. The homology arms can be about 10 bp to about 4 kb, e.g., about 10 bp to about 20 bp, about 10 bp to about 50 bp, about 10 bp to about 100 bp, about 10 bp to about 200 bp, about 10 bp to about 500 bp, about 10 bp to about 1 kb, about 10 bp to about 2 kb, about 10 bp to about 4 kb, about 100 bp to about 200 bp, about 100 bp to about 500 bp, about 100 bp to about 1 kb, about 100 bp to about 2 kb, about 100 bp to about 4 kb, about 500 bp to about 1 kb, about 500 bp to about 2 kb, about 500 bp to about 4 kb, about 1 kb to about 2 kb, about 1 kb to about 2 kb, about 1 kb to about 4 kb, or about 2 kb to about 4 kb.

In some embodiments, the donor template can be cloned into an expression vector. Conventional viral and non-viral based expression vectors known to those of ordinary skill in the art can be used.

In some embodiments, the target locus targeted for integration may be any locus in which it would be acceptable or desired to target integration of an exogenous polynucleotide or transgene. Non-limiting examples of a target locus include, but are not limited to, a CXCR4 gene, an albumin gene, a SHS231 locus, an F3 gene (also known as CD142), a MICA gene, a MICB gene, a LRP1 gene (also known as CD91), a HMGB1 gene, an ABO gene, a RHD gene, a FUT1 gene, a KDM5D gene (also known as HY), a B2M gene, a CIITA gene, a TRAC gene, a TRBC gene, a CCR5 gene, a F3 (i.e., CD142) gene, a LRP1 gene, a HMGB1 gene, an ABO gene, a RHD gene, a FUT1 gene, a KDM5D (i.e., HY) gene, a PDGFRa gene, a OLIG2 gene, and/or a GFAP gene. In some embodiments, the exogenous polynucleotide can be inserted in a suitable region of the target locus (e.g. safe harbor locus), including, for example, an intron, an exon, and/or gene coding region (also known as a Coding Sequence, or “CDS”). In some embodiments, the insertion occurs in one allele of the target genomic locus. In some embodiments, the insertion occurs in both alleles of the target genomic locus. In either of these embodiments, the orientation of the transgene inserted into the target genomic locus can be either the same or the reverse of the direction of the gene in that locus.

In some embodiments, the exogenous polynucleotide is interested into an intron, exon, or coding sequence region of the safe harbor gene locus. In some embodiments, the exogenous polynucleotide is inserted into an endogenous gene wherein the insertion causes silencing or reduced expression of the endogenous gene. Exemplary genomic loci for insertion of an exogenous polynucleotide are depicted in Table 4.

TABLE 4
Exemplary genomic loci for insertion of exogenous polynucleotides

| | | | | Target region | Also |
|---|---|---|---|---|---|
| Number | species | name | Ensembl ID | for cleavage | known as |
| 1 | human | B2M | ENSG00000166710 | CDS | |
| 2 | human | CIITA | ENSG00000179583 | CDS | |
| 3 | human | TRAC | ENSG00000277734 | CDS | |
| 4 | human | PPP1R12C | ENSG00000125503 | Intron 1 and 2 | AAVS1 |
| 5 | human | CLYBL | ENSG00000125246 | Intron 2 | |
| 6 | human | CCR5 | ENSG00000160791 | Exons 1-3, | |
| | | | | introns 1-2, | |
| | | | | and CDS | |
| 7 | human | THUMPD3- | ENSG00000206573 | Intron 1 | ROSA26 |
| | | AS1 | | | |
| 8 | human | Ch-4: | | 500 bp | SHS231 |
| | | 58,976,613 | | window | |
| 9 | human | F3 | ENSG00000117525 | CDS | CD142 |
| 10 | human | MICA | ENSG00000204520 | CDS | |
| 11 | human | MICB | ENSG00000204516 | CDS | |
| 12 | human | LRP1 | ENSG00000123384 | CDS | |
| 13 | human | HMGB1 | ENSG00000189403 | CDS | |
| 14 | human | ABO | ENSG00000175164 | CDS | |
| 15 | human | RHD | ENSG00000187010 | CDS | |
| 16 | human | FUT1 | ENSG00000174951 | CDS | |
| 17 | human | KDM5D | ENSG00000012817 | CDS | HY |

In some embodiments, the target locus is a safe harbor locus. In some embodiments, a safe harbor locus is a genomic location that allows for stable expression of integrated DNA with minimal impact on nearby or adjacent endogenous genes, regulatory element and the like. In some cases, a safe harbor gene enables sustainable gene expression and can be targeted by engineered nuclease for gene modification in various cell types including primary cells, including derivatives thereof, and differentiated cells thereof. Non-limiting examples of a safe harbor locus include, but are not limited to, a CCR5 gene locus, a PPP1R12C (also known as AAVS1) gene locus, a CLYBL gene locus, and/or a Rosa gene locus (e.g., ROSA26 gene locus). n some embodiments, the safe harbor locus is selected from the group consisting of the AAVS1 locus, the CCR5 locus, and the CLYBL locus. In some cases SHS231 can be targeted as a safe harbor locus in many cell types. In some cases, certain loci can function as a safe harbor locus in certain cell types. For instance, PDGFRa is a safe harbor for glial progenitor cells (GPCs), OLIG2 is a safe harbor locus for oligodendrocytes, and GFAP is a safe harbor locus for astrocytes. It is within the level of a skilled artisan to choose an appropriate safe harbor locus depending on the particular engineered cell type. In some cases, more than one safe harbor gene can be targeted, thereby introducing more than one transgene into the genetically modified cell.

In some embodiments, there are provided methods for generating engineered cells, which comprises introducing into a source cell (e.g. a primary cell) a donor template containing a transgene or exogenous polynucleotide sequence and a DNA nuclease system including a DNA nuclease system (e.g. Cas9) and a locus-specific gRNA that comprise complementary portions (e.g. gRNA targeting sequence) specific to a CCR5 gene locus, a PPP1R12C (also known as AAVS1) gene locus, a CLYBL gene locus, and/or a Rosa gene locus (e.g., ROSA26 gene locus). In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

In some embodiments, the gRNAs used herein for HDR-mediated insertion of a transgene comprise a complementary portion (e.g. gRNA targeting sequence) that recognizes a target sequence in AAVS1. In certain of these embodiments, the target sequence is located in intron 1 of AAVS 1. AAVS1 is located at Chromosome 19: 55,090,918-55,117,637 reverse strand, and AAVS1 intron 1 (based on transcript ENSG00000125503) is located at Chromosome 19: 55,117,222-55,112,796 reverse strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 19: 55, 117,222-55, 112,796. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 19: 55,115,674. In certain embodiments, the gRNA is configured to produce a cut site at Chromosome 19: 55, 115,674, or at a position within 5, 10, 15, 20, 30, 40 or 50 nucleotides of Chromosome 19: 55, 115,674. In certain embodiments, the gRNA s GET000046, also known as “sgAAVS1-1,” described in Li et al., Nat. Methods 16:866-869 (2019). This gRNA comprises a complementary portion (e.g. gRNA targeting sequence) having the nucleic acid sequence set forth in SEQ ID NO: 26 (e.g. Table 5) and targets intron 1 of AAVS1 (also known as PPP1R12C).

In some embodiments, the gRNAs used herein for HDR-mediated insertion of a transgene comprise a complementary portion (e.g. gRNA targeting sequence) that recognizes a target sequence in CLYBL. In certain of these embodiments, the target sequence is located in intron 2 of CL YBL. CLYBL is located at Chromosome 13: 99,606,669-99,897, 134 forward strand, and CLYBL intron 2 (based on transcript ENST00000376355.7) is located at Chromosome 13: 99,773,011-99,858,860 forward strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 13: 99,773,011-99,858,860. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 13: 99,822,980. In certain embodiments, the gRNA is configured to produce a cut site at Chromosome 13: 99,822,980, or at a position within 5, 0, 15, 20, 30, 40 or 50 nucleotides of Chromosome 13: 99,822,980. In certain embodiments, the gRNA is GET000047, which comprises a complementary portion (e.g. gRNA targeting sequence) having the nucleic acid sequence set forth in SEQ ID NO: 27 (e.g. Table 5) and targets intron 2 of CLYBL. The target site is similar to the target site of the TALENs as described in Cerbini et al., PLoS One, 10(1): e0116032 (2015).

In some embodiments, the gRNAs used herein for HDR-mediated insertion of a transgene comprise a complementary portion (e.g. gRNA targeting sequence) that recognizes a target sequence in CCR5. In certain of these embodiments, the target sequence is located in exon 3 of CCR5. CCR5 is located at Chromosome 3: 46,370,854-46,376,206 forward strand, and CCR5 exon 3 (based on transcript ENST00000292303.4) is located at Chromosome 3: 46,372,892-46,376,206 forward strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 3: 46,372,892-46,376,206. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 3: 46,373,180. In certain embodiments, the gRNA is configured to produce a cut site at Chromosome 3: 46,373,180, or at a position within 5, 10, 15, 20, 30, 40, or 50 nucleotides of Chromosome 3: 46,373,180. In certain embodiments the gRNA is GET000048, also known as “crCCR5_D,” described in Mandal et al., Cell Stem Cell 15:643-652 (2014). This gRNA comprises a complementary portion having the nucleic acid sequence set forth in SEQ ID NO: 28 (e.g. Table 5) and targets exon 3 of CCR5 (alternatively annotated as exon 2 in the Ensembl genome database). See Gomez-Ospina et al., Nat. Comm. 10(1):4045 (2019).

Table 5 sets forth exemplary gRNA targeting sequences. In some embodiments, the gRNA targeting sequence may contain one or more thymines in the complementary portion sequences set forth in Table 5 are substituted with uracil. It will be understood by one of ordinary skill in the art that uracil and thymine can both be represented by ‘t’, instead of ‘u’ for uracil and ‘t’ for thymine; in the context of a ribonucleic acid, it will be understood that ‘t’ is used to represent uracil unless otherwise indicated.

TABLE 5
Exemplary gRNA targeting sequences for CCR5

| | | SEQ |
|---|---|---|
| | | ID |
| Description | Nucleic Acid Sequence | NO |
| GET000046 | (5′->3′) | 26 |
| guide | accccacagtggggccacta | |
| GET000047 | (5′->3′) | 27 |
| guide | tgttggaaggatgaggaaat | |
| GET000048 | (5′->3′) | 28 |
| guide | tcactatgctgccgcccagt | |

In some embodiments, the target locus is a locus that is desired to be knocked out in the cells. In such embodiments, such a target locus is any target locus whose disruption or elimination is desired in the cell, such as to modulate a phenotype or function of the cell. For instance, any of the gene modifications described in Section II.A to reduce expression of a target gene may be a desired target locus for targeted integration of an exogenous polynucleotide, in which the genetic disruption or knockout of a target gene and overexpression by targeted insertion of an exogenous polynucleotide may be achieved at the same target site or locus in the cell. For instance, the HDR process may be used to result in a genetic disruption to eliminate or reduce expression of (e.g. knock out) any target gene set forth in Table 1b while also integrating (e.g. knocking in) an exogenous polynucleotide into the target gene by using a donor template with flanking homology arms that are homologous to nucleic acid sequences at or near the target site of the genetic disruption.

In some embodiments, there are provided methods for generating engineered primary cells, which comprises introducing into a source cell (e.g. a primary cell) a donor template containing a transgene or exogenous polynucleotide sequence and a DNA nuclease system including a DNA nuclease system (e.g. Cas9) and a locus-specific gRNA that comprise complementary portions specific to the B2M locus, the CIITA locus, the TRAC locus, the TRBC locus. In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

In particular embodiments, the target locus is B2M. In some embodiments, the engineered primary cell comprises a genetic modification targeting the B2M gene. In some embodiments, the genetic modification targeting the B2M gene is by using a targeted nuclease system that comprises a Cas protein or a polynucleotide encoding a Cas protein, and at least one guide ribonucleic acid sequence for specifically targeting the B2M gene. In some embodiments, the at least one guide ribonucleic acid (gRNA) sequence for specifically targeting the B2M gene is selected from the group consisting of SEQ ID NOS:81240-85644 of Appendix 2 or Table 15 of WO2016/183041, the disclosure is incorporated by reference in its entirety. In some embodiments, an exogenous polynucleotide is integrated into the disrupted B2M locus by HDR by introducing a donor template containing the exogenous polynucleotide sequence with flanking homology arms homologous to sequences adjacent to the target site targeted by the gRNA.

In particular embodiments, the target locus is CIITA. In some embodiments, the engineered primary cell comprises a genetic modification targeting the CIITA gene. In some embodiments, the genetic modification targeting the CIITA gene is by a targeted nuclease system that comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS:5184-36352 of Appendix 1 or Table 12 of WO2016183041, the disclosure is incorporated by reference in its entirety. In some embodiments, an exogenous polynucleotide is integrated into the disrupted CITTA locus by HDR by introducing a donor template containing the exogenous polynucleotide sequence with flanking homology arms homologous to sequences adjacent to the target site targeted by the gRNA.

In some embodiments, the primary cell is a T cell and expression of the endogenous TRAC or TRBC locus is reduced or eliminated in the cell by gene editing methods. For instance, the HDR process may be used to result in a genetic disruption to eliminate or reduce expression of (e.g. knock out) the TRAC or a TRBC gene while also integrating (e.g. knocking in) an exogenous polynucleotide into the same locus by using a donor template with flanking homology arms that are homologous to nucleic acid sequences at or near the target site of the genetic disruption. Exemplary gRNA sequences useful for CRISPR/Cas-based targeting of genes described herein are provided in Table 6. The sequences can be found in US20160348073, the disclosure including the Sequence Listing is incorporated herein by reference in its entirety.

TABLE 6
Exemplary gRNA targeting sequences useful for targeting genes

| Gene Name | SEQ ID NO of US20160348073 |
|---|---|
| TRAC | SEQ ID NOS: 532-609 and |
| | 9102-9797 |
| TRB (also TCRB, | SEQ ID NOS: 610-765 and 9798- |
| and TRBC) | 10532 |

In some embodiments, the engineered primary cell comprises a genetic modification targeting the TRAC gene. In some embodiments, the genetic modification targeting the TRAC gene is by a targeted nuclease system that comprises a Cas protein or a polynucleotide encoding a Cas protein, and at least one guide ribonucleic acid sequence for specifically targeting the TRAC gene. In some embodiments, the at least one guide ribonucleic acid sequence (e.g. gRNA targeting sequence) for specifically targeting the TRAC gene is selected from the group consisting of SEQ ID NOS: SEQ ID NOS: 532-609 and 9102-9797 of US20160348073, the disclosure is incorporated by reference in its entirety. In some embodiments, an exogenous polynucleotide is integrated into the disrupted TRAC locus by HDR by introducing a donor template containing the exogenous polynucleotide sequence with flanking homology arms homologous to sequences adjacent to the target site targeted by the gRNA.

In some embodiments, the engineered primary cell comprises a genetic modification targeting the TRBC gene. In some embodiments, the genetic modification targeting the TRBC gene is by a targeted nuclease system that comprises a Cas protein or a polynucleotide encoding a Cas protein, and at least one guide ribonucleic acid sequence for specifically targeting the TRBC gene. In some embodiments, the at least one guide ribonucleic acid sequence (e.g. gRNA targeting sequence) for specifically targeting the TRBC gene is selected from the group consisting of SEQ ID NOS: SEQ ID NOS:610-765 and 9798-10532 of US20160348073, the disclosure is incorporated by reference in its entirety. In some embodiments, an exogenous polynucleotide is integrated into the disrupted TRBC locus by HDR by introducing a donor template containing the exogenous polynucleotide sequence with flanking homology arms homologous to sequences adjacent to the target site targeted by the gRNA.

In some embodiments, it is within the level of a skilled artisan to identify new loci and/or gRNA sequences for use in HDR-mediated integration approaches as described. For example, for CRISPR/Cas systems, when an existing gRNA for a particular locus (e.g., within a target gene, e.g. set forth in Table 1b) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in genetic disruption methods. Although the CRISPR/Cas system is described as illustrative, any HDR-mediated approaches as described can be used in this method of identifying new loci, including those using ZFNs, TALENS, meganucleases and transposases.

In some embodiments, the exogenous polynucleotide encodes an exogenous CD47 polypeptide (e.g., a human CD47 polypeptide) and the exogenous polypeptide is inserted into a safe harbor gene loci or a safe harbor site as disclosed herein or a genomic locus that causes silencing or reduced expression of the endogenous gene. In some embodiments, the exogenous polynucleotide encoding CD47 is inserted in a a CCR5 gene locus, a PPP1R12C (also known as AAVS1) gene locus, a CLYBL gene locus, and/or a Rosa gene locus (e.g., ROSA26 gene locus). In some embodiments, the polynucleotide is inserted in a B2M, CIITA, TRAC, TRBC, PD1 or CTLA4 gene locus.

### C. Cells

In some embodiments, the present disclosure provides a cell (e.g., primary cell), or population thereof, that has been engineered (or modified) in which the genome of the cell has been modified such that expression of one or more genes as described herein is reduced or deleted (e.g. one or more genes regulating expression of one or more MHC class I molecules or one or more MHC class II molecules) or in which a gene or polynucleotide is overexpressed or increased in expression (e.g. polynucleotide encoding tolerogenic factor, such as CD47). In some embodiments, the engineered primary cell comprises (i) a transgene comprising an exogenous polynucleotide encoding CD47, (ii) inactivation or disruption of both alleles of a B2M gene, and (iii) inactivation or disruption of both alleles of a CIITA gene.

In some embodiments, the cells that are engineered or modified as provided herein are primary cells.

The cell may be a vertebrate cell, for example, a mammalian cell, such as a human cell or a mouse cell. Preferably, the cell is amenable to modification. Preferably, the cell has or is believed to have therapeutic value, such that the cell may be used to treat a disease, disorder, defect or injury in a subject in need of treatment for same.

In some embodiments, the cell is isolated from embryonic or neonatal tissue. In some embodiments, the cell is a fibroblast, monocytic precursor, B cell, exocrine cell, pancreatic progenitor, endocrine progenitor, hepatoblast, myoblast, preadipocyte, progenitor cell, hepatocyte, chondrocyte, smooth muscle cell, K562 human erythroid leukemia cell line, bone cell, synovial cell, tendon cell, ligament cell, meniscus cell, adipose cell, dendritic cells, or natural killer cell. In some embodiments, the cell is a muscle cell, erythroid-megakaryocytic cell, eosinophil, iPS cell, macrophage, T cell, islet cluster, islet cell, islet beta-cell, neuron, cardiomyocyte, blood cell, endocrine progenitor, exocrine progenitor, ductal cell, acinar cell, alpha cell, beta islet cell, delta cell, PP cell, hepatocyte, cholangiocyte, or brown adipocyte. In some embodiments, the cell is a muscle cell (e.g., skeletal, smooth, or cardiac muscle cell), erythroid-megakaryocytic cell, eosinophil, iPS cell, macrophage, T cell, islet islet cluster, islet cell, beta-cell, neuron, cardiomyocyte, blood cell (e.g., red blood cell, white blood cell, or platelet), endocrine progenitor, exocrine progenitor, ductal cell, acinar cell, alpha cell, beta islet cell, delta cell, PP cell, hepatocyte, cholangiocyte, or white or brown adipocyte. In some embodiments, the cell is a hormone-secreting cell (e.g., a cell that secretes insulin, oxytocin, endorphin, vasopressin, serotonin, somatostatin, gastrin, secretin, glucagon, thyroid hormone, bombesin, cholecystokinin, testosterone, estrogen, or progesterone, renin, ghrelin, amylin, or pancreatic polypeptide), an epidermal keratinocyte, an epithelial cell (e.g., an exocrine secretory epithelial cell, a thyroid epithelial cell, a keratinizing epithelial cell, a gall bladder epithelial cell, or a surface epithelial cell of the cornea, tongue, oral cavity, esophagus, anal canal, distal urethra, or vagina), a kidney cell, a germ cell, a skeletal joint synovium cell, a periosteum cell, a bone cell (e.g., osteoclast orosteoblast), a perichondrium cell (e.g., a chondroblast or chondrocyte), a cartilage cell (e.g., chondrocyte), a fibroblast, an endothelial cell, a pericardium cell, a meningeal cell, a keratinocyte precursor cell, a keratinocyte stem cell, a pericyte, a glial cell, an ependymal cell, a cell isolated from an amniotic or placental membrane, or a serosal cell (e.g., a serosal cell lining body cavities).

In some embodiments, the cell is a somatic cell. In some embodiments, the cells are derived from skin or other organs, e.g., heart, brain or spinal cord, liver, lung, kidney, pancreas, bladder, bone marrow, spleen, intestine, or stomach. The cells can be from humans or other mammals (e.g., rodent, non-human primate, bovine, or porcine cells).

In some embodiments, the cell is a T cell, NK cell, islet cell, beta islet cell, endothelial cell, epithelial cell such as RPE, thyroid, skin, or hepatocyte. In some embodiments, the cell is an engineered primary cell that has been modified from a primary cell. In some embodiments, the cell is an engineered primary cell (e.g., an engineered primary T cell, NK cell, islet cell, beta islet cell, endothelial cell, epithelial cell such as RPE, thyroid, skin, or hepatocyte). In some embodiments, the engineered primary cell comprises (i) a transgene comprising an exogenous polynucleotide encoding CD47, (ii) inactivation or disruption of both alleles of a B2M gene, and (iii) inactivation or disruption of both alleles of a CIITA gene, wherein the engineered primary T cell, NK cell, islet cell, beta islet cell, endothelial cell, epithelial cell such as RPE, thyroid, skin, or hepatocyte.

In some embodiments, the cell is a primary T cell that is engineered to contain modifications (e.g., genetic modifications) described herein. In some embodiments, the engineered primary T cell comprises (i) a transgene comprising an exogenous polynucleotide encoding CD47, (ii) inactivation or disruption of both alleles of a B2M gene, and (iii) inactivation or disruption of both alleles of a CIITA gene. In some embodiments, the T cell can be engineered with a chimeric antigen receptor (CAR), including any as described herein. In some embodiments, the engineered (e.g., hypoimmunogenic) T cell can be used to treat a variety of indications with allogeneic cell therapy, including any as described herein, e.g. Section V. In some embodiments, the engineered (e.g., hypoimmunogenic) T cell can be used to treat cancer.

In some embodiments, the cell is a primary NK cell that is engineered to contain modifications (e.g. genetic modifications) described herein. In some embodiments, the engineered primary NK cell comprises (i) a transgene comprising an exogenous polynucleotide encoding CD47, (ii) inactivation or disruption of both alleles of a B2M gene, and (iii) inactivation or disruption of both alleles of a CIITA gene. In some embodiments, the NK cell can be engineered with a chimeric antigen receptor (CAR), including any as described herein. In some embodiments, the engineered (e.g. hypoimmunogenic) NK cell can be used to treat a variety of indications with allogeneic cell therapy, including any as described herein, e.g. Section V. In some embodiments, the engineered (e.g. hypoimmunogenic) NK cell can be used to treat cancer.

In some embodiments, the cell is a primary islet cell that is engineered to contain modifications (e.g. genetic modifications) described herein. In some embodiments, the engineered primary islet cell comprises (i) a transgene comprising an exogenous polynucleotide encoding CD47, (ii) inactivation or disruption of both alleles of a B2M gene, and (iii) inactivation or disruption of both alleles of a CIITA gene. In some embodiments, the engineered (e.g. hypoimmunogenic) islet cell can be used to treat a variety of indications with allogeneic cell therapy, including any as described herein, e.g. Section IV. In some embodiments, the engineered (e.g. hypoimmunogenic) islet cell can be used to treat diabetes, such as type I diabetes. In some embodiments, the cell is a cluster of primary islet cells, comprising primary beta islet cells.

In some embodiments, the cell is a primary beta islet cell that is engineered to contain modifications (e.g. genetic modifications) described herein. In some embodiments, the engineered primary beta islet cell comprises (i) a transgene comprising an exogenous polynucleotide encoding CD47, (ii) inactivation or disruption of both alleles of a B2M gene, and (iii) inactivation or disruption of both alleles of a CIITA gene. In some embodiments, the engineered (e.g. hypoimmunogenic) beta islet cell can be used to treat a variety of indications with allogeneic cell therapy, including any as described herein, e.g. Section V. In some embodiments, the engineered (e.g. hypoimmunogenic) beta islet cell can be used to treat diabetes, such as type I diabetes.

In some embodiments, the cell is a primary endothelial cell that is engineered to contain modifications (e.g. genetic modifications) described herein. In some embodiments, the engineered primary endothelial cell comprises (i) a transgene comprising an exogenous polynucleotide encoding CD47, (ii) inactivation or disruption of both alleles of a B2M gene, and (iii) inactivation or disruption of both alleles of a CIITA gene. In some embodiments, the engineered (e.g. hypoimmunogenic) endothelial cell can be used to treat a variety of indications with allogeneic cell therapy, including any as described herein, e.g. Section V. In some embodiments, the engineered (e.g. hypoimmunogenic) endothelial cell can be used to treat vascularization or ocular diseases.

In some embodiments, the cell is a primary epithelial cell that is engineered to contain modifications (e.g. genetic modifications) described herein. In some embodiments, the epithelial cell is a RPE. In some embodiments, the epithelial cell is a thyroid cell. In some embodiments, the epithelial cell is a skin cell. In some embodiments, the engineered primary epithelial cell comprises (i) a transgene comprising an exogenous polynucleotide encoding CD47, (ii) inactivation or disruption of both alleles of a B2M gene, and (iii) inactivation or disruption of both alleles of a CIITA gene. In some embodiments, the engineered (e.g. hypoimmunogenic) epithelial cell can be used to treat a variety of indications with allogeneic cell therapy, including any as described herein, e.g. Section V. In some embodiments, the engineered (e.g. hypoimmunogenic) epithelial cell can be used to treat a thyroid disease or skin disease.

In some embodiments, the cell is a primary hepatocyte that is engineered to contain modifications (e.g. genetic modifications) described herein. In some embodiments, the engineered primary hepatocyte comprises (i) a transgene comprising an exogenous polynucleotide encoding CD47, (ii) inactivation or disruption of both alleles of a B2M gene, and (iii) inactivation or disruption of both alleles of a CIITA gene. In some embodiments, the engineered (e.g. hypoimmunogenic) epithelial cell can be used to treat a variety of indications with allogeneic cell therapy, including any as described herein, e.g. Section IV. In some embodiments, the engineered (e.g. hypoimmunogenic) hepatocyte cell can be used to treat liver disease.

In some embodiments, the cells that are engineered or modified as provided herein are cells from a healthy subject, such as a subject that is not known or suspected of having a particular disease or condition to be treated. For instance, if primary beta islet cells are isolated or obtained from a donor subject, such as for treating diabetes, the donor subject is a healthy subject if the subject is not known or suspected of suffering from diabetes or another disease or condition.

For therapeutic application, cells prepared according to the disclosed methods can typically be supplied in the form of a pharmaceutical composition comprising an isotonic excipient, and are prepared under conditions that are sufficiently sterile for human administration. The cells can be packaged in a device or container suitable for distribution or clinical use.

1. Primary Cells

In some embodiment the cells that are engineered as provided herein comprise cells derived from primary cells obtained or isolated from one or more individual subjects or donors. In some embodiments, the cells are derived from a pool of isolated primary cells obtained from one or more (e.g. two or more, three or more, four or more, five or more, ten or more, twenty or more, fifty or more, or one hundred or more) different donor subjects. In some embodiments, the primary cells isolated or obtained from the plurality of different donor subjects (e.g. two or more, three or more, four or more, five or more, ten or more, twenty or more, fifty or more, or one hundred or more) are pooled together in a batch and are engineered in accord with the provided methods.

In some embodiments, the primary cells are from a pool of primary cells from one or more donor subjects that are different than the recipient subject (e.g., the patient administered the cells). The primary cells can be obtained from 1, 2, 3, 4, 5, 6, 7, 8, 9, 10, 20, 50, 100 or more donor subjects and pooled together. The primary cells can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the primary cells are harvested from one or a plurality of individuals, and in some instances, the primary cells or the pool of primary T cells are cultured in vitro. In some embodiments, the primary cells or the pool of primary T cells are engineered or modified in accord with the methods provided herein.

In some embodiments, the methods include obtaining or isolating a desired type of primary cell (e.g. T cells, NK cells, endothelial cell, islet cell, beta islet cell, hepatocyte or other primary cells as described herein) from individual donor subjects, pooling the cells to obtain a batch of the primary cell type, and engineering the cells by the methods provided herein. In some embodiments, the methods include obtaining or isolating a desired type of primary cell (e.g. T cells, NK cells, endothelial cell, islet cell, beta islet cell, hepatocyte or other primary cells as described herein), engineering cells of each of the individual donors by the methods provided herein, and pooling engineered (modified) cells of at least two individual samples to obtain a batch of engineered primary cells of the primary cell type.

In some embodiments, the primary cells are isolated or obtained from an individual or from a pool of primary cells isolated or obtained from more than one individual donor. The primary cells may be any type of primary cell described herein, including any described in Section II.C.3. In some embodiments, the primary cells are selected from T cells, NK cells, islet cells, beta islet cells, endothelial cells, epithelial cells such as RPE, thyroid, skin, or hepatocytes. In some embodiments, the primary cells from an individual donor or a pool of individual donors are engineered to contain modifications (e.g. genetic modifications) described herein.

In some embodiments, the engineered cell is a muscle cell (e.g., skeletal, smooth, or cardiac muscle cell), erythroid-megakaryocytic cell, eosinophil, iPS cell, macrophage, T cell, islet cluster, islet cell, beta-cell, neuron, cardiomyocyte, blood cell (e.g., red blood cell, white blood cell, or platelet), endocrine progenitor, exocrine progenitor, ductal cell, acinar cell, alpha cell, beta islet cell, delta cell, PP cell, hepatocyte, cholangiocyte, or white or brown adipocyte. In some embodiments, the cell is a hormone-secreting cell (e.g., a cell that secretes insulin, oxytocin, endorphin, vasopressin, serotonin, somatostatin, gastrin, secretin, glucagon, thyroid hormone, bombesin, cholecystokinin, testosterone, estrogen, or progesterone, renin, ghrelin, amylin, or pancreatic polypeptide), an epidermal keratinocyte, an epithelial cell (e.g., an exocrine secretory epithelial cell, a thyroid epithelial cell, a keratinizing epithelial cell, a gall bladder epithelial cell, or a surface epithelial cell of the cornea, tongue, oral cavity, esophagus, anal canal, distal urethra, or vagina), a kidney cell, a germ cell, a skeletal joint synovium cell, a periosteum cell, a bone cell (e.g., osteoclast or osteoblast), a perichondrium cell (e.g., a chondroblast or chondrocyte), a cartilage cell (e.g., chondrocyte), a fibroblast, an endothelial cell, a pericardium cell, a meningeal cell, a keratinocyte precursor cell, a keratinocyte stem cell, a pericyte, a glial cell, an ependymal cell, a cell isolated from an amniotic or placental membrane, or a serosal cell (e.g., a serosal cell lining body cavities).

Exemplary cells are described in the following subsections.

a. T Cells

In some embodiments, the cells that are engineered or modified as provided herein are primary T lymphocytes (also called T cells). In some embodiments, the primary T lymphocytes are isolated or obtained from one or more individual donor subjects, such as one or more individual healthy donor (e.g. a subject that is not known or suspected of, e.g. not exhibiting clinical signs of, a disease or infection). In some instances, the T cells are populations or subpopulations of primary T cells from one or more individuals. As will be appreciated by those in the art, methods of isolating or obtaining T lymphocytes from an individual can be achieved using known techniques. Provided herein are engineered primary T lymphocytes that contain modifications (e.g. genetic modifications) described herein for subsequent transplantation or engraftment into subjects (e.g., recipients). For instance, the engineered T cells are are administered to a subject (e.g. recipient, such as a patient), by infusion of the engineered T cells.

In some embodiments, primary T cells are obtained (e.g., harvested, extracted, removed, or taken) from a subject or an individual. In some embodiments, primary T cells are produced from a pool of T cells such that the T cells are from one or more subjects (e.g., one or more human including one or more healthy humans). In some embodiments, the pool of primary T cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of T cells does not include cells from the patient. In some embodiments, one or more of the donor subjects from which the pool of T cells is obtained are different from the patient.

Non-limiting examples of primary T cells include CD3+ T cells, CD4+ T cells, CD8+ T cells, naïve T cells, regulatory T (Treg) cells, non-regulatory T cells, Th1 cells, Th2 cells, Th9 cells, Th7 cells, T-follicular helper (Tfh) cells, cytotoxic T lymphocytes (CTL), effector T (Teff) cells, central memory T (Tcm) cells, effector memory T (Tem) cells, effector memory T cells express CD45RA (TEMRA cells), tissue-resident memory (Trm) cells, virtual memory T cells, innate memory T cells, γδ T cells, and any other subtype of T cells. In some embodiments, the primary T cells are selected from a group that includes cytotoxic T-cells, helper T-cells, memory T-cells, regulatory T-cells, tumor infiltrating lymphocytes, and combinations thereof.

Exemplary T cells of the present disclosure are selected from the group consisting of cytotoxic T cells, helper T cells, memory T cells, central memory T cells, effector memory T cells, effector memory RA T cells, regulatory T cells, tissue infiltrating lymphocytes, and combinations thereof. In many embodiments, the T cells express CCR7, CD27, CD28, and CD45RA. In some embodiments, the central T cells express CCR7, CD27, CD28, and CD45RO. In other embodiments, the effector memory T cells express PD-1, CD27, CD28, and CD45RO. In other embodiments, the effector memory RA T cells express PD-1, CD57, and CD45RA.

In some embodiments, prior to the engineering as described herein, the T cells, such as isolated primary T cells or differentiated T cells, may be subject to one or more expansion or activation step. In some embodiments, a population of T cells to be engineered are stimulated or activated by incubation with anti-CD3 and anti-CD28 antibody reagents. The anti-CD3 and anti-CD28 may suitably be provided in the form of beads coated with a mixture of these reagents. Anti-CD3 and anti-CD28 beads may suitably be provided at a ratio of 1:1 to a population of T cells to be engineered. In some embodiments, the media during the incubation may also contain one or more recombinant cytokine, such as recombinant IL-2 or recombinant IL-15.

In some embodiments, the engineered T cells described herein, such as primary T cells isolated from one or more individual donors (e.g. healthy donors), comprise T cells engineered (e.g., are modified) to express a chimeric antigen receptor including but not limited to a chimeric antigen receptor described herein. Any suitable CAR can be included in the T cells, including the CARs described herein. In some embodiments, the engineered T cells express at least one chimeric antigen receptor that specifically binds to an antigen or epitope of interest expressed on the surface of at least one of a damaged cell, a dysplastic cell, an infected cell, an immunogenic cell, an inflamed cell, a malignant cell, a metaplastic cell, a mutant cell, and combinations thereof. In other cases, the engineered T cell comprise a modification causing the cell to express at least one protein that modulates a biological effect of interest in an adjacent cell, tissue, or organ when the cell is in proximity to the adjacent cell, tissue, or organ. Useful modifications to T cells, including primary T cells, are described in detail in US2016/0348073 and WO2020/018620, the disclosures of which are incorporated herein in their entireties.

In some embodiments, the T cell includes a polynucleotide encoding a CAR, wherein the polynucleotide is inserted in a genomic locus. Any suitable method can be used to insert the CAR into the genomic locus of the T cell including lentiviral based transduction methods or gene editing methods described herein (e.g., a CRISPR/Cas system). In some embodiments, the polynucleotide is inserted into a safe harbor locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus. In some embodiments, the polynucleotide is inserted in a B2M, CIITA, TRAC, TRBC, PD1 or CTLA4 gene.

In some embodiments, the T cells described herein such as the engineered or modified T cells comprise reduced expression of an endogenous T cell receptor. In some embodiments, the TRAC or TRBC locus is disrupted or eliminated in the cell, such as by gene editing methods described herein (e.g., a CRISPR/Cas system). In some embodiments, an exogenous polynucleotide or transgene, such as a polynucleotide encoding a CAR or other polynucleotide as described, is inserted into the disrupted TRAC or TRBC locus.

In some embodiments, the T cells described herein such as the engineered or modified T cells include reduced expression of cytotoxic T-lymphocyte-associated protein 4 (CTLA4). In some embodiments, the CTLA-4 locus is disrupted or eliminated in the cell, such as by gene editing methods described herein (e.g., a CRISPR/Cas system). In some embodiments, an exogenous polynucleotide or transgene, such as a polynucleotide encoding a CAR or other exogenous polynucleotide as described, is inserted into the disrupted CTLA-4 locus.

In other embodiments, the T cells described herein such as the engineered or modified T cells include reduced expression of programmed cell death (PD1). In some embodiments, the PD1 locus is disrupted or eliminated in the cell, such as by gene editing methods described herein (e.g., a CRISPR/Cas system). In some embodiments, an exogenous polynucleotide or transgene, such as a polynucleotide encoding a CAR or other exogenous polynucleotide as described, is inserted into the disrupted PD1 locus. In certain embodiments, the T cells described herein such as the engineered or modified T cells include reduced expression of CTLA4 and PD1.

In certain embodiments, the T cells described herein such as the engineered or modified T cells include enhanced expression of PD-L1. In some embodiments, the PD-L1 locus is disrupted or eliminated in the cell, such as by gene editing methods described herein (e.g., a CRISPR/Cas system). In some embodiments, an exogenous polynucleotide or transgene, such as a polynucleotide encoding a CAR or other exogenous polynucleotide as described, is inserted into the disrupted PD-L1 locus.

In some embodiments, the present technology is directed to engineered T cells, such as primary T cells isolated from one or more individual donors (e.g. healthy donors), that overexpress a tolerogenic factor (e.g. CD47), and have reduced expression or lack expression of one or more MHC class I molecules and/or one or more MHC class II molecules (e.g., one or more MHC class I human leukocyte antigen molecules and one or more MHC class II human leukocyte antigen molecules). In certain embodiments, the engineered T cells overexpress a tolerogenic factor (e.g. CD47) and harbor a genomic modification in the B2M gene. In some embodiments, the engineered T cells overexpress a tolerogenic factor (e.g CD47) and harbor a genomic modification in the CIITA gene. In some embodiments, the engineered T cells also are engineered to express a CAR. In some embodiments, the engineered T cells have reduced expression or lack expression of TCR complex molecules, such as by a genomic modification (e.g. gene disruption) in the TRAC gene or TRBC gene. In some embodiments, T cells overexpress a tolerogenic factor (e.g. CD47) and a CAR and harbor genomic modifications that disrupt one or more of the following genes: the B2M, CIITA, TRAC and TRBC genes.

In some embodiments, the provided engineered T cells evade immune recognition. In some embodiments, the engineered T cells described herein, such as primary T cells isolated from one or more individual donors (e.g. healthy donors), do not activate an immune response in the patient (e.g., recipient upon administration). Provided are methods of treating a disease by administering a population of engineered T cells described herein to a subject (e.g., recipient) or patient in need thereof.

T cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

b. Natural Killer Cells

In some embodiments, the cells that are engineered or modified as provided herein are primary Natural Killer (NK) cells. In some embodiments, the primary NK cells are isolated or obtained from one or more individual donor subjects, such as one or more individual healthy donor (e.g. a subject that is not known or suspected of, e.g. not exhibiting clinical signs of, a disease or infection). In some instances, the NK cells are populations or subpopulations of primary NK cells from one or more individuals. As will be appreciated by those in the art, methods of isolating or obtaining NK cells from an individual can be achieved using known techniques. Provided herein are engineered primary NK cells that contain modifications (e.g. genetic modifications) described herein for subsequent transplantation or engraftment into subjects (e.g. recipients. For instance, the engineered T cells are administered to a subject (e.g. recipient, such as a patient), by infusion of the engineered NK cells into the subject.

In some embodiments, primary NK cells are obtained (e.g., harvested, extracted, removed, or taken) from a subject or an individual. In some embodiments, primary NK cells are produced from a pool of NK cells such that the NK cells are from one or more subjects (e.g., one or more human including one or more healthy humans). In some embodiments, the pool of primary NK cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the engineered NK cells). In some embodiments, the pool of NK cells does not include cells from the patient. In some embodiments, one or more of the donor subjects from which the pool of NK cells is obtained are different from the patient.

In some embodiments, NK cells, including primary NK cells isolated from one or more individual donors (e.g. healthy donors) express CD56 (e.g. CD56^{dim} or CD56^{bright}) and lack CD3 (e.g. CD3^{neg}). In some embodiments, NK cells as described herein may also express the low-affinity Fcγ receptor CD16, which mediate ADCC. In some embodiments, the NK cells also express one or more natural killer cell receptors NKG2A and NKG2D or one or more natural cytotoxicity receptors NKp46, NKp44, NKp30. For example, for the case of primary NK cells, in specific cases, the primary cells may be isolated from a starting source of NK cells, such as a sample containing peripheral blood mononuclear cells (PBMCs), by depletion of cells positive for CD3, CD14, and/or CD19. For instance, the cells may be subject to depletion using immunomagnetic beads having attached thereto antibodies to CD3, CD14, and/or CD 19, respectively), thereby producing an enriched population of NK cells. In other cases, primary NK cells may be isolated from a starting source that is a mixed population (e.g. PBMCs) by selecting cells for the presence of one or more markers on the NK cells, such as CD56, CD16, NKp46, and/or NKG2D.

In some embodiments, prior to the engineering as described herein, the NK cells, such as isolated primary NK cells, may be subject to one or more expansion or activatio nstep. In some embodiments, expansion may be achieved by culturing of the NK cells with feeder cells, such as antigen presenting cells that may or may not be irradiated. The ratio of NK cells to antigen presenting cells (APCs) in the expansion step may be of a certain number, such as 1:1, 1:1.5, 1:2, or 1:3, for example. In certain aspects, the APCs are engineered to express membrane-bound IL-21 (mblL-21). In particular aspects, the APCs are alternatively or additionally engineered to express IL-21, IL-15, and/or IL-2. In particular embodiments, the media in which the expansion step(s) occurs comprises one or more agents to facilitate expansion, such as one or more recombinant cytokines. In specific embodiments, the media comprises one or more recombinant cytokines from IL-2, IL-15, IL-18, and/or IL-21. In some embodiments, the steps for engineered the NK cells by introducing the modifications as described herein is carried out 2-12 days after initiation of the expansion, such as on or about day 2, 3, 4, 5, 6, 7, 8, 9, 10, 11 or 12.

In some embodiments, the engineered NK cells described herein, such as primary NK cells isolated from one or more individual donors (e.g. healthy donors), comprise NK cells engineered (e.g., are modified) to express a chimeric antigen receptor including but not limited to a chimeric antigen receptor described herein. Any suitable CAR can be included in the NK cells, including the CARs described herein. In some embodiments, the engineered NK cells express at least one chimeric antigen receptor that specifically binds to an antigen or epitope of interest expressed on the surface of at least one of a damaged cell, a dysplastic cell, an infected cell, an immunogenic cell, an inflamed cell, a malignant cell, a metaplastic cell, a mutant cell, and combinations thereof. In other cases, the engineered NK cell comprise a modification causing the cell to express at least one protein that modulates a biological effect of interest in an adjacent cell, tissue, or organ when the cell is in proximity to the adjacent cell, tissue, or organ.

In some embodiments, the NK cell includes a polynucleotide encoding a CAR, wherein the polynucleotide is inserted in a genomic locus. Any suitable method can be used to insert the CAR into the genomic locus of the NK cell including lentiviral based transduction methods or gene editing methods described herein (e.g., a CRISPR/Cas system). In some embodiments, the polynucleotide is inserted into a safe harbor locus, such as but not limited to, an AAVS1, CCR5, CLYBL, ROSA26, SHS231, F3 (also known as CD142), MICA, MICB, LRP1 (also known as CD91), HMGB1, ABO, RHD, FUT1, or KDM5D gene locus.

In some embodiments, the present technology is directed to engineered NK cells, such as primary NK cells isolated from one or more individual donors (e.g. healthy donors), that overexpress a tolerogenic factor (e.g. CD47), and have reduced expression or lack expression of one or more MHC class I molecules and/or one or more MHC class II molecules (e.g., one or more MHC class I human leukocyte antigen molecules and one or more MHC class II human leukocyte antigen molecules). In certain embodiments, the engineered NK cells overexpress a tolerogenic factor (e.g. CD47) and harbor a genomic modification in the B2M gene. In some embodiments, the engineered NK cells overexpress a tolerogenic factor (e.g CD47) and harbor a genomic modification in the CIITA gene. In some embodiments, the engineered NK cells also are engineered to express a CAR.

In some embodiments, the provided engineered NK cells evade immune recognition. In some embodiments, the engineered NK cells described herein, such as primary NK cells isolated from one or more individual donors (e.g. healthy donors), do not activate an immune response in the patient (e.g., recipient upon administration). Provided are methods of treating a disease by administering a population of engineered NK cells described herein to a subject (e.g., recipient) or patient in need thereof.

NK cells provided herein are useful for the treatment of suitable cancers including, but not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

c. Beta Islet Cells

In some embodiments, the cells that are engineered or modified as provided herein are primary islet cells. In some embodiments, the primary islet cell is a cluster of primary islet cells. In some embodiments, the cells that are engineered or modified as provided herein are primary beta islet cells (also referred to as pancreatic islet cells or pancreatic beta islet cells). In some embodiments, the primary beta islet cells are isolated or obtained from one or more individual donor subjects, such as one or more individual healthy donor (e.g. a subject that is not known or suspected of, e.g. not exhibiting clinical signs of, a disease or infection). As will be appreciated by those in the art, methods of isolating or obtaining beta islet cells from an individual can be achieved using known techniques. Provided herein are engineered primary beta islet cells that contain modifications (e.g. genetic modifications) described herein for subsequent transplantation or engraftment into subjects (e.g., recipients).

In some embodiments, beta islet cells are obtained (e.g., harvested, extracted, removed, or taken) from a subject or an individual. In some embodiments, primary beta islet cells are produced from a pool of beta islet cells such that the beta islet cells are from one or more subjects (e.g., one or more human including one or more healthy humans). In some embodiments, the pool of primary beta islet cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of beta islet cells does not include cells from the patient. In some embodiments, one or more of the donor subjects from which the pool of beta islets cells is obtained are different from the patient.

Additional descriptions of pancreatic islet cells including for use in the present technology are found in WO2020/018615, the disclosure is herein incorporated by reference in its entirety.

In some embodiments, the population of engineered beta islet cells, such as primary beta islet cells isolated from one or more individual donors (e.g. healthy donors) or endothelial cells isolated from one or more individual donors (e.g. healthy donors), are maintained in culture, in some cases expanded, prior to administration. In certain embodiments, the population of engineered beta islet cells are cryopreserved prior to administration.

Exemplary pancreatic islet cell types include, but are not limited to, pancreatic islet progenitor cell, immature pancreatic islet cell, mature pancreatic islet cell, and the like. In some embodiments, pancreatic cells described herein are administered to a subject to treat diabetes.

In some embodiments, the pancreatic islet cells engineered as disclosed herein, such as primary beta islet cells isolated from one or more individual donors (e.g. healthy donors), secretes insulin. In some embodiments, a pancreatic islet cell exhibits at least two characteristics of an endogenous pancreatic islet cell, for example, but not limited to, secretion of insulin in response to glucose, and expression of beta islet cell markers.

Exemplary beta islet cell markers or beta islet cell progenitor markers include, but are not limited to, c-peptide, Pdxl, glucose transporter 2 (Glut2), HNF6, VEGF, glucokinase (GCK), prohormone convertase (PC 1/3), Cdcpl, NeuroD, Ngn3, Nkx2.2, Nkx6.1, Nkx6.2, Pax4, Pax6, Ptfla, Isll, Sox9, Sox17, and FoxA2.

In some embodiments, the primary pancreatic beta islet cells may be isolated from a primary pancreatic islet, derived from primary pancreatic beta islet cells within a primary pancreatic islet, or as a component of a primary pancreatic islet. For example, primary pancreatic beta islet cells can be edited as a single beta islet cell, a population of beta islet cells, or as a component of a primary pancreatic islet (e.g., primary pancreatic beta islet cells present within the primary pancreatic islet along with other cell types). As another example, primary pancreatic beta islet cells can be administered to a patient as single beta islet cells, a population of beta islet cells, or as a component of a primary pancreatic islet (e.g., primary pancreatic beta islet cells present within the primary pancreatic islet along with other cell types). In embodiments where the pancreatic beta islet cells are present within the pancreatic islet along with other cell types, the other cell types may also be edited by the methods described herein.

In some embodiments, the primary pancreatic islet cells are dissociated from a primary islet prior to or after engineering, such as genetic engineering. Such dissociated islet cells can be clustered prior to administration to a patient and clusters can include beta islet cells as well as other cell types including but not limited to those from the primary islet. Numbers of islet cells in the cluster can vary, such as about 50, about 100, about 250, about 500, about 750, about 1000, about 1250, about 1500, about 1750, about 2000, about 2250, about 2500, about 2750, about 3000, about 3500, about 4000, about 4500, or about 5000 cells. Patients can be administered about 10, about 20, about 30, about 40, about 50, about 75, about 100, about 125, about 150, about 200, about 250, about 300, about 325, about 350, about 375, about 400, about 425, about 450, about 475, about 500, about 600, about 700, about 800, about 900, or about 1000 clusters.

In some embodiments, the primary pancreatic islet cells, isolated from one or more individual donors (e.g., healthy donors), produce insulin in response to an increase in glucose. In some embodiments, the pancreatic islet cells are beta islet cells. In some embodiments, the beta islet cells are monitored to assess glucose control abilities. Assays to monitor glucose control may include, but are not limited to, continuous blood glucose level monitoring, monitoring blood glucose levels after a period of fasting, glucose tolerance (e.g., glucose challenge) tests, glucose utilization and oxidation, insulin secretion, such as by a U-PLEX® Meso Scale Discovery (MSD) assay and/or glucose-stimulated insulin secretion (GSIS) assays, measuring the presence of specific transcription factors and pathways (e.g., homeobox transcription factor SIX2, NKX6-1, and PDX1), measuring mitochondrial respiration, and measuring changes in intracellular Ca2⁺ calcium flux, such as glucose-induced Ca²⁺ rise, Ca²⁺-activated exocytosis. Various methods of measuring glucose control are known in the art, such as those described in Velazco-Cruz et al., Cell Reports, 2020, 31, 107687; Pagliuca et al., Cell, 2014, 159(2): 428-439; Davis et al., Cell Reports, 2020, 31(6): 107623; and, Alcazar et al., Cell Transplantation, 2020, 29, the disclosures including the figures, figure legends, and description of methods are incorporated herein by reference in their entirety. In some embodiments, the beta islet cells (e.g., engineered beta islet cells) may exhibit GSIS. In some embodiments, the GSIS is measured in a perfusion GSIS assay. In some embodiments, the GSIS is dynamic GSIS comprising first and second phase dynamic insulin secretion. In some embodiments, the GSIS is static GSIS. For example, the static incubation index may be greater than at or about 1, greater than at or about 2, greater than at or about 5, greater than at or about 10 or greater than at or about 20. In various embodiments, the pancreatic islet cells secrete insulin in response to an increase in glucose. In some embodiments, the cells have a distinct morphology such as a cobblestone cell morphology and/or a diameter of about 17 pm to about 25 pm.

In some embodiments, the present technology is directed to engineered beta islet cells, such as primary beta islet cells isolated from one or more individual donors (e.g. healthy donors), that overexpress a tolerogenic factor (e.g. CD47), and have reduced expression or lack expression of one or more MHC class I molecules and/or one or more MHC class II molecules (e.g., one or more MHC class I human leukocyte antigen molecules and one or more MHC class II human leukocyte antigen molecules). In certain embodiments, the engineered beta islet cells overexpress a tolerogenic factor (e.g. CD47) and harbor a genomic modification in the B2M gene. In some embodiments, the engineered beta islet cells overexpress a tolerogenic factor (e.g. CD47) and harbor a genomic modification in the CIITA gene. In some embodiments, the engineered beta islet cells overexpress a tolerogenic factor (e.g. CD47) and harbor genomic modifications that disrupt one or more of the following genes: the B2M and CIITA genes.

In some embodiments, the provided engineered beta islet cells evade immune recognition. For example, the engineered beta islet cells may evade NK cell mediated cell killing, macrophage mediated cell killing, and/or PBMC mediated cell killing. In some embodiments, the engineered beta islet cells described herein, such as primary beta islet cells isolated from one or more individual donors (e.g. healthy donors), do not activate an immune response in the patient (e.g., recipient upon administration). For example, following transplantation, a subject receiving the engineered beta islet cells may exhibit lower levels of interferon gamma (IFNg) compared to a subject receiving wild type beta islet cells. Similarly, following transplantation, a subject receiving the engineered beta islet cells may exhibit lower levels of donor-specific antibody (DSA) binding (e.g., IgG or IgM) compared to a subject receiving wild type beta islet cells. Provided are methods of treating a disease by administering a population of engineered beta islet cells described herein to a subject (e.g., recipient) or patient in need thereof. In some embodiments, the disease is diabetes, such as Type I diabetes or Type II diabetes.

d. Endothelial Cells

In some embodiments, the cells that are engineered or modified as provided herein are primary endothelial cells. In some embodiments, the primary endothelial cells are isolated or obtained from one or more individual donor subjects, such as one or more individual healthy donor (e.g. a subject that is not known or suspected of, e.g. not exhibiting clinical signs of, a disease or infection). As will be appreciated by those in the art, methods of isolating or obtaining endothelial cells from an individual can be achieved using known techniques. Provided herein are engineered primary endothelial cell types that contain modifications (e.g. genetic modifications) described herein for subsequent transplantation or engraftment into subjects (e.g., recipients).

In some embodiments, primary endothelial cells are obtained (e.g., harvested, extracted, removed, or taken) from a subject or an individual. In some embodiments, primary endothelial cells are produced from a pool of endothelial cells such that the endothelial cells are from one or more subjects (e.g., one or more human including one or more healthy humans). In some embodiments, the pool of primary endothelial cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of endothelial cells does not include cells from the patient. In some embodiments, one or more of the donor subjects from which the pool of endothelial cells is obtained are different from the patient.

Additional descriptions of endothelial cells for use in the methods provided herein are found in WO2020/018615, the disclosure is herein incorporated by reference in its entirety.

In some embodiments, the population of engineered endothelial cells, such as primary endothelial cells isolated from one or more individual donors (e.g. healthy donors), are maintained in culture, in some cases expanded, prior to administration. In certain embodiments, the population of endothelial cells are cryopreserved prior to administration.

In some embodiments, the present technology is directed to engineered endothelial cells, such as primary endothelial cells isolated from one or more individual donors (e.g. healthy donors), that overexpress a tolerogenic factor (e.g. CD47), and have reduced expression or lack expression one or more MHC class I molecules and/or one or more MHC class II molecules (e.g., one or more MHC class I human leukocyte antigen molecules and one or more MHC class II human leukocyte antigen molecules). In certain embodiments, the engineered endothelial cells overexpress a tolerogenic factor (e.g. CD47) and harbor a genomic modification in the B2M gene. In some embodiments, the engineered endothelial cells overexpress a tolerogenic factor (e.g CD47) and harbor a genomic modification in the CIITA gene. In some embodiments, engineered endothelial cells overexpress a tolerogenic factor (e.g. CD47) and harbor genomic modifications that disrupt one or more of the following genes: the B2M and CIITA genes.

In some embodiments, the provided engineered endothelial cells evade immune recognition. In some embodiments, the engineered endothelial cells described herein, such as primary endothelial cells isolated from one or more individual donors (e.g. healthy donors, do not activate an immune response in the patient (e.g., recipient upon administration). Provided are methods of treating a disease by administering a population of engineered endothelial cells described herein to a subject (e.g., recipient) or patient in need thereof.

In some embodiments, the engineered endothelial cells, such as primary endothelial cells isolated from one or more individual donors (e.g. healthy donors), are administered to a patient, e.g., a human patient in need thereof. The engineered endothelial cells can be administered to a patient suffering from a disease or condition such as, but not limited to, cardiovascular disease, vascular disease, peripheral vascular disease, ischemic disease, myocardial infarction, congestive heart failure, peripheral vascular obstructive disease, stroke, reperfusion injury, limb ischemia, neuropathy (e.g., peripheral neuropathy or diabetic neuropathy), organ failure (e.g., liver failure, kidney failure, and the like), diabetes, rheumatoid arthritis, osteoporosis, vascular injury, tissue injury, hypertension, angina pectoris and myocardial infarction due to coronary artery disease, renal vascular hypertension, renal failure due to renal artery stenosis, claudication of the lower extremities, and the like. In certain embodiments, the patient has suffered from or is suffering from a transient ischemic attack or stroke, which in some cases, may be due to cerebrovascular disease. In some embodiments, the engineered endothelial cells are administered to treat tissue ischemia e.g., as occurs in atherosclerosis, myocardial infarction, and limb ischemia and to repair of injured blood vessels. In some instances, the cells are used in bioengineering of grafts.

For instance, the engineered endothelial cells can be used in cell therapy for the repair of ischemic tissues, formation of blood vessels and heart valves, engineering of artificial vessels, repair of damaged vessels, and inducing the formation of blood vessels in engineered tissues (e.g., prior to transplantation). Additionally, the endothelial cells can be further modified to deliver agents to target and treat tumors.

In many embodiments, provided herein is a method of repair or replacement for tissue in need of vascular cells or vascularization. The method involves administering to a human patient in need of such treatment, a composition containing the engineered endothelial cells, such as isolated primary endothelial cells or differentiated endothelial cells, to promote vascularization in such tissue. The tissue in need of vascular cells or vascularization can be a cardiac tissue, liver tissue, pancreatic tissue, renal tissue, muscle tissue, neural tissue, bone tissue, among others, which can be a tissue damaged and characterized by excess cell death, a tissue at risk for damage, or an artificially engineered tissue.

In some embodiments, vascular diseases, which may be associated with cardiac diseases or disorders can be treated by administering endothelial cells, such as but not limited to, definitive vascular endothelial cells and endocardial endothelial cells derived as described herein. Such vascular diseases include, but are not limited to, coronary artery disease, cerebrovascular disease, aortic stenosis, aortic aneurysm, peripheral artery disease, atherosclerosis, varicose veins, angiopathy, infarcted area of heart lacking coronary perfusion, non-healing wounds, diabetic or non-diabetic ulcers, or any other disease or disorder in which it is desirable to induce formation of blood vessels.

In certain embodiments, the endothelial cells are used for improving prosthetic implants (e.g., vessels made of synthetic materials such as Dacron and Gortex.) which are used in vascular reconstructive surgery. For example, prosthetic arterial grafts are often used to replace diseased arteries which perfuse vital organs or limbs. In other embodiments, the engineered endothelial cells are used to cover the surface of prosthetic heart valves to decrease the risk of the formation of emboli by making the valve surface less thrombogenic.

The endothelial cells outlined can be transplanted into the patient using well known surgical techniques for grafting tissue and/or isolated cells into a vessel. In some embodiments, the cells are introduced into the patient's heart tissue by injection (e.g., intramyocardial injection, intracoronary injection, trans-endocardial injection, trans-epicardial injection, percutaneous injection), infusion, grafting, and implantation.

Administration (delivery) of the endothelial cells includes, but is not limited to, subcutaneous or parenteral including intravenous, intraarterial (e.g., intracoronary), intramuscular, intraperitoneal, intramyocardial, trans-endocardial, trans-epicardial, intranasal administration as well as intrathecal, and infusion techniques.

As will be appreciated by those in the art, the cells are transplanted using techniques known in the art that depends on both the cell type and the ultimate use of these cells. In some embodiments, the cells provided herein are transplanted either intravenously or by injection at particular locations in the patient. When transplanted at particular locations, the cells may be suspended in a gel matrix to prevent dispersion while they take hold.

Exemplary endothelial cell types include, but are not limited to, a capillary endothelial cell, vascular endothelial cell, aortic endothelial cell, arterial endothelial cell, venous endothelial cell, renal endothelial cell, brain endothelial cell, liver endothelial cell, and the like.

The endothelial cells outlined herein, such as isolated primary endothelial cells, can express one or more endothelial cell markers. Non-limiting examples of such markers include VE-cadherin (CD 144), ACE (angiotensin-converting enzyme) (CD 143), BNH9/BNF13, CD31, CD34, CD54 (ICAM-1), CD62E (E-Selectin), CD105 (Endoglin), CD146, Endocan (ESM-1), Endoglyx-1, Endomucin, Eotaxin-3, EPAS1 (Endothelial PAS domain protein 1), Factor VIII related antigen, FLI-1, Flk-1 (KDR, VEGFR-2), FLT-1 (VEGFR-1), GATA2, GBP-1(guanylate-binding protein-1), GRO-alpha, HEX, ICAM-2 (intercellular adhesion molecule 2), LM02, LYVE-1, MRB (magic roundabout), Nucleolin, PAL-E (pathologische anatomie Leiden-endothelium), RTKs, sVCAM-1, TALI, TEM1 (Tumor endothelial marker 1), TEM5 (Tumor endothelial marker 5), TEM7 (Tumor endothelial marker 7), thrombomodulin (TM, CD141), VCAM-1 (vascular cell adhesion molecule-1) (CD106), VEGF, vWF (von Willebrand factor), ZO-1, endothelial cell-selective adhesion molecule (ESAM), CD102, CD93, CD184, CD304, and DLL4.

In some embodiments, the endothelial cells are further genetically modified to express an exogenous gene encoding a protein of interest such as but not limited to an enzyme, hormone, receptor, ligand, or drug that is useful for treating a disorder/condition or ameliorating symptoms of the disorder/condition. Standard methods for genetically modifying endothelial cells are described, e.g., in U.S. Pat. No. 5,674,722.

Such endothelial cells can be used to provide constitutive synthesis and delivery of polypeptides or proteins, which are useful in prevention or treatment of disease. In this way, the polypeptide is secreted directly into the bloodstream or other area of the body (e.g., central nervous system) of the individual. In some embodiments, the endothelial cells can be modified to secrete insulin, a blood clotting factor (e.g., Factor VIII or von Willebrand Factor), alpha-1 antitrypsin, adenosine deaminase, tissue plasminogen activator, interleukins (e.g., IL-1, IL-2, IL-3), and the like.

In certain embodiments, the endothelial cells can be modified in a way that improves their performance in the context of an implanted graft. Non-limiting illustrative examples include secretion or expression of a thrombolytic agent to prevent intraluminal clot formation, secretion of an inhibitor of smooth muscle proliferation to prevent luminal stenosis due to smooth muscle hypertrophy, and expression and/or secretion of an endothelial cell mitogen or autocrine factor to stimulate endothelial cell proliferation and improve the extent or duration of the endothelial cell lining of the graft lumen.

In some embodiments, the engineered endothelial cells are utilized for delivery of therapeutic levels of a secreted product to a specific organ or limb. For example, a vascular implant lined with endothelial cells engineered (transduced) in vitro can be grafted into a specific organ or limb. The secreted product of the transduced endothelial cells will be delivered in high concentrations to the perfused tissue, thereby achieving a desired effect to a targeted anatomical location.

In other embodiments, the endothelial cells are further genetically modified to contain a gene that disrupts or inhibits angiogenesis when expressed by endothelial cells in a vascularizing tumor. In some cases, the endothelial cells can also be genetically modified to express any one of the selectable suicide genes described herein which allows for negative selection of grafted endothelial cells upon completion of tumor treatment.

In some embodiments, endothelial cells described herein, such as isolated primary endothelial cells, are administered to a recipient subject to treat a vascular disorder selected from the group consisting of vascular injury, cardiovascular disease, vascular disease, peripheral vascular disease, ischemic disease, myocardial infarction, congestive heart failure, peripheral vascular obstructive disease, hypertension, ischemic tissue injury, reperfusion injury, limb ischemia, stroke, neuropathy (e.g., peripheral neuropathy or diabetic neuropathy), organ failure (e.g., liver failure, kidney failure, and the like), diabetes, rheumatoid arthritis, osteoporosis, cerebrovascular disease, hypertension, angina pectoris and myocardial infarction due to coronary artery disease, renal vascular hypertension, renal failure due to renal artery stenosis, claudication of the lower extremities, other vascular condition or disease.

e. Epithelial Cells

1) Retinal Pigmented Epithelium (RPE) Cells

In some embodiments, the cells that are engineered or modified as provided herein are primary retinal pigmented epithelium (RPE) cells. In some embodiments, the primary RPE cells are isolated or obtained from one or more individual donor subjects, such as one or more individual healthy donor (e.g. a subject that is not known or suspected of, e.g. not exhibiting clinical signs of, a disease or infection). As will be appreciated by those in the art, methods of isolating or obtaining RPE cells from an individual can be achieved using known techniques. Provided herein are engineered primary RPE cells that contain modifications (e.g. genetic modifications) described herein for subsequent transplantation or engraftment into subjects (e.g., recipients).

In some embodiments, primary RPE cells are obtained (e.g., harvested, extracted, removed, or taken) from a subject or an individual. In some embodiments, primary RPE cells are produced from a pool of RPE cells such that the RPE cells are from one or more subjects (e.g., one or more human including one or more healthy humans). In some embodiments, the pool of primary RPE cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of RPE cells does not include cells from the patient. In some embodiments, one or more of the donor subjects from which the pool of RPE cells is obtained are different from the patient.

Additional descriptions of RPE cells, including methods for their use in the present technology, are found in WO2020/018615, the disclosure is herein incorporated by reference in its entirety.

In some embodiments, the population of engineered RPE cells, such as primary RPE cells isolated from one or more individual donors (e.g. healthy donors), are maintained in culture, in some cases expanded, prior to administration. In certain embodiments, the population of RPE cells are cryopreserved prior to administration.

Exemplary RPE cell types include, but are not limited to, retinal pigmented epithelium (RPE) cell, RPE progenitor cell, immature RPE cell, mature RPE cell, functional RPE cell, and the like.

In some embodiments, the RPE cells, such as primary RPE cells isolated from one or more individual donors (e.g. healthy donors), have a genetic expression profile similar or substantially similar to that of native RPE cells. Such RPE cells may possess the polygonal, planar sheet morphology of native RPE cells when grown to confluence on a planar substrate.

In some embodiments, the present technology is directed to engineered RPE cells, such as primary RPE cells isolated from one or more individual donors (e.g. healthy donors), that overexpress a tolerogenic factor (e.g. CD47), and have reduced expression or lack expression of one or more MHC class I molecules and/or one or more MHC class II molecules (e.g., one or more MHC class I human leukocyte antigen molecules and one or more MHC class II human leukocyte antigen molecules). In certain embodiments, the engineered RPE cells overexpress a tolerogenic factor (e.g. CD47) and harbor a genomic modification in the B2M gene. In some embodiments, the engineered RPE cells overexpress a tolerogenic factor (e.g. CD47) and harbor a genomic modification in the CIITA gene. In some embodiments, engineered RPE cells overexpress a tolerogenic factor (e.g. CD47) and harbor genomic modifications that disrupt one or more of the following genes: the B2M and CIITA genes.

In some embodiments, the provided engineered RPE cells evade immune recognition. In some embodiments, the engineered RPE cells described herein, such as primary RPE cells isolated from one or more individual donors (e.g. healthy donors), do not activate an immune response in the patient (e.g., recipient upon administration). Provided are methods of treating a disease by administering a population of engineered RPE cells described herein to a subject (e.g., recipient) or patient in need thereof.

The RPE cells can be implanted into a patient suffering from macular degeneration or a patient having damaged RPE cells. In some embodiments, the patient has age-related macular degeneration (AMD), early AMD, intermediate AMD, late AMD, non-neovascular age-related macular degeneration, dry macular degeneration (dry age-related macular degeneration), wet macular degeneration (wet age-real ted macular degeneration), juvenile macular degeneration (JMD) (e.g., Stargardt disease, Best disease, and juvenile retinoschisis), Leber's Congenital Ameurosis, or retinitis pigmentosa. In other embodiments, the patient suffers from retinal detachment.

2) Thyroid Cells

In some embodiments, the cells that are engineered or modified as provided herein are primary thyroid cells. In some embodiments, the primary thyroid cells are isolated or obtained from one or more individual donor subjects, such as one or more individual healthy donor (e.g. a subject that is not known or suspected of, e.g. not exhibiting clinical signs of, a disease or infection). As will be appreciated by those in the art, methods of isolating or obtaining thyroid cells from an individual can be achieved using known techniques. Provided herein are engineered primary thyroid cells that contain modifications (e.g. genetic modifications) described herein for subsequent transplantation or engraftment into subjects (e.g., recipients).

In some embodiments, primary thyroid cells are obtained (e.g., harvested, extracted, removed, or taken) from a subject or an individual. In some embodiments, primary thyroid cells are produced from a pool of thyroid cells such that the thyroid cells are from one or more subjects (e.g., one or more human including one or more healthy humans). In some embodiments, the pool of primary thyroid cells is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of thyroid cells does not include cells from the patient. In some embodiments, one or more of the donor subjects from which the pool of thyroid cells is obtained are different from the patient.

In some embodiments, the present technology is directed to engineered thyroid cells, such as primary thyroid cells isolated from one or more individual donors (e.g. healthy donors), that overexpress a tolerogenic factor (e.g. CD47), and have reduced expression or lack expression of one or more MHC class I molecules and/or one or more MHC class II molecules (e.g., one or more MHC class I human leukocyte antigen molecules and one or more MHC class II human leukocyte antigen molecules). In certain embodiments, the engineered thyroid cells overexpress a tolerogenic factor (e.g. CD47) and harbor a genomic modification in the B2M gene. In some embodiments, the engineered thyroid cells overexpress a tolerogenic factor (e.g. CD47) and harbor a genomic modification in the CIITA gene. In some embodiments, engineered thyroid cells overexpress a tolerogenic factor (e.g. CD47) and harbor genomic modifications that disrupt one or more of the following genes: the B2M and CIITA genes.

In some embodiments, the provided engineered thyroid cells evade immune recognition. In some embodiments, the engineered thyroid cells described herein, such as primary thyroid cells isolated from one or more individual donors (e.g. healthy donors), do not activate an immune response in the patient (e.g., recipient upon administration). Provided are methods of treating a disease by administering a population of engineered endothelial cells described herein to a subject (e.g., recipient) or patient in need thereof.

f. Hepatocytes

In some embodiments, the cells that are engineered or modified as provided herein are primary hepatocytes. In some embodiments, the primary hepatocytes are isolated or obtained from one or more individual donor subjects, such as one or more individual healthy donor (e.g. a subject that is not known or suspected of, e.g. not exhibiting clinical signs of, a disease or infection). As will be appreciated by those in the art, methods of isolating or obtaining hepatocytes from an individual can be achieved using known techniques. Provided herein are engineered primary hepatocytes that contain modifications (e.g. genetic modifications) described herein for subsequent transplantation or engraftment into subjects (e.g., recipients). In some embodiments, engineered primary hepatocytes can be administered as a cell therapy to address loss of the hepatocyte functioning or cirrhosis of the liver.

In some embodiments, primary hepatocytes are obtained (e.g., harvested, extracted, removed, or taken) from a subject or an individual. In some embodiments, primary hepatocytes are produced from a pool of hepatocytes such that the hepatocytes are from one or more subjects (e.g., one or more human including one or more healthy humans). In some embodiments, the pool of primary hepatocytes is from 1-100, 1-50, 1-20, 1-10, 1 or more, 2 or more, 3 or more, 4 or more, 5 or more, 10 or more, 20 or more, 30 or more, 40 or more, 50 or more, or 100 or more subjects. In some embodiments, the donor subject is different from the patient (e.g., the recipient that is administered the therapeutic cells). In some embodiments, the pool of hepatocytes does not include cells from the patient. In some embodiments, one or more of the donor subjects from which the pool of hepatocytes is obtained are different from the patient.

In some embodiments, the population of engineered hepatocytes, such as primary hepatocytes isolated from one or more individual donors (e.g. healthy donors), are maintained in culture, in some cases expanded, prior to administration. In certain embodiments, the population of hepatocytes are cryopreserved prior to administration.

In some embodiments, the present technology is directed to engineered hepatocytes, such as primary hepatocytes isolated from one or more individual donors (e.g. healthy donors), that overexpress a tolerogenic factor (e.g. CD47), and have reduced expression or lack expression of one or more MHC class I molecules and/or one or more MHC class II molecules (e.g., one or more MHC class I human leukocyte antigen molecules and one or more MHC class II human leukocyte antigen molecules). In certain embodiments, the engineered hepatocytes overexpress a tolerogenic factor (e.g. CD47) and harbor a genomic modification in the B2M gene. In some embodiments, the engineered hepatocytes overexpress a tolerogenic factor (e.g. CD47) and harbor a genomic modification in the CIITA gene. In some embodiments, engineered hepatocytes overexpress a tolerogenic factor (e.g. CD47) and harbor genomic modifications that disrupt one or more of the following genes: the B2M and CIITA genes.

In some embodiments, the provided engineered hepatocytes evade immune recognition. In some embodiments, the engineered hepatocytes described herein, such as primary hepatocytes isolated from one or more individual donors (e.g. healthy donors), do not activate an immune response in the patient (e.g., recipient upon administration). Provided are methods of treating a disease by administering a population of engineered hepatocytes described herein to a subject (e.g., recipient) or patient in need thereof.

g. Cardiac Cells

Provided herein are cardiac cell types for subsequent transplantation or engraftment into subjects (e.g., recipients).

In some embodiments, cardiac cells described herein are administered to a recipient subject to treat a cardiac disorder selected from the group consisting of pediatric cardiomyopathy, age-related cardiomyopathy, dilated cardiomyopathy, hypertrophic cardiomyopathy, restrictive cardiomyopathy, chronic ischemic cardiomyopathy, peripartum cardiomyopathy, inflammatory cardiomyopathy, idiopathic cardiomyopathy, other cardiomyopathy, myocardial ischemic reperfusion injury, ventricular dysfunction, heart failure, congestive heart failure, coronary artery disease, end-stage heart disease, atherosclerosis, ischemia, hypertension, restenosis, angina pectoris, rheumatic heart, arterial inflammation, cardiovascular disease, myocardial infarction, myocardial ischemia, congestive heart failure, myocardial infarction, cardiac ischemia, cardiac injury, myocardial ischemia, vascular disease, acquired heart disease, congenital heart disease, atherosclerosis, coronary artery disease, dysfunctional conduction systems, dysfunctional coronary arteries, pulmonary hypertension, cardiac arrhythmias, muscular dystrophy, muscle mass abnormality, muscle degeneration, myocarditis, infective myocarditis, drug- or toxin-induced muscle abnormalities, hypersensitivity myocarditis, and autoimmune endocarditis.

Accordingly, provided herein are methods for the treatment and prevention of a cardiac injury or a cardiac disease or disorder in a subject in need thereof. The methods described herein can be used to treat, ameliorate, prevent or slow the progression of a number of cardiac diseases or their symptoms, such as those resulting in pathological damage to the structure and/or function of the heart. The terms “cardiac disease,” “cardiac disorder,” and “cardiac injury,” are used interchangeably herein and refer to a condition and/or disorder relating to the heart, including the valves, endothelium, infarcted zones, or other components or structures of the heart. Such cardiac diseases or cardiac-related disease include, but are not limited to, myocardial infarction, heart failure, cardiomyopathy, congenital heart defect, heart valve disease or dysfunction, endocarditis, rheumatic fever, mitral valve prolapse, infective endocarditis, hypertrophic cardiomyopathy, dilated cardiomyopathy, myocarditis, cardiomegaly, and/or mitral insufficiency, among others.

In some embodiments, the population of engineered cardiac cells are maintained in culture, in some cases expanded, prior to administration. In certain embodiments, the population of cardiac cells are cryopreserved prior to administration.

In some embodiments, the present technology is directed to engineered cardiac cells that overexpress a tolerogenic factor (e.g. CD47), and have reduced expression or lack expression of one or more MHC class I molecules and/or one or more MHC class II molecules (e.g., one or more MHC class I human leukocyte antigen molecules and one or more MHC class II human leukocyte antigen molecules). In certain embodiments, the engineered cardiac cells overexpress a tolerogenic factor (e.g. CD47) and harbor a genomic modification in the B2M gene. In some embodiments, the engineered cardiac cells overexpress a tolerogenic factor (e.g CD47) and harbor a genomic modification in the CIITA gene. In some embodiments, engineered cardiac cells overexpress a tolerogenic factor (e.g. CD47) and harbor genomic modifications that disrupt one or more of the following genes: the B2M and CIITA genes.

In some embodiments, the provided engineered cardiac cells evade immune recognition. In some embodiments, the engineered cardiac cells described herein do not activate an immune response in the patient (e.g., recipient upon administration). Provided are methods of treating a disease by administering a population of engineered cardiac cells described herein to a subject (e.g., recipient) or patient in need thereof.

In some embodiments, the administration comprises implantation into the subject's heart tissue, intravenous injection, intraarterial injection, intracoronary injection, intramuscular injection, intraperitoneal injection, intramyocardial injection, trans-endocardial injection, trans-epicardial injection, or infusion.

In some embodiments, the patient administered the engineered cardiac cells is also administered a cardiac drug. Illustrative examples of cardiac drugs that are suitable for use in combination therapy include, but are not limited to, growth factors, polynucleotides encoding growth factors, angiogenic agents, calcium channel blockers, antihypertensive agents, antimitotic agents, inotropic agents, anti-atherogenic agents, anti-coagulants, beta-blockers, anti-arhythmic agents, anti-inflammatory agents, vasodilators, thrombolytic agents, cardiac glycosides, antibiotics, antiviral agents, antifungal agents, agents that inhibit protozoans, nitrates, angiotensin converting enzyme (ACE) inhibitors, angiotensin II receptor antagonist, brain natriuretic peptide (BNP); antineoplastic agents, steroids, and the like.

The effects of therapy according to the methods provided herein can be monitored in a variety of ways. For instance, an electrocardiogram (ECG) or holier monitor can be utilized to determine the efficacy of treatment. An ECG is a measure of the heart rhythms and electrical impulses, and is a very effective and non-invasive way to determine if therapy has improved or maintained, prevented, or slowed degradation of the electrical conduction in a subject's heart. The use of a holier monitor, a portable ECG that can be worn for long periods of time to monitor heart abnormalities, arrhythmia disorders, and the like, is also a reliable method to assess the effectiveness of therapy. An ECG or nuclear study can be used to determine improvement in ventricular function.

h. Neural Cells

Provided herein are different neural cell types that are useful for subsequent transplantation or engraftment into recipient subjects. Exemplary neural cell types include, but are not limited to, cerebral endothelial cells, neurons (e.g., dopaminergic neurons), glial cells, and the like.

In some embodiments, the population of engineered neural cells are maintained in culture, in some cases expanded, prior to administration. In certain embodiments, the population of neural cells are cryopreserved prior to administration.

In some embodiments, the present technology is directed to engineered neural cells that overexpress a tolerogenic factor (e.g. CD47), and have reduced expression or lack expression of one or more MHC class I molecules and/or one or more MHC class II molecules (e.g., one or more MHC class I human leukocyte antigen molecules and one or more MHC class II human leukocyte antigen molecules). In certain embodiments, the engineered neural cells overexpress a tolerogenic factor (e.g. CD47) and harbor a genomic modification in the B2M gene. In some embodiments, the engineered neural cells overexpress a tolerogenic factor (e.g CD47) and harbor a genomic modification in the CIITA gene. In some embodiments, engineered neural cells overexpress a tolerogenic factor (e.g. CD47) and harbor genomic modifications that disrupt one or more of the following genes: the B2M and CIITA genes.

In some embodiments, the provided engineered neural cells evade immune recognition. In some embodiments, the engineered neural cells described herein do not activate an immune response in the patient (e.g., recipient upon administration). Provided are methods of treating a disease by administering a population of engineered neural cells described herein to a subject (e.g., recipient) or patient in need thereof.

In some embodiments, neural cells are administered to a subject to treat Parkinson's disease, Huntington disease, multiple sclerosis, other neurodegenerative disease or condition, attention deficit hyperactivity disorder (ADHD), Tourette Syndrome (TS), schizophrenia, psychosis, depression, other neuropsychiatric disorder. In some embodiments, neural cells described herein are administered to a subject to treat or ameliorate stroke. In some embodiments, the neurons and glial cells are administered to a subject with amyotrophic lateral sclerosis (ALS).

1) Cerebral Endothelial Cells

In some embodiments, cerebral endothelial cells are administered to alleviate the symptoms or effects of cerebral hemorrhage. In some embodiments, dopaminergic neurons are administered to a patient with Parkinson's disease. In some embodiments, noradrenergic neurons, GABAergic interneurons are administered to a patient who has experienced an epileptic seizure. In some embodiments, motor neurons, interneurons, Schwann cells, oligodendrocytes, and microglia are administered to a patient who has experienced a spinal cord injury.

2) Dopaminergic Neurons

In some embodiments, HIP cells described herein are dopaminergic neurons.

In some cases, the term “dopaminergic neurons” includes neuronal cells which express tyrosine hydroxylase (TH), the rate-limiting enzyme for dopamine synthesis. In some embodiments, dopaminergic neurons secrete the neurotransmitter dopamine, and have little or no expression of dopamine hydroxylase. A dopaminergic (DA) neuron can express one or more of the following markers: neuron-specific enolase (NSE), 1-aromatic amino acid decarboxylase, vesicular monoamine transporter 2, dopamine transporter, Nurr-1, and dopamine-2 receptor (D2 receptor).

In some embodiments, the DA neurons are administered to a patient, e.g., human patient to treat a neurodegenerative disease or condition. In some cases, the neurodegenerative disease or condition is selected from the group consisting of Parkinson's disease, Huntington disease, and multiple sclerosis. In other embodiments, the DA neurons are used to treat or ameliorate one or more symptoms of a neuropsychiatric disorder, such as attention deficit hyperactivity disorder (ADHD), Tourette Syndrome (TS), schizophrenia, psychosis, and depression. In yet other embodiments, the DA neurons are used to treat a patient with impaired DA neurons.

In some embodiments, the differentiated DA neurons are transplanted either intravenously or by injection at particular locations in the patient. In some embodiments, the DA cells are transplanted into the substantia nigra (particularly in or adjacent of the compact region), the ventral tegmental area (VTA), the caudate, the putamen, the nucleus accumbens, the subthalamic nucleus, or any combination thereof, of the brain to replace the DA neurons whose degeneration resulted in Parkinson's disease. The DA cells can be injected into the target area as a cell suspension. Alternatively, the DA cells can be embedded in a support matrix or scaffold when contained in such a delivery device. In some embodiments, the scaffold is biodegradable. In other embodiments, the scaffold is not biodegradable. The scaffold can comprise natural or synthetic (artificial) materials.

The delivery of the DA neurons can be achieved by using a suitable vehicle such as, but not limited to, liposomes, microparticles, or microcapsules. In other embodiments, the DA neurons are administered in a pharmaceutical composition comprising an isotonic excipient. The pharmaceutical composition is prepared under conditions that are sufficiently sterile for human administration. In some embodiments, the DA are supplied in the form of a pharmaceutical composition.

3) Glial Cells

In some embodiments, the neural cells described include glial cells such as, but not limited to, microglia, astrocytes, oligodendrocytes, ependymal cells and Schwann cells, glial precursors, and glial progenitors.

The efficacy of neural cell transplants for spinal cord injury can be assessed in, for example, a rat model for acutely injured spinal cord, as described by McDonald, et al., Nat. Med., 1999, 5:1410) and Kim, et al., Nature, 2002, 418:50. For instance, successful transplants may show transplant-derived cells present in the lesion 2-5 weeks later, differentiated into astrocytes, oligodendrocytes, and/or neurons, and migrating along the spinal cord from the lesioned end, and an improvement in gait, coordination, and weight-bearing. Specific animal models are selected based on the neural cell type and neurological disease or condition to be treated.

The neural cells can be administered in a manner that permits them to engraft to the intended tissue site and reconstitute or regenerate the functionally deficient area. For instance, neural cells can be transplanted directly into parenchymal or intrathecal sites of the central nervous system, according to the disease being treated. In some embodiments, any of the neural cells described herein including cerebral endothelial cells, neurons, dopaminergic neurons, ependymal cells, astrocytes, microglial cells, oligodendrocytes, and Schwann cells are injected into a patient by way of intravenous, intraspinal, intracerebroventricular, intrathecal, intra-arterial, intramuscular, intraperitoneal, subcutaneous, intramuscular, intra-abdominal, intraocular, retrobulbar and combinations thereof. In some embodiments, the cells are injected or deposited in the form of a bolus injection or continuous infusion. In certain embodiments, the neural cells are administered by injection into the brain, apposite the brain, and combinations thereof. The injection can be made, for example, through a burr hole made in the subject's skull. Suitable sites for administration of the neural cell to the brain include, but are not limited to, the cerebral ventricle, lateral ventricles, cisterna magna, putamen, nucleus basalis, hippocampus cortex, striatum, caudate regions of the brain and combinations thereof.

Additional descriptions of neural cells including dopaminergic neurons for use in the present technology are found in WO2020/018615, the disclosure is herein incorporated by reference in its entirety.

2. Stem Cells

In some embodiments, the cell is a stem cell or progenitor cell (e.g., iPSC, embryonic stem cell, hematopoietic stem cell, mesenchymal stem cell, endothelial stem cell, epithelial stem cell, adipose stem or progenitor cells, germline stem cells, lung stem or progenitor cells, mammary stem cells, olfactory adult stem cells, hair follicle stem cells, multipotent stem cells, amniotic stem cells, cord blood stem cells, or neural stem or progenitor cells). In some embodiments, the stem cells are adult stem cells (e.g., somatic stem cells or tissue specific stem cells). In some embodiments, the stem or progenitor cell is capable of being differentiated (e.g., the stem cell is totipotent, pluripotent, or multipotent). In some embodiments, the cell is manipulated (e.g., converted or differentiated) into a muscle cell, erythroid-megakaryocytic cell, eosinophil, iPS cell, macrophage, T cell, islet beta-cell, neuron, cardiomyocyte, blood cell, endocrine progenitor, exocrine progenitor, ductal cell, acinar cell, alpha cell, beta cell, delta cell, PP cell, hepatocyte, cholangiocyte, or brownadipocyte.

In some embodiments, the cells that are engineered as provided herein are induced pluripotent stem cells or are engineered cell that are derived from or differentiated from induced pluripotent stem cells. The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al, World J. Stem Cells 7(1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogeneous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POU5F1), KLF4, MYC, NANOG, LIN28, and SV40L T antigen. In general, these reprogramming factor genes are provided on episomal vectors such as are known in the art and commercially available.

In some embodiments, the hosts cells used for transfecting the one or more reprogramming factors are non-pluripotent stem cells. In general, as is known in the art, iPSCs are made from non-pluripotent cells such as, but not limited to, blood cells, fibroblasts, etc., by transiently expressing the reprogramming factors as described herein. In some embodiments, the non-pluripotent cells, such as fibroblasts, are obtained or isolated from one or more individual subjects or donors prior to reprogramming the cells. In some embodiments, iPSCs are made from a pool of isolated non-pluripotent stems cells, e.g. fibroblasts, obtained from one or more (e.g. two or more, three or more, four or more, five or more, ten or more, twenty or more, fifty or more, or one hundred or more) different donor subjects. In some embodiments, the non-pluripotent cells, such as fibroblasts, are isolated or obtained from a plurality of different donor subjects (e.g. two or more, three or more, four or more, five or more, ten or more, twenty or more, fifty or more, or one hundred or more), pooled together in a batch, reprogrammed as iPSCs and are engineered in accord with the provided methods.

In some embodiments, the iPSCs are derived from, such as by transiently transfecting one or more reprogramming factors into cells from a pool of non-pluripotent cells (e.g. fibroblasts) from one or more donor subjects that are different than the recipient subject (e.g., the patient administered the cells). The non-pluripotent cells (e.g. fibroblasts) to be induced to iPSCs can be obtained from 1, 2, 3, 4, 5, 6, 7, 8, 9, 10, 20, 50, 100 or more donor subjects and pooled together. The non-pluripotent cells (e.g. fibroblasts) can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the non-pluripotent cells (e.g. fibroblasts) are harvested from one or a plurality of individuals, and in some instances, the non-pluripotent cells (e.g. fibroblasts) or the pool of non-pluripotent cells (e.g. fibroblasts) are cultured in vitro and transfected with one or more reprogramming factors to induce generation of iPSCs. In some embodiments, the non-pluripotent cells (e.g. fibroblasts) or the pool of non-pluripotent cells (e.g. fibroblasts) are engineered or modified in accord with the methods provided herein. In some embodiments, the engineered iPSCs or a pool of engineered iPSCs are then subjected to a differentiation process for differentiation into any cells of an organism and tissue.

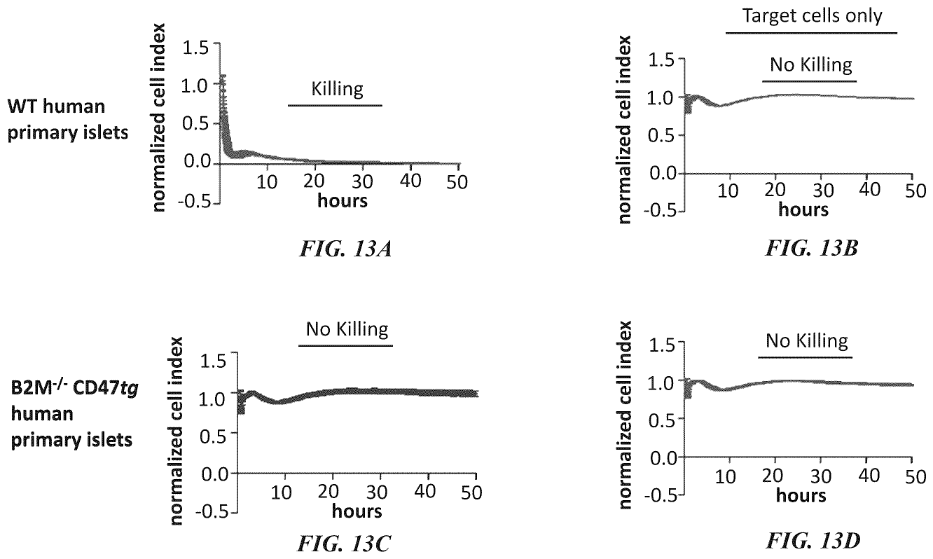

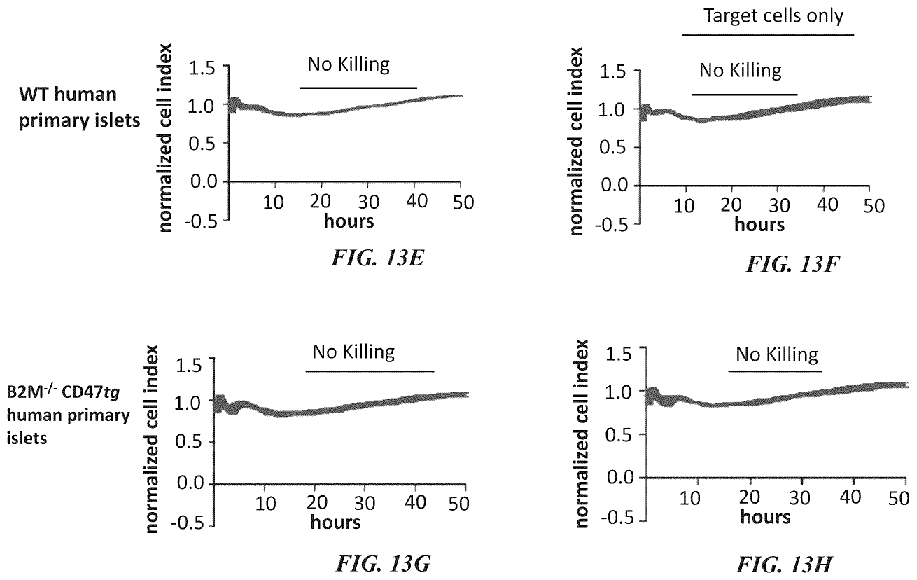

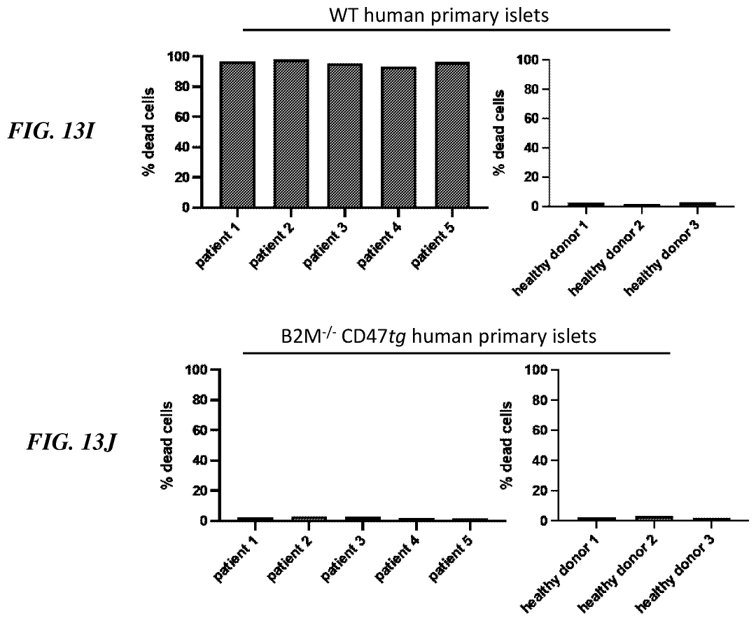

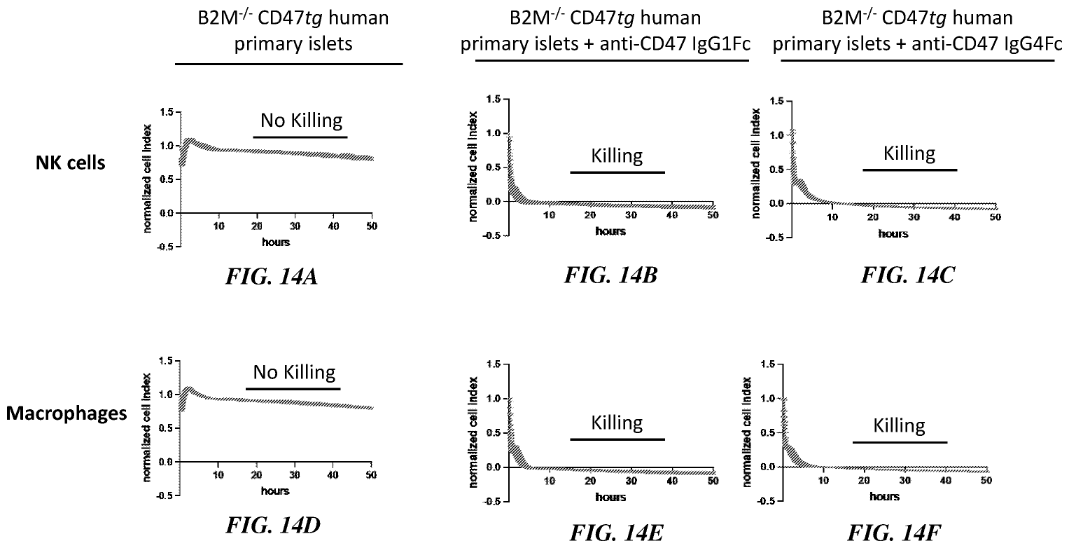

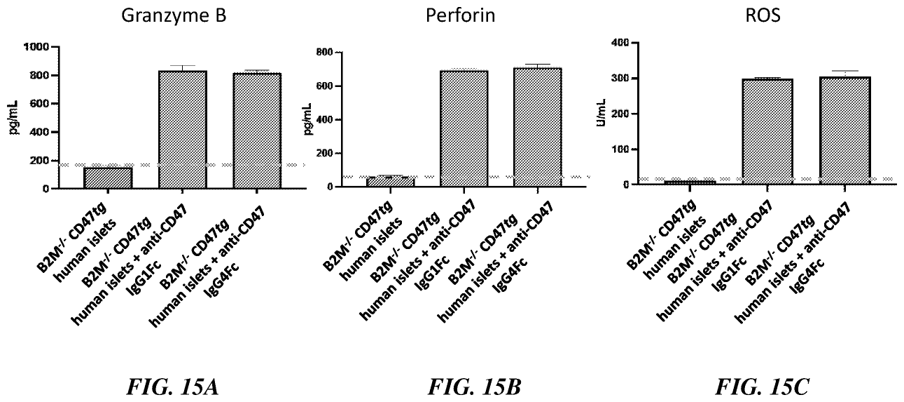

Once the engineered iPSCs cells have been generated, they may be assayed for their hypoimmunogenicity and/or retention of pluripotency as is described in WO2016183041 and WO2018132783. In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g. teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of engineered or modified iPSCs is determined using an allogeneic humanized immunodeficient mouse model. In some instances, the engineered or modified iPSCs are transplanted into an allogeneic humanized NSG-SGM3 mouse and assayed for cell rejection, cell survival, and teratoma formation. In some instances, grafted engineered iPSCs or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

Similarly, the retention of pluripotency is tested in a number of ways. In one embodiment, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

Once the engineered pluripotent stem cells (engineered iPSCs) have been generated, they can be maintained in an undifferentiated state as is known for maintaining iPSCs. For example, the cells can be cultured on Matrigel using culture media that prevents differentiation and maintains pluripotency. In addition, they can be in culture medium under conditions to maintain pluripotency.

Any of the pluripotent stem cells described herein can be differentiated into any cells of an organism and tissue. In an aspect, provided herein are engineered cells that are differentiated into different cell types from iPSCs for subsequent transplantation into recipient subjects. Differentiation can be assayed as is known in the art, generally by evaluating the presence of cell-specific markers. As will be appreciated by those in the art, the differentiated engineered (e.g. hypoimmunogenic) pluripotent cell derivatives can be transplanted using techniques known in the art that depends on both the cell type and the ultimate use of these cells. In some embodiments, the iPSCs may be differentiated to any type of cell described herein. In some embodiments, the iPSCs are differentiated into cell types selected from T cells, NK cells, beta islet cells, endothelial cells, epithelial cells such as RPE, thyroid, skin, or hepatocytes. In some embodiments, host cells such as non-pluripotent cells (e.g. fibroblasts) from an individual donor or a pool of individual donors are isolated or obtained, generated into iPSCs in which the iPSCs are then engineered to contain modifications (e.g. genetic modifications) described herein and then differentiated into a desired cell type.

3. ABO Blood Type and Rh Antigen Expression

Blood products can be classified into different groups according to the presence or absence of antigens on the surface of every red blood cell in a person's body (ABO Blood Type). The A, B, AB, and A1 antigens are determined by the sequence of oligosaccharides on the glycoproteins of erythrocytes. The genes in the blood group antigen group provide instructions for making antigen proteins. Blood group antigen proteins serve a variety of functions within the cell membrane of red blood cells. These protein functions include transporting other proteins and molecules into and out of the cell, maintaining cell structure, attaching to other cells and molecules, and participating in chemical reactions.

The Rhesus Factor (Rh) blood group is the second most important blood group system, after the ABO blood group system The Rh blood group system consists of 49 defined blood group antigens, among which five antigens, D, C, c, E, and e, are the most important. Rh(D) status of an individual is normally described with a positive or negative suffix after the ABO type. The terms “Rh factor,” “Rh positive,” and “Rh negative” refer to the Rh(D) antigen only. Antibodies to Rh antigens can be involved in hemolytic transfusion reactions and antibodies to the Rh(D) and Rh(c) antigens confer significant risk of hemolytic disease of the fetus and newborn. ABO antibodies develop in early life in every human. However, rhesus antibodies in Rh− humans typically develop only when the person is sensitized. This can occur, for example, by giving birth to a Rh+ baby or by receiving an Rh+ blood transfusion.

A, B, H, and Rh antigens are major determinants of histocompatibility between donor and recipient for blood, tissue and cellular transplantation. A glycosyltransferase activity encoded by the ABO gene is responsible for producing A, B, AB, O histo-blood group antigens, which are displayed on the surface of cells. Group A individuals encode an ABO gene product with specificity to produce a(1,3)N-acetylgalactosaminyltransferase activity and group B individuals with specificity to produce a(1, 3) galactosyltransferase activity. Type O individuals do not produce a functional galactosyltransferase at all and thus do not produce either modification. Type AB individuals harbor one copy of each and produce both types of modifications. The enzyme products of the ABO gene act on the H antigen as a substrate, and thus type O individuals whom lack ABO activity present an unmodified H antigen and are thus often referred to as type O(H).

The H antigen itself is the product of an a(1,2)fucosyltransferase enzyme, which is encoded by the FUT1 gene. In very rare individuals there exists a loss of the H antigen entirely as a result of a disruption of the FUT1 gene and no substrate will exist for ABO to produce A or B histo-blood types. These individuals are said to be of the Bombay histo-blood type. The Rh antigen is encoded by the RHD gene, and individuals who are Rh negative harbor a deletion or disruption of the RHD gene.

In some embodiments, the cells or population of cells provided herein are ABO type O Rh factor negative. In some embodiments, ABO type O Rh factor negative cells described herein are derived from an ABO type O Rh factor negative donor. In some embodiments, ABO type O Rh factor negative cells described herein are engineered to lack presentation of ABO type A, ABO type B, or Rh factor antigens. In some embodiments, ABO type O and/or Rh negative cells comprise partial or complete inactivation of an ABO gene (e.g., by deleterious variation of the ABO gene or by insertion of an exon 6 258delG variation of the ABO gene), and/or expression of an RHD gene is partially or fully inactivated by a deleterious variation of the RHD gene. In some embodiments, ABO type O Rh negative cells comprise partial or complete inactivation of a FUT1 gene and/or expression of an RHD gene is partially or fully inactivated by a deleterious variation of the RHD gene. In some embodiments, an engineered ABO type O and/or Rh factor negative cell is generated using gene editing to modify, for instance, a type A cell to a type O cell, a type B cell to a type O cell, a type AB cell to a type O cell, a type A+ cell to a type O− cell, a type A− cell to a type O− cell, a type AB+ cell to a type O− cell, a type AB− cell to a type O− cell, a type B+ cell to a type O− cell, and a type B− cell to a type O− cell. Exemplary engineered ABO type O Rh factor negative cells and methods of generating same are described in WO2021/146222, the content of which is herein incorporated by reference in its entirety.

4. Sex Chromosomes

In certain aspects, cells having a sex chromosome may express certain antigens (e.g., Y antigens), and recipients may have a preexisting sensitivity to such antigens. For example, in some embodiments, a female who has been pregnant with a male fetus may reject cells from a male donor. Thus, in some embodiments, the donor is a male and the recipient is a male. In some embodiments, the donor is a female and the recipient is a female. In some embodiments, the engineered cell comprises a modification reducing expression of an antigen, such as Protocadherin Y and/or Neuroligin Y. In some embodiments, the gene encoding protocadheren Y (PCDH11Y; Ensembl ID ENSG00000099715) is reduced or eliminated, e.g. knocked out, in the engineered cell. In some embodiments, the gene encoding Neuroligin Y (NLGN4Y; Ensembl ID ENSG00000165246) is reduced or eliminated, e.g. knocked out, in the engineered cell. Any method for reducing or eliminating expression of a gene can be used, such as any described herein. In some embodiments, PCDH11Y and/or NLGN4Y is reduced or eliminated in the engineered cell by nuclease-mediated gene editing methods such as using CRISPR/Cas systems.

### D. Exemplary Embodiments of Engineered Primary Cells

In some embodiments, the engineered primary cells and populations thereof are engineered primary cells. In some embodiments, the engineered primary cell is a human cell or an animal cell. In some embodiments, the engineered primary cell is a primary cell isolated from a donor subject (e.g., a healthy donor subject not suspected of having a disease or condition at the time the donor sample is obtained from the individual donor subject). In some embodiments, the engineered primary cell is selected from a beta islet cell, B cell, T cell, NK cell, retinal pigmented epithelium cell, glial progenitor cell, endothelial cell, hepatocyte, thyroid cell, skin cell, and blood cell (e.g., plasma cell or platelet). In some embodiments, the engineered primary cell is selected from a T cell and a NK cell, and further comprises a chimeric antigen receptor (CAR). In some embodiments, the engineered primary cell is ABO blood group type O. In some embodiments, the engineered primary cell is Rhesus factor negative (Rh−).

In some embodiments, the engineered primary cells and populations thereof exhibit increased expression of CD47, and reduced expression of one or more molecules of the one or more MHC class I complex and/or one or more MHC class II complex. In some embodiments, the engineered primary cells and populations thereof exhibit increased expression of CD47 and reduced expression of one or more molecules of the one or more MHC class I complex. In some embodiments, the engineered primary cells and populations thereof exhibit increased expression of CD47 and reduced expression of one or more molecules of the one or more MHC class II complex. In some embodiments, the engineered primary cells and populations thereof exhibit increased expression of CD47 and reduced expression of one or more molecules of the one or more MHC class I and one or more MHC class II complexes. In some embodiments, the modification(s) that confer overexpression comprise increased surface expression, and/or the modifications that reduce expression comprise reduced surface expression. In some embodiments, the engineered primary cell is selected from an islet cell, a beta islet cell, B cell, T cell, NK cell, retinal pigmented epithelium cell, glial progenitor cell, endothelial cell, hepatocyte, thyroid cell, skin cell, and blood cell (e.g., plasma cell or platelet). In some embodiments, the engineered primary cell is selected from a T cell and a NK cell, and further comprises a chimeric antigen receptor (CAR). In some embodiments, the engineered primary cell is ABO blood group type O. In some embodiments, the engineered primary cell is Rhesus factor negative (Rh−).

In some embodiments, the engineered primary cells and populations thereof exhibit increased expression of CD47 and reduced expression of B2M. In some embodiments, the cells and populations thereof exhibit increased expression of CD47 and reduced expression of CIITA. In some embodiments, the cells and populations thereof exhibit increased expression of CD47 and reduced expression of NLRC5. In some embodiments, the cells and populations thereof exhibit increased expression of CD47 and reduced expression of one or more molecules of B2M and CIITA. In some embodiments, the cells and populations thereof exhibit increased expression of CD47 and reduced expression of one or more molecules of B2M and NLRC5. In some embodiments, the cells and populations thereof exhibit increased expression of CD47 and reduced expression of one or more molecules of CIITA and NLRC5. In some embodiments, the cells and populations thereof exhibit increased expression of CD47 and reduced expression of one or more molecules of B2M, CIITA and NLRC5. Any of the engineered primary cells described herein can also exhibit increased expression of one or more factors selected from the group including, but not limited to, CD47, A20/TNFAIP3, C1-Inhibitor, CCL21, CCL22, CD16, CD16 Fc receptor, CD24, CD27, CD35, CD39, CD46, CD52, CD55, CD59, CD200, CR1, CTLA4-Ig, DUX4, FasL, H2-M3, HLA-C, HLA-E, HLA-E heavy chain, HLA-G, PD-L1, IDO1, IL-10, IL15-RF, IL-35, MANF, Mfge8, PD-1, PD-L1, or Serpinb9. For instance, any of the engineered primary cells described herein can also exhibit increased expression of one or more factors selected from the group including, but not limited to, CD47, CD35, CD16 Fc receptor, CD16, CD52, IL15-RF, H2-M3, DUX4, CD24, CD27, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, and Serpinb9. In some embodiments, the modification(s) that increase expression comprise increased surface expression, and/or the modifications that reduce expression comprise reduced surface expression. In some embodiments, the engineered primary cell is selected from an islet cell, beta islet cell, B cell, T cell, NK cell, retinal pigmented epithelium cell, glial progenitor cell, endothelial cell, hepatocyte, thyroid cell, skin cell, and blood cell (e.g., plasma cell or platelet). In some embodiments, the engineered primary cell is selected from a T cell and a NK cell, and further comprises a chimeric antigen receptor (CAR). In some embodiments, the engineered primary cell is ABO blood group type O. In some embodiments, the engineered primary cell is Rhesus factor negative (Rh−).

In some embodiments, the engineered primary cells and populations thereof exhibit increased expression of CD47, optionally at least one other tolerogenic factor, and reduced expression of one or more molecules of the MHC class I complex. In some embodiments, the engineered primary cells and populations thereof exhibit increased expression of CD47, optionally at least one other tolerogenic factor, and reduced expression of one or more molecules of the MHC class II complex. In some embodiments, the engineered primary cells and populations thereof exhibit increased expression of CD47, optionally at least one other tolerogenic factor, and reduced expression of one or more molecules of the MHC class II and one or more molecules of the MHC class II complexes. In some embodiments, the engineered primary cells and populations thereof exhibit increased expression of CD47, optionally at least one other tolerogenic factor, and reduced expression of B2M. In some embodiments, the engineered primary cells and populations thereof exhibit increased expression of CD47 optionally, at least one other tolerogenic factor, and reduced expression of CIITA. In some embodiments, the engineered primary cells and populations thereof exhibit increased expression of CD47, optionally at least one other tolerogenic factor, and reduced expression of one or more molecules of B2M and CIITA. In some embodiments, the cells and populations thereof exhibit increased expression of CD47 and at least one other tolerogenic factor, and reduced expression of one or more molecules of B2M and NLRC5. In some embodiments, the cells and populations thereof exhibit increased expression of CD47 and at least one other tolerogenic factor, and reduced expression of one or more molecules of CIITA and NLRC5. In some embodiments, the cells and populations thereof exhibit increased expression of CD47 and at least one other tolerogenic factor, and reduced expression of one or more molecules of B2M, CIITA and NLRC5. In some embodiments, a tolerogenic factor includes any from the group including, but not limited to CD47, CD35, CD16 Fc receptor, CD16, CD52, IL15-RF, H2-M3, DUX4, CD24, CD27, CD200, HLA-C, HLA-E, HLA-E heavy chain, HLA-G, PD-L1, IDO1, CTLA4-Ig, C1-Inhibitor, IL-10, IL-35, IL-39, FasL, CCL21, CCL22, Mfge8, and Serpinb9). In some embodiments, the modification(s) that confer overexpression comprise increased surface expression, and/or the modifications that reduce expression comprise reduced surface expression. In some embodiments, the engineered primary cell is selected from an islet cell, beta islet cell, B cell, T cell, NK cell, retinal pigmented epithelium cell, glial progenitor cell, endothelial cell, hepatocyte, thyroid cell, skin cell, and blood cell (e.g., plasma cell or platelet). In some embodiments, the engineered primary cell is selected from a T cell and a NK cell, and further comprises a chimeric antigen receptor (CAR). In some embodiments, the engineered primary cell is ABO blood group type O. In some embodiments, the engineered primary cell is Rhesus factor negative (Rh−).

In some embodiments, the engineered primary cells and populations thereof exhibit overexpression of CD47, reduced expression B2M, and reduced expression of CIITA. In some embodiments, the reduced expression of B2M comprises reduced protein expression of B2M. In some embodiments, the reduced expression of B2M comprises reduced protein expression of B2M. In some embodiments, the reduced expression of B2M comprises eliminated protein expression of B2M. In some embodiments, the reduced expression of B2M comprises inactivation or disruption of both alleles of the B2M gene. In some embodiments, the reduced expression of B2M comprises inactivation or disruption of all B2M coding sequences in the cell. In some embodiments, the inactivation or disruption of B2M comprises an indel in the B2M gene or a deletion of a contiguous stretch of genomic DNA of the B2M gene. In some embodiments, the B2M gene is knocked out. In some embodiments, the reduced expression of CIITA comprises reduced protein expression of CIITA. In some embodiments, the reduced expression of CIITA comprises eliminated protein expression of CIITA. In some embodiments, the reduced expression of CIITA comprises inactivation or disruption of both alleles of the CIITA gene. In some embodiments, the reduced expression of CIITA comprises inactivation or disruption of all CIITA coding sequences in the cell. In some embodiments, the inactivation or disruption of CIITA comprises an indel in the CIITA gene or a deletion of a contiguous stretch of genomic DNA of the CIITA gene. In some embodiments, the CIITA gene is knocked out.

In some embodiments, the modification(s) that increase expression comprise increased surface expression, and/or the modifications that reduce expression comprise reduced surface expression.

In some embodiments, the engineered primary cell is selected from an islet cell, beta islet cell, B cell, T cell, NK cell, retinal pigmented epithelium cell, glial progenitor cell, endothelial cell, hepatocyte, thyroid cell, skin cell, and blood cell (e.g., plasma cell or platelet). In some embodiments, the engineered primary cell is selected from a T cell and a NK cell, and further comprises a chimeric antigen receptor (CAR). In some embodiments, the engineered primary cell is ABO blood group type O. In some embodiments, the engineered primary cell is Rhesus factor negative (Rh−).

One skilled in the art will appreciate that levels of expression such as increased (e.g., overexpression) or reduced expression of a gene, protein or molecule can be referenced or compared to a comparable cell. In some embodiments, an engineered primary cell having increased expression of CD47 refers to a modified primary cell having a higher level of CD47 protein compared to an unmodified primary cell. In some embodiments, an engineered primary cell having reduced expression of B2M refers to a modified primary cell having a lower level of B2M protein compared to an unmodified primary cell. In some embodiments, an engineered primary cell having reduced expression of CIITA refers to a modified primary cell having a lower level of CIITA protein compared to an unmodified primary cell.

In one embodiment, provided herein are engineered primary cells (e.g., primary cells) expressing exogenous CD47 polypeptides and having reduced expression of either one or more MHC class I complex proteins, one or more MHC class II complex proteins, or any combination of MHC class I and class II complex proteins. In another embodiment, the engineered primary cells express exogenous CD47 polypeptides and express reduced levels of B2M and CIITA polypeptides. In some embodiments, the engineered primary cells express exogenous CD47 polypeptides and possess modifications (e.g., genetic modifications) of the B2M and CIITA genes. In some instances, the modifications (e.g., genetic modifications) inactivate the B2M and CIITA genes. In some embodiments, the engineered primary cell is selected from an islet cell, beta islet cell, B cell, T cell, NK cell, retinal pigmented epithelium cell, glial progenitor cell, endothelial cell, hepatocyte, thyroid cell, skin cell, and blood cell (e.g., plasma cell or platelet). In some embodiments, the engineered primary cell is selected from a T cell and a NK cell, and further comprises a chimeric antigen receptor (CAR). In some embodiments, the engineered primary cell is ABO blood group type O. In some embodiments, the engineered primary cell is Rhesus factor negative (Rh−).

In some embodiments, provided herein are methods of generating an engineered primary cell (e.g., primary cell), wherein the method comprises reducing or eliminating the expression of one or more MHC class I molecules and/or one or more MHC class II molecules in the cell; and increasing the expression (e.g., overexpressing) CD47 in the cell. In some embodiments, the method comprises reducing or eliminating the expression of one or more MHC class I molecules. In some embodiments, the method comprises reducing or eliminating the expression of one or more MHC class I molecules and/or one or more MHC class II molecules. In some embodiments, the modification(s) that increase expression comprise increased surface expression, and/or the modifications that reduce expression comprise reduced surface expression. In some embodiments, the modification that increases expression of CD47 comprises an exogenous polynucleotide encoding the CD47 protein that is linked to a promoter. In some embodiments, the exogenous polynucleotide encoding CD47 is integrated into the genome of the engineered primary cell. In some embodiments, the integration is by is by targeted insertion into a target genomic locus of the cell, optionally wherein the targeted insertion is by nuclease-mediated gene editing with homology-directed repair. In some embodiments, the modification that reduces one or more MHC class I molecules protein expression and/or one or more MHC class II molecules protein expression is by nuclease-mediated gene editing. In some embodiments, the nuclease-mediated gene editing is by a zinc finger nuclease (ZFN), a TAL-effector nuclease (TALEN), or a CRISPR-Cas combination that targets the target genomic locus, optionally wherein the Cas is Cas9. In some embodiments, the engineered primary cell is a hypo-immunogenic primary cell. In some embodiments, the engineered primary cell is selected from an islet cell, beta islet cell, B cell, T cell, NK cell, retinal pigmented epithelium cell, glial progenitor cell, endothelial cell, hepatocyte, thyroid cell, skin cell, and blood cell (e.g., plasma cell or platelet). In some embodiments, the engineered primary cell is selected from a T cell and a NK cell, and further a chimeric antigen receptor (CAR). In some embodiments, the engineered primary cell is ABO blood group type O. In some embodiments, the engineered primary cell is Rhesus factor negative (Rh−).

In some embodiments, provided herein are methods of generating an engineered primary cell (e.g., primary cell), wherein the method comprises reducing or eliminating the expression of B2M; and increasing the expression (e.g., overexpressing) of CD47 in the cell. In some embodiments, the method comprises introducing a modification that reduces or eliminates the expression of B2M. In some embodiments, the modification that reduces or eliminates B2M expression comprises inactivation or disruption of both alleles of the B2M gene. In some embodiments, the modification that reduces or eliminates B2M comprises inactivation or disruption of all B2M coding sequences in the cell. In some embodiments, the inactivation or disruption comprises an indel in the B2M gene or a deletion of a contiguous stretch of genomic DNA of the B2M gene. In some embodiments, the indel is a frameshift mutation. In some embodiments, the B2M gene is knocked out. In some embodiments, the modification that reduces or eliminates B2M expression comprises reducing or eliminating B2M protein expression by nuclease-mediated gene editing. In some embodiments, the nuclease-mediated gene editing is by a zinc finger nuclease (ZFN), a TAL-effector nuclease (TALEN), or a CRISPR-Cas combination that targets the B2M gene, optionally wherein the Cas is Cas9. In some embodiments, the nuclease-mediated gene editing is by a CRISPR-Cas combination and the CRISPR-Cas combination comprises a guide RNA (gRNA) having a targeting domain that is complementary to at least one target site within the B2M gene. In some embodiments, the CRISPR-Cas combination is a ribonucleoprotein (RNP) complex comprising the gRNA and a Cas protein. In some embodiments, the method further comprises reducing or eliminating the expression of CIITA in the cell. In some embodiments, the method comprises introducing a modification that reduces or eliminates the expression of CITA. In some embodiments, the modification that reduces or eliminates CIITA expression comprises inactivation or disruption of both alleles of the CIITA gene. In some embodiments, the modification that reduces or eliminates CIITA comprises inactivation or disruption of all CIITA coding sequences in the cell. In some embodiments, the inactivation or disruption comprises an indel in the CIITA gene or a deletion of a contiguous stretch of genomic DNA of the CIITA gene. In some embodiments, the indel is a frameshift mutation. In some embodiments, the CIITA gene is knocked out. In some embodiments, the modification that increases expression of CD47 comprises an exogenous polynucleotide encoding the CD47 protein that is linked to a promoter. In some embodiments, the exogenous polynucleotide encoding CD47 is integrated into the genome of the engineered primary cell. In some embodiments, the integration is by is by targeted insertion into a target genomic locus of the cell, optionally wherein the targeted insertion is by nuclease-mediated gene editing with homology-directed repair. In some embodiments, the modification that reduces one or more MHC class I molecules protein expression and/or one or more MHC class II molecules protein expression is by nuclease-mediated gene editing. In some embodiments, the nuclease-mediated gene editing is by a zinc finger nuclease (ZFN), a TAL-effector nuclease (TALEN), or a CRISPR-Cas combination that targets the target genomic locus, optionally wherein the Cas is Cas9. In some embodiments, the modification(s) that increase expression comprise increased surface expression, and/or the modifications that reduce expression comprise reduced surface expression. In some embodiments, the engineered primary cell is a hypo-immunogenic primary cell. In some embodiments, the engineered primary cell is selected from an islet cell, beta islet cell, B cell, T cell, NK cell, retinal pigmented epithelium cell, glial progenitor cell, endothelial cell, hepatocyte, thyroid cell, skin cell, and blood cell (e.g., plasma cell or platelet). In some embodiments, the engineered primary cell is selected from a T cell and a NK cell, and further comprisies a chimeric antigen receptor (CAR). In some embodiments, the engineered primary cell is ABO blood group type O. In some embodiments, the engineered primary cell is is Rhesus factor negative (Rh−).

In some aspects, provided herein is a composition comprising a population of engineered primary cells, such as any of the engineered primary cells described herein. In some embodiments, there is provided a composition comprising a population of engineered primary islet cells, wherein the engineered primary islet cells comprise: (i) a transgene comprising an exogenous polynucleotide encoding CD47 and (ii) inactivation or disruption of both alleles of a B2M gene. In some embodiments, there is provided a composition comprising a population of engineered primary beta islet cells, wherein the engineered primary beta islet cells comprise: (i) a transgene comprising an exogenous polynucleotide encoding CD47 and (ii) inactivation or disruption of both alleles of a B2M gene. In some embodiments, there is provided a composition comprising a population of engineered primary T cells, wherein the engineered primary T cells comprise: (i) a transgene comprising an exogenous polynucleotide encoding CD47 and (ii) inactivation or disruption of both alleles of a B2M gene. In some embodiments, there is provided a composition comprising a population of engineered primary thyroid cells, wherein the engineered primary thyroid cells comprise: (i) a transgene comprising an exogenous polynucleotide encoding CD47 and (ii) inactivation or disruption of both alleles of a B2M gene. In some embodiments, there is provided a composition comprising a population of engineered primary skin cells, wherein the engineered primary skin cells comprise: (i) a transgene comprising an exogenous polynucleotide encoding CD47 and (ii) inactivation or disruption of both alleles of a B2M gene. In some embodiments, there is provided a composition comprising a population of engineered primary endothelial cells, wherein the engineered primary endothelial cells comprise: (i) a transgene comprising an exogenous polynucleotide encoding CD47 and (ii) inactivation or disruption of both alleles of a B2M gene. In some embodiments, there is provided a composition comprising a population of engineered primary retinal pigmented epithelium cells, wherein the engineered primary retinal pigmented epithelium cells comprise: (i) a transgene comprising an exogenous polynucleotide encoding CD47 and (ii) inactivation or disruption of both alleles of a B2M gene. In any of the preceding embodiments of the engineered primary cells, the cells may further comprise inactivation or disruption of both alleles of a CIITA gene.

### E. Assays for Hypoimmunogenic Phenotypes

In some embodiments, the engineered primary cells provided herein can be assessed or assessed for hypoimmunogenecity. In some embodiments, hypoimmunogenecity of a cell can be determined by evaluating the immunogenicity of the cell such as the cell's ability to elicit adaptive and innate immune responses. Such immune response can be measured using assays recognized by those skilled in the art. In some embodiments, an immune response assay measures the effect of a hypoimmunogenic cell on T cell proliferation, T cell activation, T cell killing, NK cell proliferation, NK cell activation, and macrophage activity. In some cases, hypoimmunogenic cells and derivatives thereof undergo decreased killing by T cells and/or NK cells upon administration to a subject. In some cases, hypoimmunogenic cells and derivatives thereof undergo decreased killing by macrophages upon administration to a subject. In some cases, hypoimmunogenic cells and derivatives thereof undergo decreased killing by peripheral blood mononuclear cells (PBMCs) upon administration to a subject. In some instances, the cells and derivatives thereof show decreased macrophage engulfment compared to an unmodified or wildtype cell. In some embodiments, a hypoimmunogenic cell elicits a reduced or diminished immune response in a recipient subject compared to a corresponding unmodified wild-type cell. In some embodiments, a hypoimmunogenic cell is nonimmunogenic or fails to elicit an immune response in a recipient subject.

Once the hypoimmunogenic cells have been generated, they may be assayed for their hypoimmunogenicity, engraftment, and function, as is described in WO2016183041 and WO2018132783.

The hypoimmunogenic cells are administered in a manner that permits them to engraft to the intended tissue site and reconstitute or regenerate the functionally deficient area. In some embodiments, the hypoimmunogenic cells are assayed for engraftment (e.g., successful engraftment). In some embodiments, the engraftment of the hypoimmunogenic cells is evaluated after a pre-selected amount of time. In some embodiments, the engrafted cells are monitored for cell survival. For example, the cell survival may be monitored via bioluminescence imaging (BLI), wherein the cells are transduced with a luciferase expression construct for monitoring cell survival. In some embodiments, the engrafted cells are visualized by immunostaining and imaging methods known in the art. In some embodiments, the engrafted cells express known biomarkers that may be detected to determine successful engraftment. For example, flow cytometry may be used to determine the surface expression of particular biomarkers. In some embodiments, the hypoimmunogenic cells are engrafted to the intended tissue site as expected (e.g., successful engraftment of the hypoimmunogenic cells). In some embodiments, the hypoimmunogenic cells are engrafted to the intended tissue site as needed, such as at a site of cellular deficiency. In some embodiments, the hypoimmunogenic cells are engrafted to the intended tissue site in the same manner as a non-engineered primary cell (e.g., a primary cell not comprising modification(s)) would be engrafted to the intended tissue site. In some embodiments, the hypoimmunogenic cells are assayed for function. In some embodiments, the hypoimmunogenic cells are assayed for function prior to their engraftment to the intended tissue site. In some embodiments, the hypoimmunogenic cells are assayed for function following engraftment to the intended tissue site. In some embodiments, the function of the hypoimmunogenic cells is evaluated after a pre-selected amount. In some embodiments, the function of the engrafted cells is evaluated by the ability of the cells to produce a detectable phenotype. For example, engrafted islet cells and/or beta islet cells function may be evaluated based on the restoration of lost glucose control due to diabetes. In some embodiments, the function of the hypoimmunogenic cells is as expected (e.g., successful function of the hypoimmunogenic cells while avoiding antibody-mediated rejection). In some embodiments, the function of the hypoimmunogenic cells is as needed, such as sufficient function at a site of cellular deficiency while avoiding antibody-mediated rejection. In some embodiments, the hypoimmunogenic cells function in the same manner as a non-engineered primary cell (e.g., a primary cell not comprising modification(s)) would function, while avoiding antibody-mediated rejection.

In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic cell growth (e.g. teratomas) that escape the host immune system. In some instances, hypoimmunogenic cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by ELISPOT, ELISA, FACS, PCR, or mass cytometry (CYTOF). B cell responses or antibody responses are assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of hypoimmunogenic cells is determined using an allogeneic humanized immunodeficient mouse model. In some instances, the hypoimmunogenic cells are transplanted into an allogeneic humanized NSG-SGM3 mouse and assayed for cell rejection, cell survival, and teratoma formation. In some instances, grafted hypoimmunogenic cells display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116(21), 10441-10446, the disclosures including the figures, figure legends, and description of methods are incorporated herein by reference in their entirety.

As will be appreciated by those in the art, the successful reduction of the one or more MHC class I molecules function (HLA I when the cells are derived from human cells) in the pluripotent cells can be measured using techniques known in the art and as described below; for example, FACS techniques using labeled antibodies that bind the HLA complex; for example, using commercially available HLA-A, B, C antibodies that bind to the alpha chain of the human major histocompatibility HLA Class I antigens.

In addition, the cells can be tested to confirm that the HLA I complex is not expressed on the cell surface. This may be assayed by FACS analysis using antibodies to one or more HLA cell surface components as discussed above.

The successful reduction of the one or more MHC class II molecules function (HLA II when the cells are derived from human cells) in the pluripotent cells or their derivatives can be measured using techniques known in the art such as Western blotting using antibodies to the protein, FACS techniques, RT-PCR techniques, etc.

In addition, the cells can be tested to confirm that the HLA II complex is not expressed on the cell surface. Again, this assay is done as is known in the art (See FIG. 21 of WO2018132783, for example) and generally is done using either Western Blots or FACS analysis based on commercial antibodies that bind to human HLA Class II molecules HLA-DR, DP and most DQ antigens.

In addition to the reduction of HLA I and II (or MHC class I molecules and class II molecules), the hypoimmunogenic cells provided herein have a reduced susceptibility to macrophage phagocytosis and NK cell killing. The resulting hypoimmunogenic cells “escape” the immune macrophage and innate pathways due to the expression of one or more CD24 transgenes.

### F. Methods for Generating Engineered Primary Cells

Provided herein are methods for modifying a cell by modifying expression of a gene in the cell by gene editing. In some embodiments, the methods include a step of incubating the cells with motion in connection with modifying the cells. In some embodiments, contacting cells with one or more reagents for modifying gene expression in cells followed by subjecting the cells to motion (e.g. shaking or undulating motion) can enhance or promote the efficiency of the modification of the cells. In some embodiments, the methods can be used to promote or enhance modification of cells. In some embodiments, the methods may reduce expression of a gene, such as by genetic disruption that inactivates or deletes an endogenous gene, and/or can be used to increase expression of a gene, such as overexpressing a gene in a cell. In some aspects, the methods can be used for reducing expression of one or more MHC class I molecules and/or one or more MHC class II molecules as described herein. In some aspects, the methods can be used for increasing expression of one or more tolerogenic factors, such as CD47, as described herein.

In some embodiments, provided herein is a method of modification of a population of cells, in which the method includes: i) contacting a population of cells with one or more reagents to modify gene expression in cells of the population; and ii) subjecting the population of cells to motion after contact with the one or more reagents, compared to a similar method in which the cells are not subjected to motion, e.g. in which the cells are incubated under static conditions. In some embodiments, the method enhances or promotes the modification of cells in the population. In some embodiments, the method increases viability of cells in the population, compared to a similar method in which the cells are not subjected to motion, e.g. in which the cells are incubated under static conditions.

In some embodiments, the cells to be engineered may be a cell as described herein, such as described in Section II. C.

In some embodiments, the population of cells are primary cells. In some embodiments, the population of cells are primary cells selected from the group consisting of islet cells, immune cells, B cells, T cells, natural killer (NK) cells, natural killer T (NKT) cells, macrophages, endothelial cells, muscle cells, cardiac muscle cells, smooth muscle cells, skeletal muscle cells, hepatocytes, a glial progenitor cells, dopaminergic neurons, retinal pigment epithelial cells, thyroid cells, skin cells, glial progenitor cells, neural cells, cardiac cells, and blood cells.

In some embodiments, the population of cells are cells derived from stem cells. In some embodiments, the stem cells are selected from the group consisting of a pluripotent stem cell (PSC), an induced pluripotent stem cell, an embryonic stem cell, a hematopoietic stem cell, a mesenchymal stem cell, an endothelial stem cell, an epithelial stem cell, an adipose stem cell, a germline stem cell, a lung stem cell, a cord blood stem cell, and a multipotent stem cell. In some embodiments, the stem cells are pluripotent stem cells. In some embodiments, the stem cells are induced pluripotent stem cells, mesenchymal stem cells (MSCs), hematopoietic stem cells (HSCs), or embryonic stem cells (ESCs). In some embodiments, the population of cells are cell differentiated from stem cells, or progenitors thereof, in which the differentiated cells are islet cells, immune cells, B cells, T cells, natural killer (NK) cells, natural killer T (NKT) cells, macrophages, endothelial cells, muscle cells, cardiac muscle cells, smooth muscle cells, skeletal muscle cells, hepatocytes, a glial progenitor cells, dopaminergic neurons, retinal pigment epithelial cells, thyroid cells, skin cells, glial progenitor cells, neural cells, cardiac cells, or blood cells.

In some embodiments, many existing gene editing and cell engineering methods are performed entirely in static culture, which, in some aspects, is believed to reduce further stress on the cells. However, observations indicate that such methods may not always be ideal. In some aspects, incubating or culturing the cells in a manner that more closely matches how the cells are present in vivo can improve efficiency of modification of the cells, such as by gene editing methods. For instance, an improvement in cell viability and/or efficiency of modification or gene editing can be achieved by culturing or incubation the cells in suspension (e.g. non-adherent) conditions) while subjecting the cells to motion. In some embodiments, contacting the cells to motion can produce cell aggregates that provide for cell-to-cell contact and formation of 3-D clusters that enhances the viability and fitness of cells and an overall increase in efficiency of genetic modification of the cells.

In some embodiments, the population of cells are primary cells that are naturally present in a 3D network in vivo. In some embodiments, the provided methods are carried out so that the population of cells are in suspension. In some embodiments, if the cells are naturally present in a culture or aggregate, a suspension of cells can be produced by dissociating cells from an adherent culture or a cell cluster prior to the contacting.

In some embodiments, the population of cells are in a vessel that has a low-attachment surface. In some embodiments, the population of cells are in a non-adherent culture vessel. In some embodiments, a vessel, such as one with a low-attachment surface (e.g. non-adherent culture vessel) includes a culture vessel to which cell attachment is reduced or limited, such as for a period of time. A non-adherent culture vessel may contain a low attachment or ultra-low attachment surface, such as may be accomplished by treating the surface with a substance to prevent cell attachment, such as a hydrogel (e.g. a neutrally charged and/or hydrophilic hydrogel) and/or a surfactant (e.g. pluronic acid). A non-adherent culture vessel may contain rounded or concave wells, and/or microwells (e.g. Aggrewells™). In some embodiments, a non-adherent culture vessel is an Aggrewell™ plate. For non-adherent culture vessels, use of an enzyme to remove cells from the culture vessel may not be required.

In some embodiments, a non-adherent culture vessel is a culture vessel with a low or ultra-low attachment surface, such as to inhibit or reduce cell attachment. In some embodiments, culturing cells in a non-adherent culture vessel does not prevent all cells of the culture from attaching the surface of the culture vessel.

In some embodiments, a non-adherent culture vessel is a culture vessel with an ultra-low attachment surface. In some aspects, an ultra-low attachment surface may inhibit cell attachment for a period of time. In some embodiments, an ultra-low attachment surface may inhibit cell attachment for the period of time necessary to obtain confluent growth of the same cell type on an adherent surface. In some embodiments, the ultra-low attachment surface is coated or treated with a substance to prevent cell attachment, such as a hydrogel layer (e.g., a neutrally charged and/or hydrophilic hydrogel layer). In some embodiments, a non-adherent culture vessel is coated or treated with a surfactant prior to the first incubation. In some embodiments, the surfactant is pluronic acid.

In some embodiments, the vessel is a plate, a dish, a flask, a bioreactor or a bag. In some embodiments, the vessel is a plate, such as a multi-well plate. In some embodiments, the vessel is a 6-well, a 24-well plate, a 48-well plate or a 96-well plate. In some embodiments, the culture vessel is a 6-well plate. In some embodiments, the wells of the multi-well plate further include micro-wells. In some any of the provided embodiments, a vessel, such as a multi-well plate, has round or concave wells and/or microwells. In any of the provided embodiments, a vessel, such as a multi-well plate, does not have corners or seams.

In some embodiments, a vessel allows for three-dimensional formation of cell aggregates. In some embodiments, cells are cultured in a vessel, such as a multi-well plate, and subjected to motion to produce cell aggregates or clusters. In some embodiments, subjecting the cells to motion promotes the formation of aggregates. In some embodiments, subjecting the cells to motion forms cell clusters.

In some embodiments, the population of cells are cultured under conditions to maintain their viability. It is within the level of a skilled artisan to choose appropriate temperature, C02 and oxygen conditions in order to provide the necessary environment for cell culture and viability. In some embodiments, the volume of the media can be minimized or reduced to lessen the diffusive barrier to oxygen delivery to cells. In some embodiments, the population of cells are maintained in a vessel in a minimum volume of media sufficient to cover the cells. It is within the level of a skilled artisan to determine the appropriate volume of media to support cell culture and viability. As an example, a standard working volume for a 6-well plate is 3.0 mL to 5.0 mL, however volume can be reduced to between at or about 1 mL and 2 mL to sufficiently cover the cells and provide for an appropriate culture to support growth of cells. In some cases, using more medium may increase the medium depth and static nature of the environment, and can slow diffusion of oxygen to cells—which may not be ideal.

In some embodiments, the methods can further include at least a period or portion of culturing that is carried out under static conditions. In such embodiments, the cells are not subjected to motion but are kept imobile or fixed. In some embodiments, the cells can be incubated under static conditions during the contacting with the one or more reagents for modification or gene editing and before the cells are subjected to motion. In some embodiments, the cells can be incubated under static conditions after subjecting the cells to motion.

In some embodiments, the one or more reagents can include at least two different reagents. In some embodiments, each of the at least two different reagents is for modulating expression of a different gene. In some cases, at least a first one or more reagents may be reagents for reducing expression of one or more MHC class I molecules and/or MHC class II molecules, such as described, and at least a second one or more reagents may be reagents for increasing expression of one or more tolerogenic factors, e.g. CD47. In some embodiments, the steps of the method can be repeated. In some embodiments, one or more reagents in the first iteration of the method are different from the one or more reagents in the repeated iteration of the method.

In some embodiments, the methods provided herein also can include selecting cells that have the desired modifications, such as gene edits. In some embodiments, methods of selecting cells with modifications can be by flow cytometry, such as by positive or negative selection of desired cells.

In some aspects, provided herein is a method for gene editing primary islet cells. The method of gene editing primary islet cells comprises dissociating a primary islet cluster into a suspension of primary beta islet cells, wherein the primary islet cluster comprises primary beta islet cells. Primary beta islet cells of the suspension are then modified (e.g., by introducing one or more modifications into the cell to reduce expression of one or more genes encoding an endogenous protein in the cell and/or introducing one or more modifications to increase expression of a heterologous proteins in the cell). The modified primary beta islet cells may be incubated under conditions for re-clustering the modified primary beta islet cells into an islet, wherein at least a portion of the incubating is carried out with motion.

The term “motion” as used herein refers to moving a cell (e.g., a modified primary beta islet cell). For example, a cell may be moved in a circular motion, moved side-to-side, moved up and down, and/or inverted. In some embodiments, the motion is shaking. In some embodiments, the shaking comprises an orbital shaking. In some embodiments, the shaking comprises bidirectional linear movement. In some embodiments, the shaking comprisies linear movement. The motion may be accomplished by various methods known in the art, such as but not limited to, an orbital shaker, a reciprocating shaker, a gyratory rocker, a microplate shaker, a benchtop shaker, and/or a vortex. In some embodiments, the motion is accomplished using an orbital shaker. In some embodiments, the orbital shaker is a Belly Dancer Orbital Shaker (IBI Scientific). In some embodiments, the motion improves gene editing efficiency compared to a method that does not employ motion in a method of generating an engineered primary cell. For example, the motion may result in better gene targeting, improved expression and/or improved decrease in gene expression, and/or an increased number of cells targeted by the method. In some embodiments, the motion improves the gene editing efficiency in a method of generating an engineered primary cell greater than about 0.1-fold, such as greater than any of about 0.2-fold, 0.3-fold, 0.4-fold, 0.5-fold, 1-fold, 2-fold, 3-fold, 4-fold, 5-fold, 10-fold, 20-fold, 50-fold, 100-fold, or more, compared to a method of generating an engineered primary cell that does not employ motion. In some embodiments, the motion improves the gene editing efficiency in a method of generating an engineered primary cell between about 0.1-fold and about 100-fold, such as between about 0.1-fold and about 10-fold, about 0.5-fold and about 50-fold, and about 10-fold and about 100-fold.

In some embodiments, by subjecting the cells to motion, the amount of time necessary to contact the cells with one more reagents for gene editing can be reduced. In some embodiments, the contacting with one or more reagents for modification or gene editing is for less than two days prior to subjecting the cells to motion. In some embodiments, the contacting is carried out for 30 second to 24 hours prior to subjecting the cells to motion. In some embodiments, the contacting is carried out for at or about 1 minute, 5 minutes, 10 minutes, 20 minutes, 30 minutes, 40 minutes, 50 minutes, 1 hour, 2 hours, 4 hours, 6 hours, 12 hours, 24 hours, or any value between any of the foregoing. In some embodiments, the contacting is carried out for 1 minute to 60 minuties prior to subjecting the cells to motion. In some embodiments, the contacting is carried out for 2 minutes to 30 minutes prior to subjecting the cells to motion. In some embodiments, the contacting is carried out for 5 minutes to 15 minutes prior to subjecting the cells to motion.

In some embodiments, provided herein is a method for gene editing primary islet cells, the method comprising: i) dissociating a primary islet cluster into a suspension of primary beta islet cells; ii) modifying primary beta islet cells of the suspension; and iii) incubating the modified primary beta islet cells under conditions for reclustering the modified primary beta islet cells into an islet, wherein at least a portion of the incubating is carried out with motion. In some embodiments, the primary islet cluster is a human primary islet cluster. In some embodiments, the primary islet cluster is a human primary cadaveric islet cluster. In some embodiments, the primary cell is a primary beta islet cell. In some embodiments, the suspension is a single cell suspension.

The methods for gene editing primary islet cells provided herein may comprise one or more steps of dissociating (e.g., dissociating a primary islet cluster into a suspension of primary cells) and one or more steps of reclustering (e.g., incubating modified primary cells under conditions for reclustering the primary cells into an islet). In some embodiments, the method of gene editing primary islet cells comprises about one, two, three, four, five, or more, steps of dissociating. In some embodiments, the method of gene editing primary islet cells comprises about one, two, three, four, five, or more, steps of reclusting. In some embodiments, each of the one or more steps of reclustering is performed after each of the one or more steps of dissociating. In some embodiments, the method of gene editing primary islet cells comprises a period of time between performing a step of dissociating and a step of reclustering. In some embodiments, the period of time between performing the step of dissociating and the step of reclustering is between about 1 min and about 10 days, such as between about 1 minutes (min) and about 10 hour (h), between about 5 h and about 24 h, and between about 24 h and about 10 days. In some embodiments, the period of time between performing the step of dissociating and the step of reclustering is greater than about 1 min, such as greater than any of about 5 min, 10 min, 30 min, 1 h, 2 h, 3 h, 4 h, 5 h, 10 h, 24 h, 48 h, 5 days, 10 days, or greater. In some embodiments, the period of time between performing the step of dissociating and the step of reclustering is less than about 10 days, such as less than any of about 5 days, 48 h, 24 h, 10 h, 5 h, 4 h, 3 h, 2 h, 1 h, 30 min, 10 min, 5 min, or less.

In some embodiments, incubating the modified primary beta islet cells under conditions for re-clustering the modified primary beta islet cells into an islet is performed at least once. In some embodiments, incubating the modified primary beta islet cells under conditions for re-clustering the modified primary beta islet cells into an islet is performed intermittently, wherein each incubating step is performed after modifying primary beta islet cells of the suspension. In some embodiments, incubating the modified primary beta islet cells under conditions for re-clustering the modified primary beta islet cells into an islet is performed twice, wherein a first incubating step is performed after modifying primary beta islet cells of the suspension to reduce expression of one or more genes encoding an endogenous protein in the cell, and wherein a second incubating step is performed after modifying primary beta islet cells of the suspension to increase expression of one or more heterologous proteins in the cell. In some embodiments, incubating the modified primary beta islet cells under conditions for re-clustering the modified primary beta islet cells into an islet is performed twice, wherein a first incubating step is performed after modifying primary beta islet cells of the suspension to reduce expression of a human B2M gene and a human CIITA gene in the cell, and wherein a second incubating step is performed after modifying primary beta islet cells of the suspension to increase expression of CD47 in the cell.

In some embodiments, dissociating a primary islet cluster into a suspension of primary beta islet cells is by a cell dissociation solution. In some embodiments, the cell dissociation solution is applied to the primary islet cluster. In some embodiments, the cell dissociation solution is applied to the primary islet cluster for between about 1 minute (min) and about 20 mins, such as between about 1 min and about 5 min, about 3 min and about 10 min, about 8 min and about 15 min, and about 12 min and about 20 min. In some embodiments, the cell dissociation solution is applied to the primary islet cluster for greater than about 1 min, such as greater than any of about 2 min, 3 min, 4 min, 5 min, 6 min, 7 min, 8 min, 9 min, 10 min, 15 min, 20 min, or greater. In some embodiments, the cell dissociation solution is applied to the primary islet cluster for less than about 20 min, such as less than any of about 15 min, 10 min, 9 min, 8 min, 7 min, 6 min, 5 min, 4 min, 3 min, 2 min, 1 min, or less. In some embodiments, the cell dissociation solution is applied to the primary islet cluster for about 10 min. In some embodiments, the cell dissociation solution is applied to the primary islet cluster at a temperature of between about 30° C. and about 40° C., such as between about 30° C. and about 35° C., about 33° C. and about 38° C., and about 35° C. and about 40° C. In some embodiments, the cell dissociation solution is applied to the primary islet cluster at a temperature of greater than about 30° C., such as greater than any of about 31° C., 32° C., 33° C., 34° C., 35° C., 36° C., 37° C., 38° C., 39° C., 40° C., or greater. In some embodiments, the cell dissociation solution is applied to the primary islet cluster at a temperature of less than about 40° C., such as less than any of about 39° C., 38° C., 37° C., 36° C., 35° C., 34° C., 33° C., 32° C., 31° C., 30° C., or less. In some embodiments, the cell dissociation solution is applied to the primary islet cluster at a temperature of about 37° C. In some embodiments, the cell dissociation solution is applied to the primary islet cluster at a temperature of about 37° C. for about 10 min. A cell dissociation solution may comprise a solution of proteolytic and collagenolytic enzymes. In some embodiments, the cell dissociation solution is an ACCUMAX™ cell detachment solution.

In some embodiments, a dissociated suspension of primary beta islet cells is modified. In some embodiments, the modifying comprises genetic engineering. In some embodiments, the suspension primary beta islet cells is modified following dissociation from a primary islet cluster. In some embodiments, the modifying comprises introducing one more modifications into the cell to reduce expression of one or more genes encoding an endogenous protein in the cell. In some embodiments, the modifying comprises introducing one or more modifications into the cell to reduce expression of one or more MHC class I molecules. In some embodiments, the modifying comprises introducing one or more modifications into the cell to reduce expression of one or more MHC class II molecules. In some embodiments, the modifying comprises introducing one or more modifications into the cell to reduce expression of one or more MHC class I molecules and one or more MHC class II molecules. In some embodiments, the modification that reduces expression of one or more MHC class I molecules is a modification that reduces expression of B2M. In some embodiments, the modification that reduces expression of one or more MHC class II molecules is a modification that reduces expression of CIITA.

In some embodiments, reducing expression of one or more genes encoding an endogenous protein in the cell is by introducing a gene-editing system into the cell. In some embodiments, the gene-editing system comprises a sequence specific nuclease. In some embodiments, the gene-editing system comprises an RNA-guided nuclease. In some embodiments, the sequence specific nuclease is selected from the group consisting of a RNA-guided DNA endonuclease, a meganuclease, a transcription activator-like effector nuclease (TALEN), and a zinc-finger nuclease (ZFN). In some embodiments, the RNA-guided nuclease comprises a Cas nuclease and a guide RNA. In some embodiments, the RNA-guided-nuclease is a Type II or Type V Cas protein. In some embodiments, the RNA-guided-nuclease is a Cas9 homologue or a Cpf1 homologue.

In some embodiments, the RNA-guided nuclease comprises a Cas9 nuclease and a single gRNA targeting the human B2M gene. In some embodiments, the single gRNA targeting the human B2M gene comprises the nucleic acid sequence of CGUGAGUAAACCUGAAUCUU (SEQ ID NO: 33). In some embodiments, the RNA-guided nuclease comprises a Cas9 nuclease and a single gRNA targeting the human CIITA gene. In some embodiments, the single gRNA targeting the human CIITA gene comprises the nucleic acid sequence of CGAUAUUGGCAUAAGCCUCCC (SEQ ID NO: 34). In some embodiments, the single gRNA targeting the human B2M gene is introduced to the cell before the single gRNA targeting the human CIITA gene. In some embodiments, the single gRNA targeting the human CIITA gene is introduced to the cell before the single gRNA targeting the human B2M gene. In some embodiments, the single gRNA targeting the human B2M gene is introduced to the cell concurrently with the single gRNA targeting the human CIITA gene. In some embodiments, the one or more modifications to reduce expression of one or more genes encoding an endogenous protein in the cell are introduced into the cell by electroporation. In some embodiments, the cell is electroporated with a ribonucleoprotein complex containing a Cas9 enzyme and a single gRNA targeting the human B2M gene. In some embodiments, the cell is electroporated with a ribonucleoprotein complex containing a Cas9 enzyme and a single gRNA targeting the human CIITA gene.

In some embodiments, the modifying comprises introducing one more modifications into the cell to increase expression of one or more heterologous proteins in the cell. In some embodiments, the modifying comprises introducing one or more modifications to increase expression of one or more tolerogenic factor. In some embodiments, the one or more tolerogenic factor is selected from the group consisting of CD47, CD27, CD200, HLA-C, HLA-E, HLA-E heavy chain, HLA-G, PD-L1, IDO1, CTLA4-Ig, C1-Inhibitor, IL-10, IL-35, FASL, CCL21, MFGE8, and SERPINB9, and any combination thereof. In some embodiments, the one or more tolerogenic factor is selected from the group consisting of CD47, PD-L1, HLA-E, HLA-G, CCL21, FASL, SERPINB9, CD200, MFGE8, and any combination thereof. In some embodiments, at least one of the one or more tolerogenic factor is CD47.

In some embodiments, increasing expression of one or more heterologous proteins in the cell is by an exogenous polynucleotide. In some embodiments, the exogenous polynucleotide is operably linked to a promoter. In some embodiments, the promoter is a constitutive promoter. In some embodiments, the promoter is selected from the group consisting of the CAG promoter, the cytomegalovirus (CMV) promoter, the EF1a promoter, the PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, and the Rous sarcoma virus (RSV) promoter. In some embodiments, the exogenous polynucleotide is integrated into the genome of the cell. In some embodiments, the exogenous polynucleotide is a multicistronic vector. In some embodiments, the integration is by non-targeted insertion into the genome of the cell, optionally by introduction of the exogenous polynucleotide into the cell using a lentiviral vector. In some embodiments, the integration is by targeted insertion into a target genomic locus of the cell.

In some embodiments, increasing expression of one or more heterologous proteins in the cell is by transducing the cells with a lentiviral vector encoding CD47 and luciferase under control of the CAG promoter. In some embodiments, the cells are transduced with the lentiviral vector in the presence of protamine sulfate. In some embodiments, transduction with the lentiviral vector is performed using centrifugation (e.g., “spinfection”). In some embodiments, the cells are centrifuged in the presence of the lentiviral vector in the presence of protamine sulfate at about 300×g for about 15 min.

In some embodiments, a method for gene editing primary cells provided herein further comprises selecting for a modified islet. In some embodiments, the selected modified islet has been modified to reduce expression of one or more genes encoding an endogenous protein in the cell (e.g., a human B2M gene and/or a human CIITA gene). In some embodiments, the selected modified islet has been modified to increase expression of one or more heterologous proteins in the cell (e.g., CD47). In some embodiments, the selected modified islet has been modified to reduce expression of one or more genes encoding an endogenous protein in the cell and to increase expression of one or more heterologous proteins in the cell. In some embodiments, the selecting comprises fluorescence-activated cell sorting (FACS). In some embodiments, the FACS comprises use of a BD FACSAria™ III cell sorter.

In some embodiments, the modified islets are dissociated into single primary islet cells for FACS using a cell dissociation solution, such as any of the cell dissociation solutions described herein. In some embodiments, the cell dissociation solution is an ACCUMAX™ cell detachment solution. In some embodiments, the primary beta islet cell has been modified to reduce expression of one or more genes encoding an endogenous protein in the cell is selected for using a cell dissociation solution with an anti-HLA-A,B,C antibody or an IgG1 isotype-matched control antibody, and an anti-HLA-DR,DP,DQ antibody or an IgG2a isotype-matched control antibody. In some embodiments, the double negative primary islet cells are sorted using FACS. In some embodiments, the sorted, double negative primary islet cells are re-plated for re-clustering using incubation. In some embodiments, the primary beta islet cell has been modified to reduce expression of one or more genes encoding an endogenous protein (e.g., human B2M gene and human CIITA gene) in the cell and to increase expression of one or more heterologous proteins in the cell is selected for using a cell dissociation solution with an anti-CD47 antibody or an IgG1 isotype-matched control antibody. In some embodiments, the modified islet cells are selected if they have at least about a 20-fold increase in expression, such at least about 21-fold, 22-fold, 23-fold, 24-fold, 25-fold, or greater increase in expression of one or more heterologous proteins in the cell compared (e.g., CD47) compared to isotype control. In some embodiments, the sorted, double negative primary islet cells that are positive for CD47 are re-plated for re-clustering using incubation.

In some embodiments, the modified primary beta islet cells are incubated under conditions for re-clustering the modified primary beta islet cells into an islet. In some embodiments, the incubating is performed following the modifying of the primary beta islet cells. In some embodiments, the incubating comprises incubating the modified primary beta islet cells in human islet cell culture media. In some embodiments, the human islet cell culture media is PIM(S) media (Prodo). In some embodiments, the incubating comprises a first incubation under static conditions followed by the incubating with motion. In some embodiments, the incubating comprises the incubating with motion followed by a second incubation under static conditions. In some embodiments, the incubating comprises the incubating with motion and incubation under static conditions.

In some embodiments, the modified primary beta islet cells are statically incubated for between about 30 min and about 2 hours (h), such as between about 30 min and about 1 h, about 45 min and about 1.5 h, and about 1 h and about 2 h. In some embodiments, the modified primary beta islet cells are statically incubated for greater than about 30 min, such as greater than any of about 35 min, 40 min, 45 min, 50 min, 55 min, 1 h, 1.25 h, 1.5 h, 1.75 h, 2 h, or greater. In some embodiments, the modified primary beta islet cells are statically incubated for less than about 2 h, such as less than any of about 1.75 h, 1.5 h, 1.25 h, 1 h, 55 min, 50 min, 45 min, 40 min, 35 min, 30 min, or less. In some embodiments, the modified primary beta islet cells are statically incubated for about 1 h. In some embodiments, the modified primary beta islet cells are statically incubated at a temperature of between about 30° C. and about 40° C., such as between about 30° C. and about 35° C., about 33° C. and about 38° C., and about 35° C. and about 40° C. In some embodiments, the modified primary beta islet cells are statically incubated at a temperature of greater than about 30° C., such as greater than any of about 31° C., 32° C., 33° C., 34° C., 35° C., 36° C., 37° C., 38° C., 39° C., 40° C., or greater. In some embodiments, the modified primary beta islet cells are statically incubated at a temperature of less than about 40° C., such as less than any of about 39° C., 38° C., 37° C., 36° C., 35° C., 34° C., 33° C., 32° C., 31° C., 30° C., or less. In some embodiments, the modified primary beta islet cells are statically incubated at a temperature of about 37° C. for about 1 h. In some embodiments, the static incubation is performed in between about 2% and about 8% CO₂, such as between about 2% and about 4% CO₂, about 3% and about 6% CO₂, and about 5% and about 8% CO₂. In some embodiments, the static incubation is performed in greater than about 2% CO₂, such as greater than any of about 3% CO₂, 4% CO₂, 5% CO₂, 6% CO₂, 7% CO₂, 8% CO₂, or greater. In some embodiments, the static incubation is performed in less than about 8% CO₂, such as less than any of about 7% CO₂, 6% CO₂, 5% CO₂, 4% CO₂, 3% CO₂, 2% CO₂, or less. In some embodiments, the static incubation is performed in about 5% CO₂. In some embodiments, the modified primary beta islet cells are statically incubated at about 37° C. for about 1 h in about 5% CO₂.

In some embodiments, the modified primary beta islet cells are incubated with motion after static incubation (e.g., static incubation at about 37° C. for about 1 h in about 5% CO₂). In some embodiments, the motion incubation allows for re-clustering the modified primary beta islet cells into an islet. In some embodiments, the motion incubation is performed for between about 24 h and about 96 h, such as between about 24 h and about 48 h, about 48 h and about 72 h, and about 28 h and about 96 h. In some embodiments, the motion incubation is performed for greater than about 24 h, such as greater than any of about 36 h, 48 h, 60 h, 72 h, 84 h, 96 h, or greater. In some embodiments, the motion incubation is performed for less than about 96 h, such as less than any of about 84 h, 72 h, 60 h, 28 h, 36 h, 24 h, or less. In some embodiments, the motion incubation is performed for about 72 h. In some embodiments, the motion incubation is performed for about 48 h, followed by a complete media change, which is followed by an additional 24 h of motion incubation. In some embodiments, the modified primary beta islet cells are incubated with motion on a Belly Dancer Orbital Shaker (IBI Scientific, Dubuque, IA) for human primary islet cell re-clustering.

In some embodiments, the motion incubation is performed at a speed of between about 1 revolution per minute (RPM) to about 200 RPM, such as between about 1 RPM to about 25 RPM, about 15 RPM to about 50 RPM, about 30 RPM to about 75 RPM, and about 50 RPM to about 200 RPM, and about 85 RPM to about 95 RPM. In some embodiments, the motion incubation is performed at a speed of greater than about 1 RPM, such as greater than any of about 5 RPM, 10 RPM, 20 RPM, 30 RPM, 40 RPM, 50 RPM, 60 RPM, 70 RPM, 80 RPM, 90 RPM, 100 RPM, 125 RPM, 150 RPM, 175 RPM, 200 RPM, or greater. In some embodiments, the motion incubation is performed at a speed of less than about 200 RPM, such as less than any of about 175 RPM, 150 RPM, 125 RPM, 100 RPM, 90 RPM, 80 RPM, 70 RPM, 60 RPM, 50 RPM, 40 RPM, 30 RPM, 20 RPM, 10 RPM, 5 RPM, 1 RPM, or less. In some embodiments, the motion incubation is performed at a speed of between about 85 RPM and about 95 RPM. In some embodiments, the motion incubation is performed at a pitch of between about 0° and about 8°, such as between about 0° and about 4°, about 2° and about 5°, and about 4° and about 8°. In some embodiments, the motion incubation is performed at a pitch of greater than about 0°, such as greater than any of about 1°, 2°, 3°, 4°, 5°, 6°, 7°, 8°, or greater. In some embodiments, the motion incubation is performed at a pitch of less than about 8°, such as greater than any of about 7°, 6°, 5°, 4°, 3°, 2°, 1°, or less.

In some embodiments, steps i)-iii) of a method for gene editing primary islet cells provided herein are repeated. In some embodiments, the modifying in the first iteration of the method is different from the modifying in the repeated iteration of the method. In some embodiments, the modifying in the first iteration of the method comprises introducing one more modifications into the cell to reduce expression of one or more genes encoding an endogenous protein in the cell (e.g., introducing one or more modifications into the cell to reduce expression of one or more MHC class I molecules and/or one or more MHC class II molecules, e.g., one or more modifications into the cell to reduce expression of a human B2M gene and/or a human CIITA gene). In some embodiments, the modifying in the repeated iteration of the method comprises introducing one more modifications into the cell to increase expression of one or more heterologous proteins in the cell (e.g., one or more tolerogenic factor, e.g., CD47).

In some embodiments, the modifying is a first modifying in which the re-clustered islet cells are a first modified islet engineered with a first modification and wherein the method further comprises: iv) dissociating the first modified islet into a suspension of modified primary beta islet cells; v) further modifying the modified primary islet cells of the suspension with a second modification; and vi) incubating the further modified primary beta islet cells under conditions for re-clustering into a second modified islet comprising a second modification, wherein at least a portion of the incubating is carried out with motion. In some embodiments, the modifying in the first modifying comprises introducing one more modifications into the cell to reduce expression of one or more genes encoding an endogenous protein in the cell (e.g., introducing one or more modifications into the cell to reduce expression of one or more MHC class I molecules and/or one or more MHC class II molecules, e.g., introducing one or more modifications into the cell to reduce expression of a human B2M gene and/or a human CIITA gene). In some embodiments, the further modifying comprises introducing one more modifications into the cell to increase expression of one or more heterologous proteins in the cell (e.g., one or more tolerogenic factor, e.g., CD47). In some embodiments, a method for gene editing primary cells provided herein further comprises selecting for the first modified islet. In some embodiments, the selecting comprises FACS.

In some embodiments, the second modified islet is used for transplantation. In some embodiments, the second modified islet is used for treating diseases or conditions in a subject, such as any of the diseases or conditions described herein.

### III. Populations of Engineered Cells and Pharmaceutical Compositions

Provided herein are populations engineered cells, such as engineered primary cells, containing a plurality of the provided engineered cells, such as engineered primary cells. In some cases, the population of cells comprises a mixture of cells. In some cases, at least about 30% of cells in the population comprise a set of modifications described herein. In some cases, the population of cells comprises one or more different cell types.

In some embodiments, the population comprises a mixture of islet cells. In some embodiments, the population comprises a mixture of pancreatic islet cells, including two or more different cell types selected from the group consisting of pancreatic beta cells, pancreatic alpha cells, and pancreatic gamma cells. In some cases, the population comprises pancreatic alpha, beta, and gamma cells. In some cases, the population comprises primary cells. In some embodiments, the population comprises cells differentiated from stem cells or progenitor cells (e.g, cells differentiated from an induced pluripotent stem cell, an embryonic stem cell, a hematopoietic stem cell, a mesenchymal stem cell, an endothelial stem cell, an epithelial stem cell, an adipose stem cell, a germline stem cell, a lung stem cell, a cord blood stem cell, a pluripotent stem cell (PSC), and a multipotent stem cell).

In some embodiments, the population of engineered primary cells are derived from cells pooled from more than one donor subject. In some embodiments, each of the more than one donor subjects are healthy subjects or are not suspected of having a disease or condition at the time the donor sample is obtained from the donor subject. In some embodiments, at least 50%, 60%, 70%, 80%, 90%, 95%, 96%, 97%, 98%, 99%, 99.9%, or 99.99% of cells in the population comprise the modifications. In some embodiments, the population of engineered primary cells are selected from a beta islet cell, B cell, T cell, NK cell, retinal pigmented epithelium cell, glial progenitor cell, endothelial cell, hepatocyte, thyroid cell, skin cell, and blood cell (e.g., plasma cell or platelet).

In some embodiments, at least about any of 50%, 60%, 70%, 80%, 90%, 95%, 96%, 97%, 98%, 99%, 99.9%, or 99.99% of cells in the population comprise reduced expression of one or more MHC class I molecules and/or one or more MHC class II molecules relative to relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise reduced expression of B2M and/or CIITA relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise reduced expression of B2M and CIITA relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise one or more alterations that inactivate both alleles of a B2M gene relative to an unmodified or unaltered cell of the same cell type that does not comprise the one or more modifications. In some embodiments, at least about any of 50%, 60%, 70%, 80%, 90%, 95%, 96%, 97%, 98%, 99%, 99.9%, or 99.99% of cells in the population comprise one or more alterations that inactivate both alleles of a CIITA gene relative to an unmodified or unaltered cell of the same cell type that does not comprise the one or more modifications.

In some embodiments, at least 50%, 60%, 70%, 80%, 90%, 95%, 96%, 97%, 98%, 99%, 99.9%, or 99.99% of cells in the population comprise one or more alterations that inactivate both alleles of an endogenous B2M gene. In some embodiments, at least 50%, 60%, 70%, 80%, 90%, 95%, 96%, 97%, 98%, 99%, 99.9%, or 99.99% of cells in the population comprise one or more alterations that inactivate both alleles of an endogenous CIITA gene.

Also provided herein are compositions comprising the engineered cells, such as engineered primary cells. Also provided herein are compositions comprising populations of engineered cells, such as engineered primary cells. In some embodiments, the compositions are pharmaceutical compositions. In some embodiments, the pharmaceutical composition provided herein further include a pharmaceutically acceptable excipient or carrier. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g., neutral buffer saline or phosphate buffered saline). In some embodiments, the pharmaceutical composition can contain one or more excipients for modifying, maintaining or preserving, for example, the pH, osmolarity, viscosity, clarity, color, isotonicity, odor, sterility, stability, rate of dissolution or release, adsorption, or penetration of the composition. In some aspects, a skilled artisan understands that a pharmaceutical composition containing cells may differ from a pharmaceutical composition containing a protein.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The pharmaceutical composition in some embodiments contains engineered cells, such as primary cells, as described herein in amounts effective to treat or prevent the disease or condition, such as a therapeutically effective or prophylactically effective amount. In some embodiments, the pharmaceutical composition contains engineered primary cells as described herein in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, engineered primary cells as described herein are administered using standard administration techniques, formulations, and/or devices. In some embodiments, engineered primary cells as described herein aare administered using standard administration techniques, formulations, and/or devices. Provided are formulations and devices, such as syringes and vials, for storage and administration of the compositions. Engineered primary cells can be administered via localized injection, including catheter administration, systemic injection, localized injection, intravenous injection, or parenteral administration. When administering a therapeutic composition (e.g., a pharmaceutical composition containing an engineered primary cell), it will generally be formulated in a unit dosage injectable form (solution, suspension, emulsion).

Formulations include those for intravenous, intraperitoneal, or subcutaneous, administration. In some embodiments, the cell populations are administered parenterally. The term “parenteral,” as used herein, includes intravenous, intramuscular, subcutaneous, rectal, vaginal, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, or dispersions, which may in some aspects be buffered to a selected pH. Liquid compositions are somewhat more convenient to administer, especially by injection. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like.

In some embodiments, a pharmaceutically acceptable carrier can include all solvents, dispersion media, coatings, antibacterial and antifungal agents, isotonic and absorption delaying agents, and the like, compatible with pharmaceutical administration (Gennaro, 2000, Remington: The science and practice of pharmacy, Lippincott, Williams & Wilkins, Philadelphia, PA). Examples of such carriers or diluents include, but are not limited to, water, saline, Ringer's solutions, dextrose solution, and 5% human serum albumin. Liposomes and non-aqueous vehicles such as fixed oils may also be used. Supplementary active compounds can also be incorporated into the compositions. The pharmaceutical carrier should be one that is suitable for the engineered primary cells, such as a saline solution, a dextrose solution or a solution comprising human serum albumin. In some embodiments, the pharmaceutically acceptable carrier or vehicle for such compositions is any non-toxic aqueous solution in which the engineered primary cells can be maintained, or remain viable, for a time sufficient to allow administration of live cells. For example, the pharmaceutically acceptable carrier or vehicle can be a saline solution or buffered saline solution.

In some embodiments, the composition, including pharmaceutical composition, is sterile. In some embodiments, isolation, enrichment, or culturing of the cells is carried out in a closed or sterile environment, for example and for instance in a sterile culture bag, to minimize error, user handling and/or contamination. In some embodiments, sterility may be readily accomplished, e.g., by filtration through sterile filtration membranes. In some embodiments, culturing is carried out using a gas permeable culture vessel. In some embodiments, culturing is carried out using a bioreactor.

Also provided herein are compositions that are suitable for cryopreserving the provided engineered primary cells. In some embodiments, the provided engineered primary cells are cryopreserved in a cryopreservation medium. In some embodiments, the cryopreservation medium is a serum free cryopreservation medium. In some embodiments, the composition comprises a cryoprotectant. In some embodiments, the cryoprotectant is or comprises DMSO and/or s glycerol. In some embodiments, the cryopreservation medium is between at or about 5% and at or about 10% DMSO (v/v). In some embodiments, the cryopreservation medium is at or about 5% DMSO (v/v). In some embodiments, the cryopreservation medium is at or about 6% DMSO (v/v). In some embodiments, the cryopreservation medium is at or about 7% DMSO (v/v). In some embodiments, the cryopreservation medium is at or about 7.5% DMSO (v/v). In some embodiments, the cryopreservation medium is at or about 8% DMSO (v/v). In some embodiments, the cryopreservation medium is at or about 9% DMSO (v/v). In some embodiments, the cryopreservation medium is at or about 10% DMSO (v/v). In some embodiments, the cryopreservation medium contains a commercially available cryopreservation solution (CryoStor™ CS10). CryoStor™ CS10 is a cryopreservation medium containing 10% dimethyl sulfoxide (DMSO). In some embodiments, compositions formulated for cryopreservation can be stored at low temperatures, such as ultra low temperatures, for example, storage with temperature ranges from −40° C. to −150° C., such as or about 80° C.±6.0° C.

In some embodiments, the pharmaceutical composition comprises engineered primary cells described herein and a pharmaceutically acceptable carrier comprising 31.25% (v/v) Plasma-Lyte A, 31.25% (v/v) of 5% dextrose/0.45% sodium chloride, 10% dextran 40 (LMD)/5% dextrose, 20% (v/v) of 25% human serum albumin (HSA), and 7.5% (v/v) dimethylsulfoxide (DMSO).

In some embodiments, the cryopreserved engineered primary cells are prepared for administration by thawing. In some cases, the engineered primary cells can be administered to a subject immediately after thawing. In such an embodiment, the composition is ready-to-use without any further processing. In other cases, the engineered primary cells are further processed after thawing, such as by resuspension with a pharmaceutically acceptable carrier, incubation with an activating or stimulating agent, or are activated washed and resuspended in a pharmaceutically acceptable buffer prior to administration to a subject.

### IV. Kits, Components, and Articles of Manufacture

In some aspects, provided herein are kits, components, and compositions (such as consumables) of the methods, devices, and systems described herein. In some embodiments, the kit comprises instructions for use according to the disclosure herein.

In some embodiments, provided herein is a kit or composition comprising a population of engineered primary cells described herein. In some embodiments, provided herein is a kit or combination, comprising: a population of cells comprising a plurality of engineered primary cells, wherein the engineered primary cells comprise modifications that (i) increase expression of CD47, and (ii) reduce expression of one or more MHC class I molecules and/or one or more MHC class II molecules (e.g., one or more MHC class I human leukocyte antigen molecules and one or more MHC class II human leukocyte antigen molecules), wherein the increased expression of (i) and the reduced expression of (ii) is relative to a cell of the same cell type that does not comprise the modifications. In some embodiments, the components of the kit may be administered simultaneously. In some embodiments, the components of the kit may be administered sequentially.

In some embodiments of the invention, there is provided an article of manufacture containing materials useful for clinical transplantation therapies, including cell therapies. In some embodiments, the articles of manufacture contain material useful for the treatment of cellular deficiencies, such as but not limited to diabetes (e.g., Type I diabetes), vascular conditions or disease, autoimmune thyroiditis, live disease (e.g., cirrhosis of the liver), corneal disease (e.g., Fuchs dystrophy or congenital hereditary endothelial dystrophy), kidney disease, and cancer (e.g., B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer). The article of manufacture can comprise a container and a label or package insert on or associated with the container. Suitable containers include, for example, bottles, vials, syringes, etc. (e.g., glass or plastic containers) Generally, the container holds a composition which is effective for allogeneic cell therapy, and may have a sterile access port (for example the container may be an intravenous solution bag or a vial having a stopper pierceable by a hypodermic injection needle). At least component in the pharmaceutical composition is a population of engineered primary cells, such as any of the engineered primary cells provided herein. The label or package insert indicates that the composition is used for treating the particular condition. The label or package insert will further comprise instructions for administering the pharmaceutical composition to the patient. In some embodiments, the article of manufacture comprises a combination treatment.

The article of manufacture and/or kit may further comprise a package insert. The insert refers to instructions customarily included in commercial packages of therapeutic products that contain information about the indications, usage, dosage, administration, contraindications and/or warnings concerning the use of such therapeutic products.

### V. Methods of Treatment

Provided herein are compositions and methods relating to the provided cell compositions comprising a population of engineered cells described herein for use in treating diseases or conditions in a subject. Provided herein is a method of treating a patient by administering a population engineered cells described herein. In some embodiments, the engineered cells are engineered primary cells. In some embodiments, the population of cells are formulated for administration in a pharmaceutical composition, such as any described here. Such methods and uses include therapeutic methods and uses, for example, involving administration of the population of engineered cells, such as engineered primary cells, or compositions containing the same, to a subject having a disease, condition, or disorder. It is within the level of a skilled artisan to choose the appropriate engineered primary cells as provided herein for a particular disease indication. In some embodiments, the cells or pharmaceutical composition thereof is administered in an effective amount to effect treatment of the disease or disorder. Uses include uses of the engineered primary cells or pharmaceutical compositions thereof in such methods and treatments, and in the preparation of a medicament in order to carry out such therapeutic methods. In some embodiments, the methods thereby treat the disease or condition or disorder in the subject.

The engineered cells, such as engineered primary cells, provided herein can be administered to any suitable patients including, for example, a candidate for a cellular therapy for the treatment of a disease or disorder. Candidates for cellular therapy include any patient having a disease or condition that may potentially benefit from the therapeutic effects of the subject engineered primary cells provided herein. In some embodiments, the patient is an allogeneic recipient of the administered cells. In some embodiments, the provided engineered cells, such as engineered primary cells, are effective for use in allogeneic cell therapy. A candidate who benefits from the therapeutic effects of the subject engineered cells, such as engineered primary cells, provided herein exhibit an elimination, reduction or amelioration of ta disease or condition.

In some embodiments, the engineered primary cells as provided herein, including those produced by any of the methods provided herein, can be used in cell therapy. Therapeutic cells outlined herein are useful to treat a disorder such as, but not limited to, a cancer, a genetic disorder, a chronic infectious disease, an autoimmune disorder, a neurological disorder, and the like.

In some embodiments, the patient has a cellular deficiency. As used herein, a “cellular deficiency” refers to any disease or condition that causes a dysfunction or loss of a population of cells in the patient, wherein the patient is unable to naturally replace or regenerate the population of cells. Exemplary cellular deficiencies include, but are not limited to, autoimmune diseases (e.g., multiple sclerosis, myasthenia gravis, rheumatoid arthritis, diabetes, systemic lupus and erythematosus), neurodegenerative diseases (e.g., Huntington's disease and Parkinson's disease), cardiovascular conditions and diseases, vascular conditions and diseases, corneal conditions and diseases, liver conditions and diseases, thyroid conditions and diseases, and kidney conditions and diseases. In some embodiments, the patient administered the engineered primary cells has a cancer. Exemplary cancers that can be treated by the engineered primary cells provided herein include, but are not limited to, B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer. In certain embodiments, the cancer patient is treated by administration of an engineered CAR T-cell provided herein.

In some embodiments, the cellular deficiency is associated with diabetes or the cellular therapy is for the treatment of diabetes, optionally wherein the diabetes is Type I diabetes. In some embodiments, the population of engineered primary cells is a population of islet cells, including beta islet cells. In some embodiments, the islet cells are selected from the group consisting of an islet progenitor cell, an immature islet cell, and a mature islet cell. In some embodiments, the method comprises administering to the patient a composition comprising a population of engineered primary beta islet cells, wherein the engineered beta islet cells comprise: (i) a transgene comprising an exogenous polynucleotide encoding CD47, and (ii) inactivation or disruption of both alleles of a B2M gene. In some embodiments, the engineered primary beta islet cells comprise inactivation or disruption of both alleles of a CIITA gene.

The engineered primary beta islet cells described herein may improve glucose tolerance in a subject. Glucose tolerance may be measured by any suitable method, such as those described herein (e.g., insulin secretion assays). In some embodiments, the engineered primary beta islet cell exhibits glucose-stimulated insulin secretion (GSIS). Thus, in some embodiments, the improved glucose tolerance is measured in a GSIS perfusion assay. Glucose intolerance is related to insulin resistance, and can cause diabetes (e.g., Type 1 diabetes and Type II diabetes). Therefore, in some embodiments, provided is a method of treating diabetes comprising administering an engineered primary beta islet cell, or a composition comprising a population of engineered primary beta islet cells, to a subject in need thereof. In some embodiments, the subject is a diabetic patient. In some embodiments, the subject has Type I diabetes. In some embodiments, the subject has Type II diabetes. Specifically, in some embodiments, provided is a method of improving glucose tolerance in a subject, the method comprising administering an engineered primary beta islet cell, or a composition comprising a population of engineered primary beta islet cells, to a subject in need thereof. In some embodiments, glucose tolerance is improved relative to the subject's glucose tolerance prior to administration of the islet cells. In some embodiments, the beta islet cells reduce exogenous insulin usage in the subject. In some embodiments, glucose tolerance is improved as measured by HbA1c levels. In some embodiments, the subject is fasting. In some embodiments, the islet cells improve insulin secretion in the subject. In some embodiments, insulin secretion is improved relative to the subject's insulin secretion prior to administration of the islet cells.

The engineered primary beta islet cells may not induce and adaptive immune response in the subject. In some embodiments, the adaptive immune response is assessed using ELISPOT. For example, the adaptive immune response may be assessed by measuring the levels of IFNg cytokine secretion by CD8+ T cells. In some embodiments, the engineered primary beta islet cells exhibit lower levels of IFNg compared to wild type primary beta islet cells, such as any of about 400-fold, 300-fold, 200-fold, 100-fold, 50-fold, 25-fold, and 10-fold lower levels of IFNg compared to wild type primary beta islet cells. In some embodiments, the adaptive immune response is assessed using flow cytometry. For example, in some embodiments, the adaptive immune response is assessed by measuring the levels donor specific antibody (DSA) IgG or IgM. In some embodiments, the engineered primary beta islet cells exhibit lower levels of DSA levels compared to wild type primary beta islet cells, such as any of about 2-fold, 1.5-fold, and 1-fold lower levels of DSA compared to wild type primary beta islet cells.

In some embodiments, the cellular deficiency is is diabetes, cancer, vascularization disorders, ocular disease, thyroid disease, skin diseases, and liver diseases.

In some embodiments, the cellular deficiency is associated with a vascular condition or disease or the cellular therapy is for the treatment of a vascular condition or disease. In some embodiments, the population of cells is a population of endothelial cells. In some embodiments, the method comprises administering to the patient a composition comprising a population of engineered primary endothelial cells, wherein the engineered primary endothelial cells comprise: (i) a transgene comprising an exogenous polynucleotide encoding CD47 and (ii) inactivation or disruption of both alleles of a B2M gene. In some embodiments, the engineered primary hepatocyte cells comprise inactivation or disruption of both alleles of a CIITA gene.

In some embodiments, the cellular deficiency is associated with autoimmune thyroiditis or the cellular therapy is for the treatment of autoimmune thyroiditis. In some embodiments, the population of cells is a population of thyroid progenitor cells. In some embodiments, the method comprises administering to the patient a composition comprising a population of engineered primary thryroid progenitor cells, wherein the engineered primary thyroid progenitor cells comprise: (i) a transgene comprising an exogenous polynucleotide encoding CD47 and (ii) inactivation or disruption of both alleles of a B2M gene. In some embodiments, the engineered primary hepatocyte cells comprise inactivation or disruption of both alleles of a CIITA gene.

In some embodiments, the cellular deficiency is associated with a liver disease or the cellular therapy is for the treatment of liver disease. In some embodiments, the liver disease comprises cirrhosis of the liver. In some embodiments, the population of cells is a population of hepatocytes. In some embodiments, the population of cells is a population of hepatic progenitor cells. In some embodiments, the method comprises administering to the patient a composition comprising a population of engineered primary hepatocyte cells, wherein the engineered primary hepatocyte cells comprise: (i) a transgene comprising an exogenous polynucleotide encoding CD47 and (ii) inactivation or disruption of both alleles of a B2M gene. In some embodiments, the engineered primary hepatocyte cells comprise inactivation or disruption of both alleles of a CIITA gene. In some embodiments, the method comprises administering to the patient a composition comprising a population of engineered primary hepatic progenitor cells, wherein the engineered primary hepatic progenitor cells comprise: (i) a transgene comprising an exogenous polynucleotide encoding CD47 and (ii) inactivation or disruption of both alleles of a B2M gene. In some embodiments, the engineered primary hepatocyte cells comprise inactivation or disruption of both alleles of a CIITA gene.

In some embodiments, the cellular deficiency is associated with a corneal disease or the cellular therapy is for the treatment of corneal disease. In some embodiments, the comeal disease is Fuchs dystrophy or congenital hereditary endothelial dystrophy. In some embodiments, the population of cells is a population of primary corneal endothelial progenitor cells or primary comeal endothelial cells. In some embodiments, the population of cells is a primary optic cell. In some embodiments, the method comprises administering to the patient a composition comprising a population of engineered primary comeal endothelial progenitor cells or engineered primary comeal endothelial cells, wherein the engineered primary comeal endothelial progenitor cells or engineered primary corneal endothelial cells comprise: (i) a transgene comprising an exogenous polynucleotide encoding CD47 and (ii) inactivation or disruption of both alleles of a B2M gene. In some embodiments, the engineered primary hepatocyte cells comprise inactivation or disruption of both alleles of a CIITA gene. In some embodiments, the method comprises administering to the patient a composition comprising a population of engineered primary optic cells, wherein the engineered primary optic cells comprise: (i) a transgene comprising an exogenous polynucleotide encoding CD47 and (ii) inactivation or disruption of both alleles of a B2M gene. In some embodiments, the engineered primary hepatocyte cells comprise inactivation or disruption of both alleles of a CIITA gene.

In some embodiments, the cellular deficiency is associated with a kidney disease or the cellular therapy is for the treatment of a kidney disease. In some embodiments, the population of cells is a population of primary renal precursor cells or primary renal cells. In some embodiments, the method comprises administering to the patient a composition comprising a population of engineered primary renal precursor cells or engineered primary renal cells, wherein the engineered engineered primary renal precursor cells or engineered primary renal cells comprise: (i) a transgene comprising an exogenous polynucleotide encoding CD47 and (ii) inactivation or disruption of both alleles of a B2M gene. In some embodiments, the engineered primary hepatocyte cells comprise inactivation or disruption of both alleles of a CIITA gene.

In some embodiments, the cellular therapy is for the treatment of a cancer. In some embodiments, the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer. In some embodiments, the population of cells is a population of primary T cells or primary NK cells. In some embodiments, the method comprises administering to the patient a composition comprising a population of engineered primary T cells or primary NK cells, wherein the engineered primary T cells or primary NK cells comprise: (i) a transgene comprising an exogenous polynucleotide encoding CD47 and (ii) inactivation or disruption of both alleles of a B2M gene. In some embodiments, the engineered primary hepatocyte cells comprise inactivation or disruption of both alleles of a CIITA gene.

In some embodiments, the cellular therapy is for the treatment of a hematopoietic disease or disorder. In some embodiments, the population of cells are hematopoietic stem cells (HSCs). HSCs are stem cells that replenish all blood cell types and to self-renew. Hematopoietic stem cells may be in particular defined as cells that keep the levels of myeloid, T and B cells at robustly detectable levels (typically more than 1% of peripheral blood cells) for 16 weeks when injected into the circulation of a recipient mouse with a depleted hematopoietic system (Schroeder (2010) Cell Stem Cell 6:203-207). In some embodiments, the hematopoietic disorder may be due to a blood disease, in particular disease involving hematopoietic cells. In some embodiments, the hematopoietic disorder is a monogenic hematopoietic disease, such as due to mutation of a single gene. In some embodiments, the hematopoietic disorder is myelodysplasia, aplastic anemia, Fanconi anemia, paroxysmal nocturnal hemoglobinuria, Sickle cell disease, Diamond Blackfan anemia, Schachman Diamond disorder, Kostmann's syndrome, chronic granulomatous disease, adrenoleukodystrophy, leukocyte adhesion deficiency, hemophilia, thalassemia, beta-thalassemia, leukaemia such as acute lymphocytic leukemia (ALL), acute myelogenous (myeloid) leukemia (AML), adult lymphoblastic leukaemia, chronic lymphocytic leukemia (CLL), B-cell chronic lymphocytic leukemia (B-CLL), chronic myeloid leukemia (CML), juvenile chronic myelogenous leukemia (CML), and juvenile myelomonocytic leukemia (JMML), severe combined immunodeficiency disease (SCID), X-linked severe combined immunodeficiency, Wiskott-Aldrich syndrome (WAS), adenosine-deaminase (ADA) deficiency, chronic granulomatous disease, Chediak-Higashi syndrome, Hodgkin's lymphoma, non-Hodgkin's lymphoma (NHL) or AIDS. In some embodiments, the subject has an autoimmune disease. In some embodiments, the autoimmune disease is acute disseminated encephalomyelitis, acute hemorrhagic leukoencephalitis, Addison's disease, Agammaglobulinemia, Alopecia areata, amyotrophic lateral sclerosis, ankylosing spondylitis, antiphospholipid syndrome, antisynthetase syndrome, atopic allergy, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune urticaria, autoimmune uveitis, Balo disease, Balo concentric sclerosis, Bechets syndrome, Berger's disease, Bickerstaffs encephalitis, Blau syndrome, bullous pemphigoid, cancer, Castleman's disease, celiac disease, chronic inflammatory demyelinating polyneuropathy, chronic recurrent multifocal osteomyelitis, Churg-Strauss syndrome, cicatricial pemphigoid, Cogan syndrome, cold agglutinin disease, complement component 2 deficiency, cranial arteritis, CREST syndrome, Crohn's disease, Cushing's syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, discoid lupus erythematosus, eczema, enthesitis-related arthritis, eosinophilic fasciitis, eosinophilic gastroenteritis, epidermolysis bullosa acquisita, erythema nodosum, essential mixed cryoglobulinemia, Evan's syndrome, firodysplasia ossificans progressiva, fibrosing aveolitis, gastritis, gastrointestinal pemphigoid, giant cell arteritis, glomerulonephritis, goodpasture's syndrome, Grave's disease, Guillain-Barre syndrome (GBS), Hashimoto's encephalitis, Hashimoto's thyroiditis, hemolytic anaemia, Henoch-Schonlein purpura, herpes gestationis, hypogammaglobulinemia, idiopathic inflammatory demyelinating disease, idiopathic pulmonary fibrosis, idiopathic thrombocytopenic purpura, IgA nephropathy, inclusion body myositis, inflammatory demyelinating polyneuropathy, interstitial cystitis, juvenile idiopathic arthritis, juvenile rheumatoid arthritis, Kawasaki's disease, Lambert-Eaton myasthenic syndrome, leukocytoclastic vasculitis, lichen planus, lichen sclerosus, linear IgA disease (LAD), Lou Gehrig's disease, lupoid hepatitis, lupus erythematosus, Majeed syndrome, Meniere's disease, microscopic polyangiitis, Miller-Fisher syndrome, mixed connective tissue disease, morphea, Mucha-Habermann disease, multiple sclerosis, myasthenia gravis, myositis, neuropyelitis optica, neuromyotonia, ocular cicatricial pemphigoid, opsoclonus myoclonus syndrome, ord thyroiditis, palindromic rheumatism, paraneoplastic cerebellar degeneration, paroxysmal nocturnal hemoglobinuria (PNH), Parry Romberg syndrome, Parsonnage-Turner syndrome, pars planitis, pemphigus, pemphigus vulgaris, permicious anemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatoid fever, sarcoidosis, Schmidt syndrome, Schnitzler syndrome, scleritis, scleroderma, Sjogren's syndrome, spondylarthropathy, Still's disease, stiff person syndrome, subacute bacterial endocarditis, Susac's syndrome, Sweet's syndrome, Sydenham chorea, sympathetic ophthalmia, Takayasu's arteritis, temporal arteritis, Tolosa-Hunt syndrome, transverse myelitis, ulcerative colitis, undifferentiated connective tissue disease, undifferentiated spondylarthropathy, vasculitis, vitiligo or Wegener's granulomatosis. In some embodiments, the target cell is from a subject having a cancer. In some embodiments, the cancer is leukemia. In some embodiments, the leukemia is B-CLL, CML or T cell based leukemia such as ALT. In some embodiments, the cancer is a myeloma. In some embodiments, the method comprises administering to the patient a composition comprising a population of engineered HSCs, wherein the engineered HSCs comprise: (i) a transgene comprising an exogenous polynucleotide encoding CD47 and (ii) inactivation or disruption of both alleles of a B2M gene. In some embodiments, the engineered primary hepatocyte cells comprise inactivation or disruption of both alleles of a CIITA gene.

In some embodiments, the engineered primary cells, or a composition containing the same, provided herein are useful for the treatment of a patient sensitized from one or more antigens present in a previous transplant such as, for example, a cell transplant, a blood transfusion, a tissue transplant, or an organ transplant. In certain embodiments, the previous transplant is an allogeneic transplant and the patient is sensitized against one or more alloantigens from the allogeneic transplant. Allogeneic transplants include, but are not limited to, allogeneic cell transplants, allogeneic blood transfusions, allogeneic tissue transplants, or allogeneic organ transplants. In some embodiments, the patient is sensitized patient who is or has been pregnant (e.g., having or having had alloimmunization in pregnancy). In certain embodiments, the patient is sensitized from one or more antigens included in a previous transplant, wherein the previous transplant is a modified human cell, tissue or organ. In some embodiments, the modified human cell, tissue or organ is a modified autologous human cell, tissue or organ. In some embodiments, the previous transplant is a non-human cell, tissue or organ. In exemplary embodiments, the previous transplant is a modified non-human cell, tissue, or organ. In certain embodiments, the previous transplant is a chimera that includes a human component. In certain embodiments, the previous transplant is a CAR T-cell. In certain embodiments, the previous transplant is an autologous transplant and the patient is sensitized against one or more autologous antigens from the autologous transplant. In certain embodiments, the previous transplant is an autologous cell, tissue or organ. In certain embodiments, the sensitized patient has an allergy and is sensitized to one or more allergens. In exemplary embodiments, the patient has a hay fever, a food allergy, an insect allergy, a drug allergy or atopic dermatitis.

In some embodiments, the patient undergoing a treatment using the provided engineered primary cells, or a composition containing the same, received a previous treatment. In some embodiments, the engineered primary cells, or a composition containing the same, are used to treat the same condition as the previous treatment. In certain embodiments, the engineered primary cells, or a composition containing the same, are used to treat a different condition from the previous treatment. In some embodiments, the engineered primary cells, or a composition containing the same, administered to the patient exhibit an enhanced therapeutic effect for the treatment of the same condition or disease treated by the previous treatment. In certain embodiments, the administered engineered primary cells, or a composition containing the same, exhibit a longer therapeutic effect for the treatment of the condition or disease in the patient as compared to the previous treatment. In exemplary embodiments, the administered cells exhibit an enhanced potency, efficacy and/or specificity against the cancer cells as compared to the previous treatment. In particular embodiments, the engineered primary cells are CAR T-cells for the treatment of a cancer.

The methods provided herein can be used as a second-line treatment for a particular condition or disease after a failed first line treatment. In some embodiments, the previous treatment is a therapeutically ineffective treatment. As used herein, a “therapeutically ineffective” treatment refers to a treatment that produces a less than desired clinical outcome in a patient. For example, with respect to a treatment for a cellular deficiency, a therapeutically ineffective treatment may refer to a treatment that does not achieve a desired level of functional cells and/or cellular activity to replace the deficient cells in a patient, and/or lacks therapeutic durability. With respect to a cancer treatment, a therapeutically ineffective treatment refers to a treatment that does not achieve a desired level of potency, efficacy and/or specificity. Therapeutic effectiveness can be measured using any suitable technique known in the art. In some embodiments, the patient produces an immune response to the previous treatment. In some embodiments, the previous treatment is a cell, tissue or organ graft that is rejected by the patient. In some embodiments, the previous treatment included a mechanically assisted treatment. In some embodiments, the mechanically assisted treatment included a hemodialysis or a ventricle assist device. In some embodiments, the patient produced an immune response to the mechanically assisted treatment. In some embodiments, the previous treatment included a population of therapeutic cells that include a safety switch that can cause the death of the therapeutic cells should they grow and divide in an undesired manner. In certain embodiments, the patient produces an immune response as a result of the safety switch induced death of therapeutic cells. In certain embodiments, the patient is sensitized from the previous treatment. In exemplary embodiments, the patient is not sensitized by the administered engineered primary cells as provided herein.

In some embodiments, the provided engineered primary cells, or compositions containing the same, are administered prior to providing a tissue, organ or partial organ transplant to a patient in need thereof. In particular embodiments, the patient does not exhibit an immune response to the engineered primary cells. In certain embodiments, the engineered primary cells are administered to the patient for the treatment of a cellular deficiency in a particular tissue or organ and the patient subsequently receives a tissue or organ transplant for the same particular tissue or organ. In such embodiments, the engineered primary cell treatment functions as a bridge therapy to the eventual tissue or organ replacement. For example, in some embodiments, the patient has a liver disorder and receives an engineered hepatocyte treatment as provided herein, prior to receiving a liver transplant. In certain embodiments, the engineered primary cells are administered to the patient for the treatment of a cellular deficiency in a particular tissue or organ and the patient subsequently receives a tissue or organ transplant for a different tissue or organ. For example, in some embodiments, the patient is a diabetes patient who is treated with engineered pancreatic beta islet cells as provided herein prior to receiving a kidney transplant. In some embodiments, the method is for the treatment of a cellular deficiency. In exemplary embodiments, the tissue or organ transplant is a heart transplant, a lung transplant, a kidney transplant, a liver transplant, a pancreas transplant, an intestine transplant, a stomach transplant, a cornea transplant, a bone marrow transplant, a blood vessel transplant, a heart valve transplant, or a bone transplant.

The methods of treating a patient are generally through administrations of engineered primary cells, or a composition containing the same, as provided herein. As will be appreciated, for all the multiple embodiments described herein related to the cells and/or the timing of therapies, the administering of the cells is accomplished by a method or route that results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. In some embodiments, the cells are administered to treat a disease or disorder, such as any disease, disorder, condition, or symptom thereof that can be alleviated by cell therapy.

In some embodiments, the population of engineered primary cells, or a composition containing the same, is administered at least 1 day, at least 2 days, at least 3 days, at least 4 days, at least 5, days, at least 6 days, at least 1 week, or at least 1 month or more after the patient is sensitized. In some embodiments, the population of engineered primary cells, or a composition containing the same, is administered at least 1 week (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) or more after the patient is sensitized or exhibits characteristics or features of sensitization. In some embodiments, the population of engineered primary cells, or a composition containing the same, is administered at least 1 month (e.g., 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, or more) or more after the patient has received the transplant (e.g., an allogeneic transplant), has been pregnant (e.g., having or having had alloimmunization in pregnancy) or is sensitized or exhibits characteristics or features of sensitization.

In some embodiments, the patient who has received a transplant, who has been pregnant (e.g., having or having had alloimmunization in pregnancy), and/or who is sensitized against an antigen (e.g., alloantigens) is administered a dosing regimen comprising a first dose administration of a population of engineered primary cells described herein, a recovery period after the first dose, and a second dose administration of a population of engineered primary cells described. In some embodiments, the composite of cell types present in the first population of cells and the second population of cells are different. In certain embodiments, the composite of cell types present in the first population of engineered primary cells and the second population of engineered primary cells are the same or substantially equivalent. In many embodiments, the first population of engineered primary cells and the second population of engineered primary cells comprises the same cell types. In some embodiments, the first population of engineered primary cells and the second population of engineered primary cells comprises different cell types. In some embodiments, the first population of engineered primary cells and the second population of engineered primary cells comprises the same percentages of cell types. In other embodiments, the first population of engineered primary cells and the second population of cells comprises different percentages of cell types.

In some embodiments, the recovery period begins following the first administration of the population of engineered primary cells or a composition containing the same, and ends when such cells are no longer present or detectable in the patient. In some embodiments, the duration of the recovery period is at least 1 week (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) or more after the initial administration of the cells. In some embodiments, the duration of the recovery period is at least 1 month (e.g., 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, or more) or more after the initial administration of the cells.

In some embodiments, the administered population of engineered cells, or a composition containing the same, is hypoimmunogenic when administered to the subject. In some embodiments, the engineered cells are hypoimmune. In some embodiments, an immune response against the engineered cells is reduced or lower by at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of the immune response produced by the administration of immunogenic cells (e.g. a population of cells of the same or similar cell type or phenotype but that do not contain the modifications, e.g. genetic modifications, of the engineered cells). In some embodiments, the administered population of engineered cells, or a composition containing the same, fails to elicit an immune response against the engineered cells in the patient.

In some embodiments, the administered population of engineered primary cells, or a composition containing the same, elicits a decreased or lower level of systemic TH1 activation in the patient. In some instances, the level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells (e.g. a population of cells of the same or similar cell type or phenotype but that do not contain the modifications, e.g. genetic modifications, of the engineered primary cells). In some embodiments, the administered population of engineered primary cells, or a composition containing the same, fails to elicit systemic TH1 activation in the patient.

In some embodiments, the administered population of engineered primary cells, or a composition containing the same, elicits a decreased or lower level of immune activation of peripheral blood mononuclear cells (PBMCs) in the patient. In some instances, the level of immune activation of PBMCs elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells (e.g. a population of cells of the same or similar cell type or phenotype but that do not contain the modifications, e.g. genetic modifications, of the engineered primary cells). In some embodiments, the administered population of engineered primary cells, or a composition containing the same, fails to elicit immune activation of PBMCs in the patient.

In some embodiments, the administered population of engineered primary cells, or a composition containing the same, elicits a decreased or lower level of donor-specific IgG antibodies in the patient. In some instances, the level of donor-specific IgG antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells (e.g. a population of cells of the same or similar cell type or phenotype but that do not contain the modifications, e.g. genetic modifications, of the engineered primary cells). In some embodiments, the administered population of engineered primary cells fails to elicit donor-specific IgG antibodies in the patient.

In some embodiments, the administered population of engineered primary cells, or a composition containing the same, elicits a decreased or lower level of IgM and IgG antibody production in the patient. In some instances, the level of IgM and IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells (e.g. a population of cells of the same or similar cell type or phenotype but that do not contain the modifications, e.g. genetic modifications, of the engineered primary cells). In some embodiments, the administered population of engineered primary cells, or a composition containing the same, fails to elicit IgM and IgG antibody production in the patient.

In some embodiments, the administered population of engineered primary cells, or a composition containing the same, elicits a decreased or lower level of cytotoxic T cell killing in the patient. In some instances, the level of cytotoxic T cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of cytotoxic T cell killing produced by the administration of immunogenic cells (e.g. a population of cells of the same or similar cell type or phenotype but that do not contain the modifications, e.g. genetic modifications, of the engineered primary cells). In some embodiments, the administered population of engineered primary cells, or a composition containing the same, fails to elicit cytotoxic T cell killing in the patient.

As discussed above, provided herein are cells that in certain embodiments can be administered to a patient sensitized against alloantigens such as MHC molecules (e.g., human leukocyte antigens). In some embodiments, the patient is or has been pregnant, e.g., with alloimmunization in pregnancy (e.g., hemolytic disease of the fetus and newborn (HDFN), neonatal alloimmune neutropenia (NAN) or fetal and neonatal alloimmune thrombocytopenia (FNAIT)). In other words, the patient has or has had a disorder or condition associated with alloimmunization in pregnancy such as, but not limited to, hemolytic disease of the fetus and newborn (HDFN), neonatal alloimmune neutropenia (NAN), and fetal and neonatal alloimmune thrombocytopenia (FNAIT). In some embodiments, the patient has received an allogeneic transplant such as, but not limited to, an allogeneic cell transplant, an allogeneic blood transfusion, an allogeneic tissue transplant, or an allogeneic organ transplant. In some embodiments, the patient exhibits memory B cells against alloantigens. In some embodiments, the patient exhibits memory T cells against alloantigens. Such patients can exhibit both memory B and memory T cells against alloantigens.

Upon administration of the cells described, the patient exhibits no systemic immune response or a reduced level of systemic immune response compared to responses to cells that are not hypoimmunogenic. In some embodiments, the patient exhibits no adaptive immune response or a reduced level of adaptive immune response compared to responses to cells that are not hypoimmunogenic. In some embodiments, the patient exhibits no innate immune response or a reduced level of innate immune response compared to responses to cells that are not hypoimmunogenic. In some embodiments, the patient exhibits no T cell response or a reduced level of T cell response compared to responses to cells that are not hypoimmunogenic. In some embodiments, the patient exhibits no B cell response or a reduced level of B cell response compared to responses to cells that are not hypoimmunogenic.

### A. Dose and Dosage Regimen

Any therapeutically effective amount of cells described herein can be included in the pharmaceutical composition, depending on the indication being treated. Non-limiting examples of the cells include primary cells (e.g. primary T cells) as described. In some embodiments, the pharmaceutical composition includes at least about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, or 5×10¹⁰ cells. In some embodiments, the pharmaceutical composition includes up to about 6.0×10⁸ cells. In some embodiments, the pharmaceutical composition includes up to about 8.0×10⁸ cells. In some embodiments, the pharmaceutical composition includes at least about 1×10²-5×10², 5×10²-1×10³, 1×10³-5×10³, 5×10³-1×10⁴, 1×10⁴-5×10⁴, 5×10⁴-1×10⁵, 1×10⁵-5×10⁵, 5×10⁵-1×10⁶, 1×10⁶-5×10⁶, 5×10⁶-1×10⁷, 1×10⁷-5×10⁷, 5×10⁷-1×10⁸, 1×10⁸-5×10⁸, 5×10⁸-1×10⁹, 1×10⁹-5×10⁹, 5×10⁹-1×10¹⁰, or 1×10¹⁰-5×10¹⁰ cells. In exemplary embodiments, the pharmaceutical composition includes from about 1.0×10⁶ to about 2.5×10⁸ cells.

In some embodiments, the pharmaceutical composition has a volume of at least 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In exemplary embodiments, the pharmaceutical composition has a volume of up to about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In exemplary embodiments, the pharmaceutical composition has a volume of about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300, 350, 400, or 500 ml. In some embodiments, the pharmaceutical composition has a volume of from about 1-50 ml, 50-100 ml, 100-150 ml, 150-200 ml, 200-250 ml, 250-300 ml, 300-350 ml, 350-400 ml, 400-450 ml, or 450-500 ml. In some embodiments, the pharmaceutical composition has a volume of from about 1-50 ml, 50-100 ml, 100-150 ml, 150-200 ml, 200-250 ml, 250-300 ml, 300-350 ml, 350-400 ml, 400-450 ml, or 450-500 ml. In some embodiments, the pharmaceutical composition has a volume of from about 1-10 ml, 10-20 ml, 20-30 ml, 30-40 ml, 40-50 ml, 50-60 ml, 60-70 ml, 70-80 ml, 70-80 ml, 80-90 ml, or 90-100 ml. In some embodiments, the pharmaceutical composition has a volume that ranges from about 5 ml to about 80 ml. In exemplary embodiments, the pharmaceutical composition has a volume that ranges from about 10 ml to about 70 ml. In many embodiments, the pharmaceutical composition has a volume that ranges from about 10 ml to about 50 ml.

The specific amount/dosage regimen will vary depending on the weight, gender, age and health of the individual; the formulation, the biochemical nature, bioactivity, bioavailability and the side effects of the cells and the number and identity of the cells in the complete therapeutic regimen.

In some embodiments, a dose of the pharmaceutical composition includes about 1.0×105 to about 2.5×10⁸ cells at a volume of about 10 mL to 50 mL and the pharmaceutical composition is administered as a single dose.

In many embodiments, the cells are T cells and the pharmaceutical composition includes from about 2.0×10⁶ to about 2.0×10⁸ cells, such as but not limited to, primary T cells. In some cases, the dose includes about 1.0×10⁵ to about 2.5×10⁸ primary T cells described herein at a volume of about 10 ml to 50 ml. In several cases, the dose includes about 1.0×10⁵ to about 2.5×10⁸ primary T cells that have been described above at a volume of about 10 ml to 50 ml. In other cases, the dose is at a range that is lower than about 1.0×10⁵ to about 2.5×10⁸ T cells, including primary T cells. In yet other cases, the dose is at a range that is higher than about 1.0×10⁵ to about 2.5×10⁸ T cells, including primary T cells.

In some embodiments, the pharmaceutical composition is administered as a single dose of from about 1.0×10⁵ to about 1.0×10⁷ engineered primary cells (such as primary cells) per kg body weight for subjects 50 kg or less. In some embodiments, the pharmaceutical composition is administered as a single dose of from about 0.5×10⁵ to about 1.0×10⁷, about 1.0×10⁵ to about 1.0×10⁷, about 1.0×10⁵ to about 1.0×10⁷, about 5.0×10⁵ to about 1×10⁷, about 1.0×10⁶ to about 1×10⁷, about 5.0×10⁶ to about 1.0×10⁷, about 1.0×10⁵ to about 5.0×10⁶, about 1.0×10⁵ to about 1.0×10⁶, about 1.0×10⁵ to about 5.0×10⁵, about 1.0×10⁵ to about 5.0×10⁶, about 2.0×10⁵ to about 5.0×10⁶, about 3.0×10⁵ to about 5.0×10⁶, about 4.0×10⁵ to about 5.0×10⁶, about 5.0×105 to about 5.0×10⁶, about 6.0×10⁵ to about 5.0×10⁶, about 7.0×10⁵ to about 5.0×10⁶, about 8.0×10⁵ to about 5.0×10⁶, or about 9.0×10⁵ to about 5.0×10⁶ cells per kg body weight for subjects 50 kg or less. In some embodiments, the dose is from about 0.2×10⁶ to about 5.0×10⁶ cells per kg body weight for subjects 50 kg or less. In many embodiments, the dose is at a range that is lower than from about 0.2×10⁶ to about 5.0×10⁶ cells per kg body weight for subjects 50 kg or less. In many embodiments, the dose is at a range that is higher than from about 0.2×10⁶ to about 5.0×10⁶ cells per kg body weight for subjects 50 kg or less. In exemplary embodiments, the single dose is at a volume of about 10 ml to 50 ml. In some embodiments, the dose is administered intravenously.

In exemplary embodiments, the cells are administered in a single dose of from about 1.0×10⁶ to about 5.0×10⁸ cells (such as primary cells) for subjects above 50 kg. In some embodiments, the pharmaceutical composition is administered as a single dose of from about 0.5×10⁶ to about 1.0×10⁹, about 1.0×10⁶ to about 1.0×10⁹, about 1.0×10⁶ to about 1.0×10⁹, about 5.0×10⁶ to about 1.0×10⁹, about 1.0×10⁷ to about 1.0×10⁹, about 5.0×10⁷ to about 1.0×10⁹, about 1.0×10⁶ to about 5.0×10⁷, about 1.0×10⁶ to about 1.0×10⁷, about 1.0×10⁶ to about 5.0×10⁷, about 1.0×10⁷ to about 5.0×10⁸, about 2.0×10⁷ to about 5.0×10⁸, about 3.0×10⁷ to about 5.0×10⁸, about 4.0×10⁷ to about 5.0×10⁸, about 5.0×10⁷ to about 5.0×10⁸, about 6.0×10⁷ to about 5.0×10⁸, about 7.0×10⁷ to about 5.0×10⁸, about 8.0×10⁷ to about 5.0×10⁸, or about 9.0×10⁷ to about 5.0×10⁸ cells per kg body weight for subjects 50 kg or less. In many embodiments, the cells are administered in a single dose of about 1.0×10⁷ to about 2.5×10⁸ cells for subjects above 50 kg. In some embodiments, the cells are administered in a single dose of a range that is less than about 1.0×10⁷ to about 2.5×10⁸ cells for subjects above 50 kg. In some embodiments, the cells are administered in a single dose of a range that is higher than about 1.0×10⁷ to about 2.5×10⁸ cells for subjects above 50 kg. In some embodiments, the dose is administered intravenously. In exemplary embodiments, the single dose is at a volume of about 10 ml to 50 ml. In some embodiments, the dose is administered intravenously.

In exemplary embodiments, the dose is administered intravenously at a rate of about 1 to 50 ml per minute, 1 to 40 ml per minute, 1 to 30 ml per minute, 1 to 20 ml per minute, 10 to 20 ml per minute, 10 to 30 ml per minute, 10 to 40 ml per minute, 10 to 50 ml per minute, 20 to 50 ml per minute, 30 to 50 ml per minute, 40 to 50 ml per minute. In numerous embodiments, the pharmaceutical composition is stored in one or more infusion bags for intravenous administration. In some embodiments, the dose is administered completely at no more than 10 minutes, 15 minutes, 20 minutes, 25 minutes, 30 minutes, 35 minutes, 40 minutes, 45 minutes, 50 minutes, 55 minutes, 60 minutes, 70 minutes, 80 minutes, 90 minutes, 120 minutes, 150 minutes, 180 minutes, 240 minutes, or 300 minutes.

In some embodiments, a single dose of the pharmaceutical composition is present in a single infusion bag. In other embodiments, a single dose of the pharmaceutical composition is divided into 2, 3, 4 or 5 separate infusion bags.

In some embodiments, the cells described herein are administered in a plurality of doses such as 2, 3, 4, 5, 6 or more doses. In some embodiments, each dose of the plurality of doses is administered to the subject ranging from 1 to 24 hours apart. In some instances, a subsequent dose is administered from about 1 hour to about 24 hours (e.g., about 1, 2, 3, 4, 5, 6, 7, 8, 9, 10, 11, 12, 13, 14, 15, 16, 17, 18, 19, 20, 21, 22, 23 or about 24 hours) after an initial or preceding dose. In some embodiments, each dose of the plurality of doses is administered to the subject ranging from about 1 day to 28 days apart. In some instances, a subsequent dose is administered from about 1 day to about 28 days (e.g., about 1, 2, 3, 4, 5, 6, 7, 8, 9, 10, 11, 12, 13, 14, 15, 16, 17, 18, 19, 20, 21, 22, 23, 24, 25, 26, 27, or about 28 days) after an initial or preceding dose. In many embodiments, each dose of the plurality of doses is administered to the subject ranging from 1 week to about 6 weeks apart. In certain instances, a subsequent dose is administered from about 1 week to about 6 weeks (e.g., about 1, 2, 3, 4, 5, or 6 weeks) after an initial or preceding dose. In several embodiments, each dose of the plurality of doses is administered to the subject ranging from about 1 month to about 12 months apart. In several instances, a subsequent dose is administered from about 1 month to about 12 months (e.g., about 1, 2, 3, 4, 5, 6, 7, 8, 9, 10, 11, or 12 months) after an initial or preceding dose.

In some embodiments, a subject is administered a first dosage regimen at a first timepoint, and then subsequently administered a second dosage regimen at a second timepoint. In some embodiments, the first dosage regimen is the same as the second dosage regimen. In other embodiments, the first dosage regimen is different than the second dosage regimen. In some instances, the number of cells in the first dosage regimen and the second dosage regimen are the same. In some instances, the number of cells in the first dosage regimen and the second dosage regimen are different. In some cases, the number of doses of the first dosage regimen and the second dosage regimen are the same. In some cases, the number of doses of the first dosage regimen and the second dosage regimen are different.

In some embodiments, the cells are engineered T cells (e.g. primary T cells) and the first dosage regimen includes engineered T cells expressing a first CAR and the second dosage regimen includes engineered T cells expressing a second CAR such that the first CAR and the second CAR are different. For instance, the first CAR and second CAR bind different target antigens. In some cases, the first CAR includes an scFv that binds an antigen and the second CAR includes an scFv that binds a different antigen. In some embodiments, the first dosage regimen includes engineered T cells expressing a first CAR and the second dosage regimen includes engineered T cells or primary T cells expressing a second CAR such that the first CAR and the second CAR are the same. The first dosage regimen can be administered to the subject at least 1 month, 2 months, 3 months, 4 months, 5 months, 6 months, 7 months, 8 months, 9 months, 10 months, 11 months, 12 months, 1-3 months, 1-6 months, 4-6 months, 3-9 months, 3-12 months, or more months apart from the second dosage regimen. In some embodiments, a subject is administered a plurality of dosage regimens during the course of a disease (e.g., cancer) and at least two of the dosage regimens comprise the same type of engineered T cells described herein. In other embodiments, at least two of the plurality of dosage regimens comprise different types of engineered T cells described herein.

CL. Immunosuppressive Agent

In some embodiments, an immunosuppressive and/or immunomodulatory agent is not administered to the patient before the first administration of the population of engineered primary cells, or a composition containing the same.

In some embodiments, an immunosuppressive and/or immunomodulatory agent may be administered to a patient received administration of engineered primary cells. In some embodiments, the immunosuppressive and/or immunomodulatory agent is administered prior to administration of the engineered primary cells. In some embodiments, the immunosuppressive and/or immunomodulatory agent is administered prior to administration of a first and/or second administration of engineered primary cells.

Non-limiting examples of an immunosuppressive and/or immunomodulatory agent include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-α and similar agents. In some embodiments, the immunosuppressive and/or immunomodulatory agent is selected from a group of immunosuppressive antibodies consisting of antibodies binding to p75 of the IL-2 receptor, antibodies binding to, for instance, MHC, CD2, CD3, CD4, CD7, CD28, B7, CD40, CD45, IFN-gamma, TNF-.alpha., IL-4, IL-5, IL-6R, IL-6, IGF, IGFR1, IL-7, IL-8, IL-10, CD11a, or CD58, and antibodies binding to any of their ligands. In some embodiments where an immunosuppressive and/or immunomodulatory agent is administered to the patient before or after the first administration of the cells, the administration is at a lower dosage than would be required for cells with one or more MHC class I molecules and/or one or more MHC class II molecules expression and without exogenous expression of CD47.

In one embodiment, such an immunosuppressive and/or immunomodulatory agent may be selected from soluble IL-15R, IL-10, B7 molecules (e.g., B7-1, B7-2, variants thereof, and fragments thereof), ICOS, and OX40, an inhibitor of a negative T cell regulator (such as an antibody against CTLA-4) and similar agents.

In some embodiments, an immunosuppressive and/or immunomodulatory agent can be administered to the patient before the first administration of the population of engineered primary cells. In some embodiments, an immunosuppressive and/or immunomodulatory agent is administered at least 1, 2, 3, 4, 5, 6, 7, 8, 9, 10, 11, 12, 13, 14 days or more before the first administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the first administration of the cells.

In particular embodiments, an immunosuppressive and/or immunomodulatory agent is not administered to the patient after the first administration of the cells, or is administered at least 1, 2, 3, 4, 5, 6, 7, 8, 9, 10, 11, 12, 13, 14 days or more after the first administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the first administration of the cells.

In some embodiments, an immunosuppressive and/or immunomodulatory agent is not administered to the patient before the administration of the population of enginered cells. In many embodiments, an immunosuppressive and/or immunomodulatory agent is administered to the patient before the first and/or second administration of the population of engineered primary cells. In some embodiments, an immunosuppressive and/or immunomodulatory agent is administered at least 1, 2, 3, 4, 5, 6, 7, 8, 9, 10, 11, 12, 13, 14 days or more before the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the first and/or second administration of the cells. In particular embodiments, an immunosuppressive and/or immunomodulatory agent is administered at least 1, 2, 3, 4, 5, 6, 7, 8, 9, 10, 11, 12, 13, 14 days or more after the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the first and/or second administration of the cells.

In some embodiments where an immunosuppressive and/or immunomodulatory agent is administered to the patient before or after the administration of the cells, the administration is at a lower dosage than would be required for immunogenic cells (e.g. a population of cells of the same or similar cell type or phenotype but that do not contain the modifications, e.g. genetic modifications, of the engineered primary cells, e.g. with one or more MHC class I molecules and/or one or more MHC class II molecules expression and without exogenous expression of CD47.

### VI. Exemplary Embodiments

The following exemplary embodiments are provided herein:

Embodiment 1. An engineered primary cell comprising modifications that (i) increase expression of one or more tolerogenic factor, and (ii) reduce expression of one or more major histocompatibility complex (MHC) class I molecules and/or one or more MHC class II molecules, wherein the increased expression of (i) and the reduced expression of (ii) is relative to a cell of the same cell type that does not comprise the modifications.

Embodiment 2. The engineered primary cell of embodiment 1, wherein the modification in (ii) reduces expression of one or more MHC class I molecules.

Embodiment 3. The engineered primary cell of embodiment 1, wherein the modifications in (ii) reduces expression of one or more MHC class I and one or more MHC class II molecules.

Embodiment 4. The engineered primary cell of any of embodiments 1-3, wherein the one or more tolerogenic factor is selected from the group consisting of CD47, CD27, CD200, HLA-C, HLA-E, HLA-E heavy chain, HLA-G, PD-L1, IDO1, CTLA4-Ig, C1-Inhibitor, IL-10, IL-35, FASL, CCL21, MFGE8, and SERPINB9, and any combination thereof.

Embodiment 5. The engineered primary cell of embodiment 4, wherein the one or more tolerogenic factor is selected from the group consisting of CD47, PD-L1, HLA-E, HLA-G, CCL21, FASL, SERPINB9, CD200, MFGE8, and any combination thereof.

Embodiment 6. The engineered primary cell of embodiment 4, wherein at least one of the one or more tolerogenic factor is CD47.

Embodiment 7. An engineered primary cell comprising modifications that (i) increase expression of CD47, and (ii) reduce expression of one or more major histocompatibility complex (MHC) class I molecules and/or one or more MHC class II molecules, wherein the increased expression of (i) and the reduced expression of (ii) is relative to a cell of the same cell type that does not comprise the modifications.

Embodiment 8. The engineered primary cell of any of embodiments 1-7, wherein the modification(s) that increase expression comprise increased surface expression, and/or the modifications that reduce expression comprise reduced surface expression.

Embodiment 9. The engineered primary cell of any of embodiments 6-8, wherein the modification that increases expression of CD47 comprises an exogenous polynucleotide encoding the CD47 protein.

Embodiment 10. The engineered primary cell of embodiment 9, wherein the exogenous polynucleotide encoding CD47 encodes a sequence of amino acids having at least 85% identity to the amino acid sequence of SEQ ID NO: 2, and reduces innate immune killing of the engineered primary cell

Embodiment 11. The engineered primary cell of embodiment 10, wherein the exogenous polynucleotide encoding CD47 encodes a sequence set forth in SEQ ID NO: 2.

Embodiment 12. The engineered primary cell of any of embodiments 7-11, wherein the exogenous polynucleotide encoding CD47 is operably linked to a promoter.

Embodiment 13. The engineered primary cell of embodiment 12, wherein the promoter is a constitutive promoter.

Embodiment 14. The engineered primary cell of embodiment 12 or 13, wherein the promoter is selected from the group consisting of the CAG promoter, the cytomegalovirus (CMV) promoter, the EF1a promoter, the PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, and the Rous sarcoma virus (RSV) promoter.

Embodiment 15. The engineered primary cell of any of embodiments 4-14, wherein the exogenous polynucleotide encoding CD47 is integrated into the genome of the engineered primary cell.

Embodiment 16. The method of embodiment 15, wherein the exogenous polynucleotide is a multicistronic vector encoding CD47 and an additional transgene encoding a second transgene.

Embodiment 17. The engineered primary cell of embodiment 15, wherein the integration is by non-targeted insertion into the genome of the engineered primary cell, optionally by introduction of the exogenous polynucleotide into the cell using a lentiviral vector.

Embodiment 18. The engineered primary cell of embodiment 15, wherein the integration is by targeted insertion into a target genomic locus of the cell.

Embodiment 19. The method of embodiment 18, wherein the target genomic locus is a safe harbor locus, a B2M gene locus, a CIITA gene locus, a TRAC gene locus, or a TRBC gene locus.

Embodiment 20. The method of embodiment 19, wherein the target genomic locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

Embodiment 21. The engineered primary cell of any of embodiments 1-20, wherein the modification that reduces expression of one or more MHC class I molecules reduces one or more MHC class I molecules protein expression.

Embodiment 22. The engineered primary cell of any of embodiments 1-21, wherein the modification that reduces expression of one or more MHC class I molecules is a modification that reduces expression of B-2 microglobulin (B2M).

Embodiment 23. The engineered primary cell of embodiment 22, wherein the modification that reduces expression of one or more MHC class I molecules comprises reduced mRNA expression of B2M.

Embodiment 24. The engineered primary cell of embodiment 22, wherein the modification that reduces expression of one or more MHC class I molecules comprises reduced protein expression of B2M.

Embodiment 25. The engineered primary cell of any of embodiments 22-24, wherein the modification eliminates B2M gene activity.

Embodiment 26. The engineered primary cell of any of embodiments 22-25, wherein the modification comprises inactivation or disruption of both alleles of the B2M gene.

Embodiment 27. The engineered primary cell of any of embodiments 22-26, wherein the modification comprises inactivation or disruption of all B2M coding sequences in the cell.

Embodiment 28. The engineered primary cell of embodiment 26 or embodiment 27, wherein the inactivation or disruption comprises an indel in the B2M gene.

Embodiment 29. The engineered primary cell of any of embodiments 22-28, wherein the modification is a frameshift mutation or a deletion of a contiguous stretch of genomic DNA of the B2M gene.

Embodiment 30. The engineered primary cell of any of embodiments 22-29, wherein the B2M gene is knocked out.

Embodiment 31. The engineered primary cell of any of embodiments 22-30, wherein the modification is by nuclease-mediated gene editing.

Embodiment 32. The engineered primary cell of embodiment 31, wherein the nuclease-mediated gene editing is by a zinc finger nuclease (ZFN), a TAL-effector nuclease (TALEN), or a CRISPR-Cas combination that targets the B2M gene, optionally wherein the Cas is Cas9.

Embodiment 33. The engineered primary cell of embodiment 32, wherein the nuclease-mediated gene editing is by a CRISPR-Cas combination and the CRISPR-Cas combination comprises a guide RNA (gRNA) having a targeting domain that is complementary to at least one target site within the B2M gene.

Embodiment 34. The engineered primary cell of embodiment 33, wherein the CRISPR-Cas combination is a ribonucleoprotein (RNP) complex comprising the gRNA and a Cas protein.

Embodiment 35. The engineered primary cell of embodiment 21, wherein the modification that reduces expression of one or more MHC class I molecules is a modification that reduces expression of an HLA-A protein, an HLA-B protein, or HLA-C protein, optionally wherein a gene encoding said HLA-A protein, an HLA-B protein, or HLA-C protein is knocked out.

Embodiment 36. The engineered primary cell of any of embodiments 1-35, wherein the modification that reduces expression of one or more MHC class II molecules reduces one or more MHC class II molecules protein expression.

Embodiment 37. The engineered primary cell of any of embodiments 1-36, wherein the modification that reduces expression of one or more MHC class II molecules is a modification that reduces expression of CIITA.

Embodiment 38. The engineered primary cell of embodiment 37, wherein the modification that reduces expression of one or more MHC class II molecules comprises reduced mRNA expression of CITA.

Embodiment 39. The engineered primary cell of embodiment 37, wherein the modification that reduces expression of one or more MHC class II molecules comprises reduced protein expression of CITA.

Embodiment 40. The engineered primary cell of any of embodiments 37-39, wherein the modification eliminates CIITA gene activity.

Embodiment 41. The engineered primary cell of any of embodiments 37-40, wherein the modification comprises inactivation or disruption of both alleles of the CIITA gene.

Embodiment 42. The engineered primary cell of any of embodiments 37-41, wherein the modification comprises inactivation or disruption of all CIITA coding sequences in the cell.

Embodiment 43. The engineered primary cell of embodiment 41 or embodiment 42, wherein the inactivation or disruption comprises an indel in the CIITA gene

Embodiment 44. The engineered primary cell of any of embodiments 37-43, wherein the indel is a frameshift mutation or a deletion of a contiguous stretch of genomic DNA of the CIITA gene.

Embodiment 45. The engineered primary cell of any of embodiments 37-44, wherein CIITA gene is knocked out.

Embodiment 46. The engineered primary cell of any of embodiments 37-45, wherein the modification is by nuclease-mediated gene editing.

Embodiment 47. The engineered primary cell of embodiment 46, wherein the nuclease-mediated gene editing is by a zinc finger nuclease (ZFN), a TAL-effector nuclease (TALEN), or a CRISPR-Cas combination that targets the CIITA gene, optionally wherein the Cas is Cas9.

Embodiment 48. The engineered primary cell of embodiment 46 or embodiment 47, wherein the nuclease-mediated gene editing is by a CRISPR-Cas combination and the CRISPR-Cas combination comprises a guide RNA (gRNA) having a targeting domain that is complementary to at least one target site within the CIITA gene.

Embodiment 49. The engineered primary cell of embodiment 48, wherein the CRISPR-Cas combination is a ribonucleoprotein (RNP) complex comprising the gRNA and a Cas protein.

Embodiment 50. The engineered primary cell of embodiment 36, wherein the modification that reduces expression of one or more MHC class II molecules is a modification that reduces expression of an HLA-DP protein, an HLA-DR protein, or HLA-DQ protein, optionally wherein a gene encoding said HLA-DP protein, an HLA-DR protein, or HLA-DQ protein is knocked out.

Embodiment 51. The engineered primary cell of any one of embodiments 1-50, wherein the engineered primary cell is a human cell or an animal cell.

Embodiment 52. The engineered primary cell of embodiment 51, wherein the engineered primary cell is a human cell.

Embodiment 53. The engineered primary cell of any of embodiments 1-52, wherein the primary cell is a cell type that is exposed to the blood.

Embodiment 54. The engineered primary cell of any one of embodiments 1-53, wherein the engineered primary cell is a primary cell isolated from a donor subject.

Embodiment 55. The engineered primary cell of embodiment 54, wherein the donor subject is healthy or is not suspected of having a disease or condition at the time the donor sample is obtained from the donor subject.

Embodiment 56. The engineered primary cell of any of embodiments 1-55, wherein the engineered primary cell is selected from an islet cell, a beta islet cell, B cell, T cell, NK cell, retinal pigmented epithelium cell, glial progenitor cell, endothelial cell, hepatocyte, thyroid cell, skin cell, and blood cell.

Embodiment 57. The engineered primary cell of any of embodiments 1-56, wherein the engineered primary cell is an endothelial cell.

Embodiment 58. The engineered primary cell of any of embodiments 1-56, wherein the engineered primary cell is an epithelial cell.

Embodiment 59. The engineered primary cell of any of embodiments 1-56, wherein the engineered primary cell is a T cell.

Embodiment 60. The engineered primary cell of any of embodiments 1-56, wherein the engineered primary cell is an NK cell.

Embodiment 61. The engineered primary cell of embodiment 59 or embodiment 60, wherein the engineered primary cell comprises a chimeric antigen receptor (CAR).

Embodiment 62. The engineered primary cell of any of embodiments 1-52, wherein the engineered primary cell is an islet cell.

Embodiment 63. The engineered primary cell of embodiment 62, wherein the islet cell is a beta islet cell.

Embodiment 64. The engineered primary cell of any of embodiments 1-52, wherein the engineered primary cell is a hepatocyte.

Embodiment 65. The engineered primary cell of any of embodiments 1-64, wherein the engineered primary cell is ABO blood group type O.

Embodiment 66. The engineered primary cell of any of embodiments 1-65, wherein the engineered primary cell is Rhesus factor negative (Rh−).

Embodiment 67. A method of generating an engineered primary cell, the method comprising:
a) reducing or eliminating the expression of one or more MHC class I molecules and/or one or more MHC class II moleculesin a primary cell; and,
b) increasing the expression of one or more tolerogenic factors in the primary cell.

Embodiment 68. The method of any of embodiments 67, wherein the one or more tolerogenic factor is selected from the group consisting of CD47, CD27, CD200, HLA-C, HLA-E, HLA-E heavy chain, HLA-G, PD-L1, IDO1, CTLA4-Ig, C1-Inhibitor, IL-10, IL-35, FASL, CCL21, MFGE8, and SERPINB9, and any combination thereof.

Embodiment 69. The method of embodiment 68, wherein the one or more tolerogenic factor is selected from the group consisting of CD47, PD-L1, HLA-E, HLA-G, CCL21, FASL, SERPINB9, CD200, MFGE8, and any combination thereof.

Embodiment 70. The method primary cell of embodiment 69, wherein at least one of the one or more tolerogenic factor is CD47.

Embodiment 71. The method of any of embodiments 67-70, wherein the method comprises reducing or eliminating the expression of one or more MHC class I molecules.

Embodiment 72. The method of any of embodiments 67-71, wherein the method comprises reducing or eliminating the expression of one or more MHC class I molecules and one or more MHC class II molecules.

Embodiment 73. A method of generating an engineered primary cell, the method comprising:
a. reducing or eliminating the expression of one or more MHC class I molecules and/or one or more MHC class II molecules in the cell; and,
b. increasing the expression of CD47 in the cell.

Embodiment 74. The method of embodiment 73, wherein the method comprises reducing or eliminating the expression of one or more MHC class I molecules.

Embodiment 75. The method of embodiment 73, wherein the method comprises reducing or eliminating the expression of one or more MHC class I molecules and one or more MHC class II molecules.

Embodiment 76. The method of any of embodiments 67-75, wherein the modification(s) that increase expression comprise increased surface expression, and/or the modifications that reduce expression comprise reduced surface expression.

Embodiment 77. The method of any of embodiments 70-75, wherein the modification that increases expression of CD47 comprises an exogenous polynucleotide encoding the CD47 protein.

Embodiment 78. The method of any of embodiment 77, wherein the exogenous polynucleotide encoding CD47 encodes a sequence of amino acids having at least 85% identity to the amino acid sequence of SEQ ID NO: 2, and reduces innate immune killing of the engineered primary cell.

Embodiment 79. The method of embodiment 78, wherein the exogenous polynucleotide encoding CD47 encodes a sequence set forth in SEQ ID NO: 2.

Embodiment 80. The method of any of embodiments 77-79, wherein the exogenous polynucleotide encoding CD47 is operably linked to a promoter.

Embodiment 81. The method of any of embodiments 77-80, wherein the exogenous polynucleotide encoding CD47 is integrated into the genome of the engineered primary cell.

Embodiment 82. The method of embodiment 81, wherein the integration is by non-targeted insertion into the genome of the engineered primary cell, optionally by introduction of the exogenous polynucleotide into the engineered primary cell using a lentiviral vector.

Embodiment 83. The method of embodiment 81, wherein the integration is by targeted insertion into a target genomic locus of the cell, optionally wherein the targeted insertion is by nuclease-mediated gene editing with homology-directed repair.

Embodiment 84. The method of embodiment 83, wherein the target genomic locus is a B2M gene locus, a CIITA gene locus, a CD142 gene locus, a TRAC gene locus, or a TRBC gene locus.

Embodiment 85. The method of embodiment 84, wherein the target genomic locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

Embodiment 86. The method of any of embodiments 83-85, wherein the nuclease-mediated gene editing is by a zinc finger nuclease (ZFN), a TAL-effector nuclease (TALEN), or a CRISPR-Cas combination that targets the target genomic locus, optionally wherein the Cas is Cas9.

Embodiment 87. The method of embodiment 86, wherein the nuclease-mediated gene editing is by a CRISPR-Cas combination and the CRISPR-Cas combination comprises a guide RNA (gRNA) having a targeting domain that is complementary to a target sequence of the target genomic locus and a homology-directed repair template comprising the exogenous polynucleotide encoding CD47.

Embodiment 88. The method of embodiment 87, wherein the CRISPR-Cas combination is a ribonucleoprotein (RNP) complex comprising the gRNA and a Cas protein.

Embodiment 89. The method of any of embodiments 67-88, wherein the engineered primary cell is a hypo-immunogenic primary cell.

Embodiment 90. The method of any of embodiments 67-89, wherein reducing or eliminating expression of one or more MHC class I molecules comprises introducing a modification that reduces one or more MHC class I molecules protein expression.

Embodiment 91. The method of any of embodiments 67-90, wherein the modification that reduces one or more MHC class I molecules protein expression comprises reduced expression of B2M.

Embodiment 92. The method of any of embodiments 67-91, wherein the modification that reduces one or more MHC class I molecules protein expression comprises reduced protein expression of B2M.

Embodiment 93. The method of embodiment 91 or embodiment 92, wherein the modification that reduces one or more MHC class I molecules protein expression eliminates B2M gene activity.

Embodiment 94. The method of any of embodiments 67-93, wherein the modification that reduces one or more MHC class I molecules expression comprises inactivation or disruption of both alleles of the B2M gene.

Embodiment 95. The method of any of embodiments 67-94, wherein the modification that reduces one or more MHC class I molecules protein expression comprises inactivation or disruption of all B2M coding sequences in the cell.

Embodiment 96. The method of embodiment 87 or embodiment 88, wherein the inactivation or disruption comprises an indel in the endogenous B2M gene or a deletion of a contiguous stretch of genomic DNA of the endogenous B2M gene.

Embodiment 97. The method of embodiment 89, wherein the indel is a frameshift mutation or a deletion of a contiguous stretch of genomic DNA of the B2M gene.

Embodiment 98. The method of any of embodiments 84-90, wherein the endogenous B2M gene is knocked out.

Embodiment 99. The method of any of embodiments 84-91, wherein the modification reduces one or more MHC class I molecules protein expression is by nuclease-mediated gene editing.

Embodiment 100. The method of embodiment 92, wherein the nuclease-mediated gene editing is by a zinc finger nuclease (ZFN), a TAL-effector nuclease (TALEN), or a CRISPR-Cas combination that targets the B2M gene, optionally wherein the Cas is Cas9.

Embodiment 101. The method of embodiment 100, wherein the nuclease-mediated gene editing is by a CRISPR-Cas combination and the CRISPR-Cas combination comprises a guide RNA (gRNA) having a targeting domain that is complementary to at least one target site within the B2M gene.

Embodiment 102. The method of embodiment 101, wherein the CRISPR-Cas combination is a ribonucleoprotein (RNP) complex comprising the gRNA and a Cas protein.

Embodiment 103. The method of embodiment 66-102, wherein the modification that reduces expression of one or more MHC class I reduces HLA-A protein expression, HLA-B protein expression, or HLA-C protein expression, optionally wherein the protein expression is reduced by knocking out a gene encoding said HLA-A protein, HLA-B protein, or HLA-C protein.

Embodiment 104. The method of any of embodiments 67-103, wherein reducing or eliminating expression of one or more MHC class II molecules comprises introducing a modification that reduces one or more MHC class II molecules protein expression.

Embodiment 105. The method of any of embodiments 67-104, wherein the genetic modification that reduces one or more MHC class II molecules protein expression comprises reduced expression of CIITA.

Embodiment 106. The method of any of embodiments 67-105, wherein the genetic modification that reduces one or more MHC class II molecules protein expression comprises reduced protein expression of CIITA.

Embodiment 107. The method of embodiment 104 or embodiment 105, wherein the modification that reduces one or more MHC class II molecules protein expression eliminates CIITA.

Embodiment 108. The method of any of embodiments 67-107, wherein the modification that reduces one or more MHC class II molecules protein expression comprises inactivation or disruption of both alleles of the CIITA gene.

Embodiment 109. The method of any of embodiments 67-108, wherein the modification comprises inactivation or disruption of all CIITA coding sequences in the cell.

Embodiment 110. The method of embodiment 108 or embodiment 109, wherein the inactivation or disruption comprises an indel in the CIITA gene or a deletion of a contiguous stretch of genomic DNA of the CIITA gene.

Embodiment 111. The method of embodiment 110, wherein the indel is a frameshift mutation or a deletion of a contiguous stretch of genomic DNA of the CIITA gene.

Embodiment 112. The method of any of embodiments 67-111, wherein the CIITA gene is knocked out.

Embodiment 113. The method of embodiment 67, the genetic modification that reduces expression of one or more MHC class II molecules reduces the expression of a HLA-DP protein, a HLA-DR protein, or a HLA-DQ protein, optionally wherein said HLA-DP protein expression, said HLA-DR protein expression, or said HLA-DQ protein expression is reduced by knocking out a gene encoding said HLA-DP protein, said HLA-DR protein, or said HLA-DQ protein.

Embodiment 114. The method of any of embodiments 67-113, wherein the engineered primary cell is a human cell or an animal cell.

Embodiment 115. The method of any of embodiments 67-114, wherein the engineered primary cell is a human cell.

Embodiment 116. The method of any of embodiments 67-115, wherein the engineered primary cell is a cell type that is exposed to the blood.

Embodiment 117. The method of any of embodiments 67-115, wherein the engineered primary cell is isolated from a donor subject.

Embodiment 118. The method of any of embodiments 67-115, wherein the engineered primary cell is selected from an islet cell, a beta islet cell, B cell, T cell, NK cell, retinal pigmented epithelium cell, glial progenitor cell, endothelial cell, hepatocyte, thyroid cell, skin cell, and blood cell.

Embodiment 119. The method of any of embodiments 67-115, wherein the engineered primary cell is an islet cell.

Embodiment 200. The method of embodiment 119, wherein, prior to step a) the primary islet cell has been dissociated from a primary islet cluster.

Embodiment 201. The method of embodiment 200, wherein the primary islet cluster is a human primary cadaveric islet cluster.

Embodiment 202. The method of embodiment 200 or embodiment 201, wherein after step a) and/or after step b) the primary islet cell is incubated under conditions for re-clustering into a modified primary islet cluster, wherein at least a portion of the incubating is carried out with motion.

Embodiment 203. The method of embodiment 202, wherein the incubating further comprises a least a portion of incubating under static conditions.

Embodiment 204. The method of embodiment 202 or embodiment 203, wherein the incubating comprises a first incubation under static conditions followed by the incubating with motion.

Embodiment 205. The method of embodiment 202 or embodiment 203, wherein the incubating comprises the incubating with motion followed by a second incubation under static conditions.

Embodiment 206. The method of any of embodiments 202-205, wherein prior to the incubating under conditions for reclustering, the method comprises selecting for islet cells that have been modified.

Embodiment 207. The method of embodiment 206, wherein the selecting is by fluorescence-activated cell sorting (FACS).

Embodiment 208. The method of any of embodiments 119-207, wherein the method comprises:
i) dissociating a primary islet cluster into a suspension of primary beta islet cells;
ii) modifying primary beta islet cells of the suspension to reduce or eliminate the expression of one or more MHC class I and/or one or more MHC class II HLA in primary beta islet cell;
iii) incubating the modified primary beta islet cells under conditions for re-clustering into a first modified primary islet cluster, wherein at least a portion of the incubating is carried out with motion;
iv) dissociating the modified] primary islet cluster into a suspension of modified primary beta islet cells;
v) further modifying the modified primary islet cells of the suspension to increase the expression of one or more tolerogenic factors in the primary cell; and
vi) incubating the further modified primary beta islet cells under conditions for re-clustering into a second modified primary islet cluster, wherein at least a portion of the incubating is carried out with motion.

Embodiment 209. The method of embodiment 66 or 208, wherein the one or more MHC class I HLA is an HLA-A protein, an HLA-B protein, or HLA-C protein.

Embodiment 210. The method of embodiment 66, 208, or 209, wherein the one or more MHC class II HLA is an HLA-DP protein, an HLA-DR protein, or an HLA-DQ protein.

Embodiment 211. The method of any of embodiments 208-210, wherein the modifying is by genetic engineering.

Embodiment 212. The method of any of embodiments 208-211, wherein the motion is shaking.

Embodiment 213. The method of embodiment 212, wherein the shaking comprises orbital motion.

Embodiment 214. The method of embodiment 212, wherein the shaking comprises bidirectional linear movement.

Embodiment 215. The method of embodiment 212 or embodiment 213, wherein the shaking is with an orbital shaker.

Embodiment 216. The method of embodiment 202-215, wherein the incubating in (iii) and/or the incubating in vi) further comprises a least a portion of incubating under static conditions.

Embodiment 217. The method of any of embodiments 202-216, wherein the incubating in iii) and/or the incubating in vi) comprises a first incubation under static conditions followed by the incubating with motion.

Embodiment 218. The method of any of embodiments 202-216, wherein the incubating comprises the incubating with motion followed by a second incubation under static conditions.

Embodiment 219. The method of any of embodiments 208-218, wherein prior to v), the method comprises selecting, from the dissociated islet cells in iv), beta islet cells that have been modified, and optionally repeating steps iii) and iv) on the selected islet cells.

Embodiment 220. The method of any of embodiments 208-218, wherein after the incubating in vi), the method comprises dissociating the second modified primary islet cluster into a suspension of modified primary beta islet cells and selecting for islet cells that have been modified.

Embodiment 221. The method of embodiment 220, wherein incubating the selected modified primary beta islet cells under conditions for re-clustering into a modified primary islet cluster, wherein at least a portion of the incubating is carried out with motion.

Embodiment 222. A method for gene editing primary islet cells, the method comprising:
i) dissociating a primary islet cluster into a suspension of primary beta islet cells;
ii) modifying primary beta islet cells of the suspension; and
iii) incubating the modified primary beta islet cells under conditions for re-clustering the modified primary beta islet cells into an islet, wherein at least a portion of the incubating is carried out with shaking.

Embodiment 223. The method of embodiment 222, wherein the primary islet cluster is a human primary cadaveric islet cluster.

Embodiment 224. The method of embodiment 222 or embodiment 223, wherein the modifying comprises introducing one more modifications into the cell to reduce expression of one or more genes encoding an endogenous protein in the cell or to increase expression of one or more heterologous proteins in the cell.

Embodiment 225. The method of embodiment 222-224, wherein the incubating in (iii) and/or the incubating in vi) further comprises a least a portion of incubating under static conditions.

Embodiment 226. The method of any of embodiments 222-225, wherein the incubating comprises a first incubation under static conditions followed by the incubating with motion.

Embodiment 227. The method of any of embodiments 222-225, wherein the incubating comprises the incubating with motion followed by a second incubation under static conditions.

Embodiment 228. The method of any of embodiments 222-227, wherein steps i)-iii) are repeated.

Embodiment 229. The method of embodiment 228, wherein the modifying in the first iteration of the method is different from the modifying in the repeated iteration of the method.

Embodiment 230. The method of any of embodiments 222-224, wherein the re-clustered islet cells are a first modified primary islet cluster and wherein the method further comprises:
iv) dissociating the first modified primary islet cluster into a suspension of modified primary beta islet cells;
v) further modifying the modified primary islet cells of the suspension; and
vi) incubating the further modified primary beta islet cells under conditions for re-clustering into a second modified primary islet cluster, wherein at least a portion of the incubating is carried out with motion.

Embodiment 231. The method of any of embodiments 222-230, wherein prior to the incubating in iii), the method comprises selecting for islet cells that have been modified.

Embodiment 232. The method of embodiment 230 or embodiment 231, wherein prior to v), the method comprises selecting, from the dissociated islet cells in iv), beta islet cells that have been modified, and optionally repeating steps iii) and iv) on the selected islet cells.

Embodiment 233. The method of embodiment 222, 226, or 227, wherein after the incubating in vi), the method comprises dissociating the second modified primary islet cluster into a suspension of modified primary islet cells and selecting for islet cells that have been modified.

Embodiment 234. The method of any of embodiments 208-233, wherein the suspension is a single cell suspension.

Embodiment 235. The method of any of embodiments 232-234, wherein incubating the selected modified primary beta islet cells under conditions for re-clustering into a modified primary islet cluster, wherein at least a portion of the incubating is carried out with motion.

Embodiment 236. The method of any of embodiments 222-235, wherein the motion is shaking.

Embodiment 237. The method of embodiment 236, wherein the shaking comprises orbital motion.

Embodiment 238. The method of embodiment 236, wherein the shaking comprises bidirectional linear movement.

Embodiment 239. The method of embodiment 236 or embodiment 237, wherein the shaking is with an orbital shaker.

Embodiment 240. The method of any of embodiments 231-239, wherein the selecting comprises fluorescence-activated cell sorting (FACS).

Embodiment 241. The method of any of embodiments 230-240, wherein one of the first modifying or further modifying comprises reducing expression of one or more genes encoding an endogenous protein in the cell and the other of the first modifying or the further modifying comprises increasing expression of one or more exogenous proteins in the cell.

Embodiment 242. The method of any of embodiments 230-240, wherein the first modifying comprises reducing expression of one or more genes encoding an endogenous protein in the cell and the further modifying comprises increasing expression of one or more exogenous proteins in the cell.

Embodiment 243. The method of any of embodiments 230-242, wherein the first modifying comprises reducing expression of one or more major histocompatibility complex (MHC) class I molecules and/or one or more MHC class II molecules.

Embodiment 244. The method of any of embodiments 208-243, wherein the modifying is genetic engineering.

Embodiment 245. The method of any of embodiments 230-244, wherein the one or more MHC class I HLA is an HLA-A protein, an HLA-B protein, or HLA-C protein.

Embodiment 246. The method of any of embodiments 230-245, wherein the one or more MHC class II HLA is an HLA-DP protein, an HLA-DR protein, or an HLA-DQ protein.

Embodiment 247. The method of any of embodiments 243-246, wherein reducing expression of one or more MHC class I molecules is by reducing expression of B-2 microglobulin (B2M).

Embodiment 248. The method of any of embodiments 243-247, wherein reducing expression of one or more MHC class II molecules is by reducing expression of CIITA.

Embodiment 249. The method of any of embodiments 230-248, wherein the further modifying comprises increasing expression of one or more tolerogenic factor in the cell.

Embodiment 250. The method of embodiment 249, wherein the one or more tolerogenic factor is selected from the group consisting of CD47, CD27, CD200, HLA-C, HLA-E, HLA-E heavy chain, HLA-G, PD-L1, IDO1, CTLA4-Ig, C1-Inhibitor, IL-10, IL-35, FASL, CCL21, MFGE8, and SERPINB9, and any combination thereof.

Embodiment 251. The method of embodiment 250, wherein the one or more tolerogenic factor is selected from the group consisting of CD47, PD-L1, HLA-E, HLA-G, CCL21, FASL, SERPINB9, CD200, MFGE8, and any combination thereof.

Embodiment 252. The method of embodiment 251, wherein at least one of the one or more tolerogenic factor is CD47.

Embodiment 253. The method of any of embodiments 224-252, wherein reducing expression of one or more genes encoding an endogenous protein in the cell is by introducing a gene-editing system into the cell.

Embodiment 254. The method of embodiment 253, wherein the gene-editing system comprises a sequence specific nuclease.

Embodiment 255. The method of embodiment 254, wherein the sequence specific nuclease is selected from the group consisting of a RNA-guided DNA endonuclease, a meganuclease, a transcription activator-like effector nuclease (TALEN), and a zinc-finger nuclease (ZFN).

Embodiment 256. The method of embodiment 255, wherein the gene-editing system comprises an RNA-guided nuclease.

Embodiment 257. The method of embodiment 255, wherein the RNA-guided nuclease comprises a Cas nuclease and a guide RNA.

Embodiment 258. The method of embodiment 256 or embodiment 257, wherein the RNA-guided-nuclease is a Type II or Type V Cas protein.

Embodiment 259. The method of embodiment 256, 257, or 258, wherein the RNA-guided-nuclease is a Cas9 homologue or a Cpf1 homologue.

Embodiment 260. The method of any of embodiments 224-259, wherein increasing expression of one or more exogenous proteins in the cell is by introducing an exogenous polynucleotide.

Embodiment 261. The method of embodiment 260, wherein the exogenous polynucleotide is operably linked to a promoter.

Embodiment 262. The method of embodiment 261, wherein the promoter is a constitutive promoter.

Embodiment 263. The method of embodiment 261 or embodiment 262, wherein the promoter is selected from the group consisting of the CAG promoter, the cytomegalovirus (CMV) promoter, the EF1a promoter, the PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein barr virus (EBV) promoter, and the Rous sarcoma virus (RSV) promoter.

Embodiment 264. The method of any of embodiments 224-259, wherein the exogenous polynucleotide is integrated into the genome of the cell.

Embodiment 265. The method of embodiment 264, wherein the exogenous polynucleotide is a multicistronic vector.

Embodiment 266. The method of embodiment 264, wherein the integration is by non-targeted insertion into the genome of the cell, optionally by introduction of the exogenous polynucleotide into the cell using a lentiviral vector.

Embodiment 267. The method of embodiment 264, wherein the integration is by targeted insertion into a target genomic locus of the cell.

Embodiment 268. The method of any of embodiments 119-267, wherein the islet cell is a beta islet cell.

Embodiment 269. The method of any of embodiments 67-115, wherein the engineered primary cell is a hepatocyte.

Embodiment 270. The method of any of embodiments 67-115, wherein the engineered primary cell is a T cell.

Embodiment 271. The method of any of embodiments 67-115, wherein the engineered primary cell is an endothelial cell.

Embodiment 272. The method of any of embodiments 67-115, wherein the engineered primary cell is a thyroid cell.

Embodiment 273. The method of any of embodiments 67-115, wherein the engineered primary cell is a skin cell.

Embodiment 274. The method of any of embodiments 67-115, wherein the engineered primary cell is a retinal pigmented epithelium cell.

Embodiment 275. An engineered primary cell produced according to the method of any of embodiments 67-274.

Embodiment 276. The engineered primary cell of embodiment 275, wherein the primary cell is an islet cell.

Embodiment 277. The engineered primary cell of embodiment 276, wherein the islet cell is a beta islet cell.

Embodiment 278. The engineered primary cell of any one of embodiments 1-66 and 275-277, wherein the engineered primary cell is capable of evading NK cell mediated cytotoxicity upon administration to a recipient patient.

Embodiment 279. The engineered primary cell of any one of embodiments 1-66 and 275-278, wherein the engineered primary cell is protected from cell lysis by mature NK cells upon administration to a recipient patient.

Embodiment 280. The engineered primary cell of any one of embodiments 1-66 and 275-279, wherein the engineered primary cell does not induce an immune response to the cell upon administration to a recipient patient.

Embodiment 281. The engineered primary cell of any one of embodiments 1-66 and 275-280, wherein the engineered primary cell does not induce a systemic inflammatory response to the cell upon administration to a recipient patient.

Embodiment 282. The engineered primary cell of any one of embodiments 1-66 and 275-281, wherein the engineered primary cell does not induce a local inflammatory response to the cell upon administration to a recipient patient.

Embodiment 283. A population of engineered primary cells comprising a plurality of the engineered primary cells of any of embodiments 1-66 and 275-282.

Embodiment 284. The population of engineered primary cells of embodiment 283, wherein the plurality of the engineered primary cells are derived from cells pooled from more than one donor subject.

Embodiment 285. The population of engineered primary cells of embodiment 284, wherein each of the more than one donor subjects are healthy subjects or are not suspected of having a disease or condition at the time the donor sample is obtained from the donor subject.

Embodiment 286. The population of any of embodiments 283-285, wherein at least 50%, 60%, 70%, 80%, 90%, 95%, 96%, 97%, 98%, 99%, 99.9%, or 99.99% of cells in the population comprise the modifications.

Embodiment 287. The population of any of embodiments 283-286, wherein at least 50%, 60%, 70%, 80%, 90%, 95%, 96%, 97%, 98%, 99%, 99.9%, or 99.99% of cells in the population comprise an exogenous polynucleotide encoding CD47.

Embodiment 288. The population of embodiment 136 or embodiment 137, wherein at least 50%, 60%, 70%, 80%, 90%, 95%, 96%, 97%, 98%, 99%, 99.9%, or 99.99% of cells in the population comprise reduced expression of one or more MHC class I molecules and/or one or more MHC class II molecules relative to a cell of the same cell type that does not comprises the modification(s).

Embodiment 289. The population of any of embodiments 283-288, wherein at least 50%, 60%, 70%, 80%, 90%, 95%, 96%, 97%, 98%, 99%, 99.9%, or 99.99% of cells in the population comprise reduced expression of B2M and/or CIITA relative to a cell of the same cell type that does not comprises the modification(s).

Embodiment 290. The population of any of embodiments 283-289, wherein at least 50%, 60%, 70%, 80%, 90%, 95%, 96%, 97%, 98%, 99%, 99.9%, or 99.99% of cells in the population comprise reduced expression of B2M relative to a cell of the same cell type that does not comprises the modification(s).

Embodiment 291. The population of any of embodiments 283-290, wherein at least 50%, 60%, 70%, 80%, 90%, 95%, 96%, 97%, 98%, 99%, 99.9%, or 99.99% of cells in the population comprise reduced expression of B2M and CIITA relative to a cell of the same cell type that does not comprises the modification(s).

Embodiment 292. The population of any of embodiments 283-291, wherein at least 50%, 60%, 70%, 80%, 90%, 95%, 96%, 97%, 98%, 99%, 99.9%, or 99.99% of cells in the population comprise one or more alterations that inactivate both alleles of an endogenous B2M gene.

Embodiment 293. The population of any of embodiments 283-292, wherein at least 50%, 60%, 70%, 80%, 90%, 95%, 96%, 97%, 98%, 99%, 99.9%, or 99.99% of cells in the population comprise one or more alterations that inactivate both alleles of an endogenous CIITA gene.

Embodiment 294. A composition comprising the population of any of embodiments 283-293.

Embodiment 295. A composition comprising an engineered primary islet cluster produced by the method of any of embodiments 119-268.

Embodiment 296. A composition comprising a population of engineered primary islet cells, wherein the engineered primary islet cells comprise: (i) a transgene comprising an exogenous polynucleotide encoding CD47 and (ii) inactivation or disruption of both alleles of a B2M gene.

Embodiment 297. The composition of embodiment 296, wherein the population of engineered primary islet cells is a cluster of primary islet cells.

Embodiment 298. The composition of embodiment 296, wherein the population of engineered primary islet cells is a population of engineered primary beta islet cells.

Embodiment 299. A composition comprising a population of engineered primary T cells, wherein the engineered primary T cells comprise: (i) a transgene comprising an exogenous polynucleotide encoding CD47 and (ii) inactivation or disruption of both alleles of a B2M gene.

Embodiment 300. A composition comprising a population of engineered primary thyroid cells, wherein the engineered primary thyroid cells comprise: (i) a transgene comprising an exogenous polynucleotide encoding CD47 and (ii) inactivation or disruption of both alleles of a B2M gene.

Embodiment 301. A composition comprising a population of engineered primary skin cells, wherein the engineered primary skin cells comprise: (i) a transgene comprising an exogenous polynucleotide encoding CD47 and (ii) inactivation or disruption of both alleles of a B2M gene.

Embodiment 302. A composition comprising a population of engineered primary endothelial cells, wherein the engineered primary endothelial cells comprise: (i) a transgene comprising an exogenous polynucleotide encoding CD47 and (ii) inactivation or disruption of both alleles of a B2M gene.

Embodiment 303. A composition comprising a population of engineered primary retinal pigmented epithelium cells, wherein the engineered primary retinal pigmented epithelium cells comprise: (i) a transgene comprising an exogenous polynucleotide encoding CD47 and (ii) inactivation or disruption of both alleles of a B2M gene.

Embodiment 304. The composition of any of embodiments 294-303, wherein engineered primary cells of the population of engineered primary cells comprise an indel in both alleles of the B2M gene.

Embodiment 305. The composition of any of embodiments 294-304, wherein the engineered primary cells of the population of engineered primary cells further comprise inactivation or disruption of both alleles of a CIITA gene.

Embodiment 306. The composition of any of embodiments 294-305, wherein engineered primary cells of the population of engineered primary cells comprise an indel in both alleles of the CIITA gene.

Embodiment 307. The composition of any of embodiments 294-306, wherein the engineered primary cells of the population of engineered primary cells have the phenotype B2M^{indel/indel}; CIITA^{indel/indel}; CD47tg.

Embodiment 308. The composition of any of embodiments 294-307, wherein the composition is a pharmaceutical composition.

Embodiment 309. The composition of any of embodiments 294-308, comprising a pharmaceutically acceptable excipient.

Embodiment 310. The composition of any of embodiments 294-309, wherein the composition is formulated in a serum-free cryopreservation medium comprising a cryoprotectant.

Embodiment 311. The composition of embodiment 309, wherein the cryoprotectant is DMSO and the cryopreservation medium is 5% to 10% DMSO (v/v).

Embodiment 312. The composition of embodiment 308 and embodiment 309, wherein the cryoprotectant is or is about 10% DMSO (v/v).

Embodiment 313. The composition of any of embodiments 294-312 that is sterile.

Embodiment 314. A container, comprising the composition of any of embodiments 294-313.

Embodiment 315. The container of embodiment 314 that is a sterile bag.

Embodiment 316. The sterile bag of embodiment 315, wherein the bag is a cryopreservation-compatible bag.

Embodiment 317. A method of treating a disease, condition, or cellular deficiency in a patient in need thereof comprising administering to the patient an effective amount of the population of any of embodiments 283-293, the composition of any of embodiments 294-307, or the pharmaceutical composition of embodiment 308.

Embodiment 318. The method of 317, wherein the population is formulated as a pharmaceutical composition comprising a pharmaceutically acceptable excipient.

Embodiment 319. The method of embodiment 317 or embodiment 318, wherein the population of cells comprises islet cells, including beta islet cells.

Embodiment 320. The method of any one of embodiments 317-319, wherein the population of islet cells is administered as a cluster of islet cells.

Embodiment 321. The method of any one of embodiments 317-320, wherein the population of islet cells is administered as a cluster of beta islet cells.

Embodiment 322. The method of any of embodiments 317-320, wherein the population of cells are hepatocytes.

Embodiment 323. The method of embodiment 317 or embodiment 318, wherein the population of cells comprises T cells.

Embodiment 324. The method of embodiment 317 or embodiment 318, wherein the population of cells comprises thyroid cells.

Embodiment 325. The method of embodiment 317 or embodiment 318, wherein the population of cells comprises skin cells.

Embodiment 326. The method of embodiment 317 or embodiment 318, wherein the population of cells comprises endothelial cells.

Embodiment 327. The method of embodiment 317 or embodiment 318, wherein the population of cells comprises retinal pigmented epithelium cells.

Embodiment 328. The method of embodiment 317-327, wherein the condition or disease is selected from the group consisting of diabetes, cancer, vascularization disorders, ocular disease, thyroid disease, skin diseases, and liver diseases.

Embodiment 329. The method of embodiment 317 or embodiment 318, wherein the cellular deficiency is associated with diabetes or the cellular therapy is for the treatment of diabetes, optionally wherein the diabetes is Type I diabetes.

Embodiment 330. The method of embodiment 329, wherein the population of cells is a population of islet cells, including beta islet cells.

Embodiment 331. The method of embodiment 330, wherein the population of cells is administered as a cluster of islet cells.

Embodiment 332. A method of treating diabetes in a patient in need thereof, the method comprising administering to the patient an effective amount of a population of islet cells of any of embodiments 283-293, the composition of any of embodiments 294-307, or the pharmaceutical composition of embodiment 308.

Embodiment 333. The method of any of embodiments 330-331, wherein the cluster of islet cells is a cluster of beta islet cells.

Embodiment 334. The method of embodiment 317 or embodiment 318, wherein the cellular deficiency is associated with a vascular condition or disease or the cellular therapy is for the treatment of a vascular condition or disease.

Embodiment 335. The method of embodiment 334, wherein the population of cells is a population of endothelial cells.

Embodiment 336. The method of embodiment 317 or embodiment 318, wherein the cellular deficiency is associated with autoimmune thyroiditis or the cellular therapy is for the treatment of autoimmune thyroiditis.

Embodiment 337. The method of embodiment 317 or embodiment 318, wherein the cellular deficiency is associated with a liver disease or the cellular therapy is for the treatment of liver disease.

Embodiment 338. The method of embodiment 337, wherein the liver disease comprises cirrhosis of the liver.

Embodiment 339. The method of embodiment 337 or embodiment 338, wherein the population of cells is a population of hepatocytes.

Embodiment 340. The method of embodiment 317 or embodiment 318, wherein the cellular deficiency is associated with a corneal disease or the cellular therapy is for the treatment of corneal disease.

Embodiment 341. The method of embodiment 340, wherein the corneal disease is Fuchs dystrophy or congenital hereditary endothelial dystrophy.

Embodiment 342. The method of embodiment 340 or embodiment 341, wherein the population of cells is a population of corneal endothelial cells.

Embodiment 343. The method of embodiment 317 or embodiment 318, wherein the cellular deficiency is associated with a kidney disease or the cellular therapy is for the treatment of a kidney disease.

Embodiment 344. The method of embodiment 343, wherein the population of cells is a population of renal cells.

Embodiment 345. The method of embodiment 317 or embodiment 318, wherein the cellular therapy is for the treatment of a cancer.

Embodiment 346. The method of embodiment 345, wherein the cancer is selected from the group consisting of B cell acute lymphoblastic leukemia (B-ALL), diffuse large B-cell lymphoma, liver cancer, pancreatic cancer, breast cancer, ovarian cancer, colorectal cancer, lung cancer, non-small cell lung cancer, acute myeloid lymphoid leukemia, multiple myeloma, gastric cancer, gastric adenocarcinoma, pancreatic adenocarcinoma, glioblastoma, neuroblastoma, lung squamous cell carcinoma, hepatocellular carcinoma, and bladder cancer.

Embodiment 347. The method of embodiment 317 or embodiment 318, wherein the population of cells is a population of T cells or NK cells.

Embodiment 348. The method of any of embodiments 317-347, wherein the cells are expanded and cryopreserved prior to administration.

Embodiment 349. The method of any of embodiments 317-348, wherein administering the population comprises intravenous injection, intramuscular injection, intravascular injection, or transplantation of the population.

Embodiment 350. The method of embodiment 349, wherein the population is transplanted via kidney capsule transplant or intramuscular injection.

Embodiment 351. The method of any of embodiments 317-350, wherein the population is derived from a donor subject, wherein the HLA type of the donor does not match the HLA type of the patient.

Embodiment 352. The method of any of embodiments 317-351, wherein the population is a human cell population and the patient is a human patient.

Embodiment 353. The method of any of embodiments 330-333, wherein the beta islet cells improve glucose tolerance in the subject.

Embodiment 354. The method of embodiment 353, wherein the subject is a diabetic patient.

Embodiment 355. The method of embodiment 354, wherein the diabetic patient has type I diabetes or type II diabetes

Embodiment 356. The method of any of embodiments 330-332 and 353-55, wherein glucose tolerance is improved relative to the subject's glucose tolerance prior to administration of the islet cells.

Embodiment 357. The method of any of embodiments 330-332 and 353-356, wherein the beta islet cells reduce exogenous insulin usage in the subject.

Embodiment 358. The method of any of embodiments 353-357, wherein glucose tolerance is improved as measured by HbA1c levels.

Embodiment 359. The method of any of embodiments 353-358, wherein the subject is fasting.

Embodiment 360. The method of any one of embodiments 330-332 and 351-360 wherein the islet cells improve insulin secretion in the subject.

Embodiment 361. The method of embodiment 360, wherein insulin secretion is improved relative to the subject's insulin secretion prior to administration of the islet cells.

Embodiment 362. The method of any of embodiments 317-361, further comprising administering one or more immunosuppressive agents to the patient.

Embodiment 363. The method of any of embodiments 317-361, where the patient has been administered one or more immunosuppressive agents.

Embodiment 364. The method of embodiment 362 or 363, wherein the one or more immunosuppressive agents are a small molecule or an antibody.

Embodiment 365. The method of any of embodiments 362-364, wherein the one or more immunosuppressive agents are selected from the group consisting of cyclosporine, azathioprine, mycophenolic acid, mycophenolate mofetil, a corticosteroids, prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin (thymosin-α), and an immunosuppressive antibody.

Embodiment 366. The method of any of embodiments 362-364, wherein the one or more immunosuppressive agents comprise cyclosporine.

Embodiment 367. The method of any of embodiments 362-364, wherein the one or more immunosuppressive agents comprise mycophenolate mofetil.

Embodiment 368. The method of any of embodiments 362-364, wherein the one or more immunosuppressive agents comprise a corticosteroid.

Embodiment 369. The method of any of embodiments 362-364, wherein the one or more immunosuppressive agents comprise cyclophosphamide.

Embodiment 370. The method of any of embodiments 362-364, wherein the one or more immunosuppressive agents comprise rapamycin.

Embodiment 371. The method of any of embodiments 362-364, wherein the one or more immunosuppressive agents comprise tacrolimus (FK-506).

Embodiment 372. The method of any of embodiments 362-364, wherein the one or more immunosuppressive agents comprise anti-thymocyte globulin.

Embodiment 373. The method of any of embodiments 362-364, wherein the one or more immunosuppressive agents are one or more immunomodulatory agents.

Embodiment 374. The method of embodiment 373, wherein the one or more immunomodulatory agents are a small molecule or an antibody.

Embodiment 375. The method of embodiment 364 or embodiment 374, wherein the antibody binds to one or more of receptors or ligands selected from the group consisting of p75 of the IL-2 receptor, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, CD58, and antibodies binding to any of their ligands.

Embodiment 376. The method of any of embodiments 362-375, wherein the one or more immunosuppressive agents are or have been administered to the patient prior to administration of the engineered cells.

Embodiment 377. The method of any of embodiments 362-376, wherein the one or more immunosuppressive agents are or have been administered to the patient at least 1, 2, 3, 4, 5, 6, 7, 8, 9, 10, 11, 12, 13, or 14 days prior to administration of the engineered cells.

Embodiment 378. The method of any of embodiments 362-376, wherein the one or more immunosuppressive agents are or have been administered to the patient at least 1 week, 2 weeks, 3 weeks, 4 weeks, 5 weeks, 6 weeks, 7 weeks, 8 weeks, 9 weeks, 10 weeks or more prior to administration of the engineered cells.

Embodiment 379. The method of any of embodiments 362-376, wherein the one or more immunosuppressive agents are or have been administered to the patient at least 1, 2, 3, 4, 5, 6, 7, 8, 9, 10, 11, 12, 13, or 14 days after administration of the engineered cells.

Embodiment 380. The method of any of embodiments 362-376, wherein the one or more immunosuppressive agents are or have been administered to the patient at least 1 week, 2 weeks, 3 weeks, 4 weeks, 5 weeks, 6 weeks, 7 weeks, 8 weeks, 9 weeks, 10 weeks, or more, after administration of the engineered cells.

Embodiment 381. The method of any of embodiments 362-376, wherein the one or more immunosuppressive agents are or have been administered to the patient on the same day as the first administration of the engineered cells.

Embodiment 382. The method of any of embodiments 362-376, wherein the one or more immunosuppressive agents are or have been administered to the patient after administration of the engineered cells.

Embodiment 383. The method of any of embodiments 362-376, wherein the one or more immunosuppressive agents are or have been administered to the patient after administration of a first and/or second administration of the engineered cells.

Embodiment 384. The method of any of embodiments 362-376, wherein the one or more immunosuppressive agents are or have been administered to the patient prior to administration of a first and/or second administration of the engineered cells.

Embodiment 385. The method of any of embodiments 362-376, wherein the one or more immunosuppressive agents are or have been administered to the patient at least 1, 2, 3, 4, 5, 6, 7, 8, 9, 10, 11, 12, 13, or 14 days prior to administration of a first and/or second administration of the engineered cells.

Embodiment 386. The method of any of embodiments 362-376, wherein the one or more immunosuppressive agents are or have been administered to the patient at least 1 week, 2 weeks, 3 weeks, 4 weeks, 5 weeks, 6 weeks, 7 weeks, 8 weeks, 9 weeks, 10 weeks or more prior to administration of a first and/or second administration of the engineered cells.

Embodiment 387. The method of any of embodiments 362-376, wherein the one or more immunosuppressive agents are or have been administered to the patient at least 1, 2, 3, 4, 5, 6, 7, 8, 9, 10, 11, 12, 13, or 14 days after administration of a first and/or second administration of the engineered cells.

Embodiment 388. The method of any of embodiments 362-376, wherein the one or more immunosuppressive agents are or have been administered to the patient at least 1 week, 2 weeks, 3 weeks, 4 weeks, 5 weeks, 6 weeks, 7 weeks, 8 weeks, 9 weeks, 10 weeks, or more, after administration of a first and/or second administration of the engineered cells.

Embodiment 389. The method of any of embodiments 362-388, wherein the one or more immunosuppressive agents are administered at a lower dosage compared to the dosage of one or more immunosuppressive agents administered to reduce immune rejection of immunogenic cells that do not comprise the modifications of the engineered cells.

Embodiment 390. The method of any of embodiments 361-389, wherein the engineered cell is capable of controlled killing of the engineered cell.

Embodiment 391. The method of any of embodiments 361-390, wherein the engineered cell comprises a suicide gene or a suicide switch.

Embodiment 392. The method of embodiment 391, wherein the suicide gene or the suicide switch induces controlled cell death in the presence of a drug or prodrug, or upon activation by a selective exogenous compound.

Embodiment 393. The method of embodiment 391 or embodiment 392, wherein the suicide gene or the suicide switch is an inducible protein capable of inducing apoptosis of the engineered cell.

Embodiment 394. The method of embodiment 393, wherein the inducible protein capable of inducing apoptosis of the engineered cell is a caspase protein.

Embodiment 395. The method of embodiment 394, wherein the caspase protein is caspase 9.

Embodiment 396. The method of embodiment 393 or embodiment 394, wherein the suicide gene or suicide switch is selected from the group consisting of cytosine deaminase (CyD), herpesvirus thymidine kinase (HSV-Tk), an inducible caspase 9 (iCaspase9), and rapamycin-activated caspase 9 (rapaCasp9).

Embodiment 397. The method of any of embodiments 391-396, wherein the suicide gene or the suicide switch is activated to induce controlled cell death after the administration of the one or more immunosuppressive agents to the patient.

Embodiment 398. The method of any of embodiments 391-396, wherein the suicide gene or the suicide switch is activated to induce controlled cell death prior to the administration of the one or more immunosuppressive agents to the patient.

Embodiment 399. The method of any of embodiments 391-398, wherein the suicide gene or the suicide switch is activated to induce controlled cell death after the administration of the engineered cell to the patient.

Embodiment 400. The method of any of embodiments 391-399, wherein the suicide gene or the suicide switch is activated to induce controlled cell death in the event of cytotoxicity or other negative consequences to the patient.

Embodiment 401. The method of any of embodiments 361-391, comprising administering an agent that allows for depletion of an engineered cell of the population of engineered cells.

Embodiment 402. The method of embodiment 401, wherein the agent that allows for depletion of the engineered cell is an antibody that recognizes a protein expressed on the surface of the engineered cell.

Embodiment 403. The method of embodiment 402, wherein the antibody is selected from the group consisting of an antibody that recognizes CCR4, CD16, CD19, CD20, CD30, EGFR, GD2, HER1, HER2, MUC1, PSMA, and RQR8.

Embodiment 404. The method of embodiment 401 or embodiment 402, wherein the antibody is selected from the group consisting of mogamulizumab, AFM13, MOR208, obinutuzumab, ublituximab, ocaratuzumab, rituximab, rituximab-Rllb, tomuzotuximab, RO5083945 (GA201), cetuximab, Hu14.18K322A, Hu14.18-IL2, Hu3F8, dinituximab, c.60C3-Rllc, and biosimilars thereof.

Embodiment 405. The method of any of embodiments 317-361 and 401-404, comprising administering an agent that recognizes the one or more tolerogenic factors on the surface of the engineered cell.

Embodiment 406. The method of embodiment 405, wherein the engineered cell is engineered to express the one or more tolerogenic factors.

Embodiment 407. The method of embodiment 405 or embodiment 406, wherein the one or more tolerogenic factors is CD47.

Embodiment 408. The method of any of embodiments 317-407, further comprising administering one or more additional therapeutic agents to the patient.

Embodiment 409. The method of any of embodiments 317-408, wherein the patient has been administered one or more additional therapeutic agents.

Embodiment 410. The method of any of embodiments 317-409, further comprising monitoring the therapeutic efficacy of the method.

Embodiment 411. The method of any of embodiments 317-410, further comprising monitoring the prophylactic efficacy of the method.

Embodiment 412. The method of embodiment 410 or embodiment 411, wherein the method is repeated until a desired suppression of one or more disease symptoms occurs.

Embodiment 413. The engineered cell of any of embodiments 1-66 and 275-282, wherein the engineered cell comprises an exogenous polynucleotide encoding a suicide gene or a suicide switch.

Embodiment 414. The engineered cell of embodiment 413, wherein the suicide gene or suicide switch is selected from the group consisting of cytosine deaminase (CyD), herpesvirus thymidine kinase (HSV-Tk), an inducible caspase 9 (iCaspase9), and rapamycin-activated caspase 9 (rapaCasp9).

Embodiment 415. The engineered cell of embodiment 413 or embodiment 414, wherein the suicide gene or suicide switch and genes associated with the suicide gene or the safety switch are expressed from a bicistronic cassette integrated into the genome of the engineered cell.

Embodiment 416. The engineered cell of any of embodiments 413-415, wherein the suicide gene or suicide switch and the one or more tolerogenic factors are expressed from a bicistronic cassette integrated into the genome of the engineered cell.

Embodiment 417. The engineered cell of embodiment 415 or embodiment 416, wherein the bicistronic cassette is integrated by non-targeted insertion into the genome of the engineered cell, optionally by introduction of the exogenous polynucleotide into the cell using a lentiviral vector.

Embodiment 418. The engineered cell of embodiment 417, wherein the bicistronic cassette is integrated by targeted insertion into a target genomic locus of the cell, optionally wherein the targeted insertion is by nuclease-mediated gene editing with homology-directed repair.

Embodiment 419. The engineered cell of any of embodiments 412-418, wherein the one or more tolerogenic factors is CD47.

Embodiment 420. The method of any of embodiments 67-274, wherein the engineered cell comprises an exogenous polynucleotide encoding a suicide gene or suicide switch.

Embodiment 421. The method of embodiment 420, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

Embodiment 422. The method of embodiment 420 or embodiment 421, wherein the suicide gene or suicide switch and genes associated with the suicide gene or the safety switch are expressed from a bicistronic cassette integrated into the genome of the engineered cell.

Embodiment 423. The method of any of embodiments 420-422, wherein the suicide gene or suicide switch and the one or more tolerogenic factors are expressed from a bicistronic cassette integrated into the genome of the engineered cell.

Embodiment 424. The method of embodiment 422 or embodiment 423, wherein the bicistronic cassette is integrated by non-targeted insertion into the genome of the engineered cell.

Embodiment 425. The method of embodiment 424, wherein the bicistronic cassette is integrated by targeted insertion into a target genomic locus of the engineered cell.

Embodiment 426. The method of any of embodiments 420-425, wherein the one or more tolerogenic factors is CD47.

Embodiment 427. The composition of any of embodiments 294-313, wherein engineered cells of the population of engineered cells comprise an exogenous polynucleotide encoding a suicide gene or a suicide switch.

Embodiment 428. The composition of embodiment 427, wherein the suicide gene or suicide switch is selected from the group consisting of cytosine deaminase (CyD), herpesvirus thymidine kinase (HSV-Tk), an inducible caspase 9 (iCaspase9), and rapamycin-activated caspase 9 (rapaCasp9).

Embodiment 429. The composition of embodiment 427 or embodiment 428, wherein the suicide gene and genes associated with the suicide gene or the safety switch are expressed from a bicistronic cassette integrated into the genome of engineered cells of the population of engineered cells.

Embodiment 430. The composition of any of embodiments 427-429, wherein the suicide gene or suicide switch and the exogenous CD47 are expressed from a bicistronic cassette integrated into the genome of the engineered cell.

Embodiment 431. The composition of embodiment 429 or embodiment 430, wherein the bicistronic cassette is integrated by non-targeted insertion into the genome, optionally by introduction of the exogenous polynucleotide into engineered cells of the population of engineered cells using a lentiviral vector.

Embodiment 432. The composition of embodiment 429 or embodiment 430, wherein the bicistronic cassette is integrated by targeted insertion into a target genomic locus of engineered cells of the population of engineered cells, optionally wherein the targeted insertion is by nuclease-mediated gene editing with homology-directed repair.

### VII. Examples

The following examples are included for illustrative purposes only and are not intended to limit the scope of the invention.

### Example 1: Survival and Function of B2M^{-/-}; CD47tg Primary Beta Islet Cells in a Transplant Study

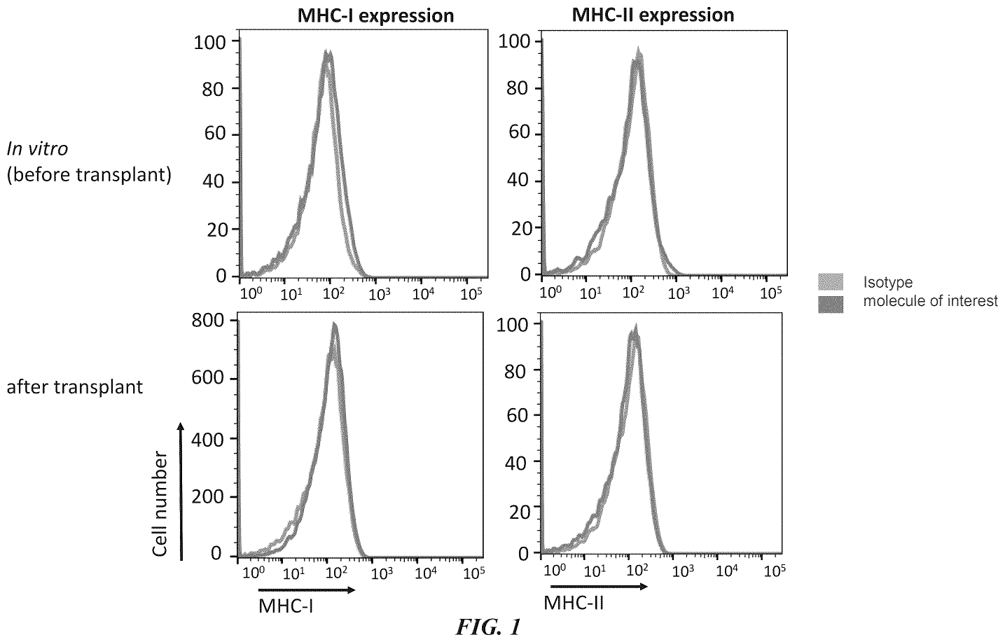

To study the effects of reducing MHC class I and MHC class II expression and increasing CD47 expression for transplant of primary beta islet cells in an allogeneic recipient, primary beta islet cells from B2M-knockout C57BL/6 (B6) mice (MHC haplotype H2^{b}) were transduced with a lentiviral vector encoding CD47 to generate mouse B2M^{-/-}; CD47tg primary beta islet cells. B2M^{-/-} primary beta islet cells isolated from C57BL/6 (B6) mice do not naturally express MHC-II molecules (FIG. 1), nor are MHC-II molecules upregulated following stimulation. The mouse B2M^{-/-}; CD47tg engineered primary beta islet cells were transplanted into BALB/c diabetes disease mouse model recipients (MHC haplotype H2^{d}). Survival and function of the transplanted mouse B2M^{-/-}; CD47tg B6 primary beta islet cells compared to transplanted mouse wild type B6 islet cells were monitored over time.

### A. Methods

Diabetes mouse model. Twenty-five BALB/c (MHC haplotype H2^{d}) mice were injected with low dose streptozotocin (STZ) (60 mg/kg i.p.) daily for 5 days (day −5 to day 0). STZ was dissolved in citrate buffer (10 mg/ml stock solution) and diluted to an injection volume of 150 μL for intraperitoneal (i.p.) injection. Phosphate buffered saline (PBS) (1 mL) i.p. injections were administered to the mice the next morning to keep the kidneys healthy.

Blood glucose measurements. Blood glucose measurements were taken 4 hours after food withdrawal according to standard protocols.

Generation of mouse primary beta islet cells. B2M^{-/-} primary beta islet cells were isolated from B2M-knockout C57BL/6 (B6) mice (MHC haplotype H2^{b}). The isolated mouse B2M^{-/-} primary beta islet cells were transduced with a lentiviral vector encoding a mouse CD47 transgene. Primary beta islet cells isolated from wild type B6 mice were used as controls (wild type islets). Both mouse wild type and mouse B2M^{-/-} CD47tg primary beta islet cells were transduced with a luciferase expression construct for monitoring cell survival via bioluminescence imaging (BLI).

Flow cytometry. Surface expression of MHC class I, MHC class II, and CD47 on primary beta islet cells were assessed by flow cytometry using antibody-specific reagents. Isotype antibodies were used as a control.

Transplant study design and administration. Twenty-five BALB/c MHC haplotype H2^{d} mice (mouse body weight 18-20 g) were randomized into five study groups (n=5 per study group) post STZ administration to induce diabetes, in which the groups differed based on the cells that were administered (mouse wild type versus mouse B2M^{-/-} CD47tg B6 primary beta islet cells) and/or the route of administration (direct injection into kidney capsule versus intramuscular, i.m. administration). The study groups were as follows: mouse wild type B6 primary beta islet (luc+) kidney capsule transplant; mouse wild type B6 primary beta islet (luc+) i.m. transplant; no transplant diabetes control; mouse B2M^{-/-} CD47tg B6 primary beta islet (luc+) kidney capsule transplant; and mouse B2M^{-/-} CD47tg B6 primary beta islet (luc+) i.m. transplant.

Islet clusters of about 1500 cells per cluster were transplanted by kidney capsule injection or i.m. injection into mice. For kidney capsule injection, 300 clusters (about 450,000 cells) were transplanted per mouse. For i.m injection, 600 clusters (about 900,000 cells) were injected per mouse. Day 0 (d0) was defined as the day of transplantation.

Cell survival was measured by bioluminescence imaging (BLI) on d0, d3, d5, d7, d9, d11, d13, d17, d21, d25 and d29 for mouse B2M^{-/-} CD47tg B6 primary beta islets (for wild type B6 primary beta islets, BLI imaging was discontinued after day 5, because no signal was detected after day 5). Glucose was measured after 4 hours of fasting prior to islet implantation (pre-STZ, d−3, d−2, and d−1) and on d0, d3, d14, d21, d28, d30, d31, d32, d36. On d29, kidneys were removed for histopathological analysis from the cohort with kidney capsule implantation. Primary beta islet cell isolation was performed as described in Li, et al. “A protocol for islet isolation from mouse pancreas.” Nat Protoc. Vol. 4, 11 (2009):1649-52, the contents of which are herein incorporated by reference in their entirety. Kidney isolation was performed as described in Mathews et al. “New mouse model to study islet transplantation in insulin-dependent diabetes mellitus.” Transplantation. Vol. 73, 8 (2006):1333-6.

CL. Results

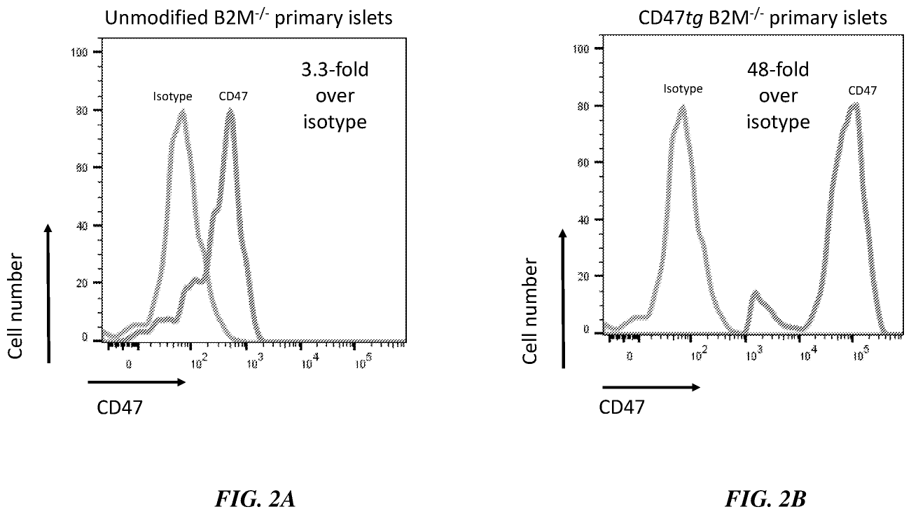

Mouse B2M^{-/-} CD47tg B6 primary beta islet cells do not express MHC-I or MHC-H, and have increased CD47 expression. To evaluate the expression of the MHC-I and MCH-II and CD47 in the mouse B2M^{-/-}; CD47tg primary beta islet cells, flow cytometry was performed. Isolated mouse B2M^{-/-}; CD47tg primary beta islet cells were negative for MHC-I and MHC-II before and after transplantation (FIG. 1). Mouse B2M^{-/-} primary beta islet cells that were not engineered to overexpress CD47 exhibited a low level surface expression of CD47 (3.3-fold over isotype control, as shown in FIG. 2A). Mouse B2M^{-/-}; CD47tg primary beta islet cells showed increased expression of CD47 (48-fold over isotype control, as shown in FIG. 2B).

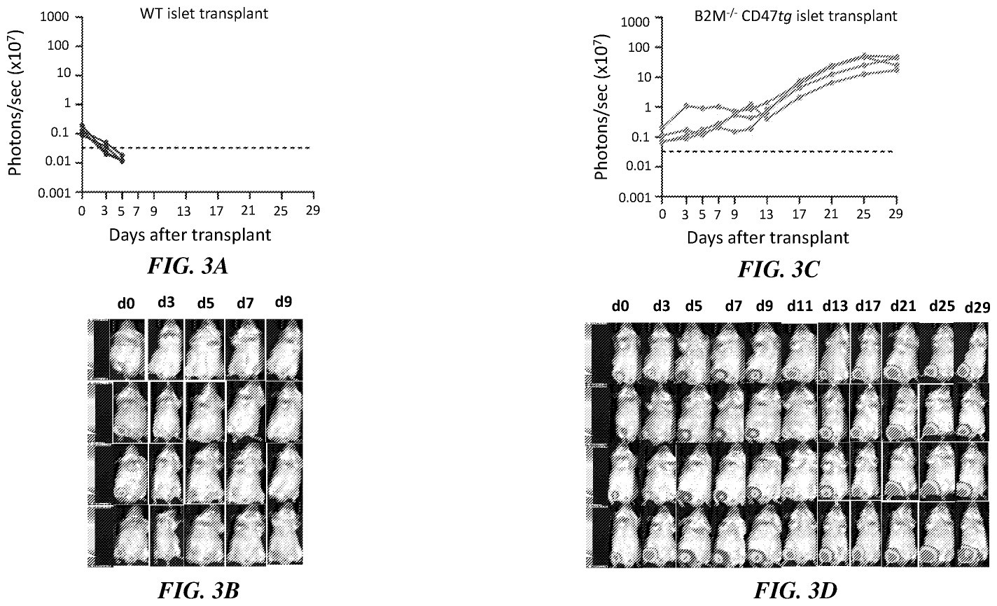

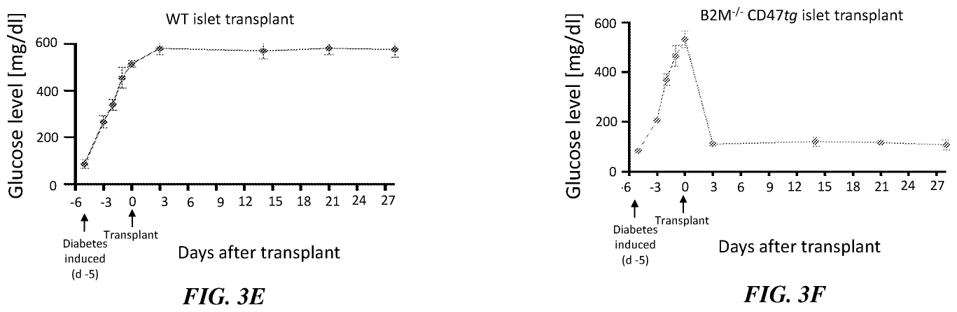

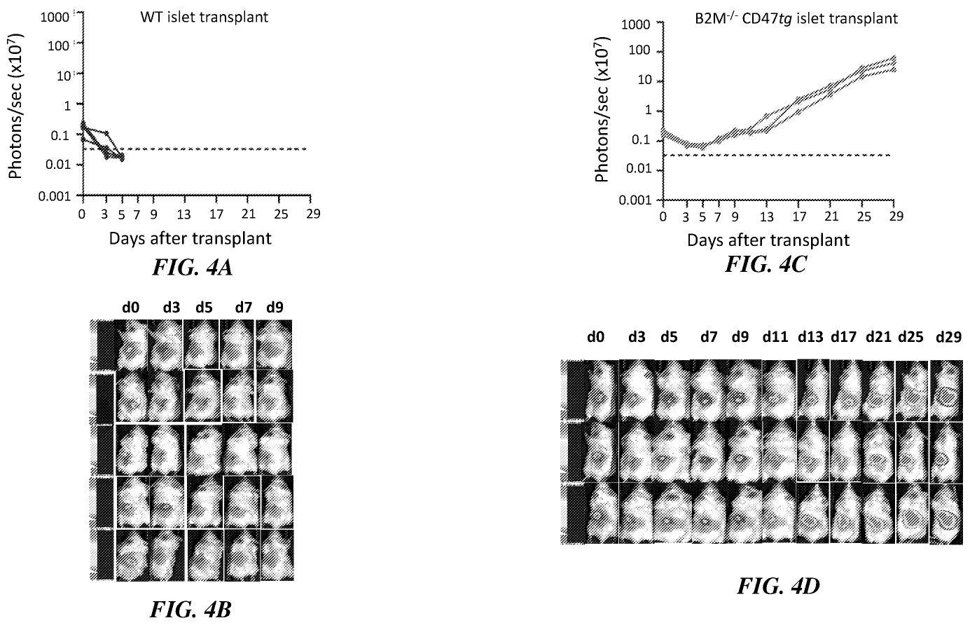

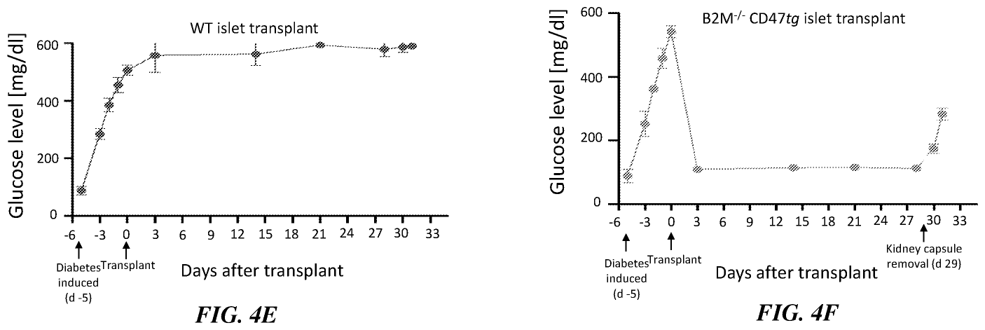

Mouse B2M^{-/-}; CD47tg primary beta islet cells survive after allogeneic transplant. Quantification of BLI imaging results of mouse primary beta islet cells after i.m. injection are shown in FIG. 3A (mouse wild type B6 primary beta islet cells) and FIG. 3C (mouse B2M^{-/-}; CD47tg B6 primary beta islet cells) and after kidney capsule injection are shown in FIG. 4A (mouse wild type B6 primary beta islet cells) and FIG. 4C (mouse B2M^{-/-}; CD47tg B6 primary beta islet cells). Corresponding BLI images of mouse primary beta islet cells after i.m. injection are shown in FIG. 3B (mouse wild type B6 primary beta islet cells) and FIG. 3D (mouse B2M^{-/-}; CD47tg B6 primary beta islet cells) and after kidney capsule injection are shown in FIG. 4B (mouse wild type B6 primary beta islet cells) and FIG. 4D (mouse B2M^{-/-}; CD47tg B6 primary beta islet cells). Bioluminescence was initially observed at the i.m. injection site for all groups following administration of the mouse primary beta islet cells. However, the number of photons detected for transplanted mouse wild type B6 primary beta islet cells rapidly declined over the first 5 days after transplant (FIG. 3A and FIG. 4A), indicating death of the mouse wild type B6 primary beta islet cells, likely due to an immune response. In contrast, the number of photons detected from transplanted mouse B2M^{-/-}; CD47tg B6 primary beta islet cells increased over the course of 29 days post-transplant, indicating survival and growth of the mouse B2M^{-/-}; CD47tg B6 primary beta islet cells (FIG. 3C and FIG. 4C).

Mouse B2M^{-/-}; CD47tg primary beta islet cells function after allogeneic transplant. To analyze function of transplanted mouse primary beta islet cells, blood glucose levels were measured at the indicated time points after transplant. Blood glucose levels were between 80 and 120 mg/dL in naive (untreated) mice and >200 mg/dL in the diabetic (STZ treated) mice. Glucose levels in diabetic (STZ treated) mice receiving i.m. injection or kidney capsule injection of wild type B6 islets remained high (>400 mg/dL) over the full study period, as shown in FIG. 3E and FIG. 4E, respectively. In contrast, blood glucose levels in diabetic (STZ treated) mice receiving i.m. injection or kidney capsule injection of mouse B2M^{-/-}; CD47tg B6 primary beta islet cells dropped soon after transplantation to non-diabetic levels (between 80 and 120 mg/dL), as shown in FIG. 3F and FIG. 4F, respectively. In the kidney capsule injection model, glucose levels began to rise following removal of the kidney on d29 (FIG. 4F).

Together, these data indicate that the mouse B2M^{-/-}; CD47tg primary beta islet cells were able to survive and function (e.g., restore lost glucose control due to diabetes) after allogeneic transplant in a diabetes disease model.

### Example 2: Immune Function of Mouse B2M^{-/-}; CD47tg Primary Beta Islet Cells in a Transplant Study

To study the immune evasion effects of reducing MHC class I and MHC class II expression and increasing CD47 expression on mouse primary beta islet cells, immune functional assays were performed on mouse B2M^{-/-}; CD47tg primary beta islet cells generated as described in Example 1 that were transplanted into BALB/c diabetes disease mouse model recipients (MHC haplotype H2^{d}). The survival and immune response elicited by transplantation of mouse B2M^{-/-}; CD47tg primary beta islet cells, compared to mouse wild type B6 primary beta islet cells, and mouse B2M^{-/-} B6 primary beta islet cells, was evaluated.

### A. Methods

Transplant study design and administration. Fifteen BALB/c MHC haplotype H2^{d} mice (mouse body weight 18-20 g) were randomized into five study groups (n=5 per study group) post STZ intramuscular (i.m.) administration to induce diabetes, in which the groups differed based on the cells that were administered (wild type versus B2M^{-/-} CD47tg B6 islet cells). The study groups were as follows: mouse wild type B6 primary beta islet cell (luc+) i.m. transplant; mouse B2M^{-/-}; CD47tg B6 primary beta islet cells (luc+) i.m. transplant and no transplant diabetes control.

Islet clusters of about 600 cells per cluster were transplanted by i.m. injection into mice. For i.m injection, 600 clusters (about 900,000 cells) were injected per mouse. Day 0 (d0) was defined as the day of transplantation. Glucose was measured after 4 hours of fasting prior to islet implantation (pre-STZ, d-3, d-2, and d-1) and on d6. On d6, mice were sacrificed for immune assay analysis.

T Cell Enzyme-Linked Immune absorbent SPOT (ELISPOT) Assay. Interferon gamma (IFNg)-secreting CD8(+) T cells in mouse primary beta islet cells were detected by ELISPOT.

Flow cytometry. Expression of donor specific antibodies (DSA) in primary beta islet cells was assessed by flow cytometry.

CL. Results

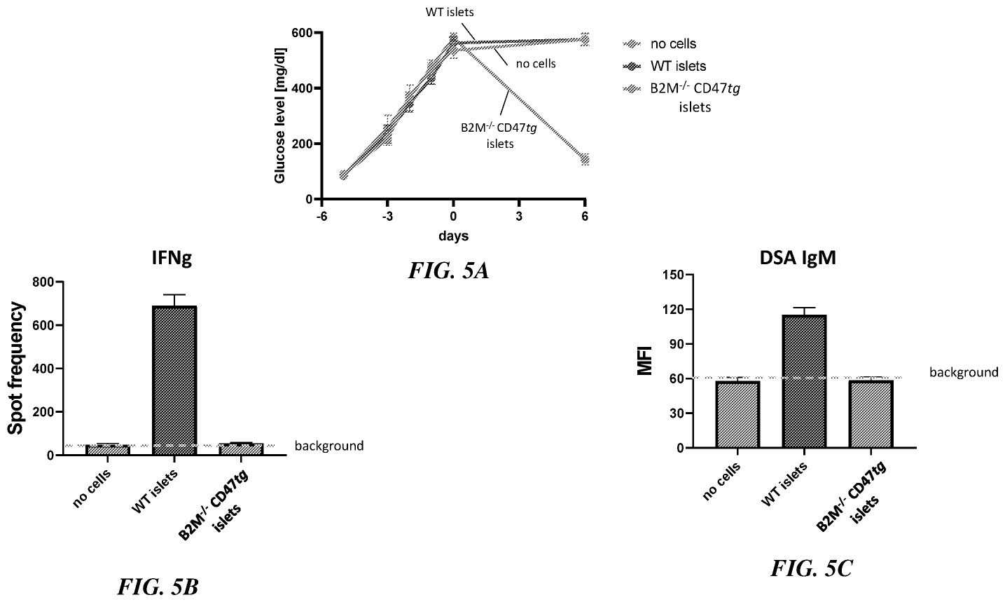

Mouse B2M^{-/-}; CD47tg primary beta islet cells function after allogeneic transplant. To analyze function of transplanted beta islet cells, blood glucose levels were measured 6 days after transplant. Glucose levels in diabetic (STZ treated) mice receiving i.m. injection of mouse wild type B6 primary beta islet cells remained high (>400 mg/dL) over the full study period, as shown in FIG. 5A. In contrast, blood glucose levels in diabetic (STZ treated) mice receiving i.m. injection of mouse B2M^{-/-}; CD47tg B6 primary beta islet cells dropped soon after transplantation, as shown in FIG. 5A. These data indicate that the mouse B2M^{-/-}; CD47tg B6 primary beta islet cells were able to function (e.g., restore lost glucose control due to diabetes) after allogeneic transplant.

Mouse B2M^{-/-}; CD47tg primary beta islet cells immune response after allogeneic transplant. To analyze the immune response to transplanted beta islet cells, an ELISPOT assay was used to evaluate the levels of IFNg cytokine secretion by CD8+ T cells. As shown in FIG. 5B, transplanted mouse B2M^{-/-}; CD47tg B6 primary beta islet cells exhibited lower levels of IFNg compared to transplanted mouse wild type primary beta islet cells. The levels of DSA IgG, measured by flow cytometry, were also lower in transplanted mouse B2M^{-/-}; CD47tg B6 primary beta islet cells compared to transplanted mouse wild type primary beta islet cells (FIG. 5C).

These data indicate that the mouse B2M^{-/-}; CD47tg B6 primary beta islet cells do not induce an immune response to the cells upon transplantation, are capable of evading NK cell mediated cytotoxicity upon administration, and are protected from antibody-mediated rejection.

### Example 3: Immune Evasion of Mouse B2M^{-/-}; CD47/Primary Beta Islet Cells in Vitro

To study the immune evasion effects of reducing MHC class I and MHC class II expression and increasing CD47 expression on primary beta islet cells, natural killer (NK) and macrophage killing assays were performed on mouse B2M^{-/-}; CD47tg B6 primary beta islet cells generated as described in Example 1. The killing of mouse B2M^{-/-}; CD47tg B6 primary beta islet cells in vitro by NKs and macrophages, compared to mouse wild type B6 primary beta islet cells and mouse B2M^{-/-} B6 primary beta islet cells, were monitored over time.

### A. Methods

NK cell culture. Human primary NK cells (StemCell Technologies) were cultured in RPMI-1640 plus 10% serum penicillin-streptomycin (pen/strep) before performing the assays.

Macrophage differentiation from peripheral blood mononuclear cells (PBMCs). PBMCs were isolated by Ficoll separation from fresh blood and were resuspended in RPMI-1640 with 10% serum pen/strep. Cells were plated in the presence of 10 ng/mL human macrophage-colony stimulating factor (M-CSF). From day six onward, human IL-2 was added to the medium for 24 h before performing assays.

NK cell and macrophage killing assay. NK cell killing assays and macrophage killing assays were performed on the XCelligence SP platform and MP platform (ACEA Biosciences) to provide for label-free monitoring of cell proliferation and viability of cells. 4×10⁵ mouse wild type B6 primary beta islet cells, mouse B2M^{-/-} B6 primary beta islet cells, or mouse B2M^{-/-}, CD47tg B6 primary beta islet cells (pooled or single clones) were plated into 96-well collagen coated E-plates. The XCelligence software was used to measure Cell Index (CI) as a measure of adhesion and hence cell killing (a decrease in cell index indicates an increase in killing of the cells). After the CI value reached 0.7, human NK cells or human macrophages were added at an effector to target (E:T) ratio of 0.5:1, 0.8:1, or 1:1 with or without 1 ng/mL human IL-2 or human IL-15. In some cases, NK cells were pretreated with human Fc receptor (FcR) block (concentration 1:5) before addition of target cells. Some wells were pretreated with an anti-MIAP410 blocking antibody (10 μg/mL, clone B6.H12, mouse IgG1,κ).

Flow cytometry. Surface expression of CD47 on primary beta islet cells were assessed by flow cytometry using antibody-specific reagents. Isotype antibodies were used as a control.

CL. Results

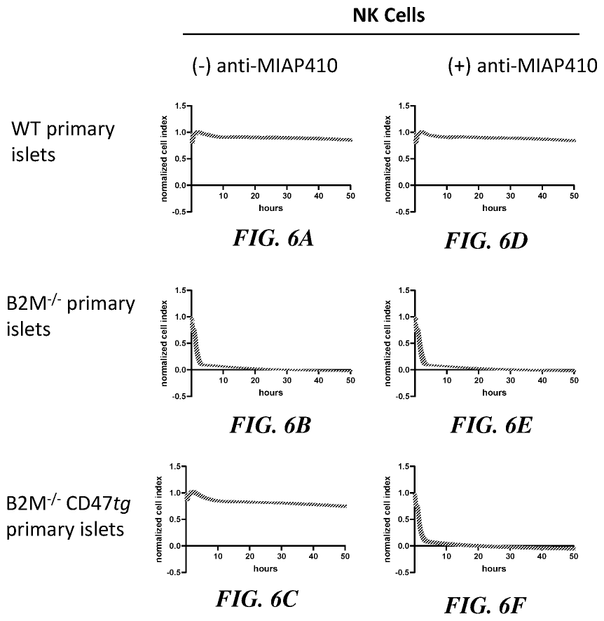

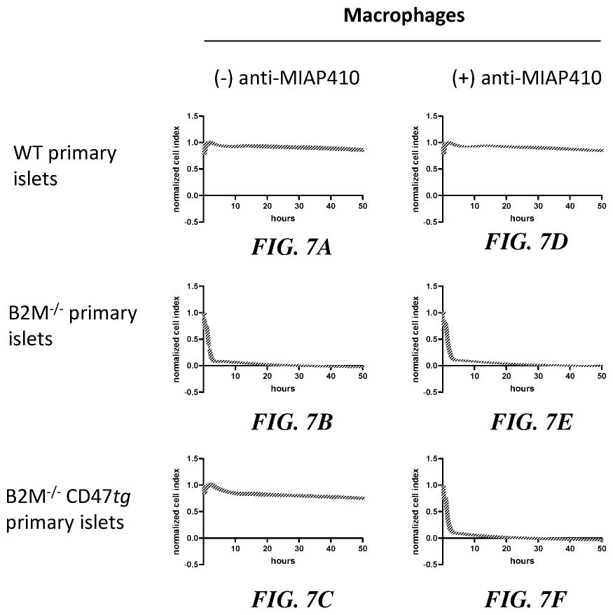

Mouse B2M^{-/-}; CD47tg primary beta islet cells evade NK cell and macrophage killing. NK− and macrophage-mediated cell killing of primary beta islet cells isolated from wild type B6 mice (mouse WT B6 primary beta islet cells) was not observed in the presence or absence of the anti-MIAP410 antibody (FIGS. 6A and 6D, NK cell; and FIGS. 7A and 7D, macrophage). In contrast, mouse B2M^{-/-} B6 primary beta islet cells were killed by the NK cells (FIG. 6B) and macrophages (FIG. 7B), indicating that the mouse B2M^{-/-} B6 primary beta islet cells were recognized as foreign cells by the NK cells and macrophages, thus resulting in cell killing. The addition of the anti-MIAP410 antibody to the co-culture of mouse B2M^{-/-} B6 primary beta islet cells and NK cells or macrophages did not further impact cell killing (FIGS. 6E and 7E).

In contrast, mouse B2M^{-/-}CD47tg B6 primary beta islet cells did not exhibit NK-mediated cell killing (FIG. 6C) or macrophage-mediated cell killing (FIG. 7C). However, in the presence of an anti-MIAP410 antibody, the CD47 protective effect was reduced and killing of the cells was observed by both NK cells (FIGS. 6F) and macrophages (FIG. 7F), similar to the results observed with the mouse B2M^{-/-} B6 primary beta islet cells that were not engineered with a CD47 transgene (FIGS. 6B and 6E, NK cell; and FIGS. 7B and 7E, macrophage). These data indicate that mouse B2M^{-/-}; CD47tg B6 primary beta islet cells effectively evade immune responses by NK cells and macrophages.

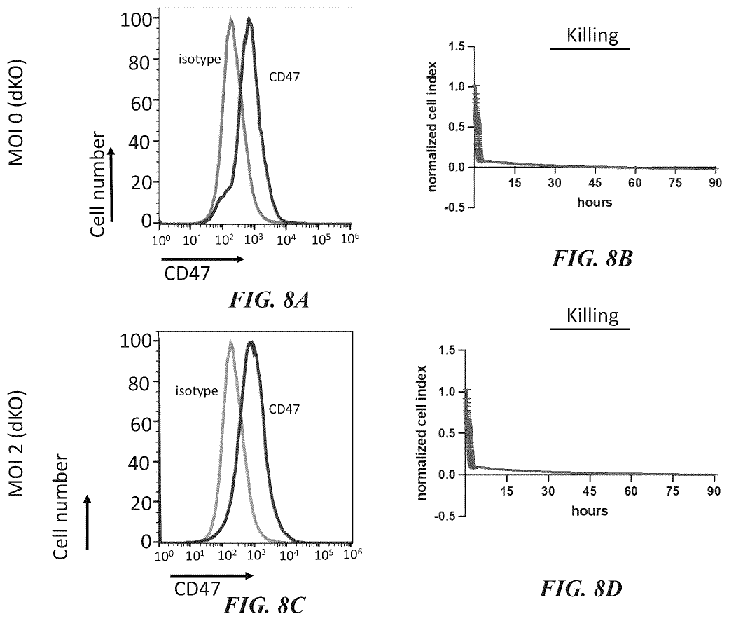

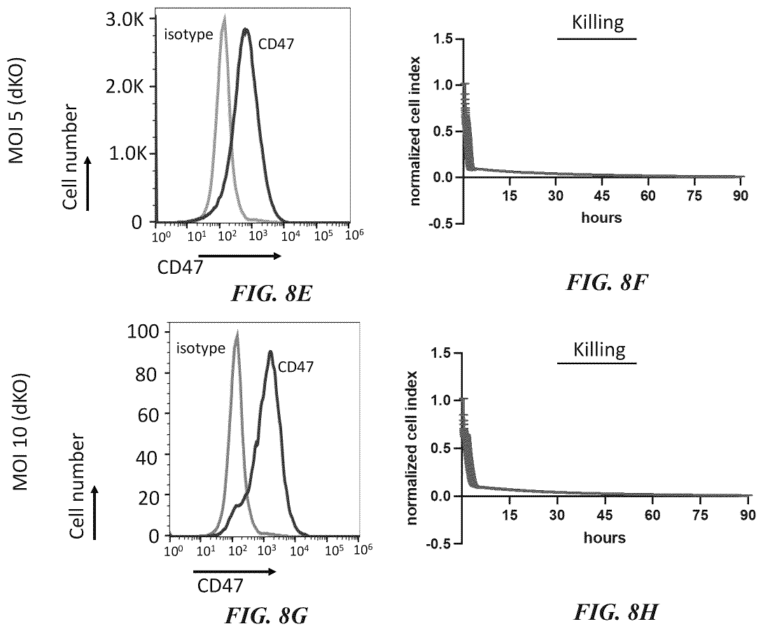

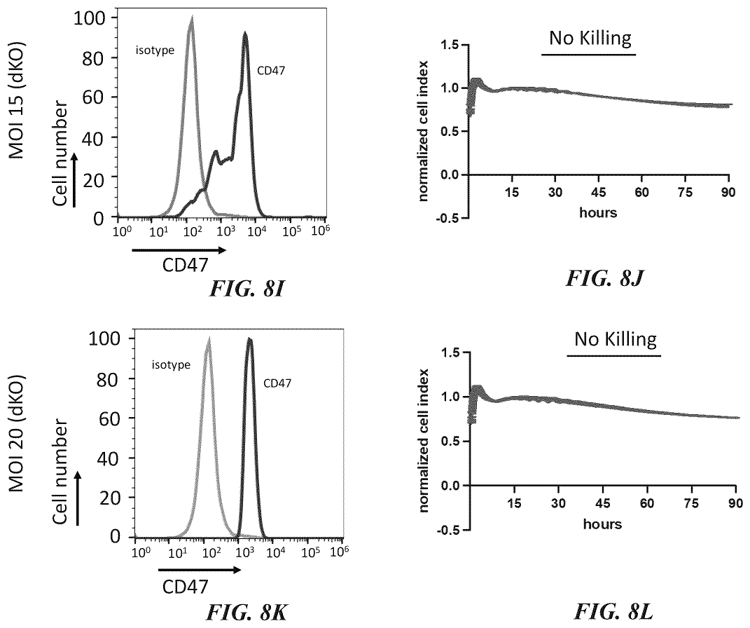

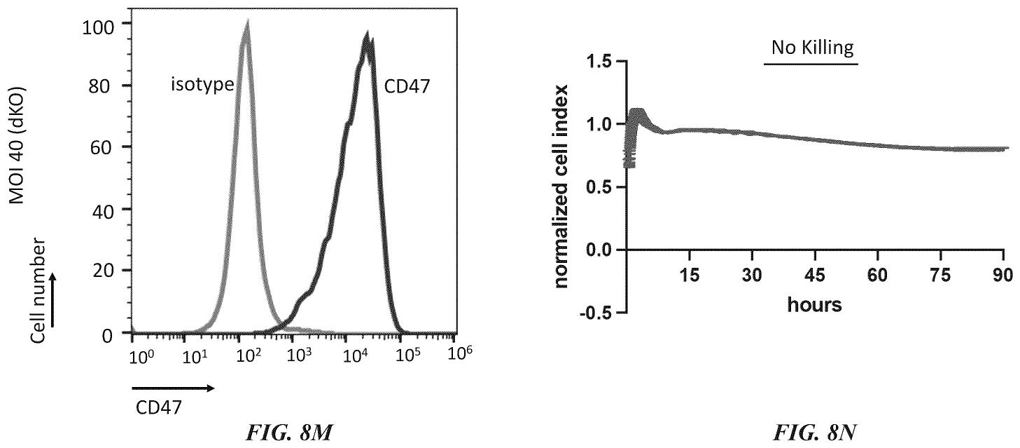

CD47 expression for mouse B2M^{-/-}; CD47tg primary beta islet cells evasion of NK cell killing. NK-mediated cell killing of various B2M^{-/-}; CD47tg primary beta islet cells isolated from wild type B6 mice (mouse WT B6 primary beta islet cells) was observed as a function of varying CD47 transgene expression. Mouse B2M^{-/-}; CD47tg B6 primary beta islet cells with lower overexpression of the CD47 transgene exhibited NK-mediated cell killing (FIGS. 8A-8H). In contrast, mouse B2M^{-/-}; CD47tg B6 primary beta islet cells with higher overexpression of the CD47 transgene did not exhibit NK-mediated cell killing (FIGS. 8I-8N). These results confirm that CD47 overexpression in primary beta islet cells is effective to evade immune responses by NK cells.

### Example 4: In Vitro Characterization of B2M^{-/-}; CD47tg Human Primary Islet Cells

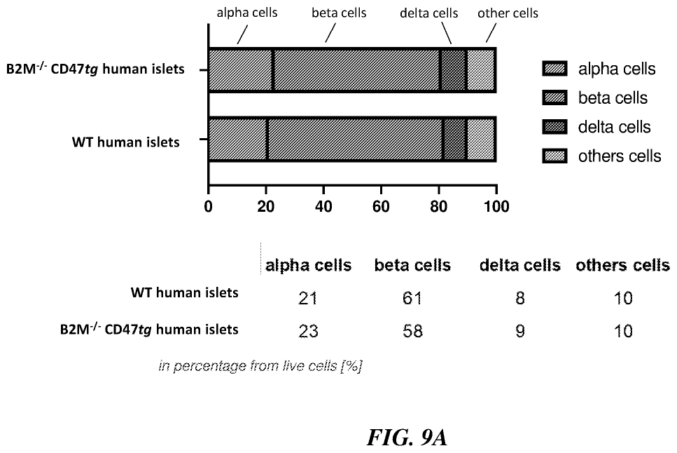

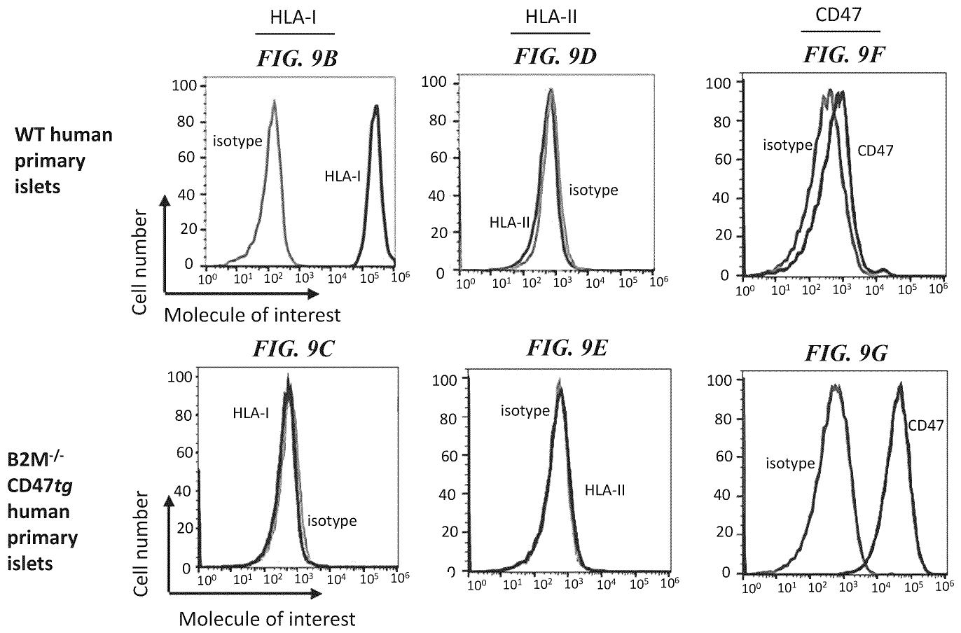

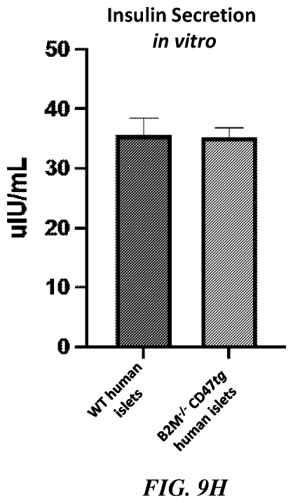

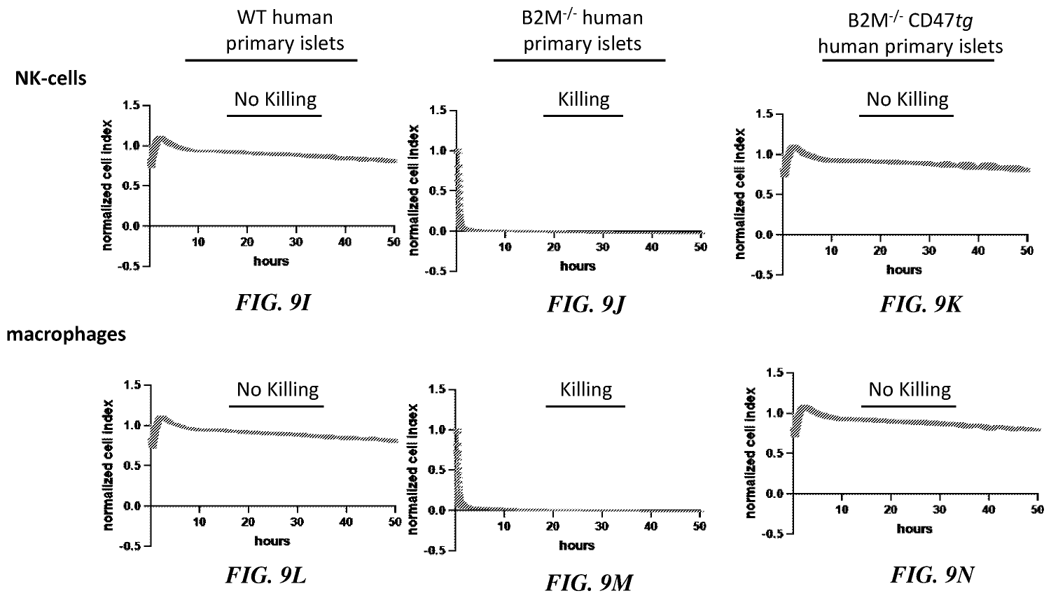

This example describes studies characterizing hypoimmune primary human beta islet cells that were engineered to (1) knock out B2M (B2M^{-/-}) to reduce HLA class I expression and (2) overexpress exogenous CD47 (CD47tg). The hypoimmune (B2M^{-/-}; CD47tg) primary beta islet cells were monitored for insulin secretion and for protection from cell killing by Natural Killer (NK) cells and macrophages, compared to wild type (WT) primary human beta islet cells or B2M^{-/-} primary human beta islet cells. WT human primary islet cells do not express HLA class II (FIG. 9C) and were therefore not engineered to alter expression of HLA class II molecules. The hypoimmune cells and the B2M^{-/-} cells were engineered from the WT cells and are therefore from the same donor.

### A. Methods

Generation of human primary islet cells and Cell Engineering. Primary beta islet cells were isolated from two cadaver donors (Donor 1 and Donor 2) using a standard technique. Such techniques are known in the art, including as described in J. Kerr-Conte et al., Transplantation, 89, 2010. To generate hypoimmune cells, the isolated cells were engineered to knockout B2M using standard CRISPR/Cas9 gene editing techniques, and were transduced with a transgene (tg) encoding exogenous CD47 protein using a lentiviral vector containing a polynucleotide encoding CD47. The hypoimmune islets were sorted by flow cytometry for cells negative for HLA class I/II and for CD47 overexpression.

Insulin secretion. A standard glucose-stimulated insulin secretion (GSIS) assay was used to measure in vitro insulin secretion. A U-PLEX® Meso Scale Discovery (MSD) assay was used to detect insulin secretion. Briefly, 100,000 cells were used in 2 mL of media, and total insulin secretion over 24 h was measured.

Flow cytometry. Surface expression of HLA class I, HLA class II, and CD47 on human primary islet cells was assessed by flow cytometry using antibody-specific reagents. Isotype antibodies were used as a control.

NK cell and macrophage killing assay. NK cell killing assays and macrophage killing assays were performed on the XCelligence SP platform and MP platform (ACEA Biosciences) to provide for label-free monitoring of cell proliferation and viability of cells. 4×10⁵ wild type human primary islet cells, B2M^{-/-} human primary islet cells, or B2M^{-/-}, CD47tg human primary islet cells (pooled or single clones) were plated into 96-well collagen coated E-plates. The XCelligence software was used to measure Cell Index (CI) as a measure of adhesion and hence cell killing (a decrease in cell index indicates an increase in killing of the cells). After the CI value reached 0.7, human primary NK cells or human macrophages differentiated from peripheral blood mononuclear cells with macrophage colony stimulating factor (M-CSF) were added at an effector to target (E:T) ratio of 1:1.

CL. Results

Results of the studies are summarized below. Representative results are shown for one donor but similar results were observed for at least two additional donors.

B2M^{-/-} CD47tg edits do not impact cellular morphology or composition of human primary islet cells. To evaluate cellular morphology, immunohistochemistry (IHC) staining for 4′,6-diamidino-2-phenylindole (DAPI), insulin, and glucagon was carried out on WT human primary islet cells and B2M^{-/-}; CD47tg human primary islet cells. The results demonstrated no differences in the morphology indicating that the B2M^{-/-}; CD47tg edits to human primary islets do not appear to impact human primary islet morphology. The Moreover, the B2M^{-/-}; CD47tg edits do not affect the human primary islet cell composition compared to WT human primary islet cell composition (FIG. 9A).

B2M^{-/-} CD47tg human primary islet cells do not express HLA-I or HLA-H, and have increased CD47 expression. To evaluate the expression of HLA-I and HLA-II and CD47 in the B2M^{-/-}; CD47tg human primary islet cells, flow cytometry was performed. B2M^{-/-}; CD47tg human primary islet cells were negative for HLA-I (FIG. 9C) and HLA-II (FIG. 9E), whereas WT human primary islet cells expressed HLA class I at high levels (FIG. 9B) and did not express HLA class II (FIG. 9D). B2M^{-/-}; CD47tg human primary islet cells showed increased expression of CD47 (48-fold and 51-fold over isotype control, as shown in FIGS. 9G), compared to WT human primary islet cells (FIG. 9F).

B2M^{-/-} CD47tg human primary islet cells retain insulin secretion capabilities. B2M^{-/-} CD47tg human primary islet cells retained similar insulin secretion capabilities to WT human primary islet cells in vitro as shown in FIG. 9H for a representative donor. These data indicate that the hypoimmune modifications to the beta islet cells do not impact insulin secretion.

B2M^{-/-}; CD47tg human primary islet cells evade NK cell and macrophage killing. WT human primary islet cells were not killed by NK cells (FIG. 9I) or macrophages (FIG. 9L). In contrast, B2M^{-/-} human primary islet cells that had reduced expression of HLA class I and HLA class II were killed by the NK cells (FIG. 9J) and macrophages (FIG. 9M), indicating that the B2M^{-/-} human primary islet cells were recognized as foreign cells by the NK cells and macrophages, thus resulting in cell killing.

In contrast, B2M^{-/-}CD47tg human primary islet cells did not exhibit NK-mediated cell killing (FIG. 9K) or macrophage-mediated cell killing (FIG. 9N). These data indicate that the hypoimmune (B2M^{-/-}; CD47tg) human primary islet cells are protected by the CD47 overexpression and are able to effectively evade immune responses by NK cells and macrophages.

### Example 5: Survival and Function of B2M^{-/-} and B2M^{-/-}; CD47tg Human Primary Islet Cells in a Diabetic Humanized Mouse Transplant Study

Hypoimmune (B2M^{-/-}; CD47tg) and double knockout (B2M^{-/-}) human primary islet cells were produced as described in Example 4, and were transplanted into an allogeneic diabetic humanized NSG-SGM3 recipient mouse. Incidence of diabetes in the mice, and survival and function of the transplanted B2M^{-/-} and B2M^{-/-}; CD47tg human primary islet cells compared to transplanted wild type human primary islet cells, were monitored over time.

### A. Methods

Transplant study design and administration. Twenty-five humanized NSG-SGM3 mice (mouse body weight 18-20 g) were randomized into study groups post STZ administration to induce diabetes, in which the groups differed based on the cells that were administered (wild type, B2M^{-/-} CD47tg, and B2M^{-/-} human beta islet cells). The study groups were as follows: wild type human islet (luc+) i.m. transplant; no transplant diabetes control; B2M^{-/-} human islet (luc+) i.m. transplant; and B2M^{-/-} CD47tg human islet (luc+) i.m. transplant.

300 human islet clusters of about 1,500 cells per cluster were transplanted by i.m. injection into mice. Day 0 (d0) was defined as the day of transplantation. Mice were monitored for bioluminescence (BLI) as an indicator of beta islet cell survival and for glucose levels after 4 hours of fasting to monitor diabetes. A glucose challenge was performed on day 29 of the study.

T Cell Enzyme-Linked Immune absorbent SPOT (ELISPOT) Assay. Interferon gamma (IFNg)-secreting CD8(+) T cells in human primary islet cells were detected by ELISPOT.

C-peptide Assay. C-peptide levels in human primary islet cells were measured using standard assays.

Splenocyte Killing Assay. Splenocyte killing assays were performed on the XCelligence SP platform and MP platform (ACEA Biosciences) to provide for label-free monitoring of cell proliferation and viability of cells. 4×10⁵ wild type human primary islet cells, B2M^{-/-} human primary islet cells, or B2M^{-/-}, CD47tg human primary islet cells (pooled or single clones) were plated into 96-well collagen coated E-plates. The XCelligence software was used to measure Cell Index (CI) as a measure of adhesion and hence cell killing (a decrease in cell index indicates an increase in killing of the cells). After the CI value reached 0.7, human splenocytes were added at an effector to target (E:T) ratio of 1:1.

Complement Dependent Cytotoxicity (CDC) Assay. B2M^{-/-}, CD47tg human primary islet cells were incubated with serum and CDC was analyzed by measuring cell lysis over time of incubation on the XCelligence MP platform (ACEA Biosciences) to provide for label-free monitoring of cell proliferation and viability of cells. Changes in impedance were reported as Cell Index (CI) (a decrease in cell index indicates an increase in lysis or killing of the cells).

CL. Results

Results of the studies are summarized below. Representative donor results are shown but similar results were observed for various donors.

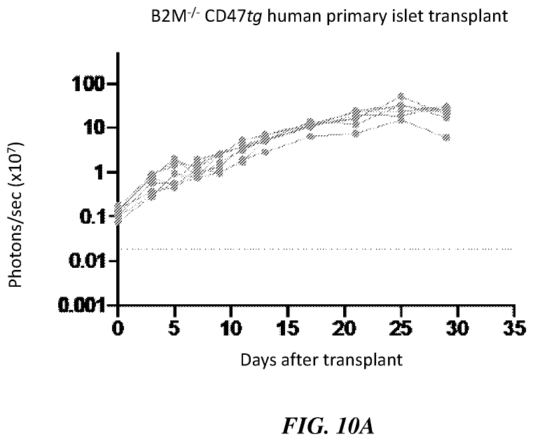

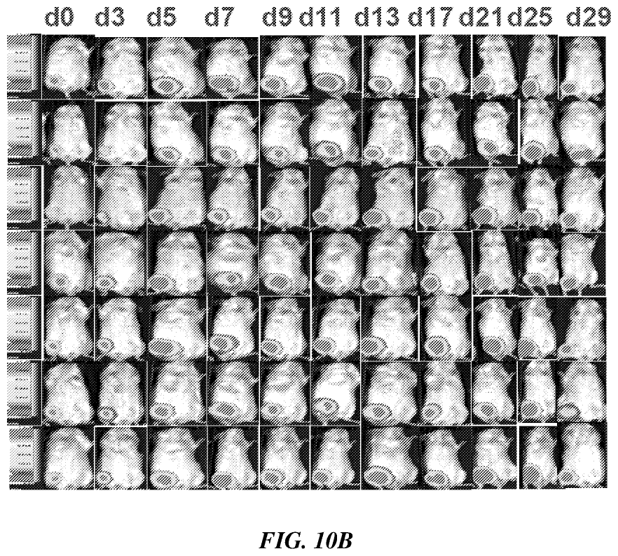

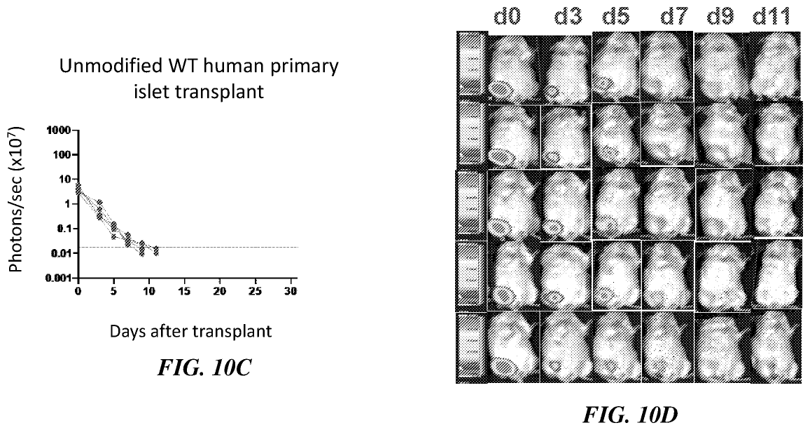

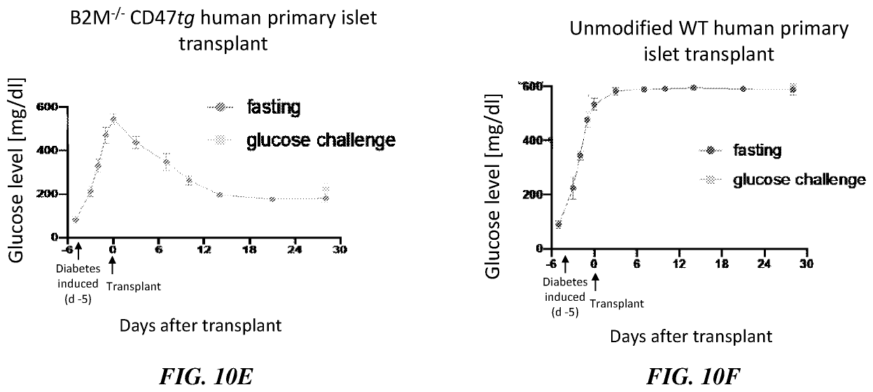

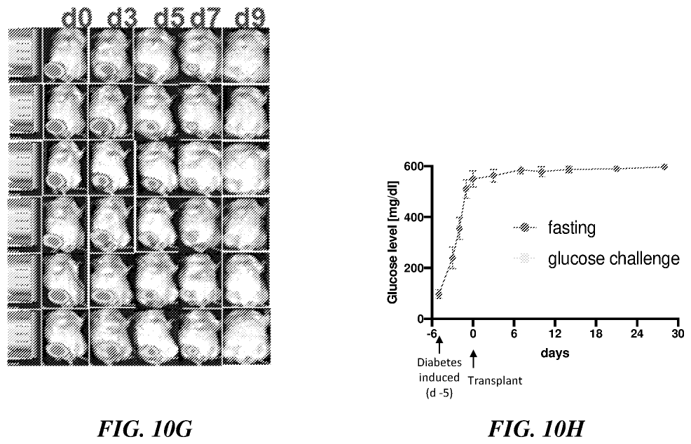

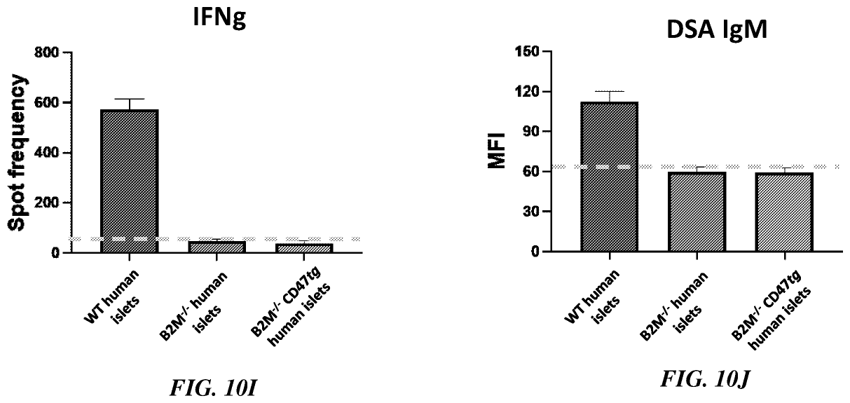

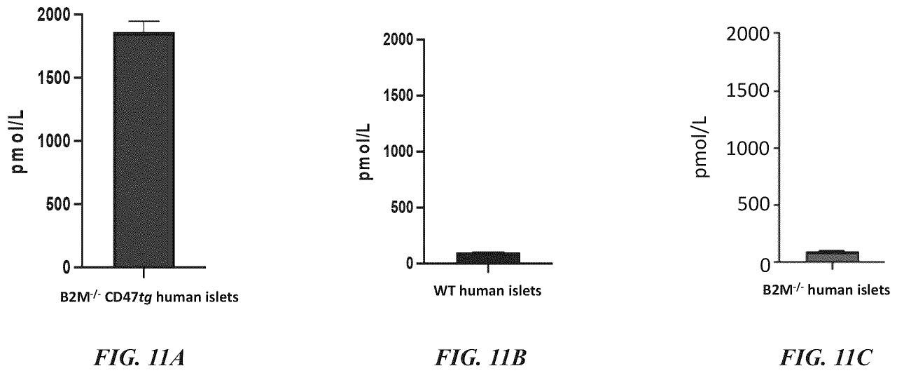

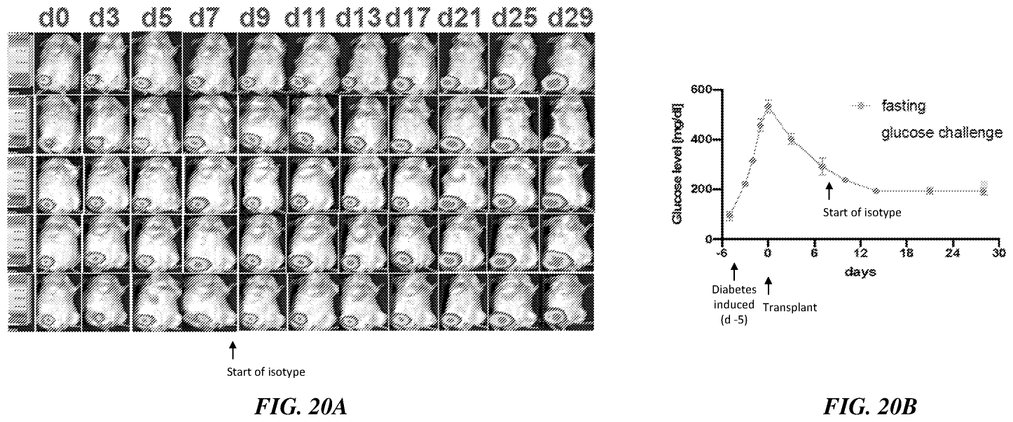

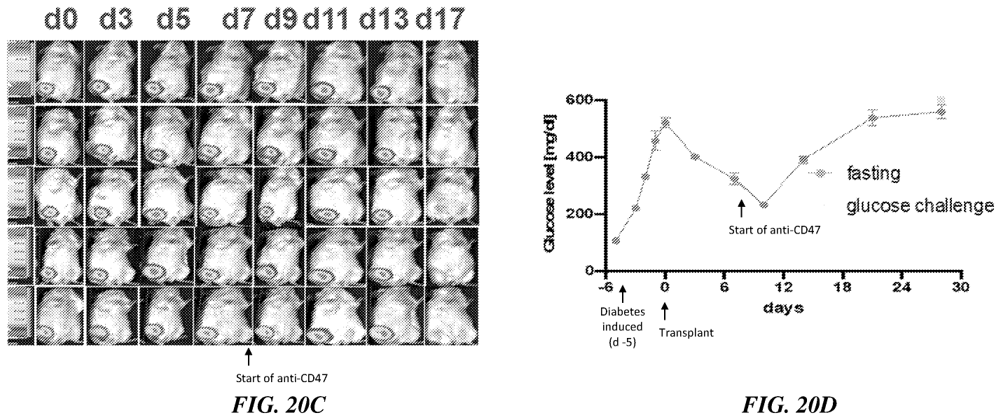

B2M^{-/-}; CD47tg human primary islet cells survive after allogeneic transplant. Quantification of BLI imaging results of human primary islet cells after i.m. injection are shown in FIG. 10A (B2M^{-/-}; CD47tg human primary islet cells) and FIG. 10C (WT human primary islet cells). Corresponding BLI images of human primary islet cells after i.m. injection are shown in FIG. 10B (WT human primary islet cells) and FIG. 10D (B2M^{-/-}; CD47tg human primary islet cells), and BLI images of B2M^{-/-} human primary islet cells are shown in FIG. 20A. Bioluminescence was initially observed at the i.m. injection site for all groups following administration of the cells. However, the number of photons detected for transplanted WT human primary islet cells rapidly declined over the first 5 days after transplant (FIGS. 10C and 10D), indicating death of the WT human primary islet cells, likely due to an immune response. Similarly to WT human primary islet cells, the number of B2M^{-/-} human primary islet cells rapidly declined over the first 5 days after transplant (FIG. 10G). In contrast, the number of photons detected from transplanted B2M^{-/-}; CD47tg human primary islet cells increased over the course of 29 days post-transplant, indicating survival and growth of the B2M^{-/-}; CD47tg human primary islet cells (FIGS. 10A and 10B). B2M-CD47tg human primary islet cells are able to survive for one month without inducing a local immune reaction (data not shown).

B2M^{-/-}; CD47tg human primary islet cells function after allogeneic transplant. To analyze certain functions of transplanted human primary islet cells, blood glucose levels were measured 6 days after transplant. Glucose levels measured about 4 hours after fasting in diabetic (STZ treated) mice receiving i.m. injection of WT human primary islet cells and B2M^{-/-} human primary islet cells and remained high (>400 mg/dL) over the full study period, as shown in FIG. 10F and FIG. 10H, respectively. In contrast, fasting blood glucose levels in diabetic (STZ treated) mice receiving i.m. injection of B2M^{-/-}; CD47tg human primary islet cells dropped soon after transplantation, as shown in FIG. 10E. Further, the mice receiving i.m. injection of B2M^{-/-}; CD47tg human primary islet cells successfully tolerated a glucose challenge on day 29 (FIG. 10E).

To further analyze function of transplanted human primary islet cells, C-peptide levels were measured following transplant. C peptide levels in diabetic (STZ treated) mice receiving i.m. injection of WT human primary islet cells and B2M^{-/-} human primary islet cells were low, as shown in FIGS. 11B and 11C, respectively. In contrast, the mice receiving i.m. injection of B2M^{-/-}; CD47tg human primary islet cells had high levels of C-protein (FIG. 11A).

These data indicate that the B2M^{-/-}; CD47tg human primary islet cells were able to function (e.g., restore lost glucose control due to diabetes) after allogeneic transplant.

B2M^{-/-}; CD47tg human primary islet cells immune response after allogeneic transplant. To analyze the immune response to transplanted human primary islet cells, an ELISPOT assay was used to evaluate the levels of IFNg cytokine secretion by CD8+ T cells. As shown in FIG. 10I transplanted B2M^{-/-} human primary islet cells and B2M^{-/-}; CD47tg human primary islet cells exhibited lower levels of IFNg compared to transplanted wild type human primary islet cells. These results are consistent with an observation that the wild-type beta islets show TH1 activation after transplantation as determined by ELISPOT. The levels of DSA IgM, measured by flow cytometry, were also lower in transplanted B2M^{-/-} human primary islet cells and transplanted B2M^{-/-}; CD47tg human primary islet cells compared to transplanted wild type primary beta islet cells, indicating that wild-type beta islets show donor-specific antibody binding (IgM) (FIG. 10J).

This data indicates that transplanted wild type human primary islet cells exhibit an adaptive immune response, while transplanted B2M^{-/-} human primary islet cells and transplanted B2M^{-/-}; CD47tg human primary islet cells do not exhibit an adaptive immune response.

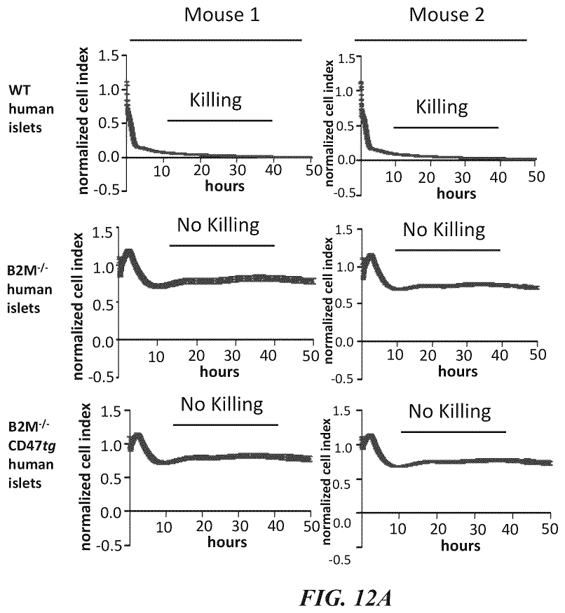

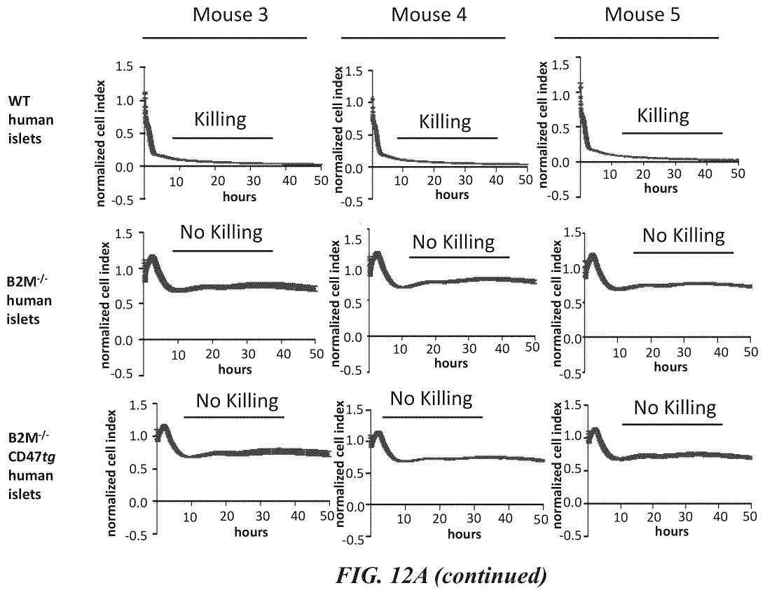

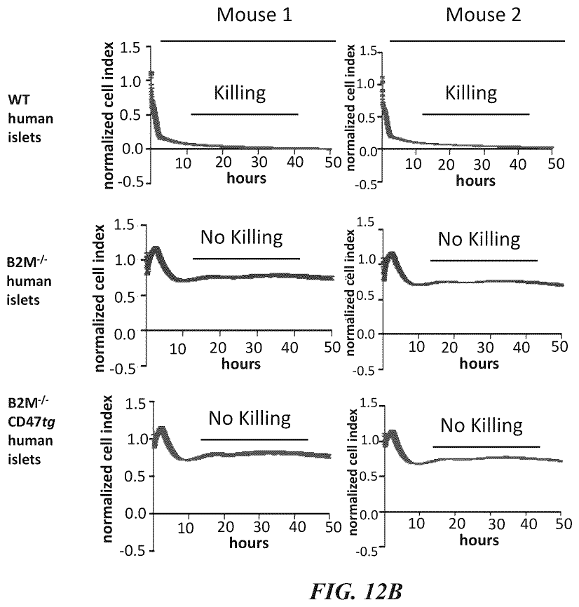

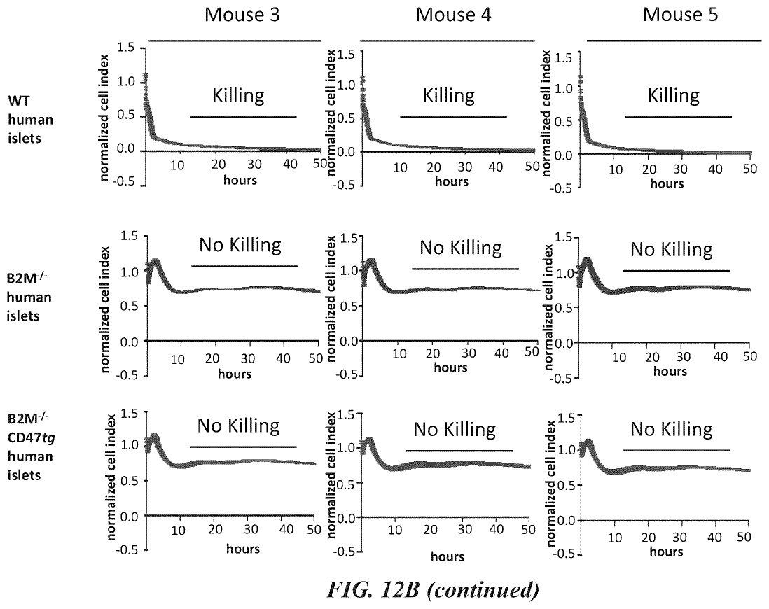

B2M^{-/-}; CD47tg and B2M^{-/-} human primary islet cells evade splenocyte killing after allogeneic transplant. Transplanted WT human primary islet cells were killed by splenocytes, as shown in the top panel of FIG. 12A. indicating that the transplanted WT human primary islet cells were recognized as foreign cells by the splenocytes, thus resulting in cell killing. In contrast, transplanted B2M^{-/-}; CD47tg human primary islet cells and transplanted B2M^{-/-} human primary islet cells did not exhibit splenocyte-mediated cell killing (FIG. 12A, middle and top panels). These data indicate that the transplanted hypoimmune (B2M^{-/-}; CD47tg) and transplanted double knockout (B2M^{-/-}) human primary islet cells are able to effectively evade immune responses by splenocytes.

B2M^{-/-}; CD47tg and B2M^{-/-} human primary islet cells are not subjected to CDC after allogeneic transplant. To further analyze the immune response to transplanted human primary islet cells, a CDC assay was used. WT human primary islet cells killed in the CDC assay, as shown in the top panel of FIG. 12B. indicating that the WT human primary islet cells were recognized resulting in cell killing. In contrast, B2M^{-/-}; CD47tg and B2M^{-/-} human primary islet cells did not exhibit CDC cell killing (FIG. 12B, middle and top panels). These data indicate that the hypoimmune (B2M^{-/-}; CD47tg) and double knockout (B2M^{-/-}) human primary islet cells are able to effectively evade CDC.

### Example 6: Immune Evasion of B2M^{-/-}; CD47/g Human Primary Islet Cells In Vitro

To study the immune evasion effects of reducing HLA class I and HLA class II expression and increasing CD47 expression on human primary islet cells, peripheral blood mononuclear cell (PBMC) killing assays were performed on B2M^{-/-}; CD47tg human primary islet cells generated as described in Example 4. PBMCs from either type-I diabetic patients or PBMCs from healthy donors were incubated with WT human primary islet cells or B2M^{-/-}; CD47tg human primary islet cells in vitro and killing was monitored over time.

### A. Methods

PBMC isolation and culture. PBMCs were isolated from five (5) type-I diabetic human patients or three (3) healthy donors by Ficoll separation from fresh blood, and were resuspended in RPMI-1640 with 10% serum pen/strep.

PBMC killing assay. PBMC cell killing assays were performed on the XCelligence SP platform and MP platform (ACEA Biosciences) to provide for label-free monitoring of cell proliferation and viability of cells. 4×10⁵ WT human primary islet cells or B2M^{-/-}, CD47tg human primary islet cells were plated into 96-well collagen coated E-plates. The XCelligence software was used to measure Cell Index (CI) as a measure of adhesion and hence cell killing (a decrease in cell index indicates an increase in killing of the cells). After the CI value reached 0.7, human PBMC cells were added at an effector to target (E:T) ratio of 1:1.

Flow cytometry. PBMC killing of human primary islet cells was assessed by flow cytometry to quantify the percentage of dead cells using PerCP-Cy5.

CL. Results

Results of the studies are summarized below. Representative results are shown for one donor but similar results were observed for both donors.

B2M^{-/-}; CD47tg human primary islet cells evade diabetic PBMC killing. Diabetic PBMCs isolated from each of the patients with type-I diabetes mediated cell killing of WT human primary islet cells; representative results are shown in FIG. 13A from PBMCs isolated from one type-I diabetic patient. PBMCs from the healthy donors did not kill WT human primary islet cells as shown for a representative healthy donor PBMCs in FIG. 13E. No killing of the WT human primary islet cells was observed in the absence of incubation with the PBMCs (FIG. 13B and FIG. 13F). Similar results were observed when killing was assessed by flow cytometry for dead cells (FIG. 13I). These results indicate that the WT human primary islet cells were recognized as foreign cells by the diabetic PBMCs, thus resulting in cell killing.

In contrast, B2M^{-/-}CD47tg human primary islet cells were not killed by diabetic PBMCs (FIG. 13C, representative results with diabetic PBMCs from one patient) or by healthy PBMCs (FIG. 13G, representative results with PBMCs from one healthy donor). No killing of the B2M^{-/-} CD47tg human primary islet cells was observed in the absence of incubation with the PBMCs (FIG. 13D and FIG. 13H). Similar results were observed when killing was assessed by flow cytometry for dead cells (FIG. 13J). These data indicate that B2M^{-/-}; CD47tg human primary islet cells effectively evade immune responses by diabetic PBMCs.

### Example 7: Assessment of CD47 Signaling in B2M^{-/-}; CD47tg Human Primary Islet Cells with Anti-CD47 Fusion Proteins In Vitro

This example describes studies characterizing CD47 blockade by hypoimmune primary human beta islet cells that were engineered to (1) knock out B2M (B2M′) to reduce HLA class I expression and (2) overexpress exogenous CD47 (CD47tg). Hypoimmune (B2M^{-/-}; CD47tg) human primary islet cells were produced as described in Example 4. Cell killing by Natural Killer (NK) cells and macrophages in the presence of anti-CD47 Fc fusion proteins (i.e., anti-CD47 IgG1Fc and anti-CD47 IgG4Fc) was assessed for the B2M^{-/-}; CD47tg human primary islet cells, compared to B2M^{-/-}; CD47tg human primary islet cells. Production of cytotoxic products and phagocytosis of macrophages due to CD47 binding were also evaluated.

### A. Methods

NK cell and macrophage killing assay. NK cell killing assays and macrophage killing assays were performed substantially as described in examples above.

Detection of cytotoxic products. Granzyme, perforin, and reactive oxygen species (ROS) were detected by ELISA using commercially available kits from Invitrogen (Human Granzyme B ELISA Kit and Human Perforin ELISA Kit) and Biosource (Human Reactive Oxygen Species ELISA Kit), according to the manufacturer's instructions.

Phagocytosis assay. Anti-CD47 antibody (100 μg/ml), was incubated with B2M^{-/-}, CD47tg human primary islet cells and macrophages. For some conditions anti-CD47 IgG1Fc or anti-CD47 IgG4Fc also was added. Phagocytosis of beta islet cells was assessed using the pHrodo™ phagocytosis assay by flow cytometry after 1 h incubation.

CL. Results

Results of the studies are summarized below. Representative results are shown for one donor but similar results were observed for both donors.

B2M^{-/-}; CD47tg human primary islet cells expressing anti-CD47 Fc fusion proteins are killed by NK cells and macrophages. B2M^{-/-}CD47tg human primary islet cells did not exhibit NK-mediated cell killing (FIG. 14A) or macrophage-mediated cell killing (FIG. 14D), indicating that the hypoimmune (B2M^{-/-}; CD47tg) human primary islet cells are protected by the CD47 overexpression and are able to effectively evade immune responses by NK cells and macrophages. Protection from cell killing was blocked by anti-CD47 IgG1Fc or anti-CD47 IgG4Fc fusion proteins, as shown for NK-mediated cell killing (FIGS. 14B and 14C, respectively) or macrophage-mediated cell killing (FIGS. 14E and 14F, respectively). These data indicate that the blocking of CD47-SIRP signaling in B2M^{-/-}CD47tg human primary islet cells results in their recognition as foreign cells by the NK cells and macrophages, thus causing in cell killing, and further confirm that overexpression of CD47 contributes to immune evasion by the modified cells.

B2M^{-/-}; CD47tg human primary islet cells produce cytotoxic products when anti-CD47 Fcfusion proteins are added. As shown in FIGS. 15A-15C, B2M^{-/-}; CD47tg human primary islet cells exhibited lower levels of granzyme B (FIG. 15A), perforin (FIG. 15B), and ROS (FIG. 15C), but the levels of these cytotoxic products were substantially higher in the presence of either anti-CD47 IgG1Fc or anti-CD47 IgG4Fc fusion proteins. As killing is mediated by release of these cytotoxic products, these data indicate that blocking of CD47-SIRP signaling by both fusion proteins, IgG1 and IgG4, induce killing of the modified primary beta islet cells.

### Example 8: Adaptive Immune Response of B2M^{-/-}; CD47/g Human Primary Islet Cells In Vitro from One Exemplary Donor

In order to investigate the macrophage cell killing mechanism of B2M^{-/-}; CD47tg human primary islet cells, and to understand how overexpression of CD47 is affecting the killing, phagocytosis assays were performed. Specifically, since HLA-I/II KO cells induce killing by macrophages due to their “missing self,” studies were carried out to reveal if the observed mechanism of cell-killing was due to phagocytosis versus release of cytotoxic products. Further, the studies were also carried out with whole, apoptotic, and necrotic cells to assess if overexpression of CD47 is impacting the clearance mechanism of dying/dead cells by macrophages.

### A. Methods

Flow cytometry. Expression of CD47 in human primary islet cells was assessed by flow cytometry.

Phagocytosis assay. Wild type human primary islet cells (whole, apoptotic, and necrotic), B2M^{-/-} human primary islet cells (whole, apoptotic, and necrotic), B2M^{-/-}; CD47tg human primary islet cells (whole, apoptotic, and necrotic) were incubated with macrophages. In some cases, an anti-CD47 IgG1 antibody was added which binds to the FcR and mediates phagocytosis. Phagocytosis of beta islet cells cells due to was assessed using the pHrodo™ phagocytosis assay by flow cytometry after 1 h incubation.

CL. Results

Results of the studies are summarized below. Representative donor results are shown.

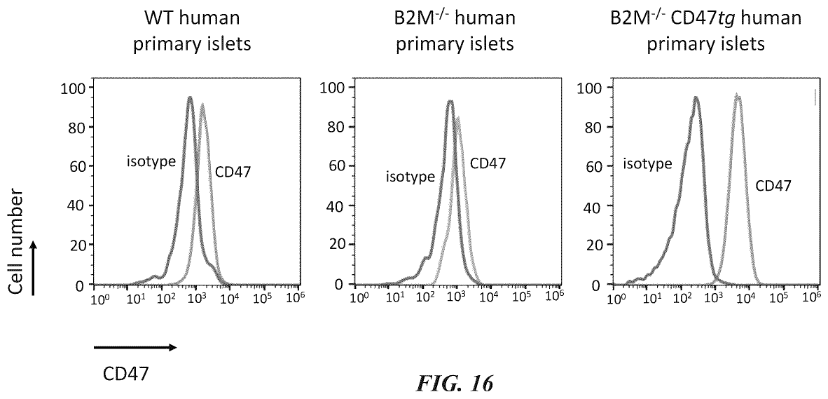

Consistent with previous results, flow cytometry confirmed that B2M^{-/-} human primary islet cells that were not engineered to overexpress CD47 exhibited a low level surface expression of CD47 (2-fold over isotype control, as shown in FIG. 16, left panel), while B2M^{-/-}; CD47tg primary beta islet cells showed increased expression of CD47 (28-fold over isotype control, as shown in FIG. 16, right panel).

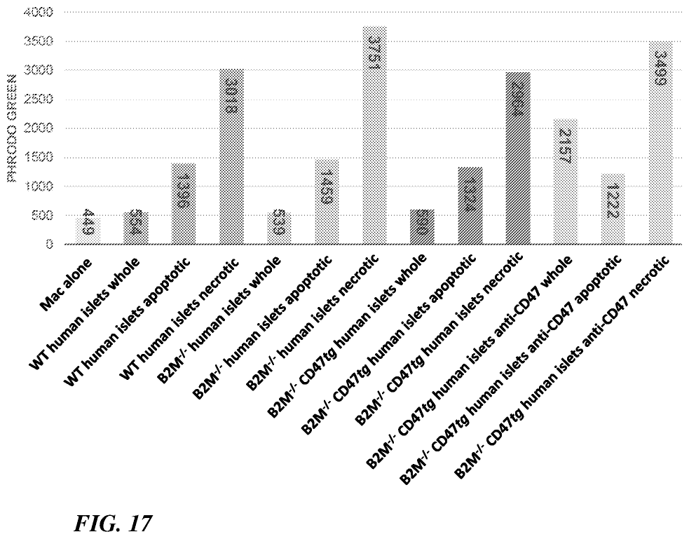

As shown in FIG. 17, whole wild type human primary islet cells, whole B2M^{-/-} human primary islet cells, and whole B2M^{-/-}; CD47tg human primary islet cells evade phagocytosis by macrophages. In contrast, necrotic and apoptotic human primary islet cells of each cell type were phagocytosed by macrophages. Thus, CD47 overexpression does not prevent phagocytosis of either apoptotic or necrotic B2M^{-/-}; CD47tg human primary islet cells, which may be important after islet rejection. Moreover, anti-CD47 IgG1 fusion protein induces phagocytosis of B2M^{-/-}; CD47tg human primary islet cells (whole, apoptotic, and necrotic).

### Example 9: Assessment of Ig-Mediated Phagocytosis of B2M^{-/-}, CD47/Human Primary Islet Cells

Anti-CD47 antibody (100 μg/ml), either IgG1 Fc or IgG4 Fc, was incubated with B2M^{-/-}, CD47tg human primary islet cells and non-human primate (NHP) macrophages. Phagocytosis of beta islet cells was assessed using the pHrodo™ phagocytosis assay by flow cytometry after 1 h incubation.

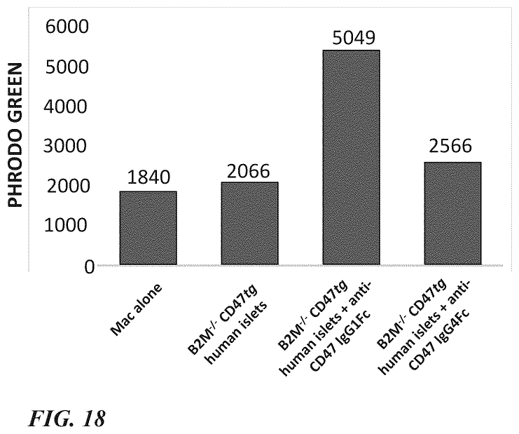

As shown in FIG. 18, B2M^{-/-}; CD47tg human primary islet cells and macrophage (Mac) alone controls were not phagocytosed by macrophages. In contrast, B2M^{-/-}; CD47tg human primary islet cells incubated with anti-CD47 IgG1 Fc, but not anti-CD47-IgG4 Fc, were phagocytosed. The observed phagocytosis was due to IgG1 Fc binding via antibody-dependent cellular phagocytosis (ADCP). IgG4 binding does not result in as strong FcR binding as IgG1, consistent with the lower levels of phagocytosis with the IgG4 Fc.

### Example 10: Survival and Function of B2M^{-/-}; CD47tg Human Primary Islet Cells in an Immune Incompetent Diabetic Mouse Transplant Study

Hypoimmune (B2M^{-/-}; CD47tg) human primary islet cells were produced as described in Example 4, and were transplanted into an immune incompetent diabetic NSG recipient mouse. Incidence of diabetes in the mice, and survival and function of the transplanted B2M^{-/-}; CD47tg human primary islet cells compared to transplanted wild type human primary islet cells, were monitored over time.

### A. Methods

Transplant study design and administration. Twenty-five NSG mice (mouse body weight 18-20 g) were randomized into study groups post STZ administration to induce diabetes, in which the groups differed based on the cells that were administered (wild type, B2M^{-/-} CD47tg, and B2M^{-/-} human islet cells). The study groups were as follows: wild type human islet (luc+) i.m. transplant; no transplant diabetes control; and B2M^{-/-} CD47tg human islet (luc+) i.m. transplant.

300 human islet clusters of about 1,500 cells per cluster were transplanted by i.m. injection into mice. Day 0 (d0) was defined as the day of transplantation. Mice were monitored for bioluminescence (BLI) as an indicator of islet cell survival and for glucose levels after 4 hours of fasting as an indicator of diabetes. A glucose challenge was performed on day 29 of the study.

C-peptide Assay. C-peptide levels in human primary islet cells were detected.

CL. Results

Results of the studies are summarized below. Representative donor results are shown but similar results were observed for various donors.

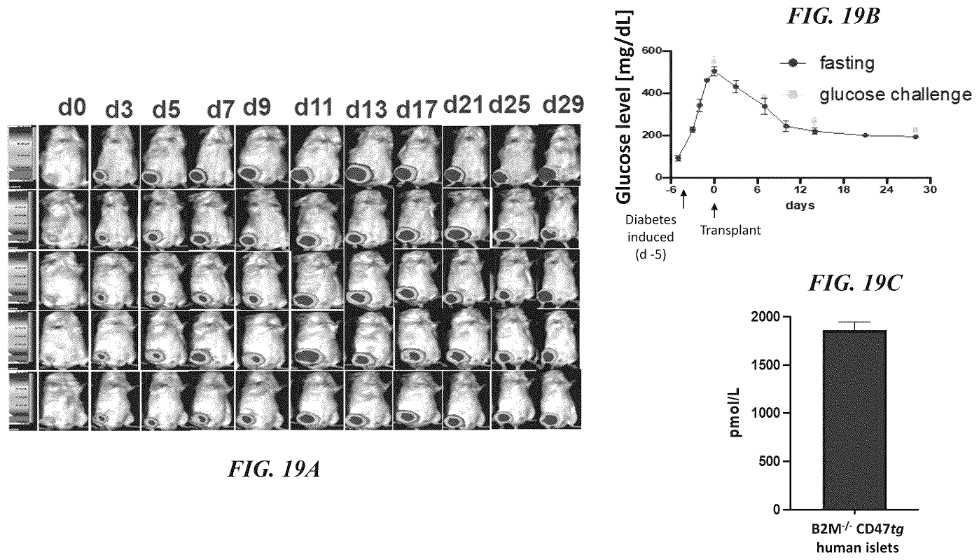

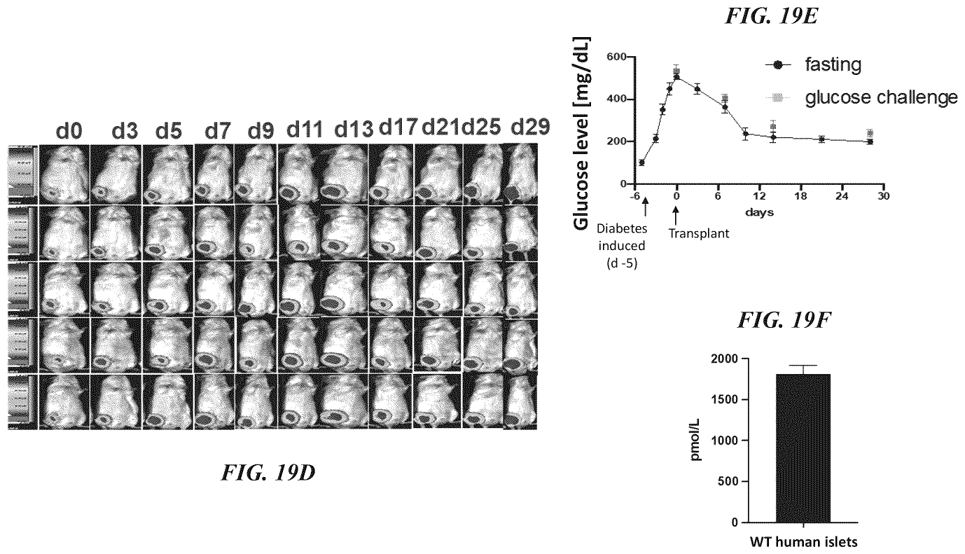

B2M^{-/-}; CD47tg human primary islet cells survive after transplant. BLI images of human primary islet cells after i.m. injection are shown in FIG. 19A (B2M^{-/-}; CD47tg human primary islet cells) and FIG. 19D (WT human primary islet cells). Bioluminescence was initially observed at the i.m. injection site for both groups following administration of the cells. The bioluminescenese detected for transplanted WT human primary islet cells was sustained over the time of the study indicating that the WT islets survive and function in immune incompetent diabetic mice (FIG. 19D). Similarly, the bioluminesence detected from transplanted B2M^{-/-}; CD47tg human primary islet cells also was sustained over the course of 29 days post-transplant, indicating survival and growth of the B2M^{-/-}; CD47tg human primary islet cells (FIG. 19A). These results further demonstrate that the HIP edits do not impair islet cell survival and function.

B2M^{-/-}; CD47tg human primary islet cells function after allogeneic transplant. To analyze certain functions of transplanted human primary islet cells, blood glucose levels were measured 6 days after transplant. In this study, for both groups of mice the fasting blood glucose levels in diabetic (STZ treated) mice receiving i.m. injection of dropped soon after transplantation, as shown in FIG. 19B (B2M^{-/-}; CD47tg human primary islet cells) and FIG. 19E (WT human primary islet cells). Further, the mice successfully tolerated a glucose challenge (FIG. 19B and FIG. 19E).

To further analyze function of transplanted human primary islet cells, C-peptide levels were measured following transplant. C-peptide levels in diabetic (STZ treated) mice receiving i.m. injection of WT human primary islet cells or B2M^{-/-}; CD47tg human primary islet cells were high mice, as shown in FIG. 19F. (FIG. 19C), respectively.

These data indicate that the edits to B2M^{-/-}; CD47tg human primary islet cells had no impact on islet survival or function in diabetic NSG mice without an immune system.

### Example 11: Assessment of CD47 Signaling in B2M^{-/-}; CIITA^{-/-}; CD47tg Human Primary Islet Cells with Anti-CD47 and Isotope Control in a Transplant Study

Hypoimmune (B2M^{-/-}; CIITA^{-/-}; CD47tg) human primary islet cells were produced as described in Example 4, with an additional knockout of CIITA using techniques described in Example 4, and were transplanted into an allogeneic diabetic humanized NSG-SGM3 recipient mouse along with anti-CD47 and isotype control. Incidence of diabetes in the mice, and survival and function of the transplanted B2M^{-/-}; CIITA^{-/-}; CD47tg human primary islet cells compared to transplanted wild type human primary islet cells, were monitored over time.

### A. Methods

Transplant study design and administration. Twenty-five humanized NSG-SGM3 mice (mouse body weight 18-20 g) were randomized into study groups post STZ administration to induce diabetes and B2M^{-/-}; CIITA^{-/-}; CD47tg human islet cells were administered. The study groups were as follows: B2M^{-/-}; CIITA^{-/-}; CD47tg human islet cell (luc+) i.m. transplant plus anti-CD47 i.m. transplant, local administration; B2M^{-/-}; CIITA^{-/-}; CD47tg human islet cell (luc+) i.m. transplant plus anti-CD47 i.m. transplant, systemic administration; B2M^{-/-}; CIITA^{-/-}; CD47tg human islet cell (luc+) i.m. transplant plus isotype control i.m. transplant, local administration; and B2M^{-/-}; CIITA^{-/-}; CD47tg human islet cell (luc+) i.m. transplant plus isotype control i.m. transplant, systemic administration.

300 human islet clusters of about 1,500 cells per cluster were transplanted by i.m. injection into mice. Day 0 (d0) was defined as the day of transplantation. Anti-CD47 or isotype control were transplanted by i.m. injection into mice on Day 8 (d8), either by local administration or systemic administration.

Mice were monitored for bioluminescence (BLI) as an indicator of islet cell survival and for glucose levels after 4 hours of fasting to monitor diabetes. A glucose challenge was performed on day 29 of the study.

CL. Results

B2M^{-/-}; CIITA^{-/-}; CD47tg human primary islet cells do not survive after allogeneic transplant when anti-CD47 is added. BLI images of i.m. transplanted B2M^{-/-};CIITA^{-/-}; CD47tg human primary islet cells after local and systemic i.m. injection with isotype control are shown in FIGS. 20A and 21A, respectively, and BLI images of i.m. transplanted B2M^{-/-}; CIITA^{-/-}; CD47tg human primary islet cells after local and systemic i.m. injection with anti-CD47 are shown in FIGS. 20C and 21C, respectively. Transplanted B2M^{-/-}; CIITA^{-/-}; CD47tg human primary islet cells continue to survive and grow in the absence of anti-CD47 (i.e., following either local or systemic addition of isotype control) (FIGS. 20A and 21A). In contrast, the transplanted B2M^{-/-}; CIITA^{-/-}; CD47tg human primary islet cells rapidly decline following the start of local or systemic administration of anti-CD47 administered on day 8 after transplant (FIGS. 20C and 21C), indicating death of the B2M^{-/-}; CIITA^{-/-}; CD47tg human primary islet cells by innate immune cells by blocking CD47.

These data indicate that the blocking of CD47-SIRP signaling in B2M^{-/-}; CIITA^{-/-}; CD47tg human primary islet cells results in their recognition as foreign cells by the immune system, thus causing in cell killing, and further confirm that overexpression of CD47 contributes to immune evasion by the modified cells.

B2M^{-/-}; CIITA^{-/-}; CD47tg human primary islet cells do not function after allogeneic transplant when anti-CD47 is added. To analyze certain functions of transplanted human primary islet cells, blood glucose levels were measured 6 days after transplant. Glucose levels measured about 4 hours after fasting in diabetic (STZ treated) mice receiving i.m. injection of B2M^{-/-}; CIITA^{-/-}; CD47tg human primary islet cells dropped soon after transplantation, as shown in FIGS. 20B, 20D, 21B, and 21D, and remained low following both local and systemic administration of isotype control (FIGS. 20B and 21B, respectively). However, the mice receiving i.m. injection of B2M^{-/-}; CIITA^{-/-}; CD47tg human primary islet cells followed by either local or systemic administration of anti-CD47 exhibited a rise in glucose levels (FIGS. 20D and 21D, respectively).

### Example 12: Assessment of B2M: CIITA^{-/-}; CD47tg Non-Human Primate Primary Beta Islet Cells in a Transplant Study

Hypoimmune (B2M^{-/-}; CIITA^{-/-}; CD47tg) non-human primate (NHP) primary beta islet cells were produced and transplanted into an allogeneic recipient NHP along. Survival and function of the transplanted B2M^{-/-}; CIITA^{-/-}; CD47tg NHP primary islet cells compared to transplanted wild type human primary beta islet cells, were monitored over time.

### A. Methods

Generation of NHP primary beta islet cells and cell engineering. Primary beta islet cells were isolated from NHP pancreas using a standard technique. Such techniques are known in the art, the isolated cells were engineered to knockout B2M and CIITA using standard CRISPR/Cas9 gene editing techniques, and were transduced with a transgene (tg) encoding exogenous CD47 protein using a lentiviral vector containing a polynucleotide encoding CD47. The hypoimmune islets were sorted by flow cytometry for cells negative for HLA class I/II and for CD47 overexpression.

Transplant study design and administration. 300 NHP islet clusters of about 1,500 cells per cluster were transplanted by i.m. injection into NHPs. Day 0 (d0) was defined as the day of transplantation.

T Cell Enzyme-Linked Immune absorbent SPOT (ELISPOT) Assay. Interferon gamma (IFNg)-secreting CD8(+) T cells in NHP primary beta islet cells were detected by ELISPOT

Flow cytometry. Expression of donor specific antibodies (DSA) in primary beta islet cells was assessed by flow cytometry.

NK cell killing assay. NK cell killing assays were performed substantially as described in examples above.

CL. Results

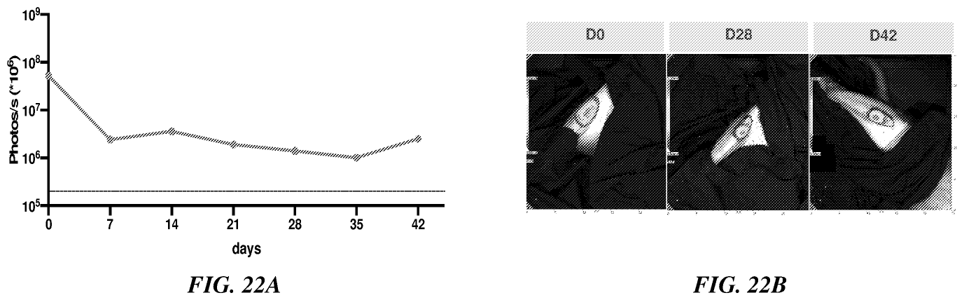

B2M^{-/-}; CIITA^{-/-}; CD47tg NHP islet cells survive after transplant. Quantification of BLI imaging results of NHP B2M^{-/-}; CIITA^{-/-}; CD47tg primary islet cells after i.m. injection are shown in FIG. 22A. Corresponding BLI images of NHP B2M^{-/-}; CIITA^{-/-}; CD47tg primary islet cells after i.m. injection are shown in FIG. 22B. Bioluminescence was initially observed at the i.m. injection site for all groups following administration of the NHP primary islet cells. The number of photons detected from transplanted NHP B2M^{-/-}; CIITA^{-/-}; CD47tg primary islet cells initially slightly decreased post-transplant, but then remained consistent over the course of 42 days post-transplant, indicating survival of the NHP B2M^{-/-}; CIITA^{-/-}; CD47tg primary islet cells (FIG. 22A).

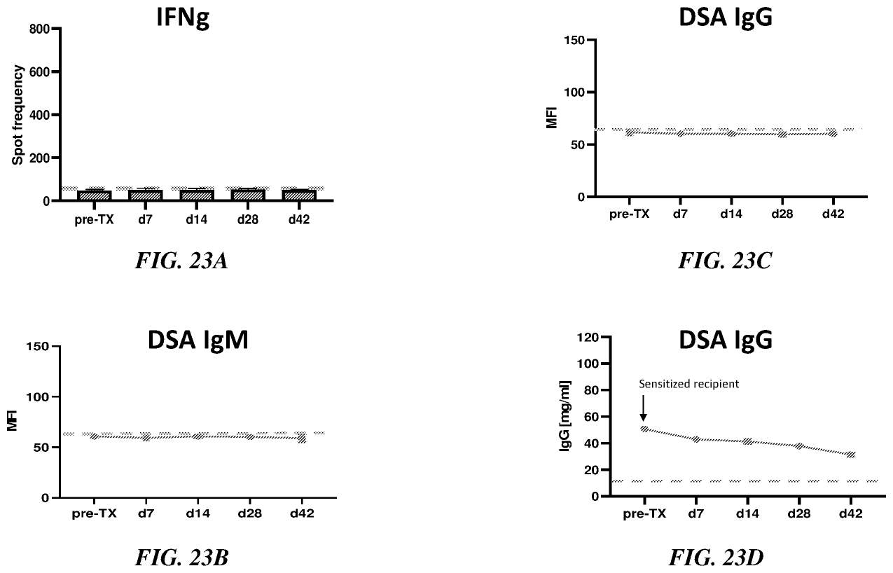

NHP B2M^{-/-}; CITA; CD47tg primary islet cells immune response after allogeneic transplant. To analyze the immune response to transplanted NHP primary beta islet cells, an ELISPOT assay was used to evaluate the levels of IFNg cytokine secretion by CD8+ T cells. As shown in FIG. 23A, transplanted NHP B2M^{-/-}; CIITA^{-/-}; CD47tg primary islet cells exhibited low levels of IFNg. The levels of DSA IgM and IgG, measured by flow cytometry, were also low in transplanted NHP B2M^{-/-}; CIITA^{-/-}; CD47tg primary islet cells (FIGS. 23B and 23C, respectively). Moreover, in a sensitized NHP recipient with high IgG antibody concentrations prior to transplant with NHP B2M^{-/-}; CIITA^{-/-}; CD47tg primary islet cells, the levels of DSA IgG decreased over the course of 42 days-post transplant (FIG. 23D).

These data indicate that the NHP B2M^{-/-}; CIITA^{-/-}; CD47tg primary islet cells do not induce an immune response to the cells upon transplantation and are protected from antibody-mediated rejection.

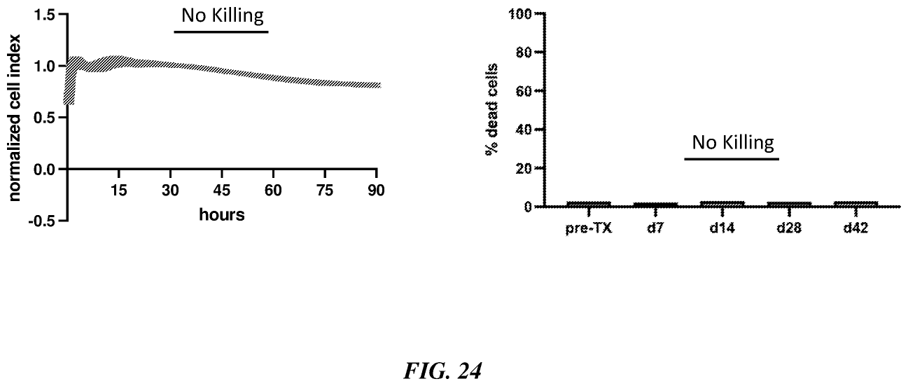

B2M^{-/-}; CIITA^{-/-}; CD47tg NHP primary islet cells evade killing by NK cells. B2M^{-/-}; CIITA^{-/-}; CD47tg NHP primary islet cells did not exhibit NK-mediated cell killing (FIG. 24), indicating that the hypoimmune (B2M^{-/-}; CIITA^{-/-}; CD47tg) NHP primary islet cells are able to effectively evade immune responses by NK cells. These data indicate that transplanted B2M^{-/-}; CIITA^{-/-}; CD47tg NHP primary islet cells are not recognized as foreign.

### Example 13: Gene Editing of Human Primary Islet Cells

Primary islet cells were isolated from a human cadaver donor using a standard technique and cells were dissociated into a suspension containing single cells using ACCUMAX™ (StemCell Technologies) cell dissociation solution for 10 min at 37° C. The dissociated cell suspension was electroporated with a ribonucleoprotein complex containing a Cas9 enzyme and a single gRNA targeting the human B2M gene and the human CIITA gene.

The human B2M gene was disrupted using a SEQ ID NO: 33 guide RNA (gRNA) sequence, and the human CII2A gene was disrupted using a SEQ ID NO: 34 gRNA sequence. Human primary islet cells were transferred into U-bottom 96-well plates containing 50,000 cells/well in PIM(S) media (Prodo) and rested for 1 h at 37° C. and 5% CO₂ before moving the plate to a Belly Dancer Orbital Shaker (IBI Scientific, Dubuque, IA) for human primary islet cell re-clustering. Complete media change was performed after 48 h, and human primary islet cell clusters were incubated on the Belly Dancer Orbital Shaker for another 24 h.

To enrich for edited islets, the re-clustered human primary islet clusters were dissociated for cell sorting into single cells using ACCUMAX™ with anti-HLA-A,B,C antibody (clone G46_2.6, BD Biosciences) or IgG1 isotype-matched control antibody (clone MOPC-21, BD Biosciences), and anti-HLA-DR,DP,DQ antibody (clone Tu3a, BD Biosciences) or IgG2a isotype-matched control antibody (clone G155-178, BD Biosciences). Double negative human primary islet cells were sorted in the BD FACSAria™ II and replated in U-bottom 96-well plates as described above for re-clustering on the Belly Dancer Orbital Shaker.

After 24 h, human primary islet cells were dissociated into single cells and were transduced with a lentiviral vector encoding CD47. The transduced human primary islet cells were re-plated in U-bottom 96-well plates as described above for re-clustering on the Belly Dancer Orbital Shaker. After 48 h, human primary islet cells were dissociated into single cells using ACCUMAX™ and underwent cell sorting for human CD47 with anti-CD47 antibody (clone B6H12, BD Biosciences) or IgG1 isotype-matched control antibody (clone MOPC-21, BD Biosciences) to select for transduced cells that overexpressed CD47. Human primary islet cells were replated in U-bottom 96-well plates as described above for re-clustering on the Belly Dancer Orbital Shaker.

A similar process was carried out except instead of incubating the cells with motion the islet cells were incubated under static conditions at 37° C. and 5% CO₂ (without motion). In this process, the static conditions resulted in impaired reclustering and low viability of only about 35%. Without wishing to be bound by theory, the provided results support that subjecting the cells to motion during the process of gene editing improves their viability and the efficiency of gene editing.

### Example 14: Assessment of B2M: CIITA^{-/-}; CD47tg Human Primary Retinal Pigment Epithelial Cells

This example describes studies characterizing hypoimmune human primary retinal pigment epithelial (RPE) cells that were engineered to (1) knock out B2M (B2M^{-/-}) to reduce HLA class I expression, (2) knock out CIITA (CIITA) to reduce HLA class II expression, and (3) overexpress exogenous CD47 (CD47tg). The hypoimmune (B2M^{-/-}; CIITA^{-/-}; CD47tg) human primary RPE cells were monitored for protection from cell killing by Natural Killer (NK) cells and macrophages, compared to wild type (WT) human primary RPE cells or double knockout (B2M^{-/-} CIITA^{-/-}) human primary RPE cells.

### A. Methods

Generation of human primary RPEs and cell engineering. Primary RPE cells were isolated from human cadaver globes and were cryopreserved. The isolated RPE cells were thawed and then plated on plates coated with Synthemax (3535 Corning, Corning NY). To generate hypoimmune cells, the plated cells were engineered to knockout B2M and CIITA using standard CRISPR/Cas9 gene editing techniques, and about two days later media was changed on the plated cells, and then cells were either left untouched or were further engineered by transduction with a transgene (tg) encoding exogenous CD47 protein using a lentiviral vector containing a polynucleotide encoding CD47. After two days the media was changed again and then cells were collected when a cobblestone morphology had formed (e.g. day 6) and were sorted by flow cytometry for cells negative for HLA class I/II (B2M^{-/-}, CIITA⁴ double knockout, dKO) and, in some cases, also for CD47 overexpression (B2M^{-/-}, CIITA^{-/-}. CD47tg)

NK cell and macrophage killing assay. NK cell killing assays and macrophage killing assays were performed on the XCelligence MP platform (ACEA Biosciences) to provide for label-free monitoring of cell proliferation and viability of cells. Unmodified (wild-type) human primary RPE, B2M^{-/-}, CIITA^{-/-} engineered human primary RPE, or B2M^{-/-}, CIITA^{-/-}, CD47tg engineered human primary RPE were plated into 96-well E-plates coated with laminin, collagen and fibronectin. The XCelligence software was used to measure Cell Index (CI) as a measure of adhesion and hence cell killing (a decrease in cell index indicates an increase in killing of the cells). After the CI value reached 0.7, human primary NK cells or human macrophages differentiated (both Stemcell Technologies) were added at an effector to target (E:T) ratio of 1:1

CL. Results

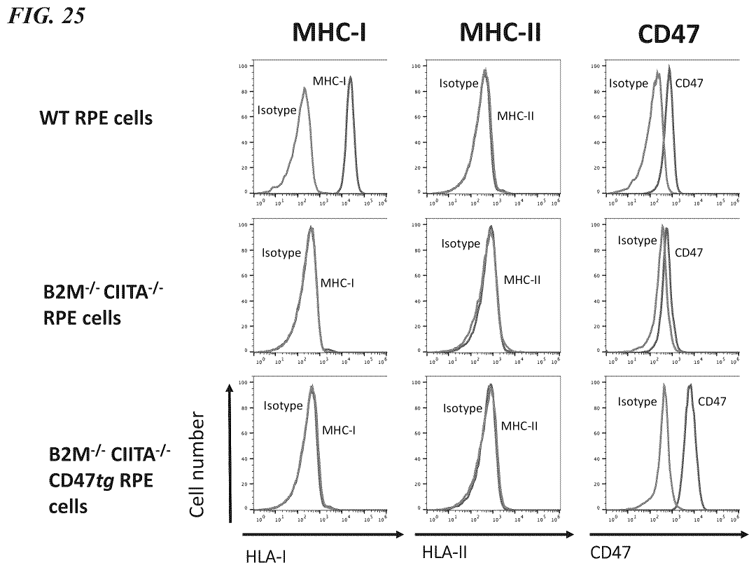

Human B2M^{-/-}; CIITA^{-/-}; CD47tg primary RPE cells do not express MHC-I or MHC-II, and have increased CD47 expression. Expression of the MHC-I and MCH-II and CD47 was assessed by flow cytometry. Isolated human primary RPE cells were positive for MHC-I, did not express MHC-II, and had low expression of CD47 (FIG. 25, top panel). Human B2M^{-/-}/CIITA^{-/-} dKO primary RPE cells and B2M^{-/-}; CIITA^{-/-}; CD47tg primary RPE cells were negative for both MHC-I and MHC-II (FIG. 25, middle and bottom panels). Only B2M^{-/-}; CIITA^{-/-}; CD47tg primary RPE cells showed increased expression of CD47 (20-fold over isotype control, as shown in FIG. 25, bottom panels).

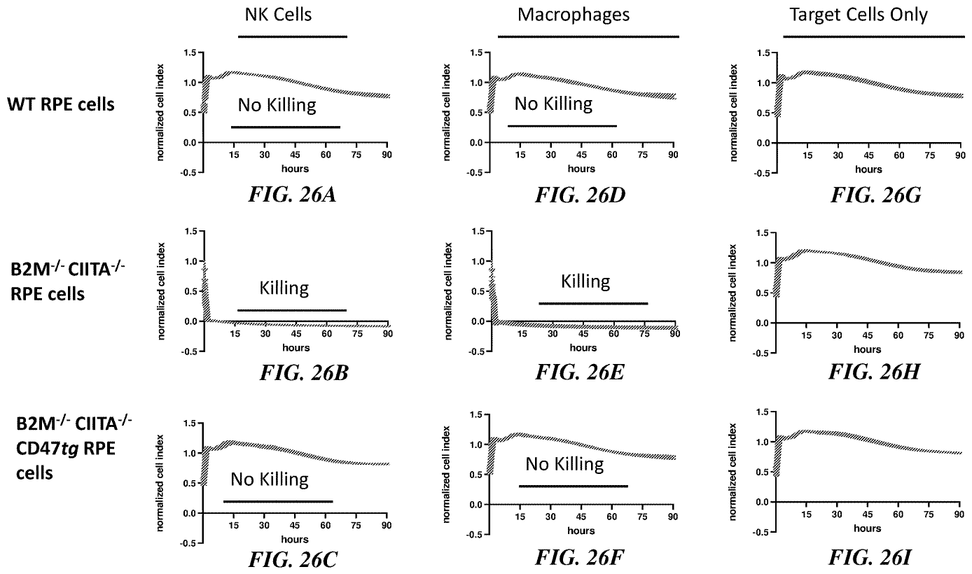

Human B2M^{-/-}; CIITA^{-/-}; CD47tg primary RPE cells evade NK cell and macrophage killing. Unmodified (wild-type) primary RPE cells were not killed by NK cells (FIG. 26A) or macrophages (FIG. 26D), consistent with resistance of wild-type cells from innate immune killing. Human B2M^{-/-} CIITA^{-/-} primary RPE cells were killed by the NK cells (FIG. 26B) and macrophages (FIG. 26E), indicating that the human B2M^{-/-} CIITA^{-/-} primary RPE cells were lacking HLA molecules on the cell surface and triggered the missing-self killing by NK cells and macrophages. In contrast, human B2M^{-/-}; CIITA^{-/-}; CD47tg primary RPE cells that further overexpressed the surface molecule CD47 did not exhibit NK-mediated cell killing (FIG. 26C) or macrophage-mediated cell killing (FIG. 26F). In the absence of innate NK cells or macrophages (target cells only), no killing of any of the RPE cells was observed (FIGS. 26G-I).

These data indicate that human B2M^{-/-}; CIITA^{-/-}; CD47tg primary RPE cells are hypoimmune and effectively evade immune responses by NK cells and macrophages.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| # | SEQUENCE | Annotation |
|---|---|---|
| 1 | QLLFNKTKSVEFTFCNDTVVIPCFVTNMEAQNTTEVYVKWKFKG | Human CD47 |
| | RDIYTFDGALNKSTVPTDFSSAKIEVSQLLKGDASLKMDKSDAV | (without signal |
| | SHTGNYTCEVTELTREGETIIELKYRVVSWFSPNENILIVIFPI | sequence); aa |
| | FAILLFWGQFGIKTLKYRSGGMDEKTIALLVAGLVITVIVIVGA | 19-323 |
| | ILFVPGEYSLKNATGLGLIVTSTGILILLHYYVFSTAIGLTSFV | |
| | IAILVIQVIAYILAVVGLSLCIAACIPMHGPLLISGLSILALAQ | |
| | LLGLVYMKFVASNQKTIQPPRKAVEEPLNAFKESKGMMNDE | |
| 2 | MWPLVAALLLGSACCGSAQLLENKTKSVEFTFCNDTVVIPCFVT | Human CD47 |
| | NMEAQNTTEVYVKWKFKGRDIYTFDGALNKSTVPTDFSSAKIEV | (with signal |
| | SQLLKGDASLKMDKSDAVSHTGNYTCEVTELTREGETIIELKYR | sequence); aa |
| | VVSWFSPNENILIVIFPIFAILLFWGQFGIKTLKYRSGGMDEKT | 1-323 |
| | IALLVAGLVITVIVIVGAILFVPGEYSLKNATGLGLIVTSTGIL | |
| | ILLHYYVESTAIGLTSFVIAILVIQVIAYILAVVGLSLCIAACI | |
| | PMHGPLLISGLSILALAQLLGLVYMKFVASNQKTIQPPRKAVEE | |
| | PLNAFKESKGMMNDE | |
| 3 | DIQMTQTTSSLSASLGDRVTISCRASQDISKYLNWYQQKPDGTV | Anti-CD19 |
| | KLLIYHTSRLHSGVPSRESGSGSGTDYSLTISNLEQEDIATYFC | scFv (FMC63) |
| | QQGNTLPYTFGGGTKLEITGSTSGSGKPGSGEGSTKGEVKLQES | |
| | GPGLVAPSQSLSVTCTVSGVSLPDYGVSWIRQPPRKGLEWLGVI | |
| | WGSETTYYNSALKSRLTIIKDNSKSQVFLKMNSLQTDDTAIYYC | |
| | AKHYYYGGSYAMDYWGQGTSVTVSS | |
| 4 | DIQMTQTTSSLSASLGDRVTISCRASQDISKYLNWYQQKPDGTV | Anti-CD19 |
| | KLLIYHTSRLHSGVPSRFSGSGSGTDYSLTISNLEQEDIATYFC | scFv (FMC63) |
| | QQGNTLPYTFGGGTKLEITGGGGSGGGGSGGGGSEVKLQESGPG | |
| | LVAPSQSLSVTCTVSGVSLPDYGVSWIRQPPRKGLEWLGVIWGS | |
| | ETTYYNSALKSRLTIIKDNSKSQVFLKMNSLQTDDTAIYYCAKH | |
| | YYYGGSYAMDYWGQGTSVTVSS | |
| 5 | ESKYGPPCPPCP | IgG4 Hinge |
| 6 | TTTPAPRPPTPAPTIASQPLSLRPE | CD8 Hinge |
| 7 | IEVMYPPPYLDNEKSNGTIIHVKGKHLCPSPLFPGPSKP | CD28 |
| 8 | ACRPAAGGAVHTRGLDFACDIYIWAPLAGTCGVLLLSLVITLYC | CD8 |
| 9 | FWVLVVVGGVLACYSLLVTVAFIIFWV | CD28 |
| 10 | FWVLVVVGGVLACYSLLVTVAFIIFWV | CD28 |
| 11 | RSKRSRLLHSDYMNMTPRRPGPTRKHYQPYAPPRDFAAYRS | CD28 |
| 12 | KRGRKKLLYIFKQPFMRPVQTTQEEDGCSCRFPEEEEGGCEL | 4-1BB |
| 13 | RVKFSRSADAPAYQQGQNQLYNELNLGRREEYDVLDKRRGRDPE | CD3zeta |
| | MGGKPRRKNPQEGLYNELQKDKMAEAYSEIGMKGERRRGKGHDG | |
| | LYQGLSTATKDTYDALHMQALPPR | |
| 14 | RVKFSRSADAPAYKQGQNQLYNELNLGRREEYDVLDKRRGRDPE | CD3zeta |
| | MGGKPRRKNPQEGLYNELQKDKMAEAYSEIGMKGERRRGKGHDG | |
| | LYQGLSTATKDTYDALHMQALPPR | |
| 15 | EGRGSLLTCGDVEENPGP | T2A |
| 16 | LEGGGEGRGSLLTCGDVEENPGPR | T2A |
| 17 | GSGATNFSLLKQAGDVEENPGP | P2A |
| 18 | ATNFSLLKQAGDVEENPGP | P2A |
| 19 | QCTNYALLKLAGDVESNPGP | E2A |
| 20 | VKQTLNFDLLKLAGDVESNPGP | F2A |
| 21 | GSGEGRGSLLTCGDVEENPGP | T2A |
| 22 | AAGSGEGRGSLLTCGDVEENPGP | T2A |
| 23 | GUUUUAGAGCUA | crRNA repeat |
| 24 | UAGCAAGUUAAAAUAAGGCUAGUCCGUUAUCAACUUGAAAAAGU | tracrRNA |
| | GGCACCGAGUCGGUGCUUU | |
| 25 | GAAA | tetraloop |
| 26 | accccacagtggggccacta | GET000046 |
| | | guide |
| 27 | tgttggaaggatgaggaaat | GET000047 |
| | | guide |
| 28 | tcactatgctgccgcccagt | GET000048 |
| | | guide |
| 29 | UCUCUCCAUGUGCAGUAGGA | ABO gRNA |
| 30 | CUGGAUGUCGGAGGAGUACG | FUT1 gRNA |
| 31 | GUCUCCGGAAACUCGAGGUG | RHD gRNA |
| 32 | ACAGUGUAGACUUGAUUGAC | F3 (CD142) |
| | | gRNA |
| 33 | CGUGAGUAAACCUGAAUCUU | B2M gRNA |
| 34 | GAUAUUGGCAUAAGCCUCCC | CIITA gRNA |
| 35 | AGAGUCUCUCAGCUGGUACA | TRAC gRNA |

1-381. (canceled)

382. A method for modifying primary islet cells, the method comprising:
i) dissociating one or more primary islet clusters into a suspension of primary islet cells;
ii) contacting the suspension of primary islet cells with one or more first reagents, wherein the one or more first reagents comprise (1) a gene editing system comprising a genome-modifying protein or a nucleic acid encoding the genome-modifying protein for disrupting one or more target genes encoding one or more endogenous proteins and/or (2) an agent comprising an exogenous polynucleotide encoding a protein; and
iii) after the contacting in step ii)(1) and/or step ii)(2), incubating the primary islet cells to produce modified islet cells, wherein at least a portion of the incubating is carried out with motion and wherein the modified islet cells are re-clustered into one or more first modified primary islet cell clusters.

383. The method of claim 382, wherein after iii), the method comprises:
iv) dissociating the one or more first modified primary islet clusters into a suspension of modified primary islet cells;
v) further contacting the suspension of modified primary islet cells with one or more second reagents, wherein the one or more second reagents comprise (i) a gene editing system comprising a genome-modifying protein or a nucleic acid encoding the genome-modifying protein for disrupting one or more target genes encoding one or more endogenous proteins and/or (ii) an agent comprising an exogenous polynucleotide encoding a protein; and
vi) after the contacting in step v)(i) and/or step v)(ii), incubating the modified islet cells to produce further modified islet cells, wherein at least a portion of the incubating is carried out with motion, and wherein the further modified islet cells are re-clustered into one or more second modified primary islet cell clusters.

384. The method of claim 383, wherein prior to v), the method comprises selecting, from the dissociated islet cells in iv), islet cells that have modified gene expression relative to the primary islet cells before the contacting.

385. The method of claim 384, wherein after selecting the islet cells that have modified expression and prior to v), the method comprises incubating the selected one or more first modified islet cells under conditions for re-clustering the cells into one or more islet clusters, wherein at least a portion of the incubating is carried out with motion, and then dissociating the selected one or more first modified primary islet clusters into a suspension of modified primary islet cells.

386. The method of claim 383, wherein after the incubating in vi), the method comprises dissociating the one or more second modified primary islet clusters into a suspension comprising the second modified primary islet cells and selecting for islet cells that have modified gene expression relative to the primary islet cells before the contacting or the further contacting.

387. The method of claim 386, wherein the method comprises incubating the selected further modified islet cells under conditions for re-clustering into one or more further modified primary islet clusters, wherein at least a portion of the incubating is carried out with motion.

388. The method of claim 382, wherein the one or more first reagents comprise the gene editing system comprising the genome-modifying protein, and wherein the one or more first reagents are for reducing cell surface expression of one or more major histocompatibility complex (MHC) class I molecules and/or are for reducing cell surface expression of one or more MHC class II molecules.

389. The method of claim 383, wherein the one or more second reagents comprise the gene editing system comprising the genome-modifying protein, and wherein the one or more second reagents are for reducing cell surface expression of one or more major histocompatibility complex (MHC) class I molecules and/or are for reducing cell surface expression of one or more MHC class II molecules.

390. The method of claim 382, wherein the genome-modifying protein comprises a sequence-specific nuclease, a CRISPR-associated transposase (CAST), prime editing, or Programmable Addition via Site-specific Targeting Elements (PASTE).

391. The method of claim 382, wherein the gene editing system comprises a Cas nuclease and one or more guide RNAs.

392. The method of claim 382, wherein the one or more target genes comprise CIITA and the one or more first reagents disrupt the CIITA gene, and/or wherein the one or more target genes comprise B2M and the one or more first reagents disrupt the B2M gene.

393. The method of claim 382, wherein the one or more first reagents comprise an exogenous polynucleotide encoding a tolerogenic factor selected from the group consisting of CD47, A20/TNFAIP3, C1 Inhibitor, CCL21, CCL22, CD16, CD16 Fc receptor, CD24, CD27, CD35, CD39, CD46, CD52, CD55, CD59, CD200, CR1, CTLA4-Ig, DUX4, FasL, H2-M3, HLA-C, HLA-E, HLA-E heavy chain, HLA-G, PD-L1, IDO1, IL-10, IL15-RF, IL-35, MANF, Mfge8, and Serpinb9.

394. The method of claim 383, wherein the one or more second reagents comprise an exogenous polynucleotide encoding a tolerogenic factor selected from the group consisting of CD47, A20/TNFAIP3, C1 Inhibitor, CCL21, CCL22, CD16, CD16 Fc receptor, CD24, CD27, CD35, CD39, CD46, CD52, CD55, CD59, CD200, CR1, CTLA4-Ig, DUX4, FasL, H2-M3, HLA-C, HLA-E, HLA-E heavy chain, HLA-G, PD-L1, IDO1, IL-10, IL15-RF, IL-35, MANF, Mfge8, and Serpinb9.

395. The method of claim 382, wherein the exogenous polynucleotide is integrated by targeted insertion into a target genomic locus of the primary islet cells and/or modified islet cells.

396. The method of claim 382, wherein the exogenous polynucleotide is integrated by non-targeted insertion into the genome of the primary islet cells.

397. The method of claim 382, wherein the incubating in iii) comprises a first incubation under static conditions of between about 30 min and about 2 hours followed by the incubating with motion.

398. The method of claim 382, wherein the motion comprises orbital motion.

399. The method of claim 382, wherein the motion comprises orbital motion, and wherein the orbital motion is at a speed of between 20 rpm and 180 rpm, inclusive.

400. The method of claim 382, wherein the genome-modifying protein is selected from the group consisting of Cas9, Cas12a (Cpf1), Cas12b, and Mad7.

401. A method for modifying primary islet cells, the method comprising:
i) dissociating one or more primary islet clusters into a suspension of primary islet cells;
ii) contacting the suspension of primary islet cells with one or more first reagents, wherein the one or more first reagents comprise a nucleic acid encoding a Cas nuclease, a first guide RNA (gRNA) targeting CIITA, and a second gRNA targeting B2M;
iii) after the contacting, incubating the primary islet cells to produce modified islet cells, wherein at least a portion of the incubating is carried out with motion, and wherein the modified islet cells are re-clustered into one or more first modified primary islet clusters;
iv) dissociating the one or more first modified primary islet clusters into a suspension of modified primary islet cells;
v) further contacting the suspension of modified primary islet cells with a lentiviral vector comprising an exogenous polynucleotide encoding CD47; and
vi) after the further contacting, incubating the modified primary islet cells to produce further modified islet cells, wherein at least a portion of the incubating is carried out with motion, and wherein the further modified islet cells are re-clustered into one or more second modified primary islet cell clusters.

402. The method of claim 401, wherein the encoded CD47 comprises a sequence of amino acids that exhibits at least 95% sequence identity to SEQ ID NO:2.

403. The method of claim 401, wherein prior to v), the method comprises selecting, from the dissociated islet cells in iv), islet cells that have modified gene expression relative to the primary islet cells before the contacting.

404. The method of claim 403, wherein after selecting the islet cells that have modified expression and prior to v), the method comprises incubating the selected one or more first modified islet cells under conditions for re-clustering the cells into one or more islet clusters, wherein at least a portion of the incubating is carried out with motion, and then dissociating the selected one or more first modified primary islet clusters into a suspension of modified primary islet cells.

405. The method of claim 401, wherein after the incubating in vi), the method comprises dissociating the one or more second modified primary islet clusters into a suspension comprising the one or more second modified primary islet cells and selecting for islet cells that have modified gene expression relative to the primary islet cells before the contacting or the further contacting.

406. The method of claim 382, wherein the contacting is carried out for 1 minute to 60 minutes prior to subjecting the primary islet cells and/or modified islet cells to motion.

407. The method of claim 403, wherein selecting the islet cells that have modified gene expression relative to the primary islet cells before the contacting comprises performing flow cytometry.

408. The method of claim 382, wherein the one or more first reagents comprise a gene editing system comprising a genome-modifying protein or a nucleic acid encoding the genome-modifying protein for disrupting one or more target genes selected from the group consisting of B2M, TAP I, NLRC5, CIITA, HLA-A, HLA-B, HLA-C, HLA-DP, HLA-DM, HLA-DOA, HLA-DOB, HLA-DQ, HLA-DR, RFX5, RFXANK, RFXAP, NFY-A, NFY-B and NFY-C.

409. A population of engineered primary islets produced by the method of claim 382.

410. A method of treating diabetes in a patient in need thereof comprising administering to the patient an effective amount of the population of claim 409.

411. The method of claim 382, wherein the motion comprises orbital motion, bidirectional linear motion, undulating motion, and/or motion with a tilt angle.
